(19) United States
(12) Patent Application Publication
(10) Pub. No.: US 2026/0286414 A1
(43) Pub. Date: Sep. 24, 2026

(54) FERMENTATION BASED METHOD FOR DOUBLE STRANDED RNA PRODUCTION

(71) Applicant: RNAISSANCE AG LLC, Shawnee Mission, KS (US)

(72) Inventors: Anil Kumar, St. Louis, MO (US); Tammy Zanker, St. Louis, MO (US); Farhad Moshiri, St. Louis, MO (US); Candace Seeve, St. Louis, MO (US)

(21) Appl. No.: 19/128,920

(22) PCT Filed: Nov. 10, 2023
(86) PCT No.: PCT/US2023/079414

§ 371 Date: May 9, 2025

Related U.S. Application Data
(60) Provisional application No. 63/424,778, filed on Nov. 11, 2022.

(51) Int. Cl. C12P 19/34; C12N 15/70

(52) U.S. Cl. CPC C12P 19/34; C12N 15/70

(57) ABSTRACT

The disclosure provides methods and tools allowing production of large quantities of double stranded RNA (dsRNA) in bacterial cells, including plasmid vectors useful for transforming gram-positive or gram-negative bacterial cells such that the cells produce high yields of target dsRNA.

### CROSS REFERENCE TO RELATED APPLICATIONS

The present application claims the benefit of U.S. Provisional Application No. 63/424,778, entitled, “FERMENTATION BASED METHOD FOR DOUBLE STRANDED RNA PRODUCTION” filed Nov. 11, 2022, the content of which is hereby incorporated by reference in its entirety.

### INCORPORATION OF SEQUENCE LISTING

The present application contains a Sequence Listing which has been submitted in .XML format via PatentCenterPatent Center and is hereby incorporated by reference in its entirety. Said WIPO Sequence Listing was created on Nov. 10, 2023, is named 066803-779123 Sequence Listing, and is 121 kilobytes in size.

### FIELD

The present disclosure generally relates to high yielding microbial plasmid vectors and their use in fermentation-based system for producing double-stranded RNA (dsRNA).

### BACKGROUND

RNAi biopesticides exploit the conserved eukaryotic gene regulatory mechanism, RNA interference, to disrupt the production of a protein essential to the survival of a target pest. The active molecule of RNAi biopesticides, a double-stranded RNA (dsRNA), can be delivered to a plant pest by exogenous application. The dsRNA, upon application is ingested and taken up by cells of a target pest where it engages the RNAi process to direct sequence-specific degradation of target mRNA transcripts. Due to the selectivity of this mode of action, and the favorable toxicity profile and lability of dsRNA, RNAi biopesticides present minimal risk to human health and the environment.

While advances in lethal RNAi gene target identification and RNA delivery have been made, the full potential of RNAi biopesticides for pest control remains unrealized due to the high cost of large-scale dsRNA production. Enormous quantities of dsRNA are required for managing agricultural pests. Approximately, 11,550 kilograms dsRNA would be required to treat 0.1% of the total global cropped area of ~1.65B hectares (Ha) with a single spray application at a rate of 7 g/Ha15,16. Development of dsRNA production technologies that can be easily scaled without specialized resources, and that utilize existing production capacities to keep costs low are required to enable widespread agricultural application of RNAi.

Existing RNA production methods include chemical synthesis, in vitro transcription, cell-free synthesis, and fermentation. Fermentation provides important advantages including low feedstock costs, scalability, and usability of existing production infrastructure and capacity, but has been impractical for commercial dsRNA production due to low yields.

Methods need to be developed to reduce dsRNA production costs to a price point that enables the development of RNAi biopesticides that are cost-competitive with chemical pesticides. A microbial fermentation-based production system that can utilize existing fermentation infrastructure and does not require specialized equipment, materials, or conditions for dsRNA production unlike in vitro transcription, cell-free, and chemical synthesis methods could pave way to cost-effective production of dsRNA.

Therefore, there is a need for developing high yielding plasmid constructs for bacterial cells for use in fermentation-based system for large scale production of dsRNA.

### SUMMARY

In some aspects, the present disclosure provides a plasmid vector comprising a nucleic acid sequence comprising: an inducible bacterial promoter operably linked to a stem-loop sequence having a 3′ end and a 5′ end and comprising a target recombinant RNA sequence; a first pac site sequence at the 3′ end, and a second pac site sequence at the 5′ end of the stem-loop sequence; an MS2 capsid protein (CP) expression cassette; and the pyrE coding sequence with a ribosome binding site (RBS) at its 5′ end and T1-T2 terminators at its 3′ end downstream of the MS2 CP expression cassette.

In some aspects, the plasmid vector comprises a coliphage T7 promoter comprising the sequence of SEQ ID NO: 1.

In some aspects, the pyrE cassette of the plasmid vector comprises RBS-pyrE cds-T1-T2 with SEQ ID NO: 2 driven by an upstream coliphage T7 promoter with SEQ ID NO: 1. In some aspects, the expression of the pyrE coding sequence comprising RBS-pyrE cds-T1-T2 is driven by a dedicated coliphage T7 promoter comprising the sequence of SEQ ID NO: 1. In some aspects, the pyrE coding sequence comprising RBS-pyrE cds-T1-T2 with a sequence of SEQ ID NO 2 is driven by a dedicated J23115 promoter comprising the sequence of SEQ ID NO: 33.

In some aspects, the target recombinant RNA in the plasmid vector is a dsRNA that specifically inhibits expression of a target gene. In some aspects, the target recombinant RNA sequence is selected from a dsRNA, a siRNA, a shRNA, a hpRNA, and a miRNA.

In some aspects, the plasmid vector of the present disclosure comprises a first pac site sequence at the 3′ end, and a second pac site sequence at the 5′ end of the stem-loop sequence. In some aspects, the first and second pac site sequences each comprise the sequence of SEQ ID NO: 3 and SEQ ID NO: 4.

In some aspects, the plasmid vector comprises the sequences of SEQ ID NOs: 1, 2, 3, 4, 5, 6, 7, 8, 33, or any combination thereof. In further aspects, the disclosed vector does not comprise an antibiotic resistance Amp-r gene or a tetracycline resistance gene. In some aspects, the disclosed vector comprises the sequence of SEQ ID NO: 43.

In some aspects, the plasmid vector of the present disclosure is expressed in a gram-positive or a gram-negative bacterial cell. In some aspects, the bacterial cell expressing the disclosed plasmid vector is an E. coli cell or a C. glutamicum cell. In some aspects, the bacterial cell expressing the disclosed plasmid vector is an E. coli cell. In some aspects, the E. coli cell is an RNAseIII-deficient E. coli strain. In some aspects, the E. coli cell is the RNAseIII-deficient E. coli strain, HT115(DE3). In some aspects, the E. coli strain is a uracil auxotroph.

In some aspects, the present disclosure comprises culturing a population of bacterial cells transformed with the plasmid vector, in a bioreactor. In some aspects, the bioreactor is selected from a fed-batch system, a semi-continuous system and a continuous culture system. In some aspects, the bioreactor is a fed-batch system. In some aspects, the bacterial cell produces the target dsRNA. In some aspects, the target dsRNA is produced at an amount of at least about 4 g/L, 5 g/L, 6 g/L, 7 g/L, 8 g/L, 9 g/L, 10 g/L, 11 g/L or 12 g/L.

In some aspects, the present disclosure comprises a method for producing a target recombinant RNA. The method comprises maintaining the bacterial culture expressing the disclosed plasmid vector, in a bioreactor for a time and under conditions sufficient to yield the target dsRNA in an amount of at least about 4 g/L, 5 g/L, 6 g/L, 7 g/L, 8 g/L, 9 g/L, 10 g/L, 11 g/L or 12 g/L. In some aspects, the bioreactor is selected from a fed-batch system, a semi-continuous system and a continuous culture system. In some aspects the bioreactor is a fed-batch system. In some aspects, the method further comprises harvesting the target dsRNA. In some aspects, the target recombinant RNA is a dsRNA that specifically inhibits expression of a target gene. In some aspects, the target recombinant RNA sequence is selected from a dsRNA, a siRNA, a shRNA, a hpRNA, and a miRNA.

Further provided herein is the use of a bacterial culture comprising a population of bacterial cells disclosed herein for production of an antibiotic marker free recombinant RNA. In some aspects, the recombinant RNA is a dsRNA, a siRNA, a shRNA, a hpRNA, and a miRNA.

### BRIEF DESCRIPTION OF FIGURES

FIG. 1A is a schematic of plasmid pAPSE10218.

FIG. 1B is a schematic of plasmid pAPSE10448.

FIG. 1C is a schematic of plasmid pAPSE10471.

FIG. 1D is a schematic of plasmid pAPSE10500.

FIG. 1E is a schematic of plasmid pAPSE10772.

FIG. 1F is a schematic of plasmid pAPSE10775.

FIG. 1G is a schematic of plasmid pAPSE10797.

FIG. 1H is a schematic of plasmid pAPSE10822.

FIG. 1I is a schematic of plasmid pAPSE10826.

FIG. 1J is a schematic of plasmid pAPSE10835.

FIG. 1K is a schematic of plasmid pAPSE10836.

FIG. 2A is a schematic of constructs used for studying the impact of expressing the MS2 CP or GFP on accumulation of dsRNA in HT115(DE3) and JM109(DE3) cells.

FIG. 2B is an image of coomassie stained SDS-PAGE gel demonstrating expression of MS2 CP and GFP in the disclosed strains.

FIG. 2C is a bargraph representing shake flask dsRNA yields from the different E. coli strains. Bars labelled a and b differ significantly.

FIG. 2D is an image of agarose gel used for dsRNA quantification from different strains.

FIG. 3 is an image of agarose gel used for examining the accumulation of MS2 CP mediated dsRNA in E. coli cells with functional mc gene. Lanes 1 to 3 contain RNA samples from three independent JM109(DE3)/pAPSE10218 cultures. Lanes 4 to 6 contain RNA samples from three independent JM109(DE3)/pAPSE10305 cultures.

FIG. 4A is a scheme illustrating the design of the different plasmid constructs showing location of the pac sites on hpRNA. The rectangles in the construct diagram represent sense and anti-sense sequence of CPB β-actin respectively.

FIG. 4B is a bargraph representing shake flask dsRNA yield data from HT115(DE3) cells expressing the different plasmid constructs. Bars labelled with the same letter do not significantly differ.

FIG. 5A is a scheme illustrating the constructs for producing CPB β-actin dsRNA as an intramolecular hpRNA from a single transcript (pAPSE10218) and an intermolecular dsRNA by bidirectional transcription (pAPSE10402).

FIG. 5B is a bargraph representing dsRNA yields of CPB β-actin hpRNA and intermolecular dsRNA from HT115(DE3) cells expressing the different plasmid constructs.

FIG. 6A is a scheme illustrating non-pyrE expressing (pAPSE10218 and pAPSE10379) and pyrE over-expressing (pAPSE10775 and pAPSE10448) constructs.

FIG. 6B is a bargraph representing shake flask dsRNA yields of constructs pAPSE10379 and pAPSE10448 carrying RIFA β-actin sequence in minimal media.

FIG. 6C is a bargraph representing shake flask dsRNA yields of constructs pAPSE10218 and pAPSE10775 carrying CPB β-actin sequence in minimal media.

FIG. 7 is a bargraph representing dsRNA yield from high-cell-density fed batch fermentation cultures.

FIG. 8 is a linegraph representing mortality of Colorado potato beetle (Leptinotarsa decemlineata (Say)) (CPB) larva in leaf disc bioassays when exposed to heat-killed E. coli HT115(DE3)/pAPSE10218 cells containing β-actin dsRNA, applied at three different concentrations or when fed untreated leaf discs. Significant differences between treatments are denoted with different letters next to the corresponding line.

FIG. 9 is a schematic of HT115(DE3)-ΔpyrE around mini-Tn10::rnc-era-reco

The figures do not limit the present inventive concept to the specific aspects disclosed and described herein. The drawings are not necessarily to scale, emphasis instead being placed on clearly illustrating principles of certain aspects of the present inventive concept.

### DETAILED DESCRIPTION

The present disclosure provides plasmid vectors and methods for improved production of large quantities of unencapsidated dsRNA in vivo. The present disclosure is based on the surprising discovery that co-expression of sequences encoding MS2 CP cassette, hpRNA, pac sites and pyrE in a plasmid vector can substantially improve the expression of dsRNA. These constructs can be expressed in bacterial cells and can be cultured in a scalable high density fed-batch fermentation bioreactor for use as a high yielding system for dsRNA production.

All publications mentioned herein are incorporated by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosure.

### Definitions

When introducing elements of the present disclosure or the preferred aspects(s) thereof, the articles “a”. “an”. “the” and “said” are intended to mean that there are one or more of the elements. The terms “comprising”, “including” and “having” are intended to be inclusive and mean that there may be additional elements other than the listed elements. Wherever the terms “comprising” or “including” are used, it should be understood the disclosure also expressly contemplates and encompasses additional aspects “consisting of” the disclosed elements, in which additional elements other than the listed elements are not included.

As used herein, the disclosure of numerical ranges by numerical endpoints includes all numbers encompassed by that range (e.g., “1 to 5” includes but is not limited to 1, 1.25, 1.5, 1.75, 2, 2.3, 2, 5, 2.8, 3, 3.1, 3.3, 3.8, 3.9, 4, 4.25, 4.5, 4.75 and 5). Unless otherwise indicated, all numbers used herein to express quantities, amounts, dimensions, measurements, and the like should be understood as encompassing the specific quantities, amounts, dimensions, measurements and so on, including those instances modified by the term “about.” For example, an amount disclosed herein as “about 1 mg/mL” would expressly include the amount of 1 mg/mL, and so on. Accordingly, unless indicated to the contrary, the numerical descriptions set forth herein may vary while remaining well within the teachings of the present disclosure. At the very least, each numerical value should be construed in view of the number of significant digits and by applying routine rounding techniques. As various changes could be made in the above-described cells and methods without departing from the scope of the disclosure, it is intended that all matter contained in the above description and in the examples given below, shall be interpreted as illustrative and not in a limiting sense.

As used herein, the terms “capsid protein” or “coat protein” refer to the coat protein of bacteriophage MS2, capable of binding the cognate bacteriophage RNA pac site with high affinity and assembling into a complex hollow tertiary structure in which the bacteriophage RNA may be entirely encapsidated within the hollow tertiary structure. The term “capsid” refers to the hollow tertiary structure formed by assembly of individual capsid proteins. An incomplete capsid is understood to mean a capsid that is not completely closed, such that no hollow tertiary structure is formed.

As used herein “dsRNA” refers to double stranded RNA comprising substantially complementary strands of RNA. A dsRNA comprises RNA sequences with sufficient internal homology to form significant secondary structures such as hairpins due to hybridization of internal complementary sequences with one another via Watson-Crick base pairing of nucleotide bases within the complementary sequences.

As used interchangeably herein, the terms “RNA interference” and “RNAi” refer to degradation of mRNA or inhibition of protein synthesis through an endogenous pathway including the DICER protein complex. DICER cleaves long double stranded RNA (dsRNA) molecules into short fragments of approximately 21 nucleotides, termed small-interfering RNA (siRNA). The siRNA is unwound into two single-stranded RNAs: the passenger strand and the guide strand. The passenger strand is degraded, and the guide strand is incorporated into the RNA-induced silencing complex (RISC). Micro ribonucleic acids (miRNAs) are usually 22 nucleotides long, therefore, very similar to siRNAs in size, however, the miRNAs are cleaved from precursor molecules containing a polynucleotide “loop” connecting the hybridized passenger and guide strands, and they may be similarly incorporated into RISC. Post-transcriptional gene silencing occurs when the guide strand binds specifically to a complementary mRNA molecule and induces cleavage by Argonaute, the catalytic component of the RISC. One possibility to generate siRNAs in cells is the expression of a precursor RNA, called hairpin RNA (hp-RNA). Transcription of hp-RNAs is usually driven by RNA-polymerase-III promoters like the U6 or H1 promoter or 17 RNA polymerase. RNA-mediated silencing using an inverted repeat of a nucleic acid or a part thereof (in this case a stretch of substantially contiguous nucleotides derived from the target gene, or from any nucleic acid capable of encoding an orthologue, paralogue or homologue of the protein of interest), preferably capable of forming a hairpin structure. The inverted repeat is cloned in an expression vector comprising control sequences. A non-coding DNA nucleic acid sequence (a spacer, for example a matrix attachment region fragment (MAR), an intron, a polylinker, etc.) is located between the two inverted nucleic acids forming the inverted repeat. After transcription of the inverted repeat, a chimeric RNA with a self-complementary structure is formed (partial or complete), his double-stranded RNA structure is referred to as the hairpin RNA (hpRNA). The hpRNA is processed by the insects into siRNAs that are incorporated into an RNA-induced silencing complex (RISC). The RISC subsequently cleaves the target mRNA transcripts, thereby substantially reducing the number of mRNA transcripts to be translated into polypeptides.

As used herein “plasmid” refers to any extrachromosomal episome capable of replication or stable maintenance within the host cell. Specifically embraced by this definition are plasmids such as pBR322, pCG1, and pACYC184 which represent the backbones of the described plasmids. Those of ordinary skill in the art will recognize that other plasmids or stably maintained viral episomes can provide the same required functions of maintenance, expression and selection and that alternatives to the basic plasmids described herein may be generated from such other plasmids or stably maintained viral episomes without undue experimentation. A key feature of the present invention is the ability to express the genes encoding a dsRNA and a capsid protein, not specific modes of replication, expression or the selective markers found on episomes containing the genes encoding the dsRNA and capsid protein. As used herein “vector” comprises a nucleic acid construct or an expression construct. A vector as described herein may be selected from any genetic element known in the art which can facilitate transfer of nucleic acids between cells, such as, but not limited to, plasmids, transposons, cosmids, chromosomes, artificial chromosomes, viruses, virions, and the like.

As used herein “pyrE” refers to a pyrimidine biosynthetic pathway enzyme orotate phosphoribosyltransferase.

As used herein “pac site” refers to a packaging site.

As used herein “unencapsdiated dsRNA” refers to double strand RNA not incorporated within capsids and includes both dsRNA associated with incomplete capsids and dsRNA with no association with bacteriophage coat protein whatsoever. The dsRNA contemplated in the present invention comprises a single RNA with two complementary domains separated by a nonhomologous recombinant spacer/loop sequence capable of forming a hairpin structure. The dsRNA can be a hairpin RNA (hRNA) or stem-loop RNA.

A polynucleotide described herein may comprise one or more nucleic acids each encoding a polypeptide, all operably linked to (i.e., in a functional relationship with) one or more regulatory sequences, such as a promoter. Such a polynucleotide may alternatively be referred to herein as a “nucleic acid construct” or “construct”.

Each nucleic acid sequence described herein by virtue of its identity or similarity percentage with a given nucleic acid sequence respectively has in a further preferred aspect an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively. The terms “homology”, “sequence identity” and the like are used interchangeably herein. Sequence identity is described herein as a relationship between two or more nucleic acid (polynucleotide) sequences, as determined by comparing the sequences. In a preferred aspect, sequence identity is calculated based on the full length (in amino acids or nucleotides) of two given SEQ ID NOS or based on a portion thereof. A portion of a full-length sequence may be referred to as a fragment, and preferably means at least 50%, 60%, 70%, 80%, 90%, or 100% of the length (in nucleotides) of a reference sequence. “Identity” also refers to the degree of sequence relatedness between two nucleic acid sequences, as the case may be, as determined by the match between strings of such sequences. The degree of sequence identity between two sequences can be determined, for example, by comparing the two sequences using computer programs commonly employed for this purpose, such as global or local alignment algorithms. Non-limiting examples include BLASTp, BLASTn, Clustal W, MAFFT, Clustal Omega, AlignMe, Praline, GAP, BESTFIT, or another suitable method or algorithm. A Needleman and Wunsch global alignment algorithm can be used to align two sequences over their entire length or part thereof (part thereof may mean at least 50%, 60%, 70%, 80%, 90% of the length of ths sequence), maximizing the number of matches and minimizes the number of gaps. Default settings can be used and preferred program is Needle for pairwise alignment (in an aspect, EMBOSS Needle 6.6.0.0, gap open penalty 10, gap extent penalty: 0.5, end gap penalty: false, end gap open penalty: 10, end gap extent penalty: 0.5 is used) and MAFFT for multiple sequence alignment.

An “expression construct” or “nucleic acid construct” carries a genome that is able to stabilize and remain episomal in a cell. Within the context of the invention, a cell may mean to encompass a cell used to make the construct or a cell wherein the construct will be administered. Alternatively, a construct is capable of integrating into a cell's genome, e.g. through homologous recombination or otherwise. A “DNA construct” or “nucleic acid construct” prepared for introduction into a particular host may include a replication system recognized by the host, an intended DNA segment encoding a desired polypeptide, and transcriptional and translational initiation and termination regulatory sequences operably linked to the polypeptide-encoding segment. By way of non-limiting example, a promoter or enhancer is operably linked to a coding sequence if it stimulates the transcription of the sequence. DNA for a signal sequence is operably linked to DNA encoding a polypeptide if it is expressed as a preprotein that participates in the secretion of a polypeptide. Generally, a DNA sequence that is operably linked are contiguous, and, in the case of a signal sequence, both contiguous and in reading frame. However, enhancers need not be contiguous with a coding sequence whose transcription they control. Linking is accomplished by ligation at convenient restriction sites or at adapters or linkers inserted in lieu thereof, or by gene synthesis

As used herein “insect” refer not only to insects but to their immature forms and larvae.

“Pharmaceutical composition” means a mixture of substances suitable for administering to an individual that includes a pharmaceutical agent. As used herein a pharmaceutical composition comprises one or more of receptors, vectors, cells disclosed herein compounded with suitable pharmaceuticals carriers or excipients.

“Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication, condition or biochemical indicia associated with a disease.

As used herein, the term “subject” refers to any organism to which dsRNA described herein are administered in accordance with the present invention e.g., for experimental, diagnostic, prophylactic, and/or therapeutic purposes. Typical subjects include animals (e.g., mammals such as mice, rats, rabbits, non-human primates, humans, insects, worms etc.). In an aspect, a subject is a human. In some aspects, a subject may be suffering from, and/or susceptible to a disease, disorder, and/or condition.

The present disclosure encompasses plasmid vectors and methods for producing large quantities of dsRNA in vivo from microbial cells. In some aspects, the plasmid vector comprises a nucleic acid sequence comprising: an inducible bacterial promoter operably linked to a stem-loop sequence having a 3′ end and a 5′ end and comprising a target dsRNA sequence; a first pac site sequence at the 3′ end, and a second pac site sequence at the 5′ end of the stem-loop sequence; an MS2 capsid protein (CP) expression cassette; and the pyrE coding sequence with a ribosome binding site (RBS) at its 5′ end and T1-T2 terminators at its 3′ end downstream of the MS2 CP expression cassette. The nucleic acid (DNA) sequences for each of the T7 promoter, MS2 capsid protein, pac sites, pyrE coding sequence with a ribosome binding site (RBS) at its 5′ end and T1-T2 terminators, T7 terminator and the expression cassettes are provided in Table 5.

In some aspects, the plasmid vector of the present disclosure is expressed in an host cell. A host cell can be a cell (e.g., bacteria, yeast cell, fungal cell, CHO, mammalian cell, etc.) that can been genetically altered, modified, or engineered, using methods and plasmids described herein. In some aspects, the host cell is a prokaryotic cell, a bacterial cell, or an eukaryotic cell. In some aspects, mammalian, insect, plant or yeast cell (for e.g., Saccharomyces sp., Pichia sp, or Schizosaccharomyces sp.) are contemplated for use in the methods disclosed herein.

In some aspects, the host cell is a bacterial cell. In some aspects, the bacterial cell is a gram-negative bacterial cell. In some aspects, a gram-negative bacteria non-limiting examples of which can include Escherichia sp., Salmonella sp., Shigella sp., Agrobacterium. Campylobacter sp., Lactobacillus sp., Neisseria sp., Legionella sp., or Pseudomonas sp. In some aspects, a gram negative bacterial cell is an Escherichia cell. In some aspects. In some aspects, the bacterial cell is an E. coli cell. In some aspects, E. coli can be a a strain that is deficient in one or more endogenous ribonuclease. For example, ribonuclease deficiency can be created by deleting, removing, knock-out, silencing, suppressing, or otherwise downregulating at least one endogenous ribonuclease. In some aspects, the E coli cell is an RNAseIII-deficient E. coli strain. In some aspects, the E. coli is RNAseIII-deficient E. coli strain, HT115(DE3).

In some aspects, the bacterial cell is a gram-positive bacterial cell. In some aspects, a gram-positive bacteria, non-limiting examples of which can include Bacillus sp., Corynebacterium sp., Lactobacillus sp., Staphylococcus sp., or Streptococcus sp. In some aspects, the gram-positive bacterial cell is a Corynebacterium cell. In some aspects, the bacterial cell is a C. glutamicum cell.

In some aspects, the bacterial cell can be engineered to comprise a promoter which may be constitutive promoters or regulated promoters. Non-limiting examples of inducible promoters and their subsequent inducers include lac (IPTG), lacUV5 (IPTG), tac (IPTG), trc (IPTG), P_{syn} (IPTG), trp (tryptophan starvation), araBAD (1-arabinose), lppa (IPTG), lpp-lac (IPTG), phoA (phosphate starvation), recA (nalidixic acid), proU (osmolarity), cst-1 (glucose starvation), teta (tretracylin), cada (pH), nar (anaerobic conditions), PL (thermal shift to 42° C.), cspA (thermal shift to 20° C.), T7 (thermal induction), T7-lac operator (IPTG), T3-lac operator (IPTG), T5-lac operator (IPTG), T4 gene32 (T4 infection), nprM-lac operator (IPTG), P_{syn}(alkyl-or halo-benzoates), Pu (alkyl-or halo-toluenes). Psal (salicylates), and VHb (oxygen).

In further aspects, the bacterial cell disclosed herein is an antibiotic marker free cell. In such aspects, bacterial cell lacks a nucleic acid sequence encoding an antibiotic selection marker. In such aspects, the bacterial cell can be grown or cultured in the absence of an antibiotic. In some aspects, the antibiotic selection marker can be, but not limited to amphotericin B, bacitracin, carbapenem, cephalosporin, ethambutol, fluoroquinolones, isonizid, cephalosporin, methicillin, oxacillin, vanomycin, streptomycin, quinolines, rifampin, rifampicin, sulfonamides, ampicillin, tetracycline, neomycin, cephalothin, erythromycin, streptomycin, kanamycin, gentamycin, penicillin, and chloramphenicol resistance genes, or other commonly used non-auxotrophic selection markers. In some aspects, the antibiotic selection marker can be a chloramphenicol resistance gene. In some aspects, the antibiotic selection marker can be a tetracycline resistance gene.

In certain aspects, a bacterial cell disclosed herein can be engineered to to induce auxotrophy. In some aspects, bacterial cells can be genetically modified to induce auxotrophy for at least one metabolite. The genetic modification can be to a gene or genes encoding an enzyme that is operative in a metabolic pathway, such as an anabolic biosynthetic pathway or catabolic utilization pathway. Preferably, the host cell has all operative genes encoding a given biocatalytic activity deleted or inactivated in order to ensure removal of the biocatalytic activity. In some aspects, bacterial cells disclosed herein can be modified to be uracil auxotrophs. In such aspects, the bacterial cells can be maintained in a culture medium comprising uracil.

In some aspects, the disclosure encompasses bacterial cells without an antibiotic resistance marker gene. In some aspects, bacterial cells are E. coli cells. In such aspects, provided herein are E. coli cells without a chloramphenicol resistance gene, tetracycline resistance gene.

In some aspects, the disclosure further encompasses transforming a bacterial cell with a plasmid vector disclosed herein. In such aspects, the bacterial cells are engineered to express a recombinant RNA molecule involved in RNAi. In some aspects, the recombinant RNA molecule involved in RNAi can be a double stranded RNA (dsRNA), a micro ribonucleic acid (miRNA), a small-interfering RNA (siRNA), a hairpin RNA (hpRNA), or a short hairpin RNA (shRNA). In some aspects, the the bacterial cells are engineered to express a dsRNA.

In certain aspects, the present disclosure further comprises culturing a population of bacterial cells transformed with the plasmid vector, in a bioreactor, wherein the bioreactor is selected from a fed-batch system, a semi-continuous system and a continuous culture system. The method comprises maintaining the bacterial culture expressing the disclosed plasmid vector, in a bioreactor for a time and under conditions sufficient to yield the target recombinant RNA molecule in an amount of at least about 4 g/L, 5 g/L, 6 g/L, 7 g/L, 8 g/L, 9 g/L, 10 g/L, 11 g/L or 12 g/L. In some aspects, the bioreactor is a fed-batch system. In some aspects, the target recombinant RNA molecule is a dsRNA, a siRNA, a shRNA, a hpRNA, or a miRNA.

### Construction of Plasmid Vector

Routine microbial and molecular cloning methods and tools, including those for generating and purifying DNA, RNA, and proteins, and for transforming host organisms and expressing recombinant proteins and nucleic acids as described herein, are fully within the capabilities of a person of ordinary skill in the art and are well described in the literature. See, e.g., Sambrook, et al., Molecular Cloning: A Laboratory Manual, 2nd ed., Cold Spring Harbor Laboratory Press, Cold Spring Harbor. N.Y. (1989); Davis, et al., Basic Methods in Molecular Biology, Elsevier Science Publishing Co., Inc., N.Y. (1986); and Ausubel, et al., Current Protocols in Molecular Biology, Greene Publ. Assoc., Wiley-Interscience, NY (1995). The disclosures in each of which are herein incorporated by reference.

In certain aspects, plasmid vector is constructed to have optimal expression of the target dsRNA. Any plasmid vectors or stably maintained viral episomes which can provide required functions of maintenance, expression and selection can be used in the present disclosure. Alternatively, plasmids with the ability to express the genes encoding a dsRNA and a capsid protein, is used and is not limited by specific modes of replication, expression or the selective markers associated with the genes encoding the dsRNA and capsid protein. In some aspects, the recombinant DNA constructs described herein are based on a common plasmid vector series derived from plasmid pBR322. Alternatively, recombinant DNA constructs are generated from others plasmids or stably maintained viral episomes. The plasmid vector can be assembled using any known method in the art. By way of non-limiting example, synthetic fragments of the constructs can be obtained and amplified using appropriate primer pairs. The amplified fragments can then be cloned into respective restriction sites of the plasmid to obtain plasmid vector comprising any or all the disclosed construct sequences. Any of the sequences provided in Table 5 can be used to construct the plasmid vector of the present disclosure.

In some aspects, the plasmid vector comprises one or more of a nucleic acid sequence comprising a bacterial promoter operably linked to a stem-loop sequence having a 3′ end and a 5′ end, a target dsRNA sequence; a first pac site sequence at the 3′ end, and a second pac site sequence at the 5′ end of the stem-loop sequence; an MS2 capsid protein (CP) expression cassette; the pyrE coding sequence, a ribosome binding site (RBS) at 5′ end and/or T1-T2 terminators at its 3′ end of the pyrE coding sequence, downstream of the MS2 CP expression cassette, or any combination thereof.

In some aspects the bacteriophage capsid protein of the plasmid vector disclosed herein is encoded by the coat protein gene of a species of leviviridae. In some aspects, the coat protein gene encodes the capsid protein of bacteriophage MS2. In some aspects, the plasmid vector comprises a MS2 CP expression cassette.

In some aspects, the MS2 CP expression cassette can further comprise a promoter selected from a constitutive or inducible transcriptional promoter. In some aspects, the non-limiting examples of a promoter can include T7. T3, Sp6 RNA, or J23115 promoter. In some aspects, the promoter is an inducible promoter. In some aspects, the inducible promoter is a T7 promoter. In some aspects, the MS2 CP expression cassette comprises a terminator. In some aspects, the terminator is a 7 terminator. In some aspects, the MS2 CP cassette comprises a T7 promoter and T7 terminator.

In some aspects, MS2 CP cassette comprises a MS CP with the nucleic acid sequence of SEQ ID NO: 5. In some aspects, MS2 CP comprises a nucleic acid sequence with at least 70% sequence identity or similarity with SEQ ID NO: 5. In some aspect, the identity or similarity is of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. In some aspects, MS2 CP cassette comprises MS2 CP with the amino acid sequence of SEQ ID NO: 9 (Table 6). In some aspects, MS2 CP comprises an amino acid sequence with at least 70% sequence identity or similarity with SEQ ID NO: 9. In some aspect, the identity or similarity is of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In certain aspects, the sequence encoding the dsRNA may be associated with and expressed from an inducible or a constitutive transcriptional promoter. In some aspects, the non-limiting examples of a promoter can include T7, T3, Sp6 RNA, or J23115 promoter. In some aspects, the sequence encoding the dsRNA may be associated with and expressed from an inducible promoter. The inducible transcriptional promoter associated with expression of the dsRNA may be the same inducible transcriptional promoter or a different transcriptional promoter from a transcriptional promoter associated with MS2 expression cassette. In certain aspects, the inducible transcriptional promoter associated with expression of dsRNA is T7 promoter.

In some aspects, the plasmid vector comprises a coliphage T7 promoter with a sequence of SEQ ID NO.1. In some aspects, coliphage T7 promoter comprises a sequence with at least 70% sequence identity or similarity with SEQ ID NO: 1. In some aspect, the identity or similarity is of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In some aspects, the plasmid vector comprises a target dsRNA sequence. The dsRNA can reduce, inhibit or suppress expression of a target gene through RNAi. In some aspects, sequences that encode dsRNA can be engineered using methods known in the art. Such sequence constructs comprise sense and anti-sense sequences which are placed in regions flanking an intron sequence in proper splicing orientation with donor and acceptor splicing sites. Alternatively, spacer sequences of various lengths can be employed to separate self-complementary regions of sequence in the construct. During processing of the gene construct transcript, intron sequences can be spliced-out, allowing sense and anti-sense sequences, as well as splice junction sequences, to bind forming dsRNA.

The RNAi polynucleotide can hybridize with the full length mRNA encoded by the target gene or hybridize to a fragment of the target RNA or DNA (the target sequence). In some aspects, the target sequence is between 1 and 500 nucleotides in length. In some aspects, the target sequence and/or the dsRNA sequence is between about 50 and 400 nucleotides in length. In some aspects, the target sequence and/or dsRNA sequence is between 100 and 300 nucleotides in length. In some aspects, the sequence of the dsRNA used for RNAi has 100% identity or similarity to the target sequence of the target gene, but can be at least 70%, 80%, 90%, 95%, 98% or 99% or more similar or identical to the target sequence. In some aspect, the identity or similarity is of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. In some aspects, dsRNA having greater than 90% or 95% sequence identity is used, such that sequence variations that might be expected due to genetic mutation, strain polymorphism, or evolutionary divergence can be tolerated.

In certain aspects dsRNA targets a specific essential gene to reduce, inhibit or suppress the expression of the gene. In some aspects, the dsRNA targets genes of an insect pest or a pathogen. In some aspects, the dsRNA targets gene of an insect pest. In some aspects, dsRNA comprises a nucleotide sequence which is complementary to at least part of a nucleotide sequence of an insect target gene. In some aspects, target genes can be one of midgut or non-midgut genes, neurohoromone gene, pheromone-biosynthesis-activating neuropeptide (PBAN)/pyrokinin gene, tubulin, vATPase, acetyl choline esterase, chitin synthase gene (e.g. CHS1 and/or CHS2), cytochrome P450 gene, Snf7, beta-actin, genes coding for inhibitors of apoptosis (e.g. IAP), ribosomal proteins (e.g. CHD3, S4 or S9) or other conserved genes or insect-specific genes.

In some aspects, the plasmid vector can further comprise a pac site sequence. In some aspects, the pac site is a MS2 pac site. In some aspects, the plasmid vector comprises a first pac site sequence at the 3′ end, and a second pac site sequence at the 5′ end of the stem-loop sequence. In some aspects, plasmid vector comprises one MS2 pac site each at the 5′ and 3′ ends of the dsRNA hairpin sequence. In some aspects, the plasmid vector is optimally designed to comprise one pac site at its 5′ and 3′ end to maximize dsRNA yield.

In some aspects, the first and second pac site sequences each of which can comprise nucleic acid sequence of SEQ ID NO. 3 and SEQ ID NO: 4, respectively. In some aspects, pac site comprises a nucleic acid sequence with at least 70% sequence identity or similarity with SEQ ID NO: 3. In some aspects, pac site comprises a sequence with at least 70% sequence identity or similarity with SEQ ID NO: 4. In some aspect, the identity or similarity is of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In some aspects, the plasmid vector comprises a pyrE coding sequence. In some aspects, the pyrE coding sequence comprises a ribosome binding site at its 5′end and T1-T2 terminators at its 3′ end. In some aspects, the pyrE construct is inserted downstream of the MS2 CP expression cassette. In some aspects, the pyrE construct is engineered to permit readthrough transcription of the pyrE coding sequence by the T7 promoter. In some aspects, the pyrE coding sequence comprising RBS-pyrE cds-T1-T2 is a cassette and is driven by a dedicated promoter. Non-limiting examples of a promoter can include T7, T3, Sp6 RNA, or J23115 promoter. In some aspects, pyrE conding sequence, or a construct comprising a pyrE is engineered to be driven by a coliphage T7 promoter. In some aspects, pyrE, or a construct comprising a pyrE is engineered to be driven by a J23115 promoter.

In some aspects, the pyrE construct of the disclosed plasmid vector comprises RBS-pyrE cds-T1-T2 comprising a nucleic acid sequence of SEQ ID NO: 2. In some aspects, RBS-pyrE cds-T1-T2 comprises a nucleic acid sequence with at least 70% sequence identity or similarity with SEQ ID NO: 2. In some aspect, the identity or similarity is of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In some aspects, pyrE or a construct comprising pyrE is driven by an upstream coliphage T7 promoter comprising a nucleic acid sequence of SEQ ID NO:1. In some aspects, coliphage T7 promoter comprises a nucleic acid sequence with at least 70% sequence identity or similarity with SEQ ID NO: 1. In some aspect, the identity or similarity is of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In some aspects, pyrE or a construct comprising pyrE is driven by a J23115 promoter comprising a nucleic acid sequence of SEQ ID NO:33. In some aspects, J23115 promoter comprises a nucleic acid sequence with at least 70% sequence identity or similarity with SEQ ID NO: 1. In some aspect, the identity or similarity is of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In some aspects, the pyrE coding sequence comprising RBS-pyrE cds-T1-T2 is a cassette and is driven by a dedicated coliphage T7 promoter comprising a nucleic acid sequence of SEQ ID NO: 1. In some aspects, the dedicated coliphage T7 promoter comprises a sequence with at least 70% sequence identity or similarity with SEQ ID NO: 1. In some aspect, the identity or similarity is of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In some aspects, the plasmid vector is the plasmid pAPSE10775 engineered to express a target recombinant RNA molecule. In some aspects, the target recombinant RNA molecule is selected from a dsRNA, a siRNA, a shRNA, a hpRNA, and a miRNA. In some aspects, the plasmid vector comprises one or more of the pac sites, MS2 CP cassette. RBS-pyrE cds-T1-T2 terminator of pAPSE10775, or any combination thereof and a target dsRNA. In some aspects, the vector plasmid consists of or comprises the sequences of SEQ ID NOs: 1, 2, 3, 4, 5, 6, 7, 8, 33, or any combination thereof. In some aspects, the vector plasmid consists of or comprises a sequence with at least 70% sequence identity or similarity with SEQ ID NOs: 1, 2, 3, 4, 5, 6, 7, 8, and/or 33. In some aspect, the identity or similarity is of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In some aspects, the plasmid vector is the plasmid pAPSE10471 engineered to express a target recombinant RNA moleculeIn some aspects, the target recombinant RNA molecule is selected from a dsRNA, a siRNA, a shRNA, a sshRNA, IshRNA, and miRNA. In some aspects, the plasmid vector comprises one or more of the pac sites, MS2 CP cassette, T7-RBS-pyrE cds-T1-T2 of pAPSE10471, or any combination thereof and a target dsRNA. In some aspects, the vector plasmid consists of or comprises the sequence of SEQ ID NO.35. In some aspects, the vector plasmid consists of or comprises a sequence with at least 70% sequence identity or similarity with SEQ ID NO.35. In some aspect, the identity or similarity is of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In further aspects, the plasmid vector disclosed herein is an antibiotic marker free plasmid. In such aspects, plasmid vector lacks a nucleic acid sequence encoding an antibiotic selection marker. In some aspects, the antibiotic selection marker can be, but not limited to amphotericin B, bacitracin, carbapenem, cephalosporin, ethambutol, fluoroquinolones, isonizid, cephalosporin, methicillin, oxacillin, vanomycin, streptomycin, quinolines, rifampin, rifampicin, sulfonamides, ampicillin, tetracycline, neomycin, cephalothin, erythromycin, streptomycin, kanamycin, gentamycin, penicillin, and chloramphenicol resistance genes, or other commonly used non-auxotrophic selection markers. In some aspects, the antibiotic selection marker can be a Amp-r gene such as a bla, beta and/or lactamase.

In some aspects, the plasmid vector is the plasmid pAPSE10836 engineered to express a target recombinant RNA molecule. In some aspects, the target recombinant RNA molecule is selected from a dsRNA, a siRNA, a shRNA, a hpRNA, and miRNA. In some aspects, the plasmid vector comprises one or more of the pac sites, MS2 CP cassette, and P-J23115-pyrE cds-T1-T2 terminator of pAPSE10836, or any combination thereof and a target dsRNA. In some aspects, the vector plasmid consists of or comprises the sequence of SEQ ID NO.43. In some aspects, the vector plasmid consists of or comprises a sequence with at least 70% sequence identity or similarity with SEQ ID NO. 43. In some aspect, the identity or similarity is of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

### Production of Recombinant RNA

In some aspects, the present disclosure further comprises transformation of the disclosed plasmid vectors into host cells. In certain aspects, the present disclosure comprises cell lines capable of expressing plasmid vectors and target recombinant RNA molecule. In some aspects, the recombinant RNA molecule is a dsRNA, siRNA, shRNA, hpRNA, or miRNA. In some aspects, the target recombinant RNA molecule is dsRNA. In some aspects, the method comprises electroporating the plasmid vector described herein into bacterial cells. The bacterial cells can be any bacteria capable of transformation. In some aspects, the bacterial cell lacks double-strand specific RNase III and/or contains an inducible T7 RNA polymerase gene. In some aspects, the bacterial cell is a gram negative bacterial cell, such as an E. coli strain, representative examples of which include K12 strains and derivatives thereof (e.g. MG1655. HT115(DE3)) and B strains (e.g. BL21(DE3), REL606). In some aspects, the bacterial cell is a gram positive bacterial cell. In certain aspects, the gram-positive bacterial cell is from the genus Corynebacterium. In some aspects, the bacterial cell is Corynebacterium glutamicum. In some aspects, the disclosed bacterial cell can be without an antibiotic resistance marker gene. In some aspects, bacterial cells are E. coli cells without a chloramphenicol resistance gene. In some aspects the bacterial cells are E coli cells without a tetracycline resistance gene. Any known method in the art for transformation of bacterial cells can be used.

In some aspects, E. coli strain HT115(DE3) with genotype F⁻, mcrA, mcrB, IN (rrnD-rrnE)1, rnc14::Tn10 (Lambda DE3 lysogen: lacUV5 promoter-T7 polymerase)) is used. After transformation, the resulting recombinant transformants are selected, by way of non-limiting example, on LB agar plates containing tetracycline and/or ampicillin. Single colonies are isolated, the presence of intact plasmid confirmed by restriction enzyme analysis and the confirmed transformed cells are saved for future use.

Further provided herein are bacterial cells comprising a vector plasmid disclosed herein. In such aspects, the bacterial cells comprise a plasmid comprising one or more of the pac sites, MS2 CP cassette, and RBS-pyrE cds-T1-T2 terminator, the target recombinant RNA, or any combination thereof, disclosed herein. In some aspects, the recombinant RNA molecule is a dsRNA, siRNA, shRNA, hpRNA, or miRNA. In some aspects, the disclosed bacterial cells may comprise the plasmid pAPSE10775, one or more of the pac sites, MS2 CP cassette, RBS-pyrE cds-T1-T2 terminator of pAPSE10775. In some aspects, the disclosed bacterial cells may comprise plasmid pAPSE10471, or one or more of the pac sites, MS2 CP cassette, T7-RBS-pyrE cds-T1-T2 of pAPSE10471. In some aspects, the disclosed bacterial cells may comprise plasmid pAPSE10836, or one or more of the pac sites, MS2 CP cassette, and P-J23115-pyrE cds-T1-T2 terminator of pAPSE10836.

In some aspects, bacterial cells are E. coli cells. In some aspects, E. coli cells are cells without a chloramphenicol resistance gene. In some aspects, the E. coli are cells without a tetracycline resistance gene. In such aspects, the E. coli cells comprise a plasmid comprising one or more of the pac sites, MS2 CP cassette, and RBS-pyrE cds-T1-T2 terminator, the target recombinant RNA, or any combination thereof, disclosed herein. In some aspects, the recombinant RNA molecule is a dsRNA, siRNA, shRNA, hpRNA, or miRNA. In some aspects, the disclosed E. coli cells may comprise the plasmid pAPSE10775, one or more of the pac sites, MS2 CP cassette, RBS-pyrE cds-T1-T2 terminator of pAPSE10775. In some aspects, the disclosed E. coli cells may comprise plasmid pAPSE10471, or one or more of the pac sites, MS2 CP cassette, T7-RBS-pyrE cds-T1-T2 of pAPSE10471. In some aspects, the disclosed E. coli cells may comprise plasmid pAPSE10836, or one or more of the pac sites, MS2 CP cassette, and P-J23115-pyrE cds-T1-T2 terminator of pAPSE10836.

In some aspects, further provided is production of dsRNA comprising culturing a population of bacterial cells transformed with the plasmid vector, in a bioreactor. The bioreactor is maintained for an adequate time and under sufficient conditions, to yield a target dsRNA.

In some aspects, the method comprises culturing transformed bacterial cells in a bioreactor. Growth of bacterial cells transformed with the plasmid vector comprising the target dsRNA may be carried out in a minimal (mineral) media or in a rich media. Such media are well known to those of ordinary skill in the art. The methods disclosed herein may be carried out using standard industrial microbiology techniques and standard fermentation procedures, so long as such methods are adapted to the specific plasmid and host cell requirements, such as providing the appropriate selection markers to retain the specific plasmid vectors, using the appropriate stimuli to induce transcription of the specific promoters at appropriate times, and maintaining the required temperature and respiratory conditions necessary for cell growth, each of which is within the working knowledge of those of ordinary skill in the art.

In some aspects, the culturing method comprises using a seed culture as an inoculum. In some aspects, the seed culture is used to inoculate a minimal medium. In some aspects, minimal medium can comprise an ammonium salt, a potassium salt, a sodium salt, a magnesium salt, a carbon source, or any combination thereof. In some aspects, the ammonium salt can be (NH4)2SO4. In some aspects, the potassium salt in the medium can be KH2PO4. In some aspects, the sodium salt in the medium can be Na2HPO4. In some aspects, the magnesium salt in the medium can be MgSO4. In some aspects, the carbon source is glucose, or a lactose. In some aspects, the minimal medium can comprise (NH4)2SO4, KH2PO4, Na2HPO4, MgSO4, glucose, or any combination thereof. In some aspects, the minimal medium can comprise (NH4)2SO4, KH2PO4, Na2HPO4, MgSO4, glucose, lactose, or any combination thereof. In some aspects, seed culture is grown for a sufficient time at adequate temperature conditions.

In further aspects, inoculum is transferred to a fermentation vessel containing adequate amount of media. In some aspects, the medium can comprise a minimal medium. In some aspects, minimal medium can comprise an ammonium salt, a potassium salt, a sodium salt, a magnesium salt, a carbon source, trace metal, or any combination thereof. In some aspects, the ammonium salt can be (NH4)2SO4. In some aspects, the potassium salt in the medium can be KH2PO4. In some aspects, sodium salt in the medium can be Na2HPO4. In some aspects, the magnesium salt in the medium can be MgSO4. In some aspects, the carbon source is glucose. In some aspects, the trace metal is a commercially available trace metal solution (for e.g., Teknova, Hollister, CA). In some aspects, the minimal medium can comprise (NH4)2SO4, KH2PO4, Na2HPO4, MgSO4, glucose and a trace metal. In some aspects, the culture is maintained with a supply of carbon source (for e.g. a glucose feed with the major salts of minimal media).

In some aspects, the minimal medium disclosed herein comprises an ammonium salt at about 0.5 to about 100 mM, for e.g. about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 7.5 mM, about 8 mM, about 8.5 mM, about 9 mM, about 9.5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, or about 100 mM. In some aspects, the ammonium salt is (NH4)2SO4, and is present in the medium at about 20 mM to about 70 mM.

In some aspects, the minimal medium disclosed herein comprises a potassium salt at about 0.5 to about 50 mM, for e.g. about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 7.5 mM, about 8 mM, about 8.5 mM, about 9 mM, about 9.5 mM, about 10 mM, about 15 mM, about 20 mM, about 25 mM, about 30 mM, about 35 mM, about 40 mM, about 45 mM, or about 50 mM. In some aspects, the potassium salt is KH2PO4, and is present in the medium at about 0.5 mM to about 20 mM.

In some aspects, the minimal medium disclosed herein comprises a sodium salt at about 0.5 to about 100 mM, for e.g. about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 7.5 mM, about 8 mM, about 8.5 mM, about 9 mM, about 9.5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, or about 100 mM. In some aspects, the sodium salt is Na2HPO4, and is present in the medium at about 0.5 mM to about 80 mM.

In some aspects, the minimal medium disclosed herein comprises a magnesium salt at about 0.01 mM to about 10 mM, for e.g. about 0.01 mM, about 0.1 mM, about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 7.5 mM, about 8 mM, about 8.5 mM, about 9 mM, about 9.5 mM, or about 10 mM. In some aspects, the magnesium salt is MgSO4, and is present in the medium at about 0.1 mM to about 10 mM.

In some aspects, the minimal medium disclosed herein comprises a carbon source at about 0.01% to about 80%, for e.g. about 0.01%, about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 15%, about 20%, about 25%, about 30%, about 35%, about 40%, about 45%, about 50%, about 55%, about 60%, about 65%, about 70%, about 75%, or about 80%. In some aspects, the carbon source is glucose, and is present in the medium at about 0.01% to about 80%. In some aspects, the carbon source is lactose, and is present in the medium at about 0.01% to about 80%.

In some aspects, the minimal medium disclosed herein comprises a commercially available trace metal solution at about 0.01 ml/L to about 20 mL/L, for e.g., about about 0.01 ml/L, about 0.1 ml/L, about 0.5 ml/L, about 1 ml/L, about 1.5 ml/L, about 2 ml/L, about 2.5 mL/L, about 3 mL/L, about 3.5 ml/L, about 4 ml/L, about 4.5 ml/L, about 5 ml/L, about 5.5 ml/L, about 6 ml/L, about 6.5 ml/L, about 7 ml/L, about 7.5 ml/L, about 8 ml/L, about 8.5 ml/L, about 9 ml/L, about 9.5 ml/L, about 10 ml/L, or about 15 ml/L, or about 20 ml/L.

In further aspects, a biotin can be added to the medium used for culture of bacterial cells disclosed herein. In some aspects, biotin is added at about about 0.1 mg/L and about 100 mg/L. For example, components may be added in a concentration of 0.1 mg/L, about 0.5 mg/L, about 1 mg/L, about 2 mg/L, about 3 mg/L, about 4 mg/L, about 5 mg/L, about 6 mg/L, about 7 mg/L, about 8 mg/L, about 9 mg/L, about 10 mg/L, about 11 mg/L, about 12 mg/L, about 13 mg/L, about 14 mg/L, about 15 mg/L, about 16 mg/L, about 17 mg/L, about 18 mg/L, about 19 mg/L, about 20 mg/L, about 25 mg/L, about 30 mg/L, about 35 mg/L, about 40 mg/L, about 45 mg/L, about 50 mg/L, about 55 mg/L, about 60 mg/L, about 65 mg/L, about 70 mg/L, about 75 mg/L, about 80 mg/L, about 85 mg/L, about 90 mg/L, or about 100 mg/L.

In some aspects, the bacterial cells can be cultured at a temperature from about 25° C. to about 45° C. In some aspects, the culture is maintained at a temperature of about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., or about 45° C.

In some aspects, the bacterial cells can be maintained for about 30 min to about 48 hours or longer. In some aspects, the bacterial cells can be maintained for 30 min, 1 hour, 2 hours, 3 hours, 4 hours, 5 hour, 6, hours, 7 hours, 8 hours, 9 hours, 10 hours, 11 hours, 12 hours, 13 hours, 14 hours, 15 hours, 15 hours, 16 hours, 17 hours, 18 hours, 19 hours, 20 hours, 21 hours, 22 hours, 23 hours, 24 hours, 48 hours or longer.

In some aspects, the culture is maintained at a pH of about 5 to about 8. In some aspects, the culture is maintained at a pH about 5, about 5.5, about 6, about 6.5, about 7, about 7.5, or about 8.

In some aspects, the culture is maintained at a desired level of dissolved oxygen (DO) for e.g. about 5% saturation to about 50% saturation. In some aspects, the DO is about 5%, about 10%, about 15%, about 20%, about 25%, about 30%, about 35%, about 40%, or about 50%.

In some aspects, after reaching a desired cell density, the culture is induced for dsRNA production. In some aspects, dsRNA production is induced with IPTG. In some aspect. IPTG is added at about 0.1 mM to about 10 mM. In some aspects, IPTG is added at about 0.01 mM, about 0.1 mM, about 0.5 mM, about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 7.5 mM, about 8 mM, about 8.5 mM, about 9 mM, about 9.5 mM, or about 10 mM.

In some aspects, the culture is maintained to achieve a desired cell density. In some aspects, the culture growth and cell density is monitored by measuring OD600. In some aspects, the cell density of the culture medium measured using OD600 is about 10 to about 90, for e.g., about 10, about 15, about 20, about 25, about 30 about 35, about 40, about 50, about 55, about 60, about 70, about 75, about 80, about 85, or about 90.

In further aspects, culture is maintained with a controlled agitation for e.g. about 100 rpm to 2000 rpm. In some aspects, the culture is maintained with a controlled agitation of about 100 rpm, 200 rpm, 300 rpm, 400 rpm, 500 rpm, 600 rpm, 700 rpm, 800 rpm, 900 rpm, 1000 rpm, 1200 rpm, 1500 rpm, 1700 rpm, 1800 rpm, or 2000 rpm.

In some aspects, the culture is performed in large scale as a shaken culture or a bioreactor culture. In some aspects, large scale culture can comprise culture of bacterial cells in cultivation vessels from about 10 liters up to about 200 m³, about 100 liter to 100 m³, more about 100 to 1000 liters. In some aspects, the cultivation vessel can comprise a bioreactor (for e.g., a stirred tank), or simple (disposable) containers like plastic bags of up to few cubic meters, for e.g., a 500 liters plastic bag (for e.g., WAVE Bioreactor).

In some aspects, the culture can be a batch cultivation comprising a discontinuous process, where the sterile growth medium with all substrates required is initially inoculated with a pure culture of bacterial cells and no additional growth medium is added during the course of operation. In some aspects, the batch process is a partially closed system, wherein the only material added and removed during the course of operation is air/gas exchange, antifoam and pH controlling agents. In some aspects, the batch cultures are continuously shaken or stirred to keep a desired degree of homogeneity of the substrates and cells and to guarantee an as high as possible oxygen transfer for aerobic cultures.

In some aspects, the culture can be a fed-batch cultivation comprising a process, where a certain amount of fresh growth-limiting substrates is continuously added to the cultivation medium to provide their low concentrations in cultivation media and to obtain control of the growth of bacterial cells.

In some aspects, the culture disclosed herein is a high cell density culture comprising a cultivation which yields a high number of bacterial cells in a defined period of cultivation. The high-cell-density value is dependent on the microbial cell and can be defined as the cell density value that is reached with gradual addition of growth-limiting substrates (fed-batch) without intoxicating the bacterial cell, i.e. a viable cell concentration of about 3×10⁹ cells/ml, or above about 1×10¹⁰ cells/ml of E. coli.

In some aspects, the bioreactor is selected from a fed-batch system, a semi-continuous system and a continuous culture system. In some aspects, the bioreactor is a fed-batch system. In some aspects, the fed-batch system is a high-cell-density fed-batch system. In some aspects, the fed-batch system is a high-cell-density fed-batch fermentation system. The disclosed method of target dsRNA production can also be modified to adapt to any existing fermentation and/or culture system infrastructure available to the user.

In some aspects, the target dsRNA is produced at an amount of at least about 4 g/L. In some aspects, the target dsRNA is produced at an amount of at least about 5 g/L. In some aspects, the target dsRNA is produced at an amount of at least about 6 g/L. In some aspects, the target dsRNA is produced at an amount of at least about 7 g/L. In some aspects, the target dsRNA is produced at an amount of at least about 8 g/L. In some aspects, the target dsRNA is produced at an amount of at least about 9 g/L. In some aspects, the target dsRNA is produced at an amount of at least about 10 g/L. In some aspects, the target dsRNA is produced at an amount of at least about 11 g/L. In some aspects, the target dsRNA is produced at an amount of at least about 12 g/L.

In further aspects, provided herein is a method of production of a target dsRNA performed in an antibiotic-free environment. In such aspects, the problems which may accompany with the use of antibiotics for the selection of plasmids such as for example concerns of the regulatory authorities, product safety, final product analytics (depletion of the antibiotic in the product) and the risks and costs associated therewith can be circumvented. By the approach according to the disclosure the selective pressure during the fermentation process is still maintained, and this without using antibiotics in the fermentation medium.

In some aspects, the method further comprises purifying the dsRNA from the bacterial cell culture. The method comprises lysing the cells to produce a lysate and purifying the dsRNA from the cellular constituents within the lysate prior to processing the purified dsRNA for application.

In some aspects, the dsRNA is processed prior to application. Such processing may include, but is not limited to, mixing with excipients, binders or fillers to improve physical handling characteristics, stabilizers to reduce degradation, or other active agents such as chemical pesticides, fungicides, defoliants or other RNAi molecules to broaden the spectrum of application targets, and may include pelletizing, spray drying or dissolving the materials into liquid carriers.

In some aspects the dsRNA is not purified from the lysate but is processed directly for application. In some aspects, the bacterial cell is not lysed but is processed directly for application and the dsRNA remains unpurified within the processed cells. In some aspects, the bacterial cells in the culture comprising the target dsRNA is killed or inactivated prior application. The bacterial cells can be killed or inactivated by heat or other known methods in the art, which do not interfere with the activity of target dsRNA.

In some aspects, the methods disclosed herein can be further modified. As used herein, “modifying the method” can comprise modifying or changing one or more features or aspects of one or more steps of a disclosed method. In an aspect, a method can be altered for e.g., by changing the amount of salts used in the medium, used in a disclosed method, or, by changing the duration of time of the culture, or by substituting for one or more of the disclosed components used in the medium with a similar or equivalent component and/or reagent.

Application of dsRNA

In some aspects the target dsRNA produced by the disclosed constructs and methods is used for non-pharmaceutical applications. In some aspects the target dsRNA produced by the disclosed constructs and methods is used for pharmaceutical applications. In some aspects, the target dsRNA is used for controlling pests, bacteria or virus that infest plants, animals and/or humans. In some aspects, the target dsRNA is used for treating disease or conditions associated plants, animals and/or humans. It is within the working knowledge of those of ordinary skill in the art to use or adapt the disclosed plasmid vector and method of target dsRNA production for any dsRNA of interest.

Further provided herein is the use of disclosed plasmid, and/or bacterial cells comprising the plasmid for antibiotic marker free production of a target recombinant RNA. In such aspects, the recombinant RNA is a dsRNA, siRNA, shRNA, hpRNA, or miRNA. In some aspects, bacterial cells are E. coli cells. In some aspects, E. coli cells are cells without a chloramphenicol resistance gene. In some aspects, the E coli are cells without a tetracycline resistance gene.

I. dsRNA for non-pharmaceutical applications

The present disclosure encompasses methods and compositions of dsRNA which can be used for agricultural control of plant viruses, parasites, insects, nematodes fungal infections or any other organisms affecting plant growth or development.

In some aspects, delivery of dsRNA is used to control insect pest. In some aspects, delivery of dsRNA induce a lethal phenotype in the insect pest.

Suitable genes to be targeted by dsRNA in the insect pest include midgut or non-midgut genes. Representative non-limiting examples of suitable insect genes that can be targeted by dsRNA include tubulin, vATPase, acetyl choline esterase, chitin synthase gene A, beta-actin, and genes coding for inhibitors of apoptosis (e.g. IAP). The targets can also be the genes disclosed in US 2009/0285784 A1, disclosure of which is herein incorporated by reference.

In some aspects, controlling insects as used herein also encompasses inhibiting viability, growth, development or reproduction of the insect, or decreasing pathogenicity or infectivity of the insect. In some aspects, controlling insects can inhibit a biological activity in an insect, resulting in one or more of the following attributes: reduction in feeding by the insect, reduction in viability of the insect, death of the insect, inhibition of differentiation and development of the insect, absence of or reduced capacity for sexual reproduction by the insect, muscle formation, juvenile hormone formation, juvenile hormone regulation, ion regulation and transport, maintenance of cell membrane potential, amino acid biosynthesis, amino acid degradation, sperm formation, pheromone synthesis, pheromone sensing, antennae formation, wing formation, leg formation, development and differentiation, egg formation, larval maturation, digestive enzyme formation, haemolymph synthesis, haemolymph maintenance, neurotransmission, cell division, energy metabolism, respiration, apoptosis, and any component of a eukaryotic cell cytoskeletal structure, such as, actins and tubulins.

In some apects, the insect pest to be controlled is selected from the order Acari, Araneae, Anoplura, Coleoptera, Collembola, Dermaptera, Dictyoptera, Diplura, Diptera, Embioptera, Ephemeroptera, Grylloblatodea, Hemiptera, Homoptera, Hymenoptera, Isoptera, Lepidoptera, Mallophaga, Mecoptera, Neuroptera, Odonata, Orthoptera, Phasmida, Plecoptera, Protura, Psocoptera, Siphonaptera, Siphunculata, Thysanura, Strepsiptera, Thysanoptera, Trichoptera, and Zoraptera. In some aspects, the insect pest to be controlled is a member of the order Coleoptera or Lepidoptera.

In some aspects, the insect pest to be controlled is a member of the order Lepidoptera. Larvae and adults of the order Lepidoptera include, but are not limited to, armyworms, cutworms, loopers and heliothines. Larvae of the order Lepidoptera include, but are not limited to, armyworms, cutworms, loopers, and heliothines in the family Noctuidae Spodoptera frugiperda JE Smith (fall armyworm); S. exigua Hlbner (beet armyworm)/S. litura Fabricius (tobacco cutworm, cluster caterpillar) Mamestra configurata Walker (bertha armyworm); M. brassicae Linnaeus (cabbage moth); Agrotis ipsilon Hufnagel (black cutworm); A. orthogonia Morrison (western cutworm); A. subterranea Fabricius (granulate cutworm); Alabama argillacea Hübner (cotton leaf worm); Trichoplusia ni Hübner (cabbage looper); Pseudoplusia includens Walker (soybean looper); Anticarsia gemmatalis Hübner (velvetbean caterpillar); Hypena scabra Fabricius (green cloverworm); Heliothis virescens Fabricius (tobacco budworm); Pseudaletia unipuncta Haworth (armyworm); Athetis mindara Barnes and Mcdunnough (rough skinned cutworm); Euxoa messona Harris (darksided cutworm); Earias insulana Boisduval (spiny bollworm); E. vittella Fabricius (spotted bollworm) Helicoverpa armigera Hübner (American bollworm); H. zea Boddie (coin earworm or cotton bollworm); Melanchra picta Harris (zebra caterpillar); Egira (Xylomyges) curialis Grote (citrus cutworm); borers, casebearers, webworms, coneworms, and skeletonizers from the family Pyralidae Ostrinia nubilalis Hübner (European corn borer); Amyelois transitella Walker (naval orangeworm); Anagasta kuehniella Zeller (Mediterranean flour moth); Cadra cautella Walker (almond moth); Chilo suppressalis Walker (rice stem borer); C. partellus, (sorghum borer); Corcyra cephalonica Stainton (rice moth); Crambus caliginosellus Clemens (corn root webworm); C. teterrellus Zincken (bluegrass webworm); Cnaphalocrocis medinalis Guenee (rice leaf roller); Desmia funeralis Hübner (grape leaffolder); Diaphania hyalinata Linnaeus (melon worm); D, nitidalis Stoll (pickleworm); Diatraea grandiosella Dyar (southwestern corn borer). D. saccharalis Fabricius (surgarcane borer) Eoreuma loftini Dyar (Mexican rice borer); Ephestia elutella Hübner (tobacco (cacao) moth) Galleria mellonella Linnaeus (greater wax moth); Herpetogramma licarsisalis Walker (sod webworm); Homoeosoma electellum Hulst (sunflower moth); Elasmopalpus lignosellus Zeller (lesser cornstalk borer); Achroia gnsella Fabricius (lesser wax moth); Loxostege sticticalis Linnaeus (beet webworm); Orthaga thyrisalis Walker (tea tree web moth); Maruca testulalis Geyer (bean pod borer); Plodia interpunctella Hübner (Indian meal moth); Scirpophaga incertulas Walker (yellow stem borer); Udea rubigalis Guenee (celery leaftier); and leafrollers, budworms, seed worms, and fruit worms in the family Tortricidae Acleris gloverana Walsingham (Western blackheaded budworm); A. variana Femald (Eastern blackheaded budworm); Archips argyrospila Walker (fruit tree leaf roller); A. rosana Linnaeus (European leaf roller); and other Archips species, Adoxophyes orana Fischer von Rosslerstamm (summer fruit tortrix moth); Cochylis hospes Walsingham (banded sunflower moth); Cydia latiferreana Walsingham (filbertworm); C. pomonella Linnaeus (coding moth); Platynota flavedana Clemens (variegated leafroller); P. stultana Walsingham (omnivorous leafroller); Lobesia botrana Denis & Schiffermiiller (European grape vine moth); Spilonota ocellana Denis & Schiffermiiller (eyespotted bud moth); Endopiza viteana Clemens (grape berry moth); Eupoecilia ambiguella Hübner (vine moth); Bonagota salubncola Meyrick (Brazilian apple leafroller); Grapholita molesta Busck (oriental fruit moth); Suleima helianthana Riley (sunflower bud moth); Argyrotaenia spp.; Choristoneura spp. Alsophila pometaria Harris (fall cankerworm); Anarsia lineatella Zeller (peach twig borer); Anisota senatoria J. E. Smith (orange striped oakworm); Antheraea pemyi Guerin-Meneville (Chinese Oak Tussah Moth); Bombyx mori Linnaeus (Silkworm); Bucculatinx thurbeiella Busck (cotton leaf perforator); Colias eurytheme Boisduval (alfalfa caterpillar); Datana integerrima Grote & Robinson (walnut caterpillar); Dendrolimus sibiricus Tschetwerikov (Siberian silk moth), Ennomos subsignaria Hübner (elm spanworm); Erannis tiliaria Harris (linden looper); Euproctis chrysorrhoea Linnaeus (browntail moth); Harrisina americana Guerin-Meneville (grapeleaf skeletonizer); Hemileuca oliviae Cockrell (range caterpillar); Hyphantria cunea Drury (fall webworm); Keiferia lycopersicella Walsingham (tomato pinworm); Lambdina fiscellaria fiscellaria Hulst (Eastern hemlock looper); L. fiscellaria L.ugubrosa Hulst (Western hemlock looper); Leucoma licis Linnaeus (satin moth); Lymantria dispar Linnaeus (gypsy moth); Manduca quinquemaculata Haworth (five spotted hawk moth, tomato homworm); M. sexta Haworth (tomato hornworm, tobacco hornworm); Operophtera brumata Linnaeus (winter moth); Paleacrita vernata Peck (spring cankerworm); Papilio cresphontes Cramer (giant swallowtail, orange dog); Phryganidia califormica Packard (California oakworm); Phyllocnistis citrella Stainton (citrus leafminer); Phyllonorvcter blancardella Fabricius (spotted tentiform leafminer); Pieris brassicae Linnaeus (large white butterfly); P. rapae Linnaeus (small white butterfly); P. napi Linnaeus (green veined white butterfly); Platyptilia carduidactyla Riley (artichoke plume moth); Plutella xylostella Linnaeus (diamondback moth); Pectinophora gossypiella Saunders (pink bollworm); Pontia protodice Boisduval & Leconte (Southern cabbageworm); Sabulodes aegrotata Guenee (omnivorous looper); Schizura concinna J. E. Smith (red humped caterpillar); Sitotroga cerealella Olivier (Angoumois grain moth); Thaumetopoea pityocampa Schiffermuller (pine processionary caterpillar); Tineola bisselliella Hummel (webbing clothesmoth); Tuta absoluta Meyrick (tomato leafminer); Yponomeuta padella Linnaeus (ermine moth); Heliothis subflexa Guenee; Malacosoma spp, and Orgyia spp. Tenebhonidae.

In some aspects, the insect pest to be controlled is a member of the order Coleoptera. Larvae and adults of the order Coleoptera include weevils from the families Anthribidae, Bruchidae, and Curculionidae (including, but not limited to: Anthonomus grandis Boheman (boll weevil); Lissorhoptrus oryzophilus Kuschel (rice water weevil); Sitophilus granarius Linnaeus (granary weevil); S, oryzae Linnaeus (rice weevil); Hypera punctata Fabricius (clover leaf weevil); Cylindrocopturus adspersus LeConte (sunflower stem weevil); Smicronyx fulvus LeConte (red sunflower seed weevil); S. sordidus LeConte (gray sunflower seed weevil); Sphenophorus maidis Chittenden (maize billbug); flea beetles, cucumber beetles, rootworms, leaf beetles, potato beetles, and leafminers in the family Chrysomelidae (including, but not limited to: Leptinotarsa decemlineata Say (Colorado potato beetle); Diabrotica virgifera virgifera LeConte (western corn rootworm); D. barberi Smith & Lawrence (northern corn rootwormj: D. undecimpunctata howardi Barber (southern corn rootworm); Chaetocnema pulicaria Melsheimer (corn flea beetle); Phyllotreta cruciferae Goeze (corn flea beetle); Colaspis brunnea Fabricius (grape colaspis); Oulema melanopus Linnaeus (cereal leaf beetle); Zygogramma exclamationis Fabricius (sunflower beetle)); beetles from the family Coccinellidae (including, but not limited to: Epilachna vafvestis Mulsant (Mexican bean beetle)); chafers and other beetles from the family Scarabaeidae (including, but not limited to: Popilliajaponica Newman (Japanese beetle); Cyclocephala borealis Arrow (northern masked chafer, white grub); C. immaculata Olivier (southern masked chafer, white grub); Rhizotrogus majalis Razoumowsky (European chafer); Phyllophaga crinita Burmeister (white grub); Ligyrus gibbosus De Geer (carrot beetle)), carpet beetles from the family Dermestidae; wireworms from the family Elatehdae, Eleodes spp., Melanotus spp.; Conoderus spp.; Limonius spp.; Agriotes spp.; Ctenicera spp.; Aeolus spp.; bark beetles from the family Scolytidae and beetles from the family Tenebhonidae.

In certain aspects, the insect pest to be controlled is a member of the order Hymenoptera such as an ant, sawfly, wasp or bee. In some aspects, the insect pest to be controlled is an ant (Formicoidea), selected from Tapinoma sessile (odorous ants). Solenopsis spp. (e.g. Solenopsis invicta (Fire Ant)), Monomorium spp. (e.g. Monomorium pharaonis (Pharaoh Ant)), Camponotus spp. (e.g. Camponotus spp (Carpenter Ants)), Lasius spp. (e.g. Lasius niger (Small Black Ant)). Tetramorium spp. (e.g. Tetramorium caespitum (Pavement Ant)), Myrmica spp. (e.g. Myrmica rubra (Red Ant)), Formica spp (wood ants), Crematogaster spp. (e.g. Crematogaster lineolata (Acrobat Ant)), Iridomyrmex spp. (e.g. Iridomyrmex humilis (Argentine Ant)), Pheidole spp. (Big Headed Ants), and Dasymutilla spp. (e.g. Dasymutilla occidentalis (Velvet Ant)).

In certain aspects, the insect pest to be controlled is a termite (Isoptera and/or Termitidae) selected from Amitermes spp. (e.g. Amitermes floridensis (Florida dark-winged subterranean termite)), Reticulitermes spp. (e.g. Reticulitermes flavipes (the eastern subterranean termite), Reticulitermes hesperus (Western Subterranean Termite)), Coptotermes spp. (e.g. Coptotermes formosanus (Formosan Subterranean Termite)). Incisitermes spp. (e.g. Incisitermes minor (Western Drywood Termite)), Neotermes spp. (e.g. Neotermes connexus (Forest Tree Termite)).

In certain aspects, the insect pest to be controlled is a member of the order Diptera such as a mosquito or fly. e.g. A. gambiae (malaria mosquito) or Ae. aegypti (yellow fever mosquito).

In certain aspects, the insect pest to be controlled is a member of the order Acari (e.g. ticks or mites) selected from the families Argasidae, Dermanyssidae, Ixodidae, Psoroptidae or Sarcoptidae and representatives of the species Amblyomma spp., Anocentor spp., Argas spp., Boophilus spp., Cheyletiella spp., Chorioptes spp., Demodex spp., Dermacentor spp., Denmanyssus spp., Haemophysalis spp., Hyalomma spp., Ixodes spp., Lynxacarus spp., Mesostigmata spp., Notoedres spp., Omithodoros spp., Omithonyssus spp., Otobius spp., Otodectes spp., Pneumonyssus spp., Psoroptes spp., Rhipicephalus spp., Sarcoptes spp., or Trombicula spp.; Anoplura (sucking and biting lice) selected from the species Bovicola spp., Haematopinus spp., Linognathus spp., Menopon spp., Pediculus spp., Pemphigus spp., Phylloxera spp., or Solenopotes spp.; Diptera (flies) such as representatives of the species Aedes spp., Anopheles spp., Calliphora spp., Chrysomyia spp., Chrysops spp., Cochliomyia spp., Culex spp., Culicoides spp., Cuterebra spp., Dermatobia spp., Gastrophilus spp., Glossina spp., Haematobia spp., Haematopota spp., Hippobosca spp., Hypoderma spp., Lucilia spp., Lyperosia spp., Melophagus spp., Oestrus spp., Phaenicia spp., Phlebotomus spp., Phormia spp., Sarcophaga spp., Simulium spp., Stomoxys spp., Tabanus spp., Tannia spp, or Tipula spp.; Mallophaga (biting lice) selected from the species Damalina spp., Felicola spp., Heterodoxus spp, or Trichodectes spp.; or Siphonaptera (wingless insects) selected from the species Ceratophyllus spp., spp., Pulex spp., or Xenopsylla spp; Cimicidae (true bugs) selected from the species Cimex spp., Tritominae spp., Rhodinius spp., or Triatoma spp.; Blattodea (cockroach) such as Blatella spp. (e.g. Blatella germanica (german cockroach)), Periplaneta spp. (e.g. Periplaneta americana (American cockroach) and Periplaneta australiasiae (Australian cockroach)), Blatta spp. (e.g. Blatta orientalis (Oriental cockroach)) and Supella spp. (e.g. Supella longipalpa (brown-banded cockroach); and other insects such as Dermaptera (earwigs), Heteroptera (e.g. bed bug), Siphonaptera (flea), Stemorrhyncha (aphids), or Zygentoma (silverfish), crickets, silverfish, booklice and beetles.

In some aspects, the dsRNA targets a plant virus or viroid. Exemplary viruses and viroids include a virus or viroid of the family; Alphaflexiviridae (Potato Virus X (PVX)); Bromoviridae (Alfalfa Mosaic Virus (AMV), Cucumber Mosaic Virus, and Brome Mosaic Virus (BMV)); Bunyaviridae (Tomato Spotted Wilt virus); Caulimoviridae (Cauliflower Mosaic Virus (CaMV) and Rice Tungro Bacilliform Virus); Closteroviridae (Citrus Tristeza Virus); Geminiviridae (Mungbean Yellow Mosaic India Virus, African Cassava Mosaic Virus. Tomato Yellow Leaf Curl Sardinia Virus. Tomato Yellow Leaf Curl Virus, and African Cassava Mosaic Virus); Luteoviridae (Barley Yellow Dwarf Virus, and Potato Leafroll Virus); Pospiviroidae (Potato Spindle Tuber Viroid); Potyviridae (Potato Virus Y (PVY), Tobacco Etch Virus (TEV), Papaya Ringspot Virus type W (PRSV-W), Plum Pox Virus (PPV), Sugarcane Mosaic Virus, Bean Common Mosaic Virus and Cassava Brown Streak Virus); Sequiviridae (Rice Tungro Spherical Virus); Tombusviridae (Maize Chlorotic Mottle Virus and Tomato Bushy Stunt Virus); and Virgaviridae (Tobacco Mosaic Virus (TMV). Tomato Mosaic Virus, Pepper Mild Mottle Virus (PMMoV) Cucumber Green Mottle Mosaic Virus) and Benyvirus (Beet Necrotic Yellow Vein Virus).

In some aspects, the dsRNA controls a plant fungus. Non-limiting examples of fungi include Magnaporthe species (Magnaporrhe oryzae), Botryhs species especially Borrytis cinema), Puccinia species, Fusarium species (Fusarium graminearum and Fusarium oxysporum), Blumeria species (Blumeria graminis f. sp Mycosphaerella species (Mycosphaerella graminicola), Colletotrichum species. Ustilago species (Ustilago maydis), Melampsora species (Melampsora lini), Phakopsora species (Phakopsora pachyrhizi), Rhizoctonia species (Rhizoctonia solani) and Aspergillus species.

In some aspects, the dsRNA controls oomycetes. Non-limiting examples of oomycetes include Phytophthora species, Phytophthora infestans, Hyaloperonospora arabidopsidis, Phytophthora ramorum, Ramorum disease. Phytophthora sojae, Phytophthora capsica, Plasmopara viticola, Phytophthora cinnamomic, Phytophthora parasitica, Pythium ultimum, Albugo candida, Aphanomyces euteiches, Albugo laibachii, Bremia lactucae, Phytophthora palmivora, Pseudoperonospora cubensis, Plasmopara halstedii, Peronophythora litchi, Peronosclerospora sorghi, Peronospora belbahrii, Phytophthora alni, Phytophthora brassicae, Phytophthora cactorum, Phytophthora meadii, Phytophthora phaseoli, Phytophthora plurivora (formerly P. citricola), Plasmopara obducens, Pythium aphanidermatum, Pvthium oligandrum, Sclerophthora rayssiae, Hyaloperonospora brassicae, Saprolegnia parasitica (fish parasite), Lagenidium giganteum (mosquito parasite), Pythium insidiosum (mammalian parasite)

dsRNA of the present disclosure can be engineered to control nematode (e.g root knot nematode) or parasitic weed (e.g. Striga asiatica L). A skilled person in art can construct dsRNA based on any organism against which protection is sought, and an appropriate sequence could readily be selected by the skilled person.

In some aspects, the bacterial cells comprising the dsRNA, or purified or partially purified dsRNA may be formulated with a suitable carrier or excipient or diluent. The formulation may be in any physical form suitable for application, such as solid form such as a powder, pellet or a bait, liquid form such as a spray, or gel, coat or paste form. In some aspects, the formulation comprises components which serve to stabilise the dsRNA and/or prevent degradation of the dsRNA during storage. In some aspects, the formulation comprises components which enhance or promote uptake of the dsRNA by the insect such as chemical agents which generally promote the uptake of RNA into cells (e.g. lipofectamine). In some aspects, the composition may also include one or more other active ingredients or ingredient that provides benefit to a plant. Such active ingredient may be, for example, an insecticide, a pesticide, a fungicide, an antibiotic, an insect repellant, an anti-parasitic agent, an anti-viral agent, or a nematicide.

II. dsRNA for Pharmaceutical Applications

In some aspects, delivery of dsRNA is used for pharmaceutical applications.

In some aspects delivery of dsRNA treats a condition, disease or disorder in a subject, wherein the dsRNA targets a gene associated with a condition, disease or disorder in a subject. The target gene is selected from the group comprising of oncogenes, cytokine genes, Inhibitors of DNA binding and cell differentiation (Id) protein genes, genes involved in development and prion genes. In some aspects, the target gene is expressed in pathogenic organisms, such as virus, viroid, bacteria, fungi or plasmodia. In some aspects, dsRNA is used in the therapy of genetically controlled diseases, such as cancer, viral diseases or Alzheimer's disease. In some aspects, the dsRNA can be used for targeting genes in a specific site or organ such as the liver, which is responsible for certain metabolic diseases or disorders such as high cholesterol levels or HIV-infected cells. In some aspects, dsRNA is used in a vaccine to prevent or protect a subject from contracting a disease or condition.

In some aspects, the disease to be treated is a cancer. The disclosed dsRNA can be used for targeted for treatment or development of treatments for cancers of any type, including solid tumors and leukemias, including: apudoma, choristoma, branchioma, malignant carcinoid syndrome, carcinoid heart disease, carcinoma (e.g., Walker, basal cell, basosquamous, Brown-Pearce, ductal, Ehrlich tumor, in situ, Krebs 2, Merkel cell, mucinous, non-small cell lung, oat cell, papillary, scirrhous, bronchiolar, bronchogenic, squamous cell, and transitional cell), histiocytic disorders, leukemia (e.g., B cell, mixed cell, null cell, T cell, T-cell chronic, HTLV-II-associated, lymphocytic acute, lymphocytic chronic, mast cell, and myeloid), histiocytosis malignant, Hodgkin disease, immunoproliferative small, non-Hodgkin lymphoma, plasmacytoma, reticuloendotheliosis, melanoma, chondroblastoma, chondroma, chondrosarcoma, fibroma, fibrosarcoma, giant cell tumors, histiocytoma, lipoma, liposarcoma, mesothe-lioma, myxoma, myxosarcoma, osteoma, osteosarcoma, Ewing sarcoma, synovioma, adenofibroma, adenolymphoma, carcinosarcoma, chordoma, cranio-pharyngioma, dysgerminoma, hamartoma, mesenchymoma, mesonephroma, myosarcoma, amelo-blastoma, cementoma, odontoma, teratoma, thymoma, trophoblastic tumor, adeno-carcinoma, adenoma, cholangioma, cholesteatoma, cylindroma, cystadenocarcinoma, cystadenoma, granulosa cell tumor, gynandroblastoma, hepatoma, hidradenoma, islet cell tumor. Leydig cell tumor, papilloma, Sertoli cell tumor, theca cell tumor, leiomyoma, leiomyosarcoma, myoblastoma, myoma, myosarcoma, rhabdomyoma, rhabdomyo-sarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaflin, angiokeratoma, angiolymphoid hype lasia with eosinophilia, angioma sclerosing, angiomatosis, glomangioma, hemangioendothelioma, hemangioma, hemangiopericytoma, hemangiosarcoma, lymphangioma, lymphangio-myoma, lymphangiosarcoma, pinealoma, carcinosarcoma, chondrosarcoma, cystosarcoma phyllodes, fibrosarcoma, hemangiosarcoma, leiomyosarcoma, leukosarcoma, liposarcoma, lymphangiosarcoma, myosarcoma, myxosarcoma, ovarian carcinoma, rhabdomyo-sarcoma, sarcoma (e.g., Ewing, experimental, Kaposi, and mast cell), neoplasms (e.g., bone, breast, digestive system, colorectal, liver, pancreatic, pituitary, testicular, orbital, head and neck, central nervous system, acoustic, pelvic, respiratory tract, and urogenital), neurofibromatosis, and cervical dysplasia.

The possible target genes for dsRNA by way of non-limiting example include developmental genes (e.g., adhesion molecules, cyclin kinase inhibitors, Wnt family members, Pax family members, Winged helix family members, Hox family members, cytokines/lymphokines and their receptors, growth/differentiation factors and their receptors, neurotransmitters and their receptors); oncogenes (e.g., ABL1. BCL1, BCL2, BCL6, CBFA2, CBL, CSF1R, ERBA, ERBB, EBRB2, ETS1, ETS1, ETV6, FGR, FOS, FYN, HCR, HRAS, JUN, KRAS, LCK, LYN, MDM2, MLL, MYB, MYC, MYCL1, MYCN, NRAS, PIM1, PML, RET, SRC, TALI, TCL3, and YES); tumor suppressor genes (e.g., APC, BRCA1, BRCA2, MADH4, MCC, NF1, NF2, RBI, TP53, and WT1); and enzymes (e.g., ACC synthases and oxidases, ACP desaturases and hydroxylases, ADP-glucose pyrophorylases. ATPases, alcohol dehydrogenases, amylases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, lipases, lipoxygenases, lysozymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, apolipoprotein B, pcsk9, and xylanase.

The dsRNA disclosed herein can be used to treat or prevent viral infections. Non-limiting examples include human retroviruses infections including HIV, members of the herpes virus family, e.g., herpes simplex and herpes zoster; cytomegalovirus or CMV (sometimes classified as a herpes-type virus), localized skin cancer with and without vital etiology, susceptible sexually transmitted viral conditions and venereal infections including chlamydia, and venereal infections in which the causative viral agent is primary or secondary to another venereal infection.

The viral pathogens can include, include, but are not limited to, Orthomyxoviruses, such as influenza virus. Retroviruses, such as RSV, HTLV-1, and HTLV-II. Herpesviruses such as EBV; CMV or herpes simplex virus; Lentiviruses, such as HIV-1 and HIV-2; Rhabdoviruses, such as rabies; Picornoviruses, such as Poliovirus; Poxviruses, such as vaccinia; Rotavirus; Parvoviruses, such as adeno-associated virus 1 and Coronavirues such as alphacoronavirus, betacoronavirus, gammacoronavirus and deltacoronavirus. Other viruses that may be targeted by the present disclosure include, but are not limited to, influenza, coxsacchie, herpes simplex virus Type I and 2, St. Louis encephalitis, Epstein-Barr, myxoviruses, JC, coxsakieviruses B, togaviruses, measles, paramyxoviruses, echoviruses, bunyaviruses, cytomegaloviruses, varicella-zoster, mumps, equine encephalitis, lymphocytic choriomeningitis, rhabodoviruses including rabies, simian virus 40, human polyoma virus, parvoviruses, papilloma viruses, primate adenoviruses, coronaviruses, retroviruses, Dengue, yellow fever, Japanese encephalitis virus, and/or BK, or any viruses of the species/family Astoviridae, Togaviridae, Flaviviridae, paramyxoviridae, arteriviruses, Rhabdoviridae, Filoviridae, orthomyxoviridae, bunyaviridae, arenaviridae, reoviridae, Bimaviridae, circoviridae, adenoviridae, Iridoviridae, Retrovirus, Herpesvirus, Hepadenovirus, Papillomavirus, and Papovavirus.

Some non-limiting examples for viral targets include but not limited to the human immunodeficiency virus Nef, Gag, Env, Tat, mutant derivatives of Tat, such as Tat-Δ31-45, and Pol and T and B cell epitopes of gp120, chimeric derivatives of HIV-1 Env and gp120, such as but not restricted to fusion between gp120 and CD4; truncated or modified derivatives of HIV-1 env, such as but not restricted to gpl40 or derivatives of HIV-1 Env and/or gp140 thereof, the hepatitis B viral targets, rotavirus, such as VP4 and VP7, influenza virus hemagglutinin or nucleoprotein, and herpes simplex virus thymidine kinase.

Infections by other pathogens may also be treated or prevented using the methods of the present disclosure, including protozoa, bacteria, yeast, and fungal infections. Non-limiting examples of bacterial pathogens include Mycobacterium spp., Helicobacter pylori, Salmonella spp., Shigella spp., E. coli. Rickettsia spp., Listeria spp., Legionella pneumoniae, Pseudomonas spp., Vibrio spp., and Borrelia burgdorferi. The dsRNA targets for bacterial infection can be from enterotoxigenic E. coli, such as the CFA/I fimbrial antigen and the nontoxic B-subunit of the heat-labile toxin; pertactin of Bordetella pertussis, adenylate cyclase-hemolysin of B. pertussis, fragment C of tetanus toxin of Clostridium tetani, OspA of Borrelia burgdorferi, protective paracrystalline-surface layer proteins of Rickettsia prowazekii and Rickettsia typhi, the listeriolysin (also known as “Llo” and “Hly”) and/or the superoxide dismutase (also know as “SOD” and “p60”) of Listeria monocytogenes, the urease of Helicobacter pylori, and the receptor-binding domain of lethal toxin and/or the protective antigen of Bacillus anthrax.

The dsRNA of the present disclosure can target parasitic pathogens, not limited to, Plasmzodium spp., such as Plasmodium falciparum; Trypanosome spp., such as Trypanosoma cruzi; Giardia spp., such as Giardia intestinalis; Boophilus spp., Babesia spp., such as Babesia microti; Entamoeba spp., such as Entamoeba histolytica; Eimeria spp., such as Eimeria maxima; Leishmania spp.; Schistosome spp., Brugia spp., Fascida spp., Dirofilaria spp., Wuchereria spp., and Onchocerea spp. For example, for intracellular parasites such as Plasmodia, pathogen-specific dsRNAs effective against the pathogen may be delivered to cells susceptible to infection.

In some aspects, dsRNA is administered as a formulation. In some aspects, the bacterial cells comprising the dsRNA, unpurified or partially purified, may be formulated with a suitable carrier or excipient or diluent. In some aspects, the present disclosure encompasses administration of dsRNA, directly.

dsRNA can be formulated and administered by several different means. For instance, a composition can generally be administered parenterally, intraperitoneally, intravascularly, transdermally, subcutaneously, or intrapulmonarily in dosage unit formulations containing conventional nontoxic pharmaceutically acceptable adjuvants, carriers, excipients, and vehicles as desired. The term parenteral as used herein includes subcutaneous, intravenous, intramuscular, intrathecal, or intrasternal injection, or infusion techniques. Formulation of pharmaceutical compositions is discussed in, for example, Hoover, John E., Remington's Pharmaceutical Sciences, Mack Publishing Co., Easton, Pa. (1975), and Liberman, H. A, and Lachman. L., Eds., Pharmaceutical Dosage Forms. Marcel Decker, New York, N.Y. (1980).

A pharmaceutical formulation as disclosed herein comprises one or more pharmaceutically acceptable excipients. Non-limiting examples of excipients include chemical enhancers, humectants, pressure sensitive adhesives, antioxidants, solubilizers, thickening agents, plasticizers, adjuvants, carriers, excipients, vehicles, coatings, and any combinations thereof. One or more excipients can be selected for oral, transdermal, parenteral, intraperitoneal, intravascular, subcutaneous, by inhalation spray, rectal, or intrapulmonary administration.

Kits for Production of dsRNA

In some aspects, the invention comprises a kit for production of dsRNA. The kit comprises a plasmid vector for transient expression of a target dsRNA in preselected cells and/or a genetically modified cells that can be cultured and maintained for the production of the target dsRNA. The kit can further comprise chemicals, equipment and instructions necessary for setting up target dsRNA production and/or administration of dsRNA for desired purposes.

### EXAMPLES

Unless specified, reagents employed in the examples are commercially available or can be prepared using commercially available instrumentation, methods, or reagents known in the art. The examples illustrate various aspects of the invention and practice of the methods of the invention. The examples are not intended to provide an exhaustive description of the many different aspects of the invention. Thus, although the invention has been described in some detail by way of illustration and example for purposes of clarity of understanding, those of ordinary skill in the art will realize readily that many changes and modifications can be made thereto without departing from the spirit or scope of the appended claims.

It is understood that the examples and aspects described herein are for illustrative purposes only and that various modifications or changes in light thereof will be suggested to persons skilled in the art and are to be included within the spirit and preview of this application and scope of the appended claims.

### Example 1

### Bacterial Strains.

Escherichia coli NEB® 5-α (NEB, Ipswich, MA) was used in all cloning experiments. E. coli HT115(DE3) with genotype F⁻, mcrA, mcrB, IN(rmD-rrnE)1, mc14::Tn10(DE3) lysogen: lacUV5 promoter-T7 polymerase) was obtained from University of Minnesota Caenorhabditis genetics center (Minneapolis, MN) and was used for dsRNA production in shake flasks and batch-fed fermentations. E. coli JM109(DE3) (Promega. Madison, WI) was used to study the impact of ribonuclease III (RNase III) on MS2 CP-mediated accumulation of dsRNA inside E. coli cells. All E. coli strains were maintained on LB media with appropriate antibiotics.

### Construction of Plasmid Vectors

Vector pBR322 was used to create the dsRNA production vector. A custom synthetic DNA fragment comprised of the T7 promoter sequence driving transcription of a single copy of the bacteriophage MS2 capsid gene followed by a T7 terminator was obtained from GenScript (Piscataway, NJ). The fragment was amplified with primer pairs P1 and P2 (Table 1) and cloned into BamHI/SphI restriction sites of pBR322 to create plasmid pAPSE10118. A second synthetic sequence comprised of the T7 promoter sequence followed by an MS2 pac site sequence, a multiple cloning site; containing in order from 5′ to 3′: AsiSI-PmeI-AscI-RsrII-NotI-PacI restriction sites, a second high affinity variant MS2 pac type sequence (C-pac), a T7 terminator, and a SphI restriction site was obtained from GenScript. This fragment was PCR amplified with primers P3 and P4 and cloned into EcoR1/EcoRV sites of pAPSE10118 creating plasmid pAPSE10136. To make the Colorado potato beetle (CPB) (Leptinotarsa decemlineata (Say)) β-actin (NCBI accession no: KJ577616) hairpin construct, a synthetic 294-bp long sequence starting 49 bp upstream of the first codon was obtained and primer pairs P5/P6 and P7/P8 were used to amplify sense and antisense sequences, respectively, along with the loop region for cloning into pAPSE10136 via Gibson assembly. The pAPSE10136 μlasmid backbone was PCR amplified with primers P9/P10 and CPB β-actin sense and antisense fragments were assembled to generate vector pAPSE10218.

To construct plasmids including hairpin constructs containing either zero pac sites (pAPSE10279), one 5′ pac site (pAPSE10338) or one 3′pac site (pAPSE10219), a series of synthetic T7promoter—T7 terminator sequences including zero, one 5′pac site or one 3′ pac site were obtained. Each was PCR amplified with primers P3/P4 and cloned into the EcoR1/EcoRV site of plasmid pAPSE10118 followed by cloning of CPB β-actin hairpin construct via Gibson assembly as described above for creating pAPSE10218. For adding the third pac site in the hairpin loop region, the reverse sense primer and the forward antisense primers were modified to add the pac site to the loop region creating plasmid pAPSE10777.

To create a red imported fire ant (RIFA) (Solenopsis invicta) β-actin hairpin construct, a synthetic sequence comprising a 300-bp long region of RIFA β-actin (NCBI accession no. XM_011175337) was used as template to amplify the sense and antisense sequences with primer pairs P11/P12 and P13/P14, respectively, along with the loop region. The construct was then introduced into pAPSE10136 via Gibson assembly creating plasmid pAPSE10379. To clone the pyrE expression cassette, the rmB T1-T2 terminator sequence was amplified with forward primer P15 carrying SalI and AvrII sites and reverse primer P16 carrying a NruI site from synthetic DNA and cloned into the SalI/NruI site of pAPSE10379 creating plasmid pAPSE10424. The pyrE coding sequence was then amplified from E. coli NEB® 5-α genomic DNA with primers P17/P18 and cloned into the SalI/AvrII site of pAPSE10424 creating plasmid pAPSE10448. Plasmid pAPSE10775 was created by excising the CPB β-actin hairpin cassette (T7Promoter-CPB βactin hairpin-T7 terminator) as a EcoRI/NotI fragment from pAPSE10218 and cloning into the EcoRI/NotI site of pAPSE10448.

To create a vector pAPSE10402 comprising the cassette for production of intermolecular dsRNA by bi-directional transcription, a synthetic DNA fragment consisting of: T7terminator-T7promoter-MS2 pac site-294 bp CPB β-actin sense strand (Genscript, Piscataway, NJ) was amplified with primers P3/P19 and cloned into EcoRI/PacI sites of vector pAPSE10218 creating vector pAPSE10400. A second synthetic DNA sequence consisting of: MS2 pac site-T7promoter-T7 terminator was amplified with primers P20/P21 and cloned into the PacI/EcoRV site of vector pAPSE10400 creating plasmid pAPSE10402. Plasmid pAPSE10776 was constructed by excising the CPB β-actin hairpin and MS2 CP cassettes as a EcoRI/SalI fragment and cloning into same site of pUC19. For constructing vector pAPSE10773, a synthetic DNA fragment comprised of the T7 promoter driving eGFP coding sequence followed by the T7 terminator was amplified using primer pairs P22/P23 and cloned into the BglII/SalI site of vector pAPSE10218. Vector pAPSE10305 was constructed by excising MS2 CP expression cassette from pAPSE10218 by EcoRV/NruI digestion and re-ligating the pAPSE10218 backbone.

Primers used for cloning are listed in Table 1 along with primer sequences. The plasmid vectors described are summarized in Table 2. All constructs were confirmed by Sanger sequencing.

TABLE 1
Primers used in the study.
Restriction site sequences are underlined.

| Primer | |
|---|---|
| ID | Primer sequence |
| P1 | ATTTAGGATCCAGATCTCGATCCCG |
| | (SEQ ID NO: 10) |
| P2 | TTTATGCATGCCGGATATAGTTCCTCC |
| | (SEQ ID NO: 11) |
| P3 | CGTCTTCAAGAATTCCGAAATTAATACG |
| | (SEQ ID NO: 12) |
| P4 | ATAATGCGCATGCCCGCCGCCCAGTC |
| | (SEQ ID NO: 13) |
| P5 | GAGGATTACCCATGTGCGATCGCGCACGAGGTTTTTC |
| | TGTCTAGTG |
| | (SEQ ID NO: 14) |
| P6 | CGATGTGGCCTGAGGAATACCTCTCTCATTGCGTTTA |
| | GATAAAGCATCCCGCTGGAAAGGGAACCAGTACTTTG |
| | TAAAGCAGCTAGAGGGTTTAAACTCATCCCAGTTGGT |
| | GATGATACC |
| | (SEQ ID NO: 15) |
| P7 | GAGAGGTATTCCTCAGGCCACATCGCTTCCTAGTTCC |
| | GCTGGGATCCATCGTTGGCGGCCGAAGCCGCCATTCC |
| | ATAGTGAGTTCTGGCGCGCCTCATCCCAGTTGGTGAT |
| | GATACC |
| | (SEQ ID NO: 16) |
| P8 | GTTCAGACCGGGTATTCGGTCCGGCACGAGGTTTTTC |
| | TGTCTAGTG |
| | (SEQ ID NO: 17) |
| P9 | CGGACCGAATACCCGGTCTGAAC |
| | (SEQ ID NO: 18) |
| P10 | GCGATCGCACATGGGTAATCCTC |
| | (SEQ ID NO: 19) |
| P11 | GAGGATTACCCATGTGCGATCGCcGATCTCTCTCCCT |
| | CGACTCTAAC |
| | (SEQ ID NO: 20) |
| P12 | CGATGTGGCCTGAGGAATACCTCTCTCATTGCGTTTA |
| | GATAAAGCATCCCGCTGGAAAGGGAACCAGTACTTTG |
| | TAAAGCAGCTAGAGGGTTTAAACCCATGTCATCCCAA |
| | TTAGTAATA |
| | (SEQ ID NO: 21) |
| P13 | GAGAGGTATTCCTCAGGCCACATCGCTTCCTAGTTCC |
| | GCTGGGATCCATCGTTGGCGGCCGAAGCCGCCATTCC |
| | ATAGTGAGTTCTGGCGCGCCCCATGTCATCCCAATTA |
| | GTAATA |
| | (SEQ ID NO: 22) |
| P14 | GTTCAGACCGGGTATTCGGTCCGCGATCTCTCTCCCT |
| | CGACTCTAAC |
| | (SEQ ID NO: 23) |
| P15 | ATTAGTCGACAATCCTAGGCAGAACGCAGAAGCTGTC |
| | TG |
| | (SEQ ID NO: 24) |
| P16 | ATAATCGCGAGTAGAAACGCAAAAAGGCCATC |
| | (SEQ ID NO: 25) |
| P17 | ATTGTCGACAGAAGGAGATATACATATGAAACCATAT |
| | CAGCGCCAGTTTATTGA |
| | (SEQ ID NO: 26) |
| P18 | TAAATCCCTAGGTTAAACGCCAAACTCTTCGCG |
| | (SEQ ID NO: 27) |
| P19 | ACTCCTTAATTAACTCTAAGTACTTCTTG |
| | (SEQ ID NO: 28) |
| P20 | GAGTTAATTAAGGAGTTCAAACATGG |
| | (SEQ ID NO: 29) |
| P21 | TGTGGATATCCGGATATAGTTCC |
| | (SEQ ID NO: 30) |
| P22 | ATCATGGCGACCACACCCGT |
| | (SEQ ID NO: 31) |
| P23 | ACTGGGTTGAAGGCTCTCAAGGG |
| | (SEQ ID NO: 32) |

TABLE 2
Summary of plasmids used in the experiments

| Plasmid ID | Description |
|---|---|
| pAPSE10218 | pBR322 replicon, contains CPB β-actin hairpin with one |
| | pac site each at 3′ and 5′ends, contains MS2 CP cassette. |
| pAPSE10219 | pBR322 replicon, contains CPB β-actin hairpin with one |
| | pac site at 3′end, contains MS2 CP cassette. |
| pAPSE10279 | pBR322 replicon, contains CPB β-actin hairpin without |
| | any pac sites, contains MS2 CP cassette. |
| pAPSE10305 | pBR322 replicon, contains CPB β-actin hairpin with one |
| | pac site each at 3′ and 5′ends, lacks MS2 CP cassette. |
| pAPSE10338 | pBR322 replicon, contains CPB β-actin hairpin with one |
| | pac site at 5′end, contains MS2 CP cassette. |
| pAPSE10379 | pBR322 replicon, contains RIFA β-actin hairpin with one |
| | pac site each at 3′ and 5′ends, contains MS2 CP cassette. |
| pAPSE10402 | pBR322 replicon, contains CPB β-actin sense strand for |
| | bidirectional transcription with two convergent T7 |
| | promoters, contains two pac sites, one each at 3′ and |
| | 5′ends, contains MS2 CP cassette. |
| pAPSE10448 | pBR322 replicon, contains RIFA β-actin hairpin with |
| | one pac site each at 3′ and 5′ends, contains MS2 CP |
| | cassette, and RBS-pyrE cds-T1-T2 terminator |
| | downstream of MS2 CP cassette. |
| pAPSE10773 | pBR322 replicon, contains CPB β-actin hairpin with one |
| | pac site each at 3′ and 5′ends, contains eGFP under the |
| | control of T7 expression cassette. |
| pAPSE10775 | pBR322 replicon, contains CPB β-actin hairpin with one |
| | pac site each at 3′ and 5′ends, contains MS2 CP cassette, |
| | and RBS-pyrE cds-T1-T2 terminator downstream of |
| | MS2 CP cassette. |
| pAPSE10776 | pUC19 replicon, contains CPB β-actin hairpin with one |
| | pac site each at 3′ and 5′ends, contains MS2 CP cassette. |
| pAPSE10777 | pBR322 replicon, contains CPB β-actin hairpin with one |
| | pac site each at 3′ and 5′ends and a third pac site |
| | in the loop region, contains MS2 CP cassette. |
| pAPSE10471 | pBR322 replicon, contains RIFA β-actin hairpin with one |
| | pac site each at 3′ and 5′ends, contains MS2 CP cassette, |
| | and T7-RBS-pyrE cds-T1-T2 terminator downstream of |
| | MS2 CP cassette, with pyrE under the control of |
| | dedicated T7 promoter |
| pAPSE10772 | pBR322 replicon, contains RIFA β-actin hairpin with one |
| | pac site each at 3′ and 5′ends, contains MS2 CP cassette, |
| | and RBS-pyrE cds-T1-T2 terminator downstream of |
| | MS2 CP cassette, carries Corynebacterium replicon |
| pAPSE10500 | pBR322 replicon, contains RIFA β-actin hairpin with one |
| | pac site each at 3′ and 5′ends, contains MS2 CP cassette, |
| | lacks pyRE, carries Corynebacterium replicon |
| pAPSE10797 | pBR322 replicon, contains CPB β-actin hairpin with one |
| | pac site each at 3′ and 5′ends, contains MS2 CP cassette, |
| | and RBS-pyrE cds-T1-T2 terminator downstream of MS2 |
| | CP cassette, carries Corynebacterium replicon |
| pAPSE10822 | Suicide vector for creating pyrE knockout, |
| | chloramphenicol cassette, SacB cassette, temperature |
| | sensitive repA101 replicon, HT115(DE3) pyrE flanks. |
| pAPSE10826 | Suicide vector for creating pyrE knockout, |
| | chloramphenicol cassette, SacB cassette, temperature |
| | sensitive repA101 replicon, HT115(DE3) pyrE flanks. |
| | recA. |
| pAPSE10835 | pBR322 replicon, contains CPB β-actin hairpin with |
| | one pac site each at 3′ and 5′ends, contains MS2 CP |
| | cassette, and P-J23115-pyrE cds-T1-T2 terminator, |
| | contains ampicillin cassette as selection marker. |
| pAPSE10836 | pBR322 replicon, contains CPB β-actin hairpin with one |
| | pac site each at 3′ and MS2 CP cassette, and P-J23115- |
| | pyrE cds-T1-T2 terminator, lacks any antibiotic 5′ends, |
| | contains selection marker. |

dsRNA Production in Shake Flask

dsRNA production in shake flask was carried out using a minimal media. A carbon source (for e.g., glucose and/or α-lactose) were added for autoinduction for dsRNA production. A single colony of the appropriate culture was transferred to 2 ml liquid LB media supplemented with appropriate antibiotic to start the seed culture. After 5 to 6 hours incubation at 37° C. 100 μl of the seed culture was used to inoculate shake flasks containing 25 ml 25% Super Broth. Shake flasks were incubated at 37° C. overnight in shaker incubators at 250 rpm. After 18 to 19 hours of incubation, culture OD₆₀₀ was recorded, and culture aliquots were collected and stored at −80° C. A 1-ml aliquot of the culture was saved for TRIzol™ RNA extraction.

Minimal media was used for shake flask RNA production. Seed cultures were grown as described above. A cell pellet obtained from the seed cultures was resuspended in 1 ml minimal media and used to inoculate shake flask containing 25 ml of minimal media. The shake flasks were incubated at 37° C. for 24 hours in shaker incubators at 250 rpm. After 24 hours of incubation, culture OD₆₀₀ was recorded, and culture aliquots were collected and stored at −8° C. A 3-ml aliquot of the culture was saved for RNA extraction.

### RNA Extraction

Cell pellets from aliquots collected from shake flask cultures or from 250-μl aliquots of fermentation broth were processed for RNA extraction as follows. Cell pellets were resuspended in cold 20 mM Tris-HCl pH 7.0 to bring volume to 500 μl. Resuspended cells were transferred to cold 2-ml bead beater tubes (Sigma, St. louis, MO) and lysed by bead beating for 2 minutes in a Biospec mini-beadbeater-16 (Biospec Products, Bartlesville. OK). After bead beating, the lysate was centrifuged at 14000 rcf for 2 minutes following incubation on ice for 5 minutes. A 100-μl aliquot of centrifuged lysate was transferred to a microcentrifuge tube containing 300-μl cold 20 mM Tris-HCl, pH 7 and 150 μl TRIzol™ (Ambion, Austin, TX) and total RNA was extracted according to the manufacturer's protocol. RNA pellet was resuspended in 200 μl 20 mM Tris-HCl. pH 7.

### RNA Quantification

Total RNA concentration was measured using nanodrop 200° C. spectrophotometer (ThermoFisher Scientific). A 5-μg aliquot of total RNA from a sample was subjected to RNAseA (NEB. Ipswich, MA) digestion for two hours followed by Proteinase K (NEB, Ipswich, MA) digestion for 1 hour. Either 100 ng (shake flask samples) or 50 ng (fermentation sample) of RNAseA/Proteinase K-digested total RNA was run on 1.5% agarose gel along with 100 bp quantitative DNA ladder (Lambda Biotech. St. Louis, MO) to quantify dsRNA content of the sample. The gel image was captured with Bio-Rad molecular imager gel doc XR+ gel imaging system and the RNA bands on the gel were quantified using Image Lab software 6.1 (Bio-rad, Hercules. CA) following the manufacturer's guidelines.

### Protein Analysis

Cell lysates produced by bead beating were used for recombinant protein analysis by SDS-PAGE with Coomassie staining. Cell lysate samples containing an approximately equal number of cells based on OD₆₀₀ were used for analyses. Each cell lysate sample (1.5 to 6 μl) was mixed with 2.5 μl NUPAGE 4×LDS sample buffer (Invitrogen, Waltham. MA), 1 μl NuPAGE sample reducing agent (Invitrogen, Waltham, MA) and the total sample volume was made up to 10 μl with 20 mM Tris HCl pH7. The prepared protein samples were incubated at 75° C. for 10 minutes followed by incubation on ice until gel loading. The protein samples were run on NuPAGE 12% PAGE gel (Invitrogen) along with SeeBlue™ Plus2 Pre-stained protein standard (Invitrogen. Waltham, MA). After the gel run, the gel was taken out of the casting tray, rinsed with Milli-Q water and subjected to Coomassie blue staining.

### Example 2

High Levels of Extracapsid dsRNA Accumulate in ΔRnc E. coli Cells Co-Expressing the Bacteriophage MS2 Coat Protein

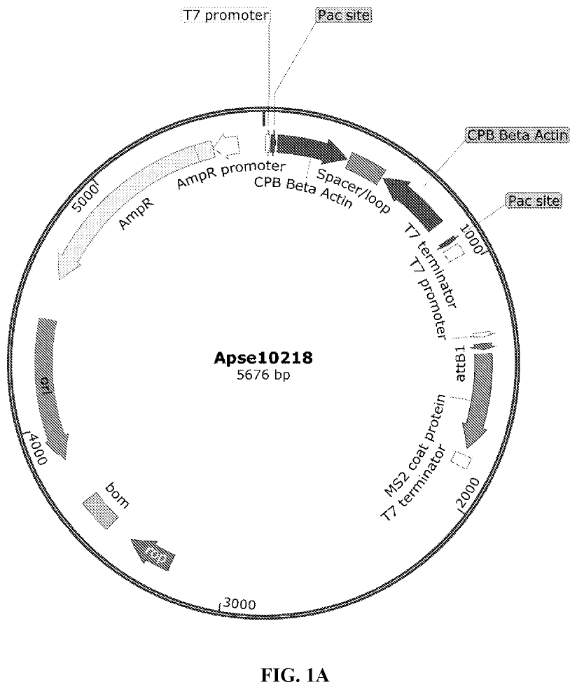

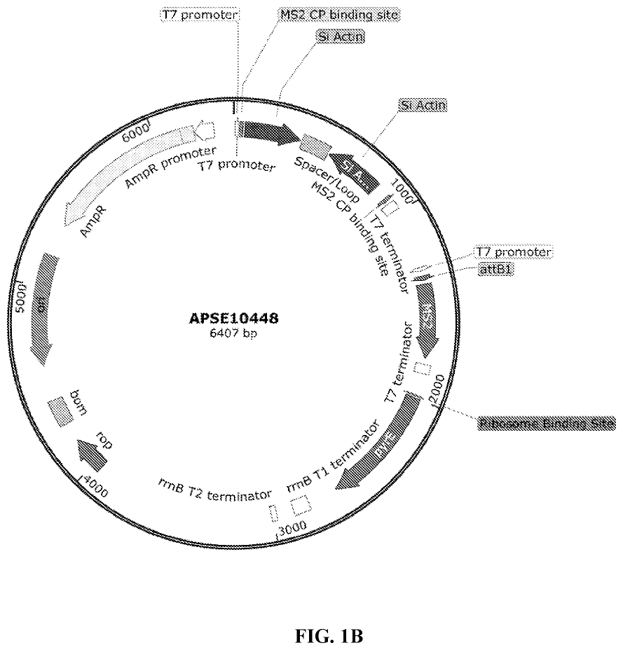

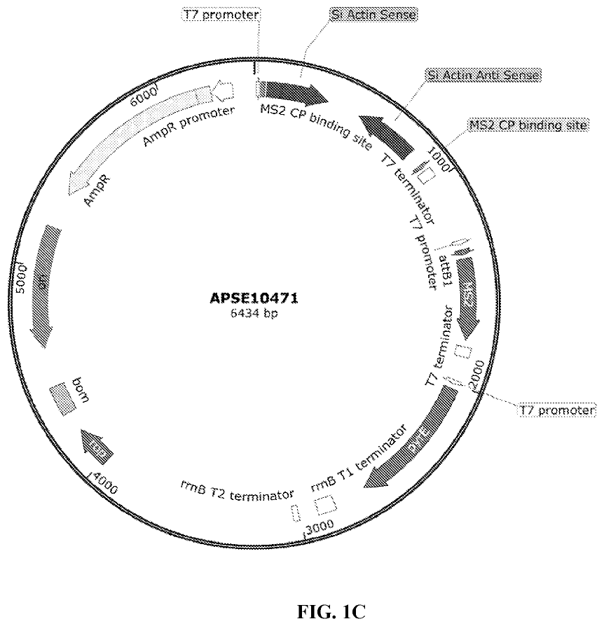

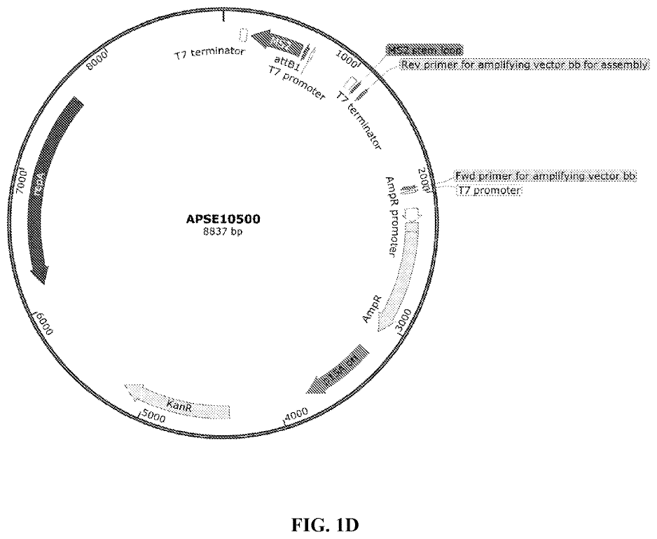

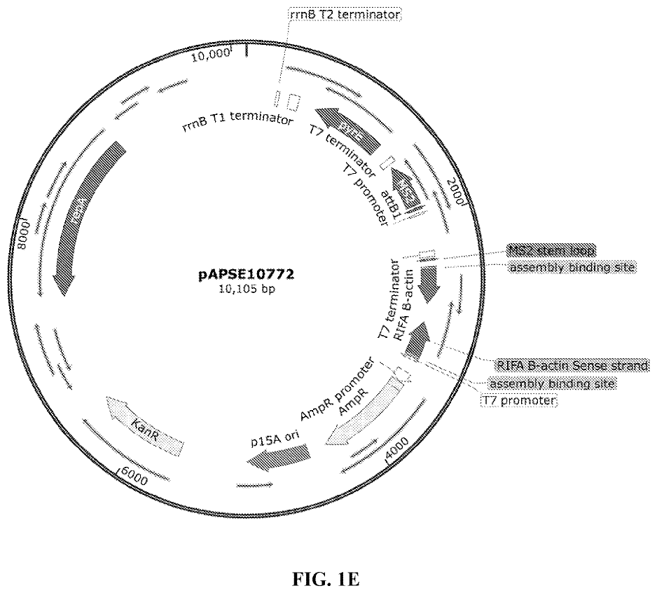

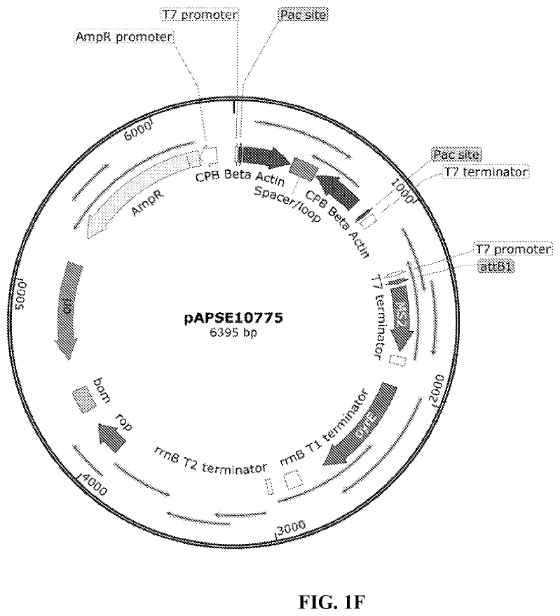

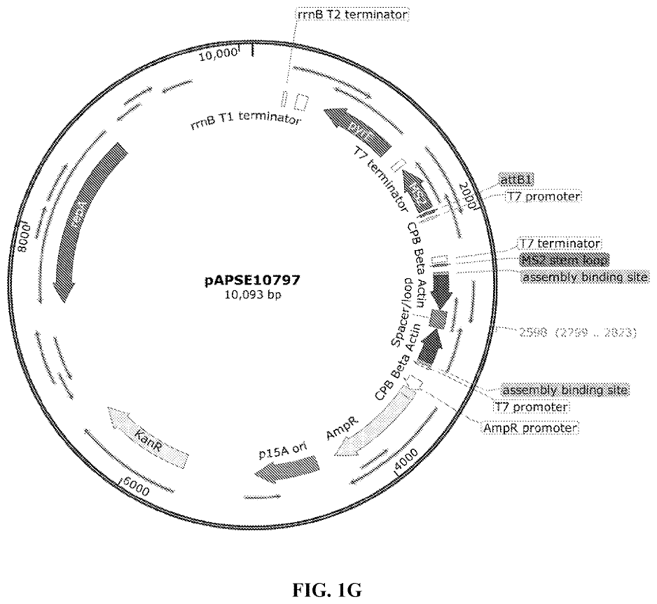

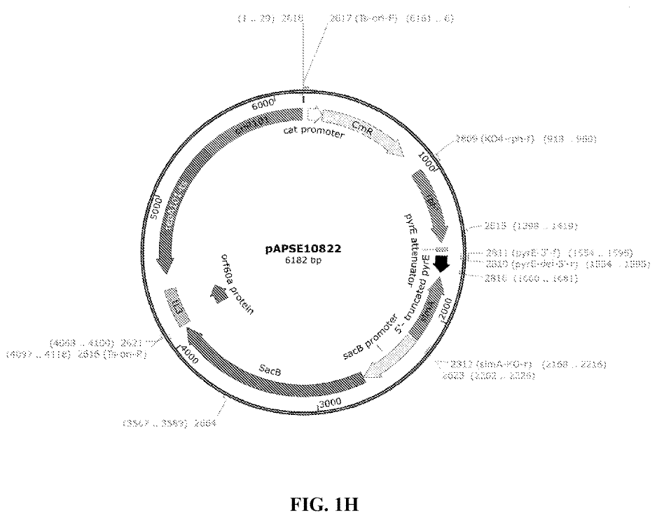

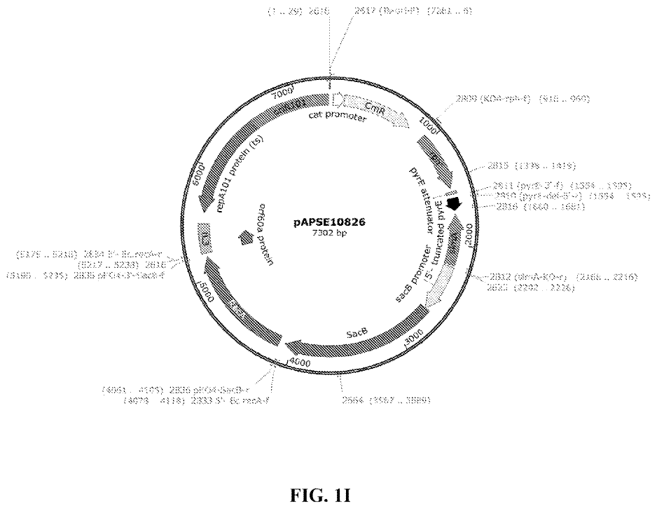

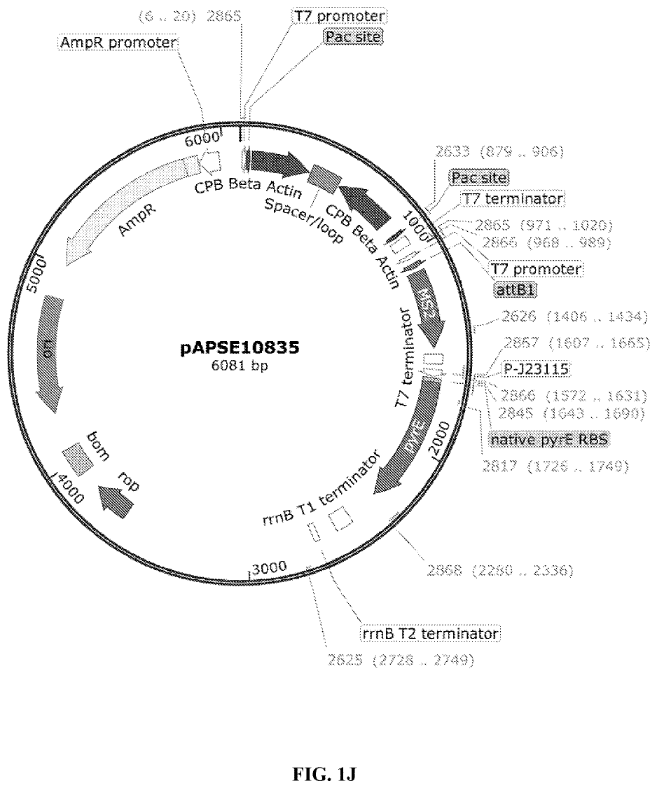

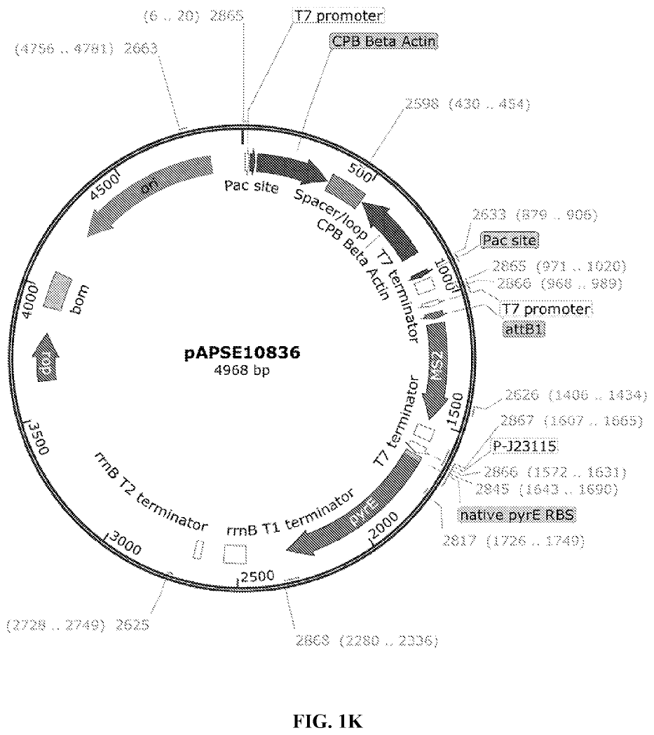

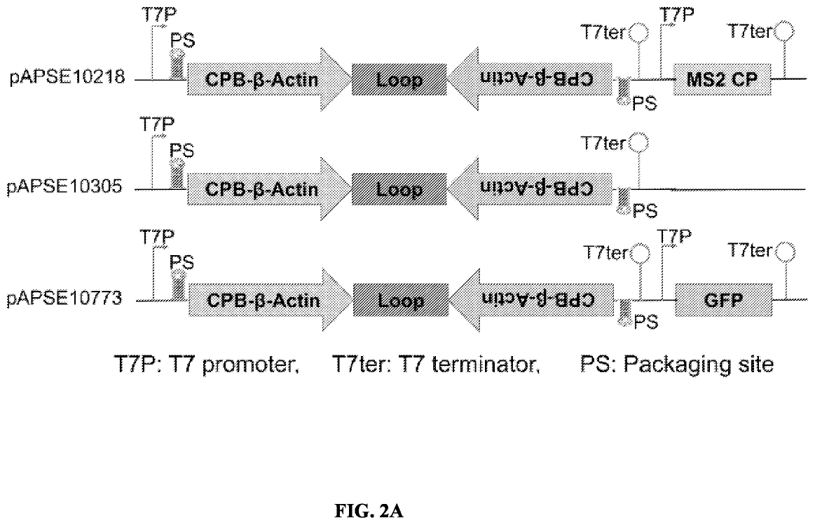

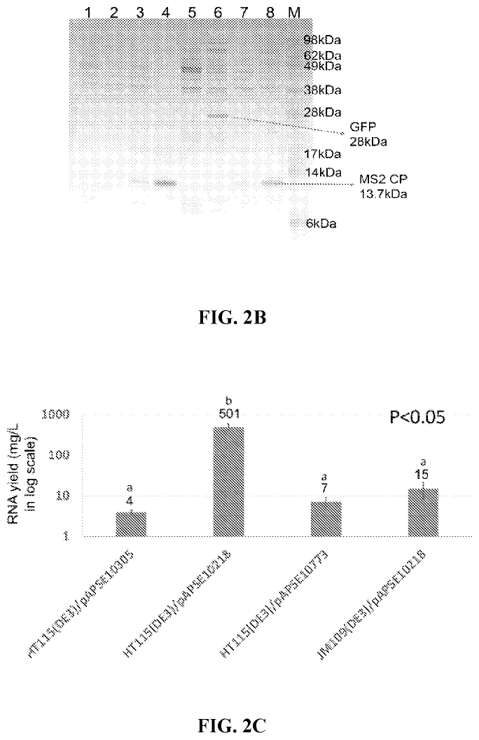

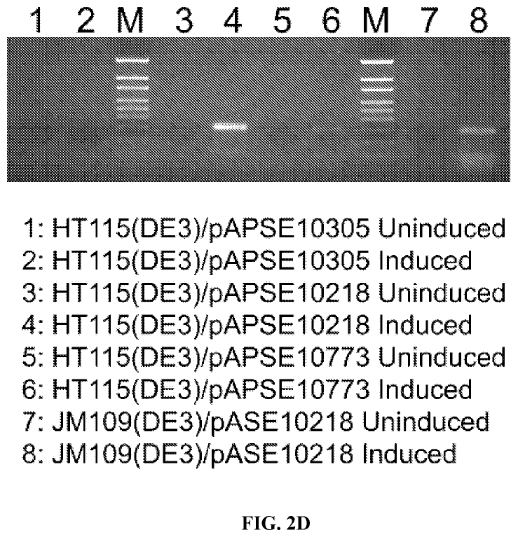

A study was conducted to determine whether dsRNA could be produced at high levels and packaged into MS2 virus-like particles (VLPs). To produce MS2 VLPs containing dsRNAs, vector pAPSE10218 (FIG. 1) was introduced into a Arnc Escherichia coli strain, HT115(DE3). The E. coli mc gene encodes a dsRNA-specific RNase (RNaseIII) and inactivation of c is required to accumulate dsRNA in E.coli. Vector pAPSE10218 co-expresses the Bacteriophage MS2(MS2) CP cassette with an 893-base hairpin RNA (hpRNA) derived from the β-actin gene of Colorado potato beetle (Leptinotarsa decemlineata (Say)) (CPB) and comprises a 19-nucleotide RNA stem-loop sequence referred as the MS2 packaging (pac) site at the 5′ and 3′ ends of the hairpin to promote capsid self-assembly and packaging of the dsRNA (FIG. 2A). MS2 VLP-encapsidated dsRNA was recovered from E coli HT115(DE3)/pAPSE10218. The average dsRNA yield of isolated encapsidated dsRNA was 5 mg/L in shake flask cultures (data not shown). Additionally, large amounts of unencapsulated (‘extracapsid’) dsRNA were also recovered from E. coli HT115(DE3)/pAPSE10218. Total shake flask dsRNA yields including both encapsidated and extracapsid dsRNA ranged from 200 to 500 mg/L.

To determine if the MS2 CP was required for dsRNA accumulation, the MS2 CP expression cassette was deleted from vector pAPSE10218 creating vector pAPSE10305 (FIG. 2A). Another vector, pAPSE10773, was created by replacing the MS2 CP coding sequence with the green fluorescent protein (GFP) coding sequence in pAPSE10218 (FIG. 2A) to test whether co-expression of another protein could also lead to dsRNA accumulation via some non-specific interaction with the dsRNA. The newly created vectors were introduced into E. coli strain HT115(DE3) and shake flask dsRNA yields were measured. MS2 CP and GFP expression was confirmed by SDS-PAGE and Coomassie staining (FIG. 23, Lanes 1-6). No recombinant protein band was visible in HT115(DE3)/pAPSE10305 cell lysates (FIG. 23). A strong band of GFP in HT115(DE3)/pAPSE10773 cell lysates and a strong band of MS2 CP in HT115(DE3)/pAPSE10218 cell lysates were visible. The average dsRNA yield of HT115(DE3)/pAPSE10305 cultures lacking the MS2 CP was 4 mg/L while HT115(DE3)/pAPSE10773 cultures co-expressing GFP with dsRNA yielded 7 mg/L on average (FIG. 2C and FIG. 2D, Lanes 1-6). The average dsRNA yield of HT115(DE3)/pAPSE10218 cultures co-expressing MS2 CP with dsRNA was significantly higher at 501 mg/L (FIG. 2C) confirming that co-expression of the MS2 CP is necessary for accumulation of dsRNA in E. coli cells.

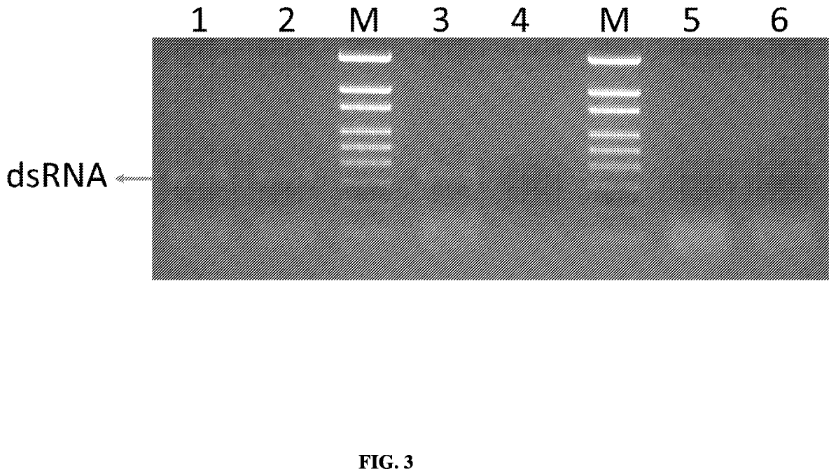

To confirm whether the MS2 CP facilitates accumulation of dsRNA by protecting dsRNA from cellular nucleases, vectors pAPSE10218 and pAPSE10305 were introduced into E. coli strain JM109(DE3) containing a functional rnc gene. The strain JM109(DE3)/pAPSE10218 which co-expresses MS2 CP and dsRNA had a low level (~15 mg/L) accumulation of dsRNA (FIG. 3). No dsRNA accumulation was not detected in the absence of MS2 CP expression in the strain JM109(DE3)/pAPSE10305 (FIG. 3). The dsRNA yield of JM109(DE3)/pAPSE10218 strain carrying a functional mc gene was 15 mg/L, significantly lower than 501 mg/L obtained with HT115(DE3)/pAPSE10218 (FIG. 2C and FIG. 2D, Lanes 3-4 and 7-8). SDS-PAGE with Coomassie staining (FIG. 2B, Lanes 3-4, 7-8) showed that JM109(DE3)/pAPSE10218 and HT115(DE3)/pAPSE10218 cultures were expressing similar levels of MS2 CP. This results from this study shows that the MS2 CP does not protect the dsRNA sufficiently to support high accumulation in the presence of a dsRNA-specific ribonuclease.

### Example 3

Presence and Number of MS2 Pac Sites Impact MS2 CP-Mediated Accumulation of dsRNA.

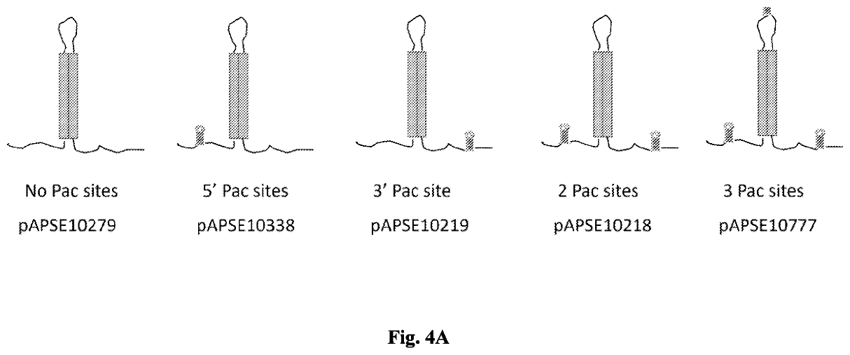

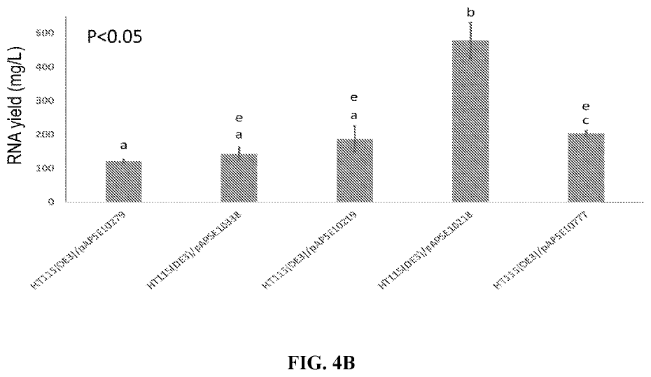

To further explore whether interactions between the dsRNA pac sites and MS2 CPs facilitate accumulation of dsRNA, vector pAPSE10218 carrying one MS2 pac sites each at the 5′ and 3′ ends of the dsRNA hairpin sequence (FIG. 2A) was used. To test whether the presence and/or number of MS2 pac sites affects dsRNA yield, four pAPSE10218 modifications were generated: 1) removal of both dsRNA pac sites (pAPSE10279), 2) removal of the 3′ dsRNA pac site (pAPSE10338), 3) removal of the 5′ dsRNA pac site (pAPSE10219), and 4) addition of a third pac site to the hairpin loop (pAPSE10777) (FIG. 4A). Each vector was introduced into E. coli HT115(DE3).

It was found that E. coli with vector pAPSE10279, which produced dsRNA lacking pac sites, accumulated quite high levels of dsRNA (121 mg/L) in the presence of the MS2 CP (FIG. 4B), and that addition of 1-2 pac sites to the hpRNA improved dsRNA accumulation. E. coli harboring either vectors pAPSE10338 or pAPSE10219, each of which produce dsRNA with a single pac site, produced 142 mg/L and 186 mg/L dsRNA, respectively. Of the constructs tested, E. coli carrying pAPSE10218, which produced hpRNA containing one pac site at its 5′ and 3′ end, was the optimal design to maximize dsRNA yield accumulated. This strain yielded 478 mg/L dsRNA, more than double the amount of dsRNA produce by any other strain in this experiment. HT115(DE3)/pAPSE10777 comprising a third pac site in the hairpin loop yielded dsRNA at levels similar to HT115(DE3)/pAPSE10219 (FIG. 43).

### Example 4

An Intramolecular Hairpin RNA (hpRNA) Accumulates to Higher Levels than an Intermolecular dsRNA.

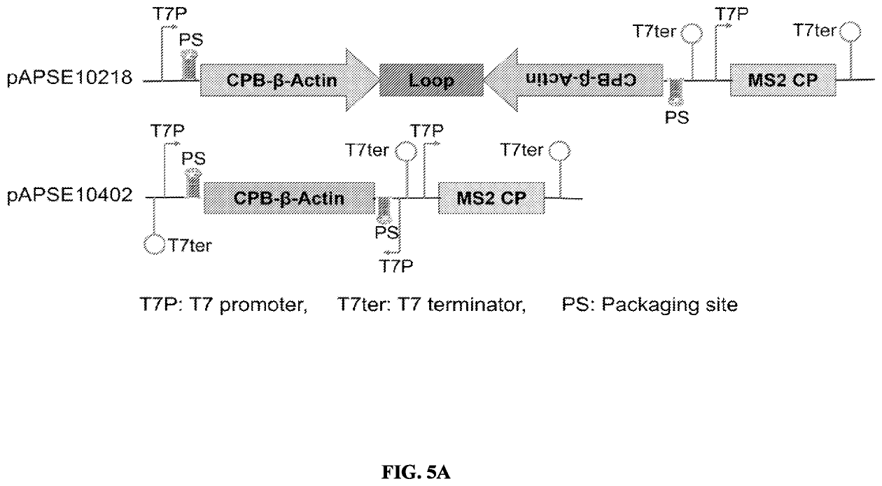

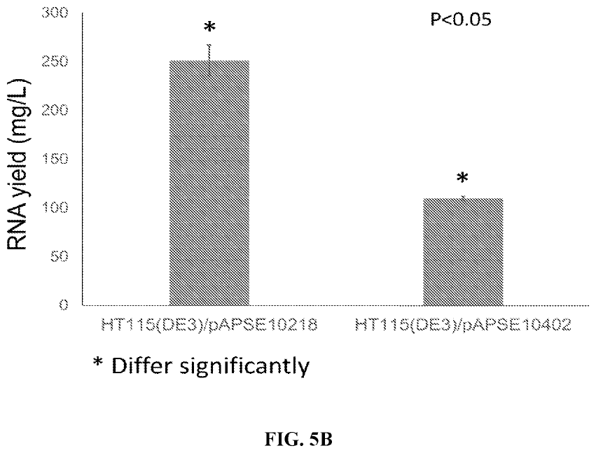

To examine the impact of the transcriptional design on dsRNA accumulation, vector pAPSE10402 was created by modifying pAPSE10218 to produce an intermolecular dsRNA comprised of a 294-bp sequence from the CPB β-actin gene flanked by a MS2 pac site at each end (FIG. 5A). The average shake flask yield of E. coli HT115(DE3)/pAPSE10218 producing the hpRNA was almost 2.5-fold higher at 251 mg/L when compared to HT115(DE3)/pAPSE10402 producing the intermolecular dsRNA which yielded 109 mg/L (FIG. 5B).

### Example 5

Supplemental Expression of the PyrE Gene Improves Shake Flask dsRNA Yield of an E. coli K-12-Derived Strain Growing in Minimal Media

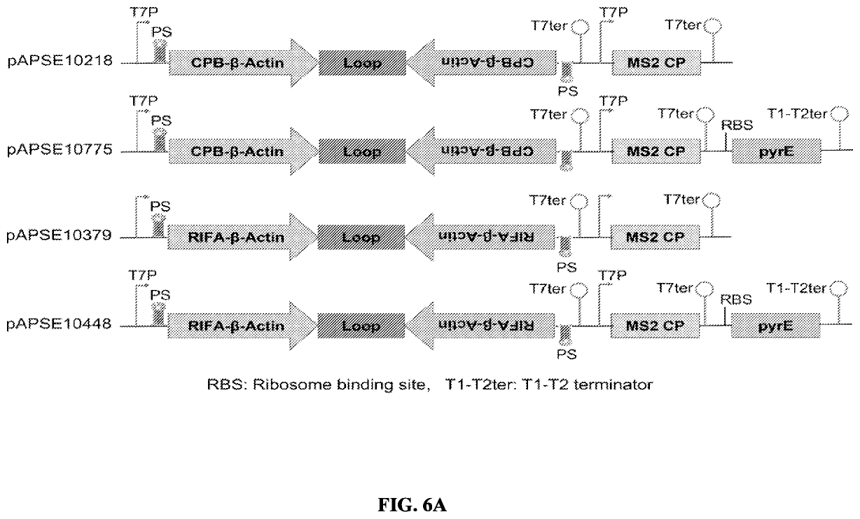

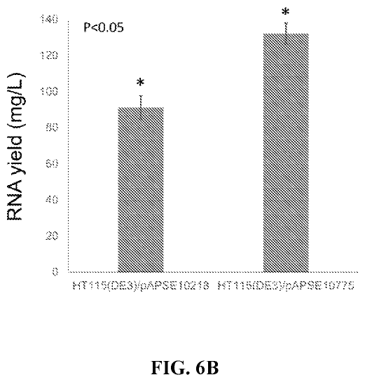

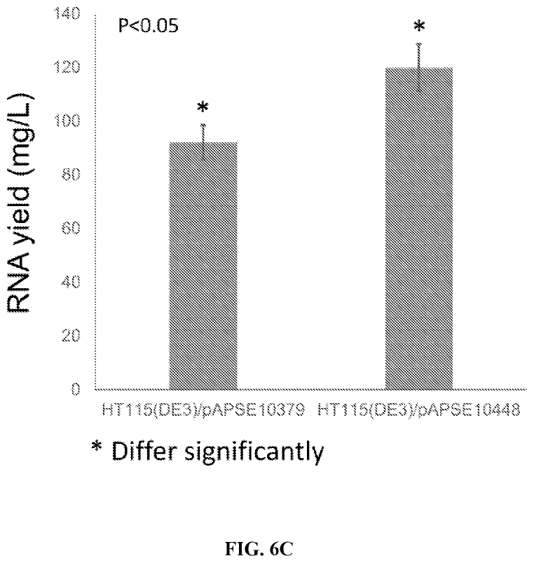

To determine whether increasing pyrE expression would increase dsRNA yields, pAPSE10218 was modified with the pyrE coding sequence including a ribosome binding site at its 5′end and T1-T2 terminators at its 3′ end. The pyrE construct was inserted downstream of the MS2 CP expression cassette to permit readthrough transcription of the pyrE coding sequence by the T7 promoter. The resulting vector, pAPSE10775 (FIG. 6A) was introduced into E. coli HT115(DE3) and evaluated for dsRNA production in minimal media.

On average, E. coli HT115(DE3)/pAPSE10775 yielded 45% more dsRNA compared to E. coli HT115(DE3)/pAPSE10218 (FIG. 6B). To confirm yield improvement with a second dsRNA sequence, the CPB β-actin hairpin sequence in vectors pAPSE10218 and pAPSE10775 was replaced with a red imported fire ant (RIFA) (Solenopsis invicta) β-actin hairpin sequence creating plasmid pAPSE10379 and pAPSE10448, respectively. Each strain for shake flask dsRNA production was grown in triplicate. Data from shake flask experiments involving more than two strains was subjected to one-way analysis of variance (ANOVA) and data from experiments involving two strains was subjected to t-test analysis. Following ANOVA analysis post-hoc test with Bonferroni correction was used to identify constructs that differed significantly for dsRNA yields. Results from the shake flask experiments using minimal media, show that E. coli HT115(DE3)/pAPSE10448 produced 30% more dsRNA relative to E. coli HT115(DE3)/pAPSE10379 (FIG. 6C).

### Example 6

Fed-Batch Fermentation of E. coli HT115(DE3) Engineered with a Plasmid Vector With Stem Loon Sequence, 3′ and 5′ Pac Sites. MS2 CP Cassette and pyrE Supplemental Expression Yields High Amounts of dsRNA

To assess the maximum yield potential of the disclosed dsRNA production system in a benchtop high-cell-density fed batch bioreactor, the pyrE-deficient strain, E. coli HT115(DE3)/pAPSE10218, and the pyrE overexpressing strain E coli HT115(DE3)/pAPSE10775 were each evaluated in three independent fermentation runs. pAPSE10775 comprises T7 promoter operably linked to CPB β-actin hairpin with one pac site each at 3′ and 5′ends, a MS2 CP cassette, and RBS-pyrE cds-T1-T2 terminator downstream of MS2 CP cassette.

Fed-batch fermentations were carried out in a 3-L vessel using a New Brunswick BioFlol 15 (Eppendorf, Enfield, CT) bench-top bioreactor with a working volume of 2 L. The seed culture for fermentation inoculum was grown in 2 ml LB media with appropriate antibiotics. After 8 hours of growth, the seed culture was used to inoculate 50 ml minimal media. Inoculum OD600 was recorded before transferring the 50-mi inoculum to the fermentation vessel containing 1.2 L of minimal media. The glucose feed consisted of 50% glucose with the major salts of minimal media. To support initial growth of culture, 5 ml glucose feed was added to the vessel prior to inoculating the fermentation vessel with the inoculum. The pH was maintained at 7.0 by addition of 30% (v/v) NH40H. Dissolved oxygen (DO) was set to 30% saturation and controlled through agitation—DO cascade during initial 16 hours of fermentation run. After 16 hours, agitation was set to 1000 rpm and DO was used to control the glucose feed. Culture growth was monitored by measuring OD600. Culture was induced for dsRNA production between OD600 of 65 to 70 with 1 mM IPTG. Fermentation samples were collected at the time of induction and every hour after induction up to 5 hours for evaluating dsRNA production. Three different fermentation runs were carried out for each strain and the dsRNA data was subjected to t-test analysis.

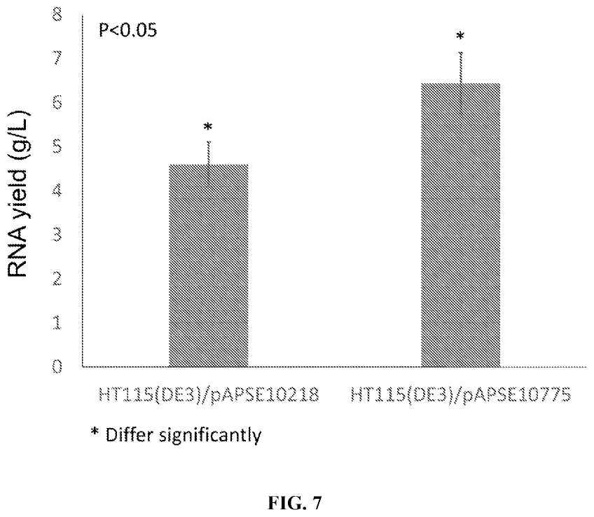

The dsRNA yields of the three HT115(DE3/pAPSE10218 fermentation runs ranged from 4 g/L to 4.8 g/L and averaged 4.69 g/L (FIG. 7). The highest dsRNA yield achieved with strain HT115(DE3)/pAPSE10775 was 11.26 g/L with an average yield of 10.12 g/L over three fermentation runs. The yield obtained is 115.78% higher than HT115(DE3)/pAPSE10218 (FIG. 7). The results verify that supplemental expression of pyrE compensates for OPRTase deficiency of K-12-derived E.coli HT115(DE3) and significantly increases dsRNA yield of cultures grown in minimal media in a high-cell-density fed batch bioreactor.

### Example 7

dsRNA Produced by Fed-Batch Fermentation of E. coli Engineered with pAPSE10775 Has High Efficacy Against CPB Larvae in Leaf Disc Bioassays

The dsRNA produced by fed-batch fermentation of E coli engineered with pAPSE10775 was evaluated for its efficacy against Colorado Potato Beetle (CPB) larvae in leaf disc bioassays.

Colorado Potato Beetle (CPB) Larvae Laboratory Bioassays were conducted by AgMetrics Group (Albion, MI). Mixed sex, first instar Colorado potato beetle larvae of similar size used in these bioassays were from a colony maintained under laboratory conditions. Potato plants (Solanum tuberosum cv. Kennebec) used in bioassays were grown in greenhouses. To kill E. coli cells containing β-actin dsRNA prior to use in bioassays, cells were incubated at 55C for 15 minutes. The dsRNA in the heat-killed sample was quantified and three different dsRNA solutions (2.5 ng/μl, 10 ng/μl, and 40 ng/μl) were prepared by the diluting the sample using Milli-Q water.

A 10-μl volume of each dsRNA solution including 0.1% Tween-20 was pipetted on to a leaf discs (D=1 cm²) and spread across the leaf surface using a glass rod to ensure even coverage. Leaf discs were allowed to air dry and then each disc was transferred to a 100-mm petri plate lined with filter paper and a single larvae was placed on each treated disc. Untreated leaf discs were provided in the negative control treatment group. Treatment groups included twenty biological replicates, each.

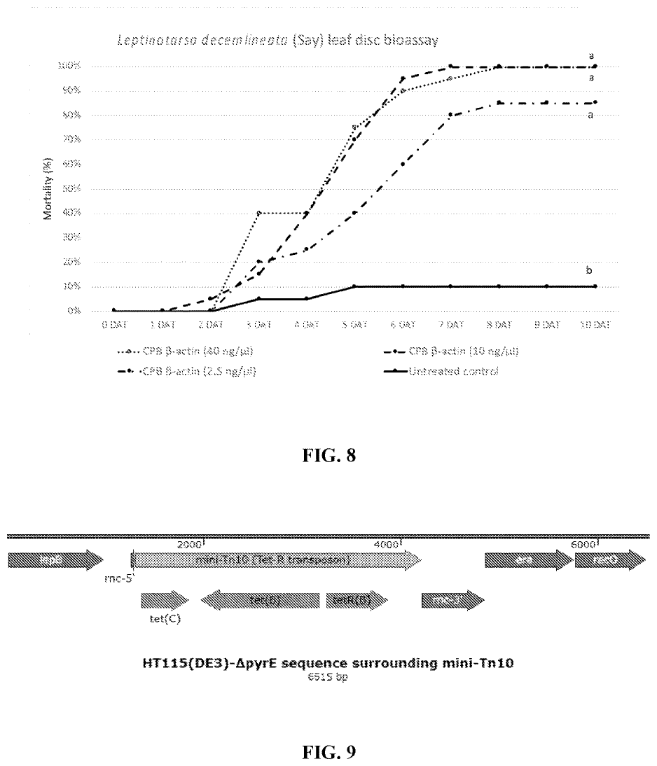

After 24 hours, residual leaf material was removed from each petri plate and replaced with a freshly cut and treated leaf disc. This process was repeated for the first four days of the bioassay. On day five, residual leaf material was removed from each petri plate, and replaced with a fresh, untreated leaf disc. This process was repeated through day ten, at which time the bioassay was ended. During the assay, the number of dead larvae in each treatment group was recorded daily. Larvae that did not respond to probing were considered dead. Data was analyzed by one-way ANOVA followed by Fisher's least significant difference test to identify significant differences among groups (p<0.05). Results indicate that the dsRNA was found to cause 100% mortality seven days after treatment when delivered at a concentration of 10 ng/μl (FIG. 8).

### Example 8

Fed-Batch Fermentation of E. Coli HT115(DE3) Engineered with a Plasmid Vector With MS2 CP Cassette and pyrE Under the Control of Dedicated T7 Promoter

To assess the yield potential of a plasmid vector having MS2 CP cassette and pyrE, with pyrE being under the control of a dedicated promoter, dsRNA production was assessed in E. coli HT115(DE3)/pAPSE10471 in a benchtop high-cell-density fed batch bioreactor. pAPSE10471 was engineered to comprise T7 promoter operably linked to RIFA B-actin with one pac site each at 3′ and 5′ends, a MS2 CP cassette, and T7-RBS-pyrE cds-T1-T2 terminator downstream of MS2 CP cassette, with pyrE under the control of dedicated T7 promoter. Fed-batch fermentation and dsRNA production was carried using methods described in example 6. The dsRNA yields of the E. coli HT115(DE3)/pAPSE10471 from the fermentation run was 5.5 g/L (Table 3). This result suggests that high dsRNA production can be attained by engineering plasmid vectors with MS2 CP cassette and having pyrE expression under the control of its own promoter.

TABLE 3
dsRNA yield in E. coli HT115(DE3) engineered with a plasmid vector
having pyrE under the control of a dedicated T7 promoter

| | | | | dsRNA |
|---|---|---|---|---|
| Bacterial | | Hairpin | | yield |
| sps. | Construct number | sequence | Construct design | (g/L) |
| E. coli | pAPSE10471 (pyrE | RIFA β- | T7P-MS2 cds-T7 | 5.5 |
| | under the control of | actin | ter-T7P-pyrE cds- | |
| | dedicated T7 P) | | T1T2 ter | |

### Example 9

Fed-Batch Fermentation of C. glutamicum Engineered with Plasmid Vectors

The dsRNA produced by fed-batch fermentation of C. glutamicum engineered with plasmid vectors was evaluated. Fed-batch fermentations were carried out in a 3-L vessel using a New Brunswick BioFlol15 (Eppendorf, Enfield. CT) bench-top bioreactor with a working volume of 2 L. The seed culture for fermentation inoculum was grown in 5 ml brain-heart infusion sorbitol (BHIS) media with appropriate antibiotics. After overnight growth at 30° C., the seed culture was used to inoculate 50 ml BHIS media and was grown at 30° C. for 6 to 8 hours. Inoculum OD600 was recorded before transferring the 50-ml inoculum to the fermentation vessel containing 1.2 L of minimal media. The glucose feed consisted of 50% glucose with the major salts of minimal media and 20 mg/L biotin. To support initial growth of culture, 5 ml glucose feed was added to the vessel prior to inoculating the fermentation vessel with the inoculum. The pH was maintained at 7.2 by addition of 30% (v/v) NH40H. Dissolved oxygen (DO) was set to 30% saturation and controlled through agitation—DO cascade during fermentation run. Culture growth was monitored by measuring OD600. Culture was induced for dsRNA production between OD600 of 75 to 80 with 0.1 mM IPTG. Fermentation samples were collected at the time of induction and every hour after induction up to 5 hours for evaluating dsRNA production.

To assess the yield potential of a plasmid vector having MS2 CP cassette and pyrE, dsRNA production was assessed in C. glutamicum transformed with pAPSE10772, pAPSE10797 or pAPSE10500 (SEQ ID NO: 36), in a benchtop high-cell-density fed batch bioreactor. pAPSE10772 (SEQ ID NO: 38) was engineered to have T7 promoter operably linked to RIFA β-actin with one pac site each at 3′ and 5′ends, a MS2 CP cassette, and RBS-pyrE cds-T1-T2 terminator downstream of MS2 CP cassette. pAPSE10797 (SEQ ID NO: 39) has a T7 promoter operably linked to CPB β-actin with one pac site each at 3′ and 5′ends, a MS2 CP cassette, and RBS-pyrE cds-T1-T2 terminator downstream of MS2 CP cassette, while pAPSE10500 has MS2 CP cassette without the pyrE construct. Table 4 summarizes the results of the dsRNA yield produced by C. glutamicum/pAPSE10772 and C. glutamicum/pAPSE10797 and the predicted yield for C. glutamicum/pAPSE10500. The dsRNA yields of the C. glutamicum/pAPSE10772 from the fermentation run was achieved at 1.1 g/L (Table 4), while the yield from C. glutamicum/pAPSE10797 was about 1 g/L (Table 4). The yield predicted for C. glutamicum/pAPSE10500, which lacks the pyrE construct is around 0.8 g/L (Table 4). These results suggests that high dsRNA production can be attained by engineering plasmid vectors co-expressing MS2 CP cassette and pyrE constructs in C. glutamicum.

TABLE 4
dsRNA yield in C. glutamicum engineered with plasmid vectors

| Bacterial | Construct | Hairpin | | dsRNA |
|---|---|---|---|---|
| sps. | number | sequence | Construct design | yield (g/L) |
| C. | pAPSE10772 | RIFA β- | T7P-MS2 cds-T7 | 1.1 |
| glutamicum | | actin | ter-pyrE cds-T1T2 | |
| | | | ter | |
| C. | pAPSE10797 | CPB β-actin | T7P-MS2 cds-T7 | 1 |
| glutamicum | | | ter-pyrE cds-T1T2 | |
| | | | ter | |
| C. | pAPSE10500 | RIFA β- | T7P-MS2 cds-T7 | 0.8* |
| glutamicum | (lacks pyrE) | actin | ter | |

*predicted yield

### Example 10

### Construction of Antibiotic Marker-Free Plasmid Backbone and Modified E. coli Strains to Allow Propagation and dsRNA Expression from Such Constructs

An antibiotic marker-free plasmid and host system for dsRNA expression was further developed. The pyrE gene was chosen to be the selectable marker for plasmid selection and maintenance. pyrE knock-out strains of E. coli and related microbes are uracil auxotrophs, but otherwise fully viable and ‘healthy’ if supplemented with uracil in the growth medium, or if complemented (in trans) with a functional pyrE gene on a plasmid.

A suicide plasmid system previously developed (Link et. al, 1997) was selected to knock-out the pyrE gene in the E. coli genome. Plasmid pAPSE10822 (SEQ ID NO: 40), a chloramphenicol-resistant plasmid containing a temperature sensitive origin of replication, and the Bacillus SacB gene for counterselection in the presence of sucrose was assembled, containing ~0.7 kb of E. coli K-12 genomic DNA sequence upstream of pyrE, and ~0.7 kb of DNA sequence downstream of pyrE (extracted from GenBank CP017979, the genome of E. coli K-12 strain W3110). The design deleted the pyrE coding sequence from amino acid 1 through 175, making it a functionally inactive truncated peptide. The integration of the knock-out vector into the genome of HT115(DE3) cells by homologous recombination and its excision following selection for sucrose resistance was performed. Sucrose-resistant, chloramphenicol sensitive E. coli colonies were screened using PCR with primer pairs that would identify the pyrE gene deletion. A colony with the related PCR patterns indicative of gene deletion was selected and tested for uracil auxotrophy by checking for growth in M9-glucose minimal agar versus minimal agar medium supplemented with 50 μg/ml uracil. The new strain was confirmed to be genetically deleted in pyrE and a uracil auxotroph. This created strain HT115(DE3)-ΔpyrE.

Strain HT115(DE3)-ΔpyrE is a recA-plus strain which as such is not useful for cloning and direct transformation with ligation mixtures to create new constructs. A new suicide plasmid pAPSE10826 (SEQ ID NO: 41) was assembled by cloning the E. coli recA coding region into the backbone of pAPSE10822. Unlike plasmid pAPSE10822, pAPSE10826 could be integrated and subsequently excised from the genome of E. coli strain NEB-5 alpha, a recA-minus strain commonly used for gene cloning and stable plasmid maintenance. Following essentially the same steps outlined above for HT115(DE3), strain NEB-5 alpha-ΔpyrE was created. The genomic deletion of pyrE and uracil auxotrophy were confirmed. By having lost the suicide plasmid backbone, the strain was again recA-minus, which was confirmed by high sensitivity to UV cell killing.

Plasmid pAPSE10775, with a promoter-less pyrE gene was unable to complement strain NEB5-ΔpyrE to allow growth in M9-glucose minimal agar plates, presumably due to insufficient pyrE expression. In order to make pyrE a suitable selectable marker (i.e. to allow plasmid selection by uracil prototrophy), pAPSE10835 (SEQ ID NO: 42) was constructed by modifying the pyrE gene in pAPSE10775 with the addition of the constitutive J23115 promoter driving pyrE expression. Transformants of pAPSE10835 in both HT115(DE3)-ΔpyrE and NEB5-ΔpyrE are selectable by growth on M9-glucose minimal medium without added uracil. pAPSE10835 also conferred ampicillin resistance.

Plasmid pAPSE10836 (SEQ ID NO: 43) was made by deleting the Amp-r gene (bla, beta lactamase) from the backbone of pAPSE10835 by BspHI restriction digestion and re-ligation, and transformation into competent NEB5-ΔpyrE cells and selection for growth on M9-glucose (without antibiotics). For growth for plasmid preps, transformants can be grown in minimal medium, as well as in LB without plasmid loss. The nucleotide sequence of pAPSE10836 was confirmed by whole plasmid sequencing.

Plasmid pAPSE10836 was transformed into competent HT115(DE3)-ΔpyrE cells and selected for growth in minimal medium, as described for NEB5-ΔpyrE. Recombinant HT115(DE3)-ΔpyrE cells with pAPSE10836 were tested for dsRNA production first in shake flasks. The shake flask dsRNA yields of the three HT115(DE3)-ΔpyrE/pAPSE10836 clones was similar to shake flask dsRNA yields of HT115(DE3)/pAPSE10775. The HT115(DE3)-ΔpyrE/pAPSE10836 clone with highest shake flask dsRNA yield was evaluated for dsRNA production in a fermenter. The fermentation dsRNA yield of HT115(DE3)-ΔpyrE/pAPSE10836 was around 5.4 g/L, which falls in the range of HT115(DE3)/pAPSE10775 fermentation dsRNA yield.

### Example 11

### Removing Antibiotic Marker Gene (Tetracycline Resistance) from the Genome of E. coli dsRNA Production Strain HT115(DE3)-ΔpyrE

The rnc/RNAseIII knock-out mutation in E. coli HT115 originates from a mini-Transposon Tn10 insertion event close to the 5′-/N-terminus of mc in the E. coli mc-era-recO operon. The transposon functionally knocks out rnc, while allowing the expression of the downstream era gene, an essential gene, making HT115 and its derivative strains suitable for dsRNA production. The presence of mini-Tn10 however, puts a Tetracycline resistance gene in the HT115 genome and its derivative strains such as HT115(DE3)-ΔpyrE.

SEQ ID NO: 44 comprises the genomic sequence of HT115(DE3)-ΔpyrE, the full sequence of the mini-Tn10 transposon and its insertion site in the mc-era-recO operon is provided in FIG. 9. The transposon insertion positions TetR(B), a regulatory gene, to be co-directional with Dmc-era-recO. The Tet(B) gene, the Tetracycline resistance gene, is positioned upstream of TetR(B), oriented in the opposite direction.

Using a similar method used to create the ΔpyrE deletion utilizing plasmid APSE10822, a ΔTet(B) deletion template can be assembled in the suicide plasmid backbone containing approximately 0.7-1 kb of sequence upstream and downstream of the Tet(B) coding sequence, with Tet(B) deleted. Alternatively, a ‘deletion plus insertion’ template can be assembled, deleting the Tet(B) coding sequence, and replacing it with the MS2 Coat Protein coding sequence, placing MS2 CP expression under the control of the medium strength constitutive Tet(B) promoter.

The insertion and subsequent excision of the ‘knock-out’ and ‘knock-out plus knock-in’ constructs in the mini-Tn10 locus of HT115(DE3)-ΔpyrE by homologous recombination will yield sucrose-resistant colonies that can be screened for sensitivity to grow in a media with 12.5 μg/ml tetracycline. Tetracycline-sensitive colonies will be screened by PCR and sequencing with primer pairs that can confirm the correct ΔTet(B) or ΔTet(B)::MS2 genomic structures.

The improved strains will be completely devoid of the Tet(B) and paired with plasmid pAPSE10836 (or variants) allowing dsRNA production by fermentation without antibiotics and no antibiotic resistance genes in the vector or host. Strain HT115(DE3)-ΔpyrE-ΔTet(B)::MS2 additionally has the capacity to provide MS2 from a chromosomal copy, allowing further modification of the dsRNA expression vectors by removing the P-T7-MS2 expression cassette from the backbone.

### Summary of Examples

The results from the studies demonstrate that the MS2 CP is required for dsRNA accumulation in E. coli HT115(DE3), a RNAseIII deficient strain that has been widely used for microbial dsRNA production. Addition of pac sites further improved dsRNA yields, with one pac site flanking the transcribed RNA being optimal for dsRNA production (FIG. 2). The data indicates that, vector constructs with pac sites are desirable to attain higher dsRNA accumulation. Further, hpRNAs accumulated to higher levels than intermolecular dsRNA using the MS2 CP-dsRNA production system. The sense and antisense fragments of a hpRNA are linked via a loop sequence which facilitates the intramolecular RNA folding and formation of a dsRNA molecule immediately after synthesis of a transcript. Supplemental expression of pyrE from dsRNA production plasmids had increased shake flask and high cell density fed-batch fermentation dsRNA yields of CPB β-actin and RIFA β-actin dsRNAs. The highest dsRNA yield achieved in high-cell-density fed batch fermentation was with the pAPSE10775 construct which comprises T7 promoter operably linked to CPB β-actin hairpin with one pac site each at 3′ and 5′ends, a MS2 CP cassette, and RBS-pyrE cds-T1-T2 terminator downstream of MS2 CP cassette construct. Fermentation runs in E. coli transformed with plasmid vectors having a MS2 CP cassette, in combination with a pyrE construct engineered to have a dedicated T7 promoter, exhibited high yields of dsRNA, suggesting an alternative configuration of pyrE construct that can be engineered to produce high yielding dsRNA plasmid vector. Additionally, examination of dsRNA production in C. glutamicum transformed with plasmid vectors having MS2 CP cassette and pyrE constructs show high yield levels confirming the feasibility of the disclosed high yielding plasmid vector in fed batch fermentation systems using C. glutamicum.

The high dsRNA yields produced with the disclosed system pave the way to reduce dsRNA production costs and aide the development of RNAi biopesticides that are cost-competitive with chemical pesticides. This is amicrobial fermentation-based production system that can utilize existing fermentation infrastructure and does not require specialized equipment, materials, or conditions for dsRNA production unlike in vitro transcription, cell-free, and chemical synthesis methods. Since the T7 RNA polymerase based inducible expression system has been evaluated in various microorganisms, this system can be expanded to other industrial microbes—including species designated as “Generally Recognized as Safe” (GRAS) by the FDA like Corynebacterium glutamicum, Bacillus subtilis and Saccharomyces cerevisiae. The dsRNAs produced using this system have been demonstrated for agriculture pest control in laboratory bioassays (FIG. 8).

TABLE 5
Nucleic Acid (DNA) Sequences used in the plasmid vector.

| Sequence | | |
|---|---|---|
| ID number | Type | Sequence |
| SEQ ID NO. 1 | T7 promoter | TAATACGACTCACTATAGG |
| SEQ ID NO. 2* | RBS-pyrE cds-T1-T2 | AGAAGGAGATATACATATGaaaccatatcagcg |
| | | ccagtttattGaatttgcgcttagcaagcaggtgttaaagtttggcg |
| | | agtttacgctgaaatccgggcgcaaaagcccctatttcttcaacgc |
| | | cgggctgtttaataccgggcgcgatctggcactgttaggccgttttt |
| | | acgctgaagcgttggtggattccggcattgagttcgatctgctgttt |
| | | ggccctgcttacaaagggatcccgattgccaccacaaccgctgtg |
| | | gcactggcggagcatcacgacctggacctgccgtactgctttaac |
| | | cgcaaagaagcaaaagaccacggtgaaggcggcaatctggtt |
| | | ggtagcgcgttacaaggacgcgtaatgctggtagatgatgtgatc |
| | | accgccggaacggcgattcgcgagtcgatggagattattcaggc |
| | | caatggcgcgacgcttgctggcgtgttgatttcgctcgatcgtcag |
| | | gaacgcgggcgcggcgagatttcggcgattcaggaagttgagc |
| | | gtgattacaactgcaaagtgatctctatcatcaccctgaaagacct |
| | | gattgcttacctggaagagaagccggaaatggcggaacatctgg |
| | | cggcggttaaggcctatcgcgaagagtttggcgtttaacctaggC |
| | | AGAACGCAGAAGCtGTCTGATAAAACAG |
| | | AATTTGCCTGGCGGCAGTAGCGCGGTGGT |
| | | CCCACCTGACCCCATGCCGAACTCAGAA |
| | | GTGAAACGCCGTAGCGCCGATGGTAGTG |
| | | TGGGGTCTCCCCATGCGAGAGTAGGGAA |
| | | CTGCCAGGCATCAAATAAAACGAAAGGC |
| | | TCAGTCGAAAGACTGGGCCTTTCGTTTTA |
| | | TCTGTTGTTTGTCGGTGAACGCTCTCCTG |
| | | AGTAGGACAAATCCGCCGGGAGCGGATT |
| | | TGAACGTTGCGAAGCAACGGCCCGGAGG |
| | | GTGGCGGGCAGGACGCCCGCCATAAACT |
| | | GCCAGGCATCAAATTAAGCAGAAGGCCA |
| | | TCCTGACGGATGGCCTTTT |
| SEQ ID NO: 3 | 3′ Pac site | ACATGAGGATCACCCATGT |
| SEQ ID NO: 4 | 5′ Pac site | ACATGAGGATCACCCATGT |
| SEQ ID NO. 5 | MS2 CP CDS | ATGGCGTCTAACTTTACCCAATTCGTTCT |
| | | GGTTGATAACGGCGGTACGGGTGACGTT |
| | | ACCGTAGCTCCGTCCAACTTCGCCAACGG |
| | | TGTTGCGGAATGGATTAGCTCTAACAGCC |
| | | GCTCTCAGGCCTACAAAGTCACGTGCTCC |
| | | GTTCGTCAGTCTAGCGCGCAGAATCGCAA |
| | | ATACACCATCAAAGTTGAAGTACCGAAA |
| | | GTCGCAACGCAGACCGTAGGCGGCGTAG |
| | | AACTCCCAGTTGCGGCCTGGCGCTCTTAC |
| | | CTCAACATGGAACTGACTATTCCGATTTT |
| | | TGCGACGAACTCCGACTGCGAACTGATTG |
| | | TTAAGGCAATGCAGGGCCTGCTGAAAGA |
| | | CGGTAATCCGATCCCATCTGCAATCGCTG |
| | | CTAACTCTGGCATTTACTAA |
| SEQ ID NO: 6 | T7 terminator | CTAGCATAACCCCTTGGGGCCTCTAAACG |
| | | GGTCTTGAGGGGTTTTTTG |
| SEQ ID NO: 7 | pBR322 origin of | TTGAGATCCTTTTTTTCTGCGCGTAATCTG |
| | replication | CTGCTTGCAAACAAAAAAACCACCGCTA |
| | | CCAGCGGTGGTTTGTTTGCCGGATCAAGA |
| | | GCTACCAACTCTTTTTCCGAAGGTAACTG |
| | | GCTTCAGCAGAGCGCAGATACCAAATAC |
| | | TGTCCTTCTAGTGTAGCCGTAGTTAGGCC |
| | | ACCACTTCAAGAACTCTGTAGCACCGCCT |
| | | ACATACCTCGCTCTGCTAATCCTGTTACC |
| | | AGTGGCTGCTGCCAGTGGCGATAAGTCGT |
| | | GTCTTACCGGGTTGGACTCAAGACGATAG |
| | | TTACCGGATAAGGCGCAGCGGTCGGGCT |
| | | GAACGGGGGGTTCGTGCACACAGCCCAG |
| | | CTTGGAGCGAACGACCTACACCGAACTG |
| | | AGATACCTACAGCGTGAGCTATGAGAAA |
| | | GCGCCACGCTTCCCGAAGGGAGAAAGGC |
| | | GGACAGGTATCCGGTAAGCGGCAGGGTC |
| | | GGAACAGGAGAGCGCACGAGGGAGCTTC |
| | | CAGGGGGAAACGCCTGGTATCTTTATAGT |
| | | CCTGTCGGGTTTCGCCACCTCTGACTTGA |
| | | GCGTCGATTTTTGTGATGCTCGTCAGGGG |
| | | GGCGGAGCCTATGGAAAAACGCCAGCAA |
| | | CGCGGCCTTTTTACGGTTCCTGGCCTTTTG |
| | | CTGGCCTTTTGCTCACATGTTCTTTCCTGC |
| | | GTTATCCCCTGATTCTGTGGATAACCGTA |
| | | TTACCGCCTTTGAGTGAGCTGATACCGCT |
| | | CGCCGCAGCCGAACGACCGAGCGCAGCG |
| | | AGTCAGTGAGCGAGGAAGCGGAAGAGCG |
| | | CCTGATGCGGTATTTTCTCCTTACGCATCT |
| | | GTGCGGTATTTCACACCGCATATGGTGCA |
| | | CTCTCAGTACAATCTGCTCTGATGCCGCA |
| | | TAGTTAAGCCAGTATACACTCCGCTATCG |
| | | CTACGTGACTGGGTCATGGCTGCGCCCCG |
| | | ACACCCGCCAACACCCGCTGACGCGCCCT |
| | | GACGGGCTTGTCTGCTCCCGGCATCCGCT |
| | | TACAGACAAGCTGTGACCGTCTCCGGGA |
| | | GCTGCATGTGTCAGAGGTTTTCACCGTCA |
| | | TCACCGAAACGCGCGAGGCAGCTGCGGT |
| | | AAAGCTCATCAGCGTGGTCGTGAAGCGA |
| | | TTCACAGATGTCTGCCTGTTCATCCGCGT |
| | | CCAGCTCGTTGAGTTTCTCCAGAAGCGTT |
| | | AATGTCTGGCTTCTGATAAAGCGGGCCAT |
| | | GTTAAGGGCGGTTTTTTCCTGTTTGGTCA |
| | | C |
| SEQ ID NO: 8 | Ampicillin | CGCGGAACCCCTATTTGTTTATTTTTCTAA |
| | expression cassette | ATACATTCAAATATGTATCCGCTCATGAG |
| | | ACAATAACCCTGATAAATGCTTCAATAAT |
| | | ATTGAAAAAGGAAGAGTATGAGTATTCA |
| | | ACATTTCCGTGTCGCCCTTATTCCCTTTTT |
| | | TGCGGCATTTTGCCTTCCTGTTTTTGCTCA |
| | | CCCAGAAACGCTGGTGAAAGTAAAAGAT |
| | | GCTGAAGATCAGTTGGGTGCACGAGTGG |
| | | GTTACATCGAACTGGATCTCAACAGCGGT |
| | | AAGATCCTTGAGAGTTTTCGCCCCGAAGA |
| | | ACGTTTTCCAATGATGAGCACTTTTAAAG |
| | | TTCTGCTATGTGGCGCGGTATTATCCCGT |
| | | GTTGACGCCGGGCAAGAGCAACTCGGTC |
| | | GCCGCATACACTATTCTCAGAATGACTTG |
| | | GTTGAGTACTCACCAGTCACAGAAAAGC |
| | | ATCTTACGGATGGCATGACAGTAAGAGA |
| | | ATTATGCAGTGCTGCCATAACCATGAGTG |
| | | ATAACACTGCGGCCAACTTACTTCTGACA |
| | | ACGATCGGAGGACCGAAGGAGCTAACCG |
| | | CTTTTTTGCACAACATGGGGGATCATGTA |
| | | ACTCGCCTTGATCGTTGGGAACCGGAGCT |
| | | GAATGAAGCCATACCAAACGACGAGCGT |
| | | GACACCACGATGCCTGCAGCAATGGCAA |
| | | CAACGTTGCGCAAACTATTAACTGGCGA |
| | | ACTACTTACTCTAGCTTCCCGGCAACAAT |
| | | TAATAGACTGGATGGAGGCGGATAAAGT |
| | | TGCAGGACCACTTCTGCGCTCGGCCCTTC |
| | | CGGCTGGCTGGTTTATTGCTGATAAATCT |
| | | GGAGCCGGTGAGCGTGGGTCTCGCGGTA |
| | | TCATTGCAGCACTGGGGCCAGATGGTAA |
| | | GCCCTCCCGTATCGTAGTTATCTACACGA |
| | | CGGGGAGTCAGGCAACTATGGATGAACG |
| | | AAATAGACAGATCGCTGAGATAGGTGCC |
| | | TCACTGATTAAGCATTGGTAA |
| SEQ ID NO: 33 | promoter J23115 | TTTATAGCTAGCTCAGCCCTTGGTACAAT |
| | | GCTAGC |
| SEQ ID NO: 34 | APSE10448 | TtCCGAAATTAATACGACTCACTATAGGG |
| | | AGACATGAGGATTACCCATGTgcgatcgccGA |
| | | TCTCTCTCCCTCGACTCTAACACCAGCGA |
| | | AAGTAACAGCCAATCAAGatgtgtgacgatgatgtt |
| | | gcggcattagtcgtggacaatgggtccggtatgtgcaaggctggat |
| | | tcgcgggggatgatgcaccacgcgctgtgtttcccagcatcgtcgg |
| | | tcgtcctcgtcatcagggtgtgatggtcggtatgggtcaaaaagaca |
| | | gttatgttggcgacgaggcgcaaagtaagagaggtatattgacact |
| | | aaagtatcctatagaacatggcattattactaattgggatgacatggG |
| | | TTTAAACCCTCTAGCTGCTTTACAAAGTA |
| | | CTGGTTCCCTTTCCAGCGGGATGCTTTAT |
| | | CTAAACGCAATGAGAGAGGTATTCCTCA |
| | | GGCCACATCGCTTCCTAGTTCCGCTGGGA |
| | | TCCATCGTTGGCGGCCGAAGCCGCCATTC |
| | | CATAGTGAGTTCTGGCGCGCCccatgtcatccca |
| | | attagtaataatgccatgttctataggatactttagtgtcaatatacctct |
| | | cttactttgcgcctcgtcgccaacataactgtctttttgacccataccg |
| | | accatcacaccctgatgacgaggacgaccgacgatgctgggaaac |
| | | acagcgcgtggtgcatcatcccccgcgaatccagccttgcacatac |
| | | cggacccattgtccacgactaatgccgcaacatcatcgtcacacat |
| | | CTTGATTGGCTGTTACTTTCGCTGGTGTTA |
| | | GAGTCGAGGGAGAGAGATCgcggaccgAAT |
| | | ACCCGGTCTGAACGAGGGCGGCCGCGGT |
| | | ACCCAAGAAGTACTTAGAGTTAATTAAG |
| | | GAGTTCAAACATGAGGATCACCCATGTC |
| | | GAAGCTCCCACACCCTAGCATAACCCCTT |
| | | GGGGCCTCTAAACGGGTCTTGAGGGGTTT |
| | | TTTGCTGAAAGGAGGAACTATATCCGGAT |
| | | ATCCACAGGACGGGTGTGGTCGCCATGA |
| | | TCGCGTAGTCGATAGTGGCTCCAAGTAGC |
| | | GAAGCGAGCAGGACTGGGCGGCGGGCAT |
| | | GCATCGTCCATTCCGACAGCATCGCCAGT |
| | | CACTATGGCGTGCTGCTAGCGCTATATGC |
| | | GTTGATGCAATTTCTATGCGCACCCGTTC |
| | | TCGGAGCACTGTCCGACCGCTTTGGCCGC |
| | | CGCCCAGTCCTGCTCGCTTCGCTACTTGG |
| | | AGCCACTATCGACTACGCGATCATGGCG |
| | | ACCACACCCGTCCTGTGGATCCAGATCTC |
| | | GATCCCGCGAAATTAATACGACTCACTAT |
| | | AGGGAGACCACAACGGTTTCCCTCTAGAT |
| | | CACAAGTTTGTACAAAAAAGCAGGCTAA |
| | | GAAGGAGATATACATATGGCGTCTAACTT |
| | | TACCCAATTCGTTCTGGTTGATAACGGCG |
| | | GTACGGGTGACGTTACCGTAGCTCCGTCC |
| | | AACTTCGCCAACGGTGTTGCGGAATGGAT |
| | | TAGCTCTAACAGCCGCTCTCAGGCCTACA |
| | | AAGTCACGTGCTCCGTTCGTCAGTCTAGC |
| | | GCGCAGAATCGCAAATACACCATCAAAG |
| | | TTGAAGTACCGAAAGTCGCAACGCAGAC |
| | | CGTAGGCGGCGTAGAACTCCCAGTTGCG |
| | | GCCTGGCGCTCTTACCTCAACATGGAACT |
| | | GACTATTCCGATTTTTGCGACGAACTCCG |
| | | ACTGCGAACTGATTGTTAAGGCAATGCA |
| | | GGGCCTGCTGAAAGACGGTAATCCGATC |
| | | CCATCTGCAATCGCTGCTAACTCTGGCAT |
| | | TTACTAATAAGCGGACGCGCTGCCACCGC |
| | | TGAGCAATAACTAGCATAACCCCTTGGG |
| | | GCCTCTAAACGGGTCTTGAGGGGTTTTTT |
| | | GCTGAAAGGAGGAACTATATCCGGCATG |
| | | CACCATTCCTTGCGGCGGCGGTGCTCAAC |
| | | GGCCTCAACCTACTACTGGGCTGCTTCCT |
| | | AATGCAGGAGTCGCATAAGGGAGAGCgtc |
| | | gacAGAAGGAGATATACATATGaaaccatatcag |
| | | cgccagtttattGaatttgcgcttagcaagcaggtgttaaagtttggc |
| | | gagtttacgctgaaatccgggcgcaaaagcccctatttcttcaacgc |
| | | cgggctgtttaataccgggcgcgatctggcactgttaggccgttttta |
| | | cgctgaagcgttggtggattccggcattgagttcgatctgctgtttgg |
| | | ccctgcttacaaagggatcccgattgccaccacaaccgctgtggca |
| | | ctggcggagcatcacgacctggacctgccgtactgctttaaccgca |
| | | aagaagcaaaagaccacggtgaaggcggcaatctggttggtagc |
| | | gcgttacaaggacgcgtaatgctggtagatgatgtgatcaccgccg |
| | | gaacggcgattcgcgagtcgatggagattattcaggccaatggcgc |
| | | gacgcttgctggcgtgttgatttcgctcgatcgtcaggaacggggc |
| | | gcggcgagatttcggcgattcaggaagttgagcgtgattacaactg |
| | | caaagtgatctctatcatcaccctgaaagacctgattgcttacctgga |
| | | agagaagccggaaalggcggaacatctggggggttaaggcct |
| | | atcgcgaagagtttggcgtttaacctaggCAGAACGCAGA |
| | | AGCtGTCTGATAAAACAGAATTTGCCTGG |
| | | CGGCAGTAGCGCGGTGGTCCCACCTGAC |
| | | CCCATGCCGAACTCAGAAGTGAAACGCC |
| | | GTAGCGCCGATGGTAGTGTGGGGTCTCCC |
| | | CATGCGAGAGTAGGGAACTGCCAGGCAT |
| | | CAAATAAAACGAAAGGCTCAGTCGAAAG |
| | | ACTGGGCCTTTCGTTTTATCTGTTGTTTGT |
| | | CGGTGAACGCTCTCCTGAGTAGGACAAA |
| | | TCCGCCGGGAGCGGATTTGAACGTTGCG |
| | | AAGCAACGGCCCGGAGGGTGGCGGGCAG |
| | | GACGCCCGCCATAAACTGCCAGGCATCA |
| | | AATTAAGCAGAAGGCCATCCTGACGGAT |
| | | GGCCTTTTTGCGTTTCTACtcgcgaCGCGAG |
| | | GCTGGATGGCCTTCCCCATTATGATTCTT |
| | | CTCGCTTCCGGCGGCATCGGGATGCCCGC |
| | | GTTGCAGGCCATGCTGTCCAGGCAGGTA |
| | | GATGACGACCATCAGGGACAGCTTCAAG |
| | | GATCGCTCGCGGCTCTTACCAGCCTAACT |
| | | TCGATCATTGGACCGCTGATCGTCACGGC |
| | | GATTTATGCCGCCTCGGCGAGCACATGGA |
| | | ACGGGTTGGCATGGATTGTAGGCGCCGC |
| | | CCTATACCTTGTCTGCCTCCCCGCGTTGC |
| | | GTCGCGGTGCATGGAGCCGGGCCACCTC |
| | | GACCTGAATGGAAGCCGGCGGCACCTCG |
| | | CTAACGGATTCACCACTCCAAGAATTGGA |
| | | GCCAATCAATTCTTGCGGAGAACTGTGAA |
| | | TGCGCAAACCAACCCTTGGCAGAACATA |
| | | TCCATCGCGTCCGCCATCTCCAGCAGCCG |
| | | CACGCGGCGCATCTCGGGCAGCGTTGGG |
| | | TCCTGGCCACGGGTGCGCATGATCGTGCT |
| | | CCTGTCGTTGAGGACCCGGCTAGGCTGGC |
| | | GGGGTTGCCTTACTGGTTAGCAGAATGAA |
| | | TCACCGATACGCGAGCGAACGTGAAGCG |
| | | ACTGCTGCTGCAAAACGTCTGCGACCTGA |
| | | GCAACAACATGAATGGTCTTCGGTTTCCG |
| | | TGTTTCGTAAAGTCTGGAAACGCGGAAGT |
| | | CAGCGCCCTGCACCATTATGTTCCGGATC |
| | | TGCATCGCAGGATGCTGCTGGCTACCCTG |
| | | TGGAACACCTACATCTGTATTAACGAAGC |
| | | GCTGGCATTGACCCTGAGTGATTTTTCTC |
| | | TGGTCCCGCCGCATCCATACCGCCAGTTG |
| | | TTTACCCTCACAACGTTCCAGTAACCGGG |
| | | CATGTTCATCATCAGTAACCCGTATCGTG |
| | | AGCATCCTCTCTCGTTTCATCGGTATCATT |
| | | ACCCCCATGAACAGAAATCCCCCTTACAC |
| | | GGAGGCATCAGTGACCAAACAGGAAAAA |
| | | ACCGCCCTTAACATGGCCCGCTTTATCAG |
| | | AAGCCAGACATTAACGCTTCTGGAGAAA |
| | | CTCAACGAGCTGGACGCGGATGAACAGG |
| | | CAGACATCTGTGAATCGCTTCACGACCAC |
| | | GCTGATGAGCTTTACCGCAGCTGCCTCGC |
| | | GCGTTTCGGTGATGACGGTGAAAACCTCT |
| | | GACACATGCAGCTCCCGGAGACGGTCAC |
| | | AGCTTGTCTGTAAGCGGATGCCGGGAGC |
| | | AGACAAGCCCGTCAGGGCGCGTCAGCGG |
| | | GTGTTGGCGGGTGTCGGGGCGCAGCCAT |
| | | GACCCAGTCACGTAGCGATAGCGGAGTG |
| | | TATACTGGCTTAACTATGCGGCATCAGAG |
| | | CAGATTGTACTGAGAGTGCACCATATGCG |
| | | GTGTGAAATACCGCACAGATGCGTAAGG |
| | | AGAAAATACCGCATCAGGCGCTCTTCCGC |
| | | TTCCTCGCTCACTGACTCGCTGCGCTCGG |
| | | TCGTTCGGCTGCGGCGAGCGGTATCAGCT |
| | | CACTCAAAGGCGGTAATACGGTTATCCAC |
| | | AGAATCAGGGGATAACGCAGGAAAGAAC |
| | | ATGTGAGCAAAAGGCCAGCAAAAGGCCA |
| | | GGAACCGTAAAAAGGCCGCGTTGCTGGC |
| | | GTTTTTCCATAGGCTCCGCCCCCCTGACG |
| | | AGCATCACAAAAATCGACGCTCAAGTCA |
| | | GAGGTGGCGAAACCCGACAGGACTATAA |
| | | AGATACCAGGCGTTTCCCCCTGGAAGCTC |
| | | CCTCGTGCGCTCTCCTGTTCCGACCCTGC |
| | | CGCTTACCGGATACCTGTCCGCCTTTCTC |
| | | CCTTCGGGAAGCGTGGCGCTTTCTCATAG |
| | | CTCACGCTGTAGGTATCTCAGTTCGGTGT |
| | | AGGTCGTTCGCTCCAAGCTGGGCTGTGTG |
| | | CACGAACCCCCCGTTCAGCCCGACCGCTG |
| | | CGCCTTATCCGGTAACTATCGTCTTGAGT |
| | | CCAACCCGGTAAGACACGACTTATCGCC |
| | | ACTGGCAGCAGCCACTGGTAACAGGATT |
| | | AGCAGAGCGAGGTATGTAGGCGGTGCTA |
| | | CAGAGTTCTTGAAGTGGTGGCCTAACTAC |
| | | GGCTACACTAGAAGGACAGTATTTGGTAT |
| | | CTGCGCTCTGCTGAAGCCAGTTACCTTCG |
| | | GAAAAAGAGTTGGTAGCTCTTGATCCGG |
| | | CAAACAAACCACCGCTGGTAGCGGTGGT |
| | | TTTTTTGTTTGCAAGCAGCAGATTACGCG |
| | | CAGAAAAAAAGGATCTCAAGAAGATCCT |
| | | TTGATCTTTTCTACGGGGTCTGACGCTCA |
| | | GTGGAACGAAAACTCACGTTAAGGGATT |
| | | TTGGTCATGAGATTATCAAAAAGGATCTT |
| | | CACCTAGATCCTTTTAAATTAAAAATGAA |
| | | GTTTTAAATCAATCTAAAGTATATATGAG |
| | | TAAACTTGGTCTGACAGTTACCAATGCTT |
| | | AATCAGTGAGGCACCTATCTCAGCGATCT |
| | | GTCTATTTCGTTCATCCATAGTTGCCTGA |
| | | CTCCCCGTCGTGTAGATAACTACGATACG |
| | | GGAGGGCTTACCATCTGGCCCCAGTGCTG |
| | | CAATGATACCGCGAGACCCACGCTCACC |
| | | GGCTCCAGATTTATCAGCAATAAACCAGC |
| | | CAGCCGGAAGGGCCGAGCGCAGAAGTGG |
| | | TCCTGCAACTTTATCCGCCTCCATCCAGT |
| | | CTATTAATTGTTGCCGGGAAGCTAGAGTA |
| | | AGTAGTTCGCCAGTTAATAGTTTGCGCAA |
| | | CGTTGTTGCCATTGCTGCAGGCATCGTGG |
| | | TGTCACGCTCGTCGTTTGGTATGGCTTCA |
| | | TTCAGCTCCGGTTCCCAACGATCAAGGCG |
| | | AGTTACATGATCCCCCATGTTGTGCAAAA |
| | | AAGCGGTTAGCTCCTTCGGTCCTCCGATC |
| | | GTTGTCAGAAGTAAGTTGGCCGCAGTGTT |
| | | ATCACTCATGGTTATGGCAGCACTGCATA |
| | | ATTCTCTTACTGTCATGCCATCCGTAAGA |
| | | TGCTTTTCTGTGACTGGTGAGTACTCAAC |
| | | CAAGTCATTCTGAGAATAGTGTATGCGGC |
| | | GACCGAGTTGCTCTTGCCCGGCGTCAACA |
| | | CGGGATAATACCGCGCCACATAGCAGAA |
| | | CTTTAAAAGTGCTCATCATTGGAAAACGT |
| | | TCTTCGGGGCGAAAACTCTCAAGGATCTT |
| | | ACCGCTGTTGAGATCCAGTTCGATGTAAC |
| | | CCACTCGTGCACCCAACTGATCTTCAGCA |
| | | TCTTTTACTTTCACCAGCGTTTCTGGGTGA |
| | | GCAAAAACAGGAAGGCAAAATGCCGCAA |
| | | AAAAGGGAATAAGGGCGACACGGAAATG |
| | | TTGAATACTCATACTCTTCCTTTTTCAATA |
| | | TTATTGAAGCATTTATCAGGGTTATTGTC |
| | | TCATGAGCGGATACATATTTGAATGTATT |
| | | TAGAAAAATAAACAAATAGGGGTTCCGC |
| | | GCACATTTCCCCGAAAAGTGCCACCTGAC |
| | | GTCTAAGAAACCATTATTATCATGACATT |
| | | AACCTATAAAAATAGGCGTATCACGAGG |
| | | CCCTTTCGTCTTCAAGAA |
| SEQ ID NO: 35 | APSE10471 | TtCCGAAATTAATACGACTCACTATAGGG |
| | | AGACATGAGGATTACCCATGTgcgatcgccGA |
| | | TCTCTCTCCCTCGACTCTAACACCAGCGA |
| | | AAGTAACAGCCAATCAAGatgtgtgacgatgatgtt |
| | | gcggcattagtcgtggacaatgggtccggtatgtgcaaggctggat |
| | | tcgcgggggatgatgcaccacgcgctgtgtttcccagcatcgtcgg |
| | | tcgtcctcgtcatcagggtgtgatggtcggtatgggtcaaaaagaca |
| | | gttatgttggcgacgaggcgcaaagtaagagaggtatattgacact |
| | | aaagtatcctatagaacatggcattattactaattgggatgacatggG |
| | | TTTAAACCCTCTAGCTGCTTTACAAAGTA |
| | | CTGGTTCCCTTTCCAGCGGGATGCTTTAT |
| | | CTAAACGCAATGAGAGAGGTATTCCTCA |
| | | GGCCACATCGCTTCCTAGTTCCGCTGGGA |
| | | TCCATCGTTGGCGGCCGAAGCCGCCATTC |
| | | CATAGTGAGTTCTGGCGCGCCccatgtcatccca |
| | | attagtaataatgccatgttctataggatactttagtgtcaatatacctct |
| | | cttactttgcgcctcgtcgccaacataactgtctttttgacccataccg |
| | | accatcacaccctgatgacgaggacgaccgacgatgctgggaaac |
| | | acagcgcgtggtgcatcatcccccgcgaatccagccttgcacatac |
| | | cggacccattgtccacgactaatgccgcaacatcatcgtcacacat |
| | | CTTGATTGGCTGTTACTTTCGCTGGTGTTA |
| | | GAGTCGAGGGAGAGAGATCgcggaccgAAT |
| | | ACCCGGTCTGAACGAGGGCGGCCGCGGT |
| | | ACCCAAGAAGTACTTAGAGTTAATTAAG |
| | | GAGTTCAAACATGAGGATCACCCATGTC |
| | | GAAGCTCCCACACCCTAGCATAACCCCTT |
| | | GGGGCCTCTAAACGGGTCTTGAGGGGTTT |
| | | TTTGCTGAAAGGAGGAACTATATCCGGAT |
| | | ATCCACAGGACGGGTGTGGTCGCCATGA |
| | | TCGCGTAGTCGATAGTGGCTCCAAGTAGC |
| | | GAAGCGAGCAGGACTGGGCGGCGGGCAT |
| | | GCATCGTCCATTCCGACAGCATCGCCAGT |
| | | CACTATGGCGTGCTGCTAGCGCTATATGC |
| | | GTTGATGCAATTTCTATGCGCACCCGTTC |
| | | TCGGAGCACTGTCCGACCGCTTTGGCCGC |
| | | CGCCCAGTCCTGCTCGCTTCGCTACTTGG |
| | | AGCCACTATCGACTACGCGATCATGGCG |
| | | ACCACACCCGTCCTGTGGATCCAGATCTC |
| | | GATCCCGCGAAATTAATACGACTCACTAT |
| | | AGGGAGACCACAACGGTTTCCCTCTAGAT |
| | | CACAAGTTTGTACAAAAAAGCAGGCTAA |
| | | GAAGGAGATATACATATGGCGTCTAACTT |
| | | TACCCAATTCGTTCTGGTTGATAACGGCG |
| | | GTACGGGTGACGTTACCGTAGCTCCGTCC |
| | | AACTTCGCCAACGGTGTTGCGGAATGGAT |
| | | TAGCTCTAACAGCCGCTCTCAGGCCTACA |
| | | AAGTCACGTGCTCCGTTCGTCAGTCTAGC |
| | | GCGCAGAATCGCAAATACACCATCAAAG |
| | | TTGAAGTACCGAAAGTCGCAACGCAGAC |
| | | CGTAGGCGGCGTAGAACTCCCAGTTGCG |
| | | GCCTGGCGCTCTTACCTCAACATGGAACT |
| | | GACTATTCCGATTTTTGCGACGAACTCCG |
| | | ACTGCGAACTGATTGTTAAGGCAATGCA |
| | | GGGCCTGCTGAAAGACGGTAATCCGATC |
| | | CCATCTGCAATCGCTGCTAACTCTGGCAT |
| | | TTACTAATAAGCGGACGCGCTGCCACCGC |
| | | TGAGCAATAACTAGCATAACCCCTTGGG |
| | | GCCTCTAAACGGGTCTTGAGGGGTTTTTT |
| | | GCTGAAAGGAGGAACTATATCCGGCATG |
| | | CACCATTCCTTGCGGCGGCGGTGCTCAAC |
| | | GGCCTCAACCTACTACTGGGCTGCTTCCT |
| | | AATGCAGGAGTCGCATAAGGGAGAGCgtc |
| | | gacTAATACGACTCACtATAGGGAGACCAC |
| | | AgaaggagATataCATATGaaaccatatcagcgccagttta |
| | | ttGaatttgcgcttagcaagcaggtgttaaagtttggcgagtttacgc |
| | | tgaaatccgggcgcaaaagcccctatttcttcaacgccgggctgttt |
| | | aataccgggcgcgatctggcactgttaggccgtttttacgctgaagc |
| | | gttggtggattccggcattgagttcgatctgctgtttggccctgcttac |
| | | aaagggatcccgattgccaccacaaccgctgtggcactggcggag |
| | | catcacgacctggacctgccgtactgctttaaccgcaaagaagcaa |
| | | aagaccacggtgaaggcggcaatctggttggtagcgcgttacaag |
| | | gacgcgtaatgctggtagatgatgtgatcaccgccggaacggcgat |
| | | tcgcgagtcgatggagattattcaggccaatggcgcgacgcttgct |
| | | ggcgtgttgatttcgctcgatcgtcaggaacgcgggcgcggcgag |
| | | atttcggcgattcaggaagttgagcgtgattacaactgcaaagtgat |
| | | ctctatcatcaccctgaaagacctgattgcttacctggaagagaagc |
| | | cggaaatggcggaacatctggcggcggttaaggcctatcgcgaag |
| | | agtttggcgtttaacctaggCAGAACGCAGAAGCtGT |
| | | CTGATAAAACAGAATTTGCCTGGCGGCA |
| | | GTAGCGCGGTGGTCCCACCTGACCCCATG |
| | | CCGAACTCAGAAGTGAAACGCCGTAGCG |
| | | CCGATGGTAGTGTGGGGTCTCCCCATGCG |
| | | AGAGTAGGGAACTGCCAGGCATCAAATA |
| | | AAACGAAAGGCTCAGTCGAAAGACTGGG |
| | | CCTTTCGTTTTATCTGTTGTTTGTCGGTGA |
| | | ACGCTCTCCTGAGTAGGACAAATCCGCCG |
| | | GGAGCGGATTTGAACGTTGCGAAGCAAC |
| | | GGCCCGGAGGGTGGCGGGCAGGACGCCC |
| | | GCCATAAACTGCCAGGCATCAAATTAAG |
| | | CAGAAGGCCATCCTGACGGATGGCCTTTT |
| | | TGCGTTTCTACtcgcgaCGCGAGGCTGGATG |
| | | GCCTTCCCCATTATGATTCTTCTCGCTTCC |
| | | GGCGGCATCGGGATGCCCGCGTTGCAGG |
| | | CCATGCTGTCCAGGCAGGTAGATGACGA |
| | | CCATCAGGGACAGCTTCAAGGATCGCTC |
| | | GCGGCTCTTACCAGCCTAACTTCGATCAT |
| | | TGGACCGCTGATCGTCACGGCGATTTATG |
| | | CCGCCTCGGCGAGCACATGGAACGGGTT |
| | | GGCATGGATTGTAGGCGCCGCCCTATACC |
| | | TTGTCTGCCTCCCCGCGTTGCGTCGCGGT |
| | | GCATGGAGCCGGGCCACCTCGACCTGAA |
| | | TGGAAGCCGGCGGCACCTCGCTAACGGA |
| | | TTCACCACTCCAAGAATTGGAGCCAATCA |
| | | ATTCTTGCGGAGAACTGTGAATGCGCAA |
| | | ACCAACCCTTGGCAGAACATATCCATCGC |
| | | GTCCGCCATCTCCAGCAGCCGCACGCGGC |
| | | GCATCTCGGGCAGCGTTGGGTCCTGGCCA |
| | | CGGGTGCGCATGATCGTGCTCCTGTCGTT |
| | | GAGGACCCGGCTAGGCTGGCGGGGTTGC |
| | | CTTACTGGTTAGCAGAATGAATCACCGAT |
| | | ACGCGAGCGAACGTGAAGCGACTGCTGC |
| | | TGCAAAACGTCTGCGACCTGAGCAACAA |
| | | CATGAATGGTCTTCGGTTTCCGTGTTTCG |
| | | TAAAGTCTGGAAACGCGGAAGTCAGCGC |
| | | CCTGCACCATTATGTTCCGGATCTGCATC |
| | | GCAGGATGCTGCTGGCTACCCTGTGGAAC |
| | | ACCTACATCTGTATTAACGAAGCGCTGGC |
| | | ATTGACCCTGAGTGATTTTTCTCTGGTCC |
| | | CGCCGCATCCATACCGCCAGTTGTTTACC |
| | | CTCACAACGTTCCAGTAACCGGGCATGTT |
| | | CATCATCAGTAACCCGTATCGTGAGCATC |
| | | CTCTCTCGTTTCATCGGTATCATTACCCCC |
| | | ATGAACAGAAATCCCCCTTACACGGAGG |
| | | CATCAGTGACCAAACAGGAAAAAACCGC |
| | | CCTTAACATGGCCCGCTTTATCAGAAGCC |
| | | AGACATTAACGCTTCTGGAGAAACTCAA |
| | | CGAGCTGGACGCGGATGAACAGGCAGAC |
| | | ATCTGTGAATCGCTTCACGACCACGCTGA |
| | | TGAGCTTTACCGCAGCTGCCTCGCGCGTT |
| | | TCGGTGATGACGGTGAAAACCTCTGACA |
| | | CATGCAGCTCCCGGAGACGGTCACAGCTT |
| | | GTCTGTAAGCGGATGCCGGGAGCAGACA |
| | | AGCCCGTCAGGGCGCGTCAGCGGGTGTT |
| | | GGCGGGTGTCGGGGCGCAGCCATGACCC |
| | | AGTCACGTAGCGATAGCGGAGTGTATAC |
| | | TGGCTTAACTATGCGGCATCAGAGCAGAT |
| | | TGTACTGAGAGTGCACCATATGCGGTGTG |
| | | AAATACCGCACAGATGCGTAAGGAGAAA |
| | | ATACCGCATCAGGCGCTCTTCCGCTTCCT |
| | | CGCTCACTGACTCGCTGCGCTCGGTCGTT |
| | | CGGCTGCGGCGAGCGGTATCAGCTCACTC |
| | | AAAGGCGGTAATACGGTTATCCACAGAA |
| | | TCAGGGGATAACGCAGGAAAGAACATGT |
| | | GAGCAAAAGGCCAGCAAAAGGCCAGGA |
| | | ACCGTAAAAAGGCCGCGTTGCTGGCGTTT |
| | | TTCCATAGGCTCCGCCCCCCTGACGAGCA |
| | | TCACAAAAATCGACGCTCAAGTCAGAGG |
| | | TGGCGAAACCCGACAGGACTATAAAGAT |
| | | ACCAGGCGTTTCCCCCTGGAAGCTCCCTC |
| | | GTGCGCTCTCCTGTTCCGACCCTGCCGCT |
| | | TACCGGATACCTGTCCGCCTTTCTCCCTTC |
| | | GGGAAGCGTGGCGCTTTCTCATAGCTCAC |
| | | GCTGTAGGTATCTCAGTTCGGTGTAGGTC |
| | | GTTCGCTCCAAGCTGGGCTGTGTGCACGA |
| | | ACCCCCCGTTCAGCCCGACCGCTGCGCCT |
| | | TATCCGGTAACTATCGTCTTGAGTCCAAC |
| | | CCGGTAAGACACGACTTATCGCCACTGGC |
| | | AGCAGCCACTGGTAACAGGATTAGCAGA |
| | | GCGAGGTATGTAGGCGGTGCTACAGAGT |
| | | TCTTGAAGTGGTGGCCTAACTACGGCTAC |
| | | ACTAGAAGGACAGTATTTGGTATCTGCGC |
| | | TCTGCTGAAGCCAGTTACCTTCGGAAAAA |
| | | GAGTTGGTAGCTCTTGATCCGGCAAACAA |
| | | ACCACCGCTGGTAGCGGTGGTTTTTTTGT |
| | | TTGCAAGCAGCAGATTACGCGCAGAAAA |
| | | AAAGGATCTCAAGAAGATCCTTTGATCTT |
| | | TTCTACGGGGTCTGACGCTCAGTGGAACG |
| | | AAAACTCACGTTAAGGGATTTTGGTCATG |
| | | AGATTATCAAAAAGGATCTTCACCTAGAT |
| | | CCTTTTAAATTAAAAATGAAGTTTTAAAT |
| | | CAATCTAAAGTATATATGAGTAAACTTGG |
| | | TCTGACAGTTACCAATGCTTAATCAGTGA |
| | | GGCACCTATCTCAGCGATCTGTCTATTTC |
| | | GTTCATCCATAGTTGCCTGACTCCCCGTC |
| | | GTGTAGATAACTACGATACGGGAGGGCT |
| | | TACCATCTGGCCCCAGTGCTGCAATGATA |
| | | CCGCGAGACCCACGCTCACCGGCTCCAG |
| | | ATTTATCAGCAATAAACCAGCCAGCCGG |
| | | AAGGGCCGAGCGCAGAAGTGGTCCTGCA |
| | | ACTTTATCCGCCTCCATCCAGTCTATTAA |
| | | TTGTTGCCGGGAAGCTAGAGTAAGTAGTT |
| | | CGCCAGTTAATAGTTTGCGCAACGTTGTT |
| | | GCCATTGCTGCAGGCATCGTGGTGTCACG |
| | | CTCGTCGTTTGGTATGGCTTCATTCAGCT |
| | | CCGGTTCCCAACGATCAAGGCGAGTTAC |
| | | ATGATCCCCCATGTTGTGCAAAAAAGCG |
| | | GTTAGCTCCTTCGGTCCTCCGATCGTTGT |
| | | CAGAAGTAAGTTGGCCGCAGTGTTATCAC |
| | | TCATGGTTATGGCAGCACTGCATAATTCT |
| | | CTTACTGTCATGCCATCCGTAAGATGCTT |
| | | TTCTGTGACTGGTGAGTACTCAACCAAGT |
| | | CATTCTGAGAATAGTGTATGCGGCGACCG |
| | | AGTTGCTCTTGCCCGGCGTCAACACGGGA |
| | | TAATACCGCGCCACATAGCAGAACTTTAA |
| | | AAGTGCTCATCATTGGAAAACGTTCTTCG |
| | | GGGCGAAAACTCTCAAGGATCTTACCGCT |
| | | GTTGAGATCCAGTTCGATGTAACCCACTC |
| | | GTGCACCCAACTGATCTTCAGCATCTTTT |
| | | ACTTTCACCAGCGTTTCTGGGTGAGCAAA |
| | | AACAGGAAGGCAAAATGCCGCAAAAAAG |
| | | GGAATAAGGGCGACACGGAAATGTTGAA |
| | | TACTCATACTCTTCCTTTTTCAATATTATT |
| | | GAAGCATTTATCAGGGTTATTGTCTCATG |
| | | AGCGGATACATATTTGAATGTATTTAGAA |
| | | AAATAAACAAATAGGGGTTCCGCGCACA |
| | | TTTCCCCGAAAAGTGCCACCTGACGTCTA |
| | | AGAAACCATTATTATCATGACATTAACCT |
| | | ATAAAAATAGGCGTATCACGAGGCCCTTT |
| | | CGTCTTCAAGAA |
| SEQ ID NO: 36 | APSE10500 | GGATCCTCTAGAGTCGACGCTCTCCCTTA |
| | | TGCGACTCCTGCATTAGGAAGCAGCCCA |
| | | GTAGTAGGTTGAGGCCGTTGAGCACCGC |
| | | CGCCGCAAGGAATGGTGCATGCCGGATA |
| | | TAGTTCCTCCTTTCAGCAAAAAACCCCTC |
| | | AAGACCCGTTTAGAGGCCCCAAGGGGTT |
| | | ATGCTAGTTATTGCTCAGCGGTGGCAGCG |
| | | CGTCCGCTTATTAGTAAATGCCAGAGTTA |
| | | GCAGCGATTGCAGATGGGATCGGATTAC |
| | | CGTCTTTCAGCAGGCCCTGCATTGCCTTA |
| | | ACAATCAGTTCGCAGTCGGAGTTCGTCGC |
| | | AAAAATCGGAATAGTCAGTTCCATGTTGA |
| | | GGTAAGAGCGCCAGGCCGCAACTGGGAG |
| | | TTCTACGCCGCCTACGGTCTGCGTTGCGA |
| | | CTTTCGGTACTTCAACTTTGATGGTGTATT |
| | | TGCGATTCTGCGCGCTAGACTGACGAACG |
| | | GAGCACGTGACTTTGTAGGCCTGAGAGC |
| | | GGCTGTTAGAGCTAATCCATTCCGCAACA |
| | | CCGTTGGCGAAGTTGGACGGAGCTACGG |
| | | TAACGTCACCCGTACCGCCGTTATCAACC |
| | | AGAACGAATTGGGTAAAGTTAGACGCCA |
| | | TATGTATATCTCCTTCTTAGCCTGCTTTTT |
| | | TGTACAAACTTGTGATCTAGAGGGAAAC |
| | | CGTTGTGGTCTCCCTATAGTGAGTCGTAT |
| | | TAATTTCGCGGGATCGAGATCTGGATCCA |
| | | CAGGACGGGTGTGGTCGCCATGATCGCG |
| | | TAGTCGATAGTGGCTCCAAGTAGCGAAG |
| | | CGAGCAGGACTGGGCGGCGGCCAAAGCG |
| | | GTCGGACAGTGCTCCGAGAACGGGTGCG |
| | | CATAGAAATTGCATCAACGCATATAGCG |
| | | CTAGCAGCACGCCATAGTGACTGGCGAT |
| | | GCTGTCGGAATGGACGATGCATGCCCGC |
| | | CGCCCAGTCCTGCTCGCTTCGCTACTTGG |
| | | AGCCACTATCGACTACGCGATCATGGCG |
| | | ACCACACCCGTCCTGTGGATATCCGGATA |
| | | TAGTTCCTCCTTTCAGCAAAAAACCCCTC |
| | | AAGACCCGTTTAGAGGCCCCAAGGGGTT |
| | | ATGCTAGGGTGTGGGAGCTTCGACATGG |
| | | GTGATCCTCATGTTTGAACTCCTTAATTA |
| | | ACTCTAAGTACTTCTTGGGTACCGCGGCC |
| | | GCCCTCGTTCAGACCGGGTATTCGGTCCG |
| | | CGATCTCTCTCCCTCGACTCTAACACCAG |
| | | CGAAAGTAACAGCCAATCAAGATGTGTG |
| | | ACGATGATGTTGCGGCATTAGTCGTGGAC |
| | | AATGGGTCCGGTATGTGCAAGGCTGGATT |
| | | CGCGGGGGATGATGCACCACGCGCTGTG |
| | | TTTCCCAGCATCGTCGGTCGTCCTCGTCA |
| | | TCAGGGTGTGATGGTCGGTATGGGTCAA |
| | | AAAGACAGTTATGTTGGCGACGAGGCGC |
| | | AAAGTAAGAGAGGTATATTGACACTAAA |
| | | GTATCCTATAGAACATGGCATTATTACTA |
| | | ATTGGGATGACATGGGGCGCGCCAGAAC |
| | | TCACTATGGAATGGCGGCTTCGGCCGCCA |
| | | ACGATGGATCCCAGCGGAACTAGGAAGC |
| | | GATGTGGCCTGAGGAATACCTCTCTCATT |
| | | GCGTTTAGATAAAGCATCCCGCTGGAAA |
| | | GGGAACCAGTACTTTGTAAAGCAGCTAG |
| | | AGGGTTTAAACCCATGTCATCCCAATTAG |
| | | TAATAATGCCATGTTCTATAGGATACTTT |
| | | AGTGTCAATATACCTCTCTTACTTTGCGC |
| | | CTCGTCGCCAACATAACTGTCTTTTTGAC |
| | | CCATACCGACCATCACACCCTGATGACGA |
| | | GGACGACCGACGATGCTGGGAAACACAG |
| | | CGCGTGGTGCATCATCCCCCGCGAATCCA |
| | | GCCTTGCACATACCGGACCCATTGTCCAC |
| | | GACTAATGCCGCAACATCATCGTCACACA |
| | | TCTTGATTGGCTGTTACTTTCGCTGGTGTT |
| | | AGAGTCGAGGGAGAGAGATCGGCGATCG |
| | | CACATGGGTAATCCTCATGTCTCCCTATA |
| | | GTGAGTCGTATTAATTTCGGGAGAATTCT |
| | | TGAAGACGAAAGGGCCTCGTGATACGCC |
| | | TATTTTTATAGGTTAATGTCATGATAATA |
| | | ATGGTTTCTTAGACGTCAGGTGGCACTTT |
| | | TCGGGGAAATGTGCGCGGAACCCCTATTT |
| | | GTTTATTTTTCTAAATACATTCAAATATGT |
| | | ATCCGCTCATGAGACAATAACCCTGATAA |
| | | ATGCTTCAATAATATTGAAAAAGGAAGA |
| | | GTATGAGTATTCAACATTTCCGTGTCGCC |
| | | CTTATTCCCTTTTTTGCGGCATTTTGCCTT |
| | | CCTGTTTTTGCTCACCCAGAAACGCTGGT |
| | | GAAAGTAAAAGATGCTGAAGATCAGTTG |
| | | GGTGCACGAGTGGGTTACATCGAACTGG |
| | | ATCTCAACAGCGGTAAGATCCTTGAGAGT |
| | | TTTCGCCCCGAAGAACGTTTTCCAATGAT |
| | | GAGCACTTTTAAAGTTCTGCTATGTGGCG |
| | | CGGTATTATCCCGTGTTGACGCCGGGCAA |
| | | GAGCAACTCGGTCGCCGCATACACTATTC |
| | | TCAGAATGACTTGGTTGAGTACTCACCAG |
| | | TCACAGAAAAGCATCTTACGGATGGCAT |
| | | GACAGTAAGAGAATTATGCAGTGCTGCC |
| | | ATAACCATGAGTGATAACACTGCGGCCA |
| | | ACTTACTTCTGACAACGATCGGAGGACCG |
| | | AAGGAGCTAACCGCTTTTTTGCACAACAT |
| | | GGGGGATCATGTAACTCGCCTTGATCGTT |
| | | GGGAACCGGAGCTGAATGAAGCCATACC |
| | | AAACGACGAGCGTGACACCACGATGCCT |
| | | GCAGCAATGGCAACAACGTTGCGCAAAC |
| | | TATTAACTGGCGAACTACTTACTCTAGCT |
| | | TCCCGGCAACAATTAATAGACTGGATGG |
| | | AGGCGGATAAAGTTGCAGGACCACTTCT |
| | | GCGCTCGGCCCTTCCGGCTGGCTGGTTTA |
| | | TTGCTGATAAATCTGGAGCCGGTGAGCGT |
| | | GGGTCTCGCGGTATCATTGCAGCACTGGG |
| | | GCCAGATGGTAAGCCCTCCCGTATCGTAG |
| | | TTATCTACACGACGGGGAGTCAGGCAAC |
| | | TATGGATGAACGAAATAGACAGATCGCT |
| | | GAGATAGGTGCCTCACTGATTAAGCATTG |
| | | GTAACTGTCAGACCAAGTTTACTCATATA |
| | | TACTTTAGATTGATTTAAAACTTCATTTTT |
| | | AATTTAAAAGGATCTAGGTGAAGATCCTT |
| | | TTTGATAATCTCATGACCAAAATCCCTTA |
| | | ACGTGAGTTTTCGTTCCACTGAGCGTCAG |
| | | ACCCCTTAATAAGATGATCTTCTTGAGAT |
| | | CGTTTTGGTCTGCGCGTAATCTCTTGCTCT |
| | | GAAAACGAAAAAACCGCCTTGCAGGGCG |
| | | GTTTTTCGAAGGTTCTCTGAGCTACCAAC |
| | | TCTTTGAACCGAGGTAACTGGCTTGGAGG |
| | | AGCGCAGTCACCAAAACTTGTCCTTTCAG |
| | | TTTAGCCTTAACCGGCGCATGACTTCAAG |
| | | ACTAACTCCTCTAAATCAATTACCAGTGG |
| | | CTGCTGCCAGTGGTGCTTTTGCATGTCTTT |
| | | CCGGGTTGGACTCAAGACGATAGTTACC |
| | | GGATAAGGCGCAGCGGTCGGACTGAACG |
| | | GGGGGTTCGTGCATACAGTCCAGCTTGGA |
| | | GCGAACTGCCTACCCGGAACTGAGTGTC |
| | | AGGCGTGGAATGAGACAAACGCGGCCAT |
| | | AACAGCGGAATGACACCGGTAAACCGAA |
| | | AGGCAGGAACAGGAGAGCGCACGAGGG |
| | | AGCCGCCAGGGGGAAACGCCTGGTATCT |
| | | TTATAGTCCTGTCGGGTTTCGCCACCACT |
| | | GATTTGAGCGTCAGATTTCGTGATGCTTG |
| | | TCAGGGGGGCGGAGCCTATGGAAAAACG |
| | | GCTTTGCCGCGGCCCTCTCACTTCCCTGTT |
| | | AAGTATCTTCCTGGCATCTTCCAGGAAAT |
| | | CTCCGCCCCGTTCGTAAGCCATTTCCGCT |
| | | CGCCGCAGTCGAACGACCGAGCGTAGCG |
| | | AGTCAGTGAGCGAGGAAGCGGAATATAT |
| | | CCTGTATCACATATTCTGCTGACGCACCG |
| | | GTGCAGCCTTTTTTCTCCTGCCACATGAA |
| | | GCACTTCACTGACACCCTCATCAGTGCCA |
| | | ACATAGTAAGCCAGTATACACTCCGCTAG |
| | | CGCTGAGGTCTGCCTCGTGAAGAAGGTGT |
| | | TGCTGACTCATACCAGGCCTGAATCGCCC |
| | | CATCATCCAGCCAGAAAGTGAGGGAGCC |
| | | ACGGTTGATGAGAGCTTTGTTGTAGGTGG |
| | | ACCAGTTGGTGATTTTGAACTTTTGCTTT |
| | | GCCACGGAACGGTCTGCGTTGTCGGGAA |
| | | GATGCGTGATCTGATCCTTCAACTCAGCA |
| | | AAAGTTCGATTTATTCAACAAAGCCACGT |
| | | TGTGTCTCAAAATCTCTGATGTTACATTG |
| | | CACAAGATAAAAATATATCATCATGAAC |
| | | AATAAAACTGTCTGCTTACATAAACAGTA |
| | | ATACAAGGGGTGTTATGAGCCATATTCAA |
| | | CGGGAAACGTCTTGCTCGAGGCCGCGATT |
| | | AAATTCCAACATGGATGCTGATTTATATG |
| | | GGTATAAATGGGCTCGCGATAATGTCGG |
| | | GCAATCAGGTGCGACAATCTATCGATTGT |
| | | ATGGGAAGCCCGATGCGCCAGAGTTGTTT |
| | | CTGAAACATGGCAAAGGTAGCGTTGCCA |
| | | ATGATGTTACAGATGAGATGGTCAGACT |
| | | AAACTGGCTGACGGAATTTATGCCTCTTC |
| | | CGACCATCAAGCATTTTATCCGTACTCCT |
| | | GATGATGCATGGTTACTCACCACTGCGAT |
| | | CCCCGGGAAAACAGCATTCCAGGTATTA |
| | | GAAGAATATCCTGATTCAGGTGAAAATA |
| | | TTGTTGATGCGCTGGCAGTGTTCCTGCGC |
| | | CGGTTGCATTCGATTCCTGTTTGTAATTGT |
| | | CCTTTTAACAGCGATCGCGTATTTCGTCT |
| | | CGCTCAGGCGCAATCACGAATGAATAAC |
| | | GGTTTGGTTGATGCGAGTGATTTTGATGA |
| | | CGAGCGTAATGGCTGGCCTGTTGAACAA |
| | | GTCTGGAAAGAAATGCATAAGCTTTTGCC |
| | | ATTCTCACCGGATTCAGTCGTCACTCATG |
| | | GTGATTTCTCACTTGATAACCTTATTTTTG |
| | | ACGAGGGGAAATTAATAGGTTGTATTGA |
| | | TGTTGGACGAGTCGGAATCGCAGACCGA |
| | | TACCAGGATCTTGCCATCCTATGGAACTG |
| | | CCTCGGTGAGTTTTCTCCTTCATTACAGA |
| | | AACGGCTTTTTCAAAAATATGGTATTGAT |
| | | AATCCTGATATGAATAAATTGCAGTTTCA |
| | | TTTGATGCTCGATGAGTTTTTCTAATCAG |
| | | AATTGGTTAATTGGTTGTAACACTGGCAG |
| | | AGCATTACGCTGACTTGACGGGACGGCG |
| | | GCTTTGTTGAATAAATCGAACTTTTGCTG |
| | | AGTTGAAGGATCAGATCACGCATCTTCCC |
| | | GACAACGCAGACCGTTCCGTGGCAAAGC |
| | | AAAAGTTCAAAATCACCAACTGGTCCAC |
| | | CTACAACAAAGCTCTCATCAACCGTGGCT |
| | | CCCTCACTTTCTGGCTGGATGATGGGGCG |
| | | ATTCAGGCCTGGTATGAGTCAGCAACACC |
| | | TTCTTCACGAGGCAGACCTCAGCGCTCAA |
| | | AGATGCAGGGGTAAAAGCTAACCGCATC |
| | | TTTACCGACAAGGCATCCGGCAGTTCAAC |
| | | AGATCGGGAAGGGCTGGATTTGCTGAGG |
| | | ATGAAGGTGGAGGAAGGTGATGTCATTC |
| | | TGGTGAAGAAGCTCGACCGTCTTGGCCGC |
| | | GACACCGCCGACATGATCCAACTGATAA |
| | | AAGAGTTTGATGCTCAGGGTGTAGCGGTT |
| | | CGGTTTATTGACGACGGGATCAGTACCGA |
| | | CGGTGATATGGGGCAAATGGTGGTCACC |
| | | ATCCTGTCGGCTGTGGCACAGGCTGAACG |
| | | CCGGAGGATCAAGTCGGTCAAGCCAAGC |
| | | GCAACCAGCGGCACCGCCGCGAGCAACG |
| | | TCGCAAGGGCGATCAGGGGACGATTTTT |
| | | GCGAAGAATTTCCACGGTAAGAATCCAA |
| | | TCTCTCGAATTTAGGGTGAAAGAAGCTTG |
| | | GCATAGGGGTGTGCACGAACTCGGTGGA |
| | | GGAAATTTCCGCGGGGCAAGGCTTCGCG |
| | | AAGCGGAGTCGCGGCAGTGGCTTTGAAG |
| | | ATCTTTGGGAGCAGTCCTTGTGCGCTTAC |
| | | GAGGTGAGCCGGTGGGGAACCGTTATCT |
| | | GCCTATGGTGTGAGCCCCCCTAGAGAGCT |
| | | TCAAGAGCAATCAGCCCGACCTAGAAAG |
| | | GAGGCCAAGAGAGAGACCCCTACGGGGG |
| | | GAACCGTTTTCTGCCTACGAGATGGCACA |
| | | TTTACTGGGAAGCTTTACGGCGTCCTCGT |
| | | GGAAGTTCAATGCCCGCAGACTTAAGTG |
| | | CTCTATTCACGGTCTGACGTGACACGCTA |
| | | AATTCAGACATAGCTTCATTGATTGTCGG |
| | | CCACGAGCCAGTCTCTCCCTCAACAGTCA |
| | | TAAACCAACCTGCAATGGTCAAGCGATTT |
| | | CCTTTAGCTTTCCTAGCTTGTCGTTGACTG |
| | | GACTTAGCTAGTTTTTCTCGCTGTGCTCG |
| | | GGCGTACTCACTGTTTGGGTCTTTCCAGC |
| | | GTTCTGCGGCCTTTTTACCGCCACGTCTTC |
| | | CCATAGTGGCCAGAGCTTTTCGCCCTCGG |
| | | CTGCTCTGCGTCTCTGTCTGACGAGCAGG |
| | | GACGACTGGCTGGCCTTTAGCGACGTAGC |
| | | CGCGCACACGTCGCGCCATCGTCTGGCGG |
| | | TCACGCATCGGCGGCAGATCAGGCTCAC |
| | | GGCCGTCTGCTCCGACCGCCTGAGCGACG |
| | | GTGTAGGCACGCTCGTAGGCGTCGATGAT |
| | | CTTGGTGTCTTTTAGGCGCTCACCAGCCG |
| | | CTTTTAACTGGTATCCCACAGTCAAAGCG |
| | | TGGCGAAAAGCCGTCTCATCACGGGCGG |
| | | CACGCCCTGGAGCAGTCCAGAGGACACG |
| | | GACGCCGTCGATCAGCTCTCCAGACGCTT |
| | | CAGCGGCGCTCGGCAGGCTTGCTTCAAGC |
| | | GTGGCAAGTGCTTTTGCTTCCGCAGTGGC |
| | | TTTTCTTGCCGCTTCGATACGTGCCCGTCC |
| | | GCTAGAAAACTCCTGCTCATAGCGTTTTT |
| | | TAGGTTTTTCTGTGCCTGAGATCATGCGA |
| | | GCAACCTCCATAAGATCAGCTAGGCGAT |
| | | CCACGCGATTGTGCTGGGCATGCCAGCG |
| | | GTACGCGGTGGGATCGTCGGAGACGTGC |
| | | AGTGGCCACCGGCTCAGCCTATGTGAAA |
| | | AAGCCTGGTCAGCGCCGAAAACGCGGGT |
| | | CATTTCCTCGGTCGTTGCAGCCAGCAGGC |
| | | GCATATTCGGGCTGCTCATGCCTGCTGCG |
| | | GCATACACCGGATCAATGAGCCAGATGA |
| | | GCTGGCATTTCCCGCTCAGTGGATTCACG |
| | | CCGATCCAAGCTGGCGCTTTTTCCAGGCG |
| | | TGCCCAGCGCTCCAAAATCGCGTAGACCT |
| | | CGGGGTTTACGTGCTCGATTTTCCCGCCG |
| | | GCCTGGTGGCTCGGCACATCAATGTCCAG |
| | | GACAAGCACGGCTGCGTGCTGCGCGTGC |
| | | GTCAGAGCAACATACTGGCACCGGGCAA |
| | | GCGATTTTGAACCAACTCGGTATAACTTC |
| | | GGCTGTGTTTCTCCCGTGTCCGGGTCTTT |
| | | GATCCAAGCGCTGGCGAAGTCGCGGGTC |
| | | TTGCTGCCCTGGAAATTTTCTCTGCCCAG |
| | | GTGAGCGAGGAATTCGCGGCGGTCTTCG |
| | | CTCGTCCAGCCACGTGATCGCAGCGCGA |
| | | GCTCGGGATGGGTGTCGAACAGATCAGC |
| | | GGAAAATTTCCAGGCCGGTGTGTCAATGT |
| | | CTCGTGAATCCGCTAGAGTCATTTTTGAG |
| | | CGCTTTCTCCCAGGTTTGGACTGGGGGTT |
| | | AGCCGACGCCCTGTGAGTTACCGCTCACG |
| | | GGGCGTTCAACATTTTTCAGGTATTCGTG |
| | | CAGCTTATCGCTTCTTGCCGCCTGTGCGC |
| | | TTTTTCGACGCGCGACGCTGCTGCCGATT |
| | | CGGTGCAGGTGGTGGCGGCGCTGACACG |
| | | TCCTGGGCGGCCACGGCCACACGAAACG |
| | | CGGCATTTACGATGTTTGTCATGCCTGCG |
| | | GGCACCGCGCCACGATCGCGGATAATTCT |
| | | CGCTGCCGCTTCCAGCTCTGTGACGACCA |
| | | TGGCCAAAATTTCGCTCGGGGGACGCACT |
| | | TCCAGCGCCATTTGCGACCTAGCCGCCTC |
| | | CAGCTCCTCGGCGTGGCGTTTGTTGGCGC |
| | | GCTCGCGGCTGGCTGCGGCACGACACGC |
| | | ATCTGAGCAATATTTTGCGCGCCGTCCTC |
| | | GCGGGTCAGGCCGGGGAGGAATCAGGCC |
| | | ACCGCAGTAGGCGCAACTGATTCGATCCT |
| | | CCACTACTGTGCGTCCTCCTGGCGCTGCC |
| | | GAGCACGCAGCTCGTCAGCCAGCTCCTCA |
| | | AGATCCGCCACGAGAGTTTCTAGGTCGCT |
| | | CGCGGCACTGGCCCAGTCTCGTGATGCTG |
| | | GCGCGTCCGTCGTATCGAGAGCTCGGAA |
| | | AAATCCGATCACCGTTTTTAAATCGACGG |
| | | CAGCATCGAGCGCGTCGGACTCCAGCGC |
| | | GACATCAGAGAGATCCATAGCTGATGAT |
| | | TCGGGCCAATTTTGGTACTTCGTCGTGAA |
| | | GGTCATGACACCATTATAACGAACGTTCG |
| | | TTAAAGTTTTTGGCGGAAAATCACGCGGC |
| | | ACGAAAATTTTCACGAAGCGGGACTTTGC |
| | | GCAGCTCAGGGGTGCTAAAAATTTTGTAT |
| | | CGCACTTGATTTTTCCGAAAGACAGATTA |
| | | TCTGCAAACGGTGTGTCGTATTTCTGGCT |
| | | TGGTTTTTAAAAAATCTGGAATCGAAAAT |
| | | TTGCGGGGCGACCGAGAAGTTTTTTACAA |
| | | AAGGCAAAAACTTTTTCGGGATCGACAG |
| | | AAATAAAACGATCGACGGTACGCAACAA |
| | | AAAAGCGTCAGGATCGCCGTAGAGCGAT |
| | | TGAAGACCGTCAACCAAAGGGGAAGCCT |
| | | CCAATCGACGCGACGCGCGCTCTACGGC |
| | | GATCCTGACGCAGATTTTTAGCTATCTGT |
| | | CGCAGCGCCCTCAGGGACAAGCCACCCG |
| | | CACAACGTCGCGAGGGCGATCAGCGACG |
| | | CCGCAGGG |
| SEQ ID NO: 37 | APSE10775 | TtCCGAAATTAATACGACTCACTATAGGG |
| | | AGACATGAGGATTACCCATGTGCGATCG |
| | | CGCACGAGGTTTTTCTGTCTAGTGAGCAG |
| | | TGTCCAACCTCAAAAGACAACATGTGTG |
| | | ACGACGATGTAGCGGCTCTTGTCGTAGAC |
| | | AATGGATCCGGTATGTGCAAAGCCGGTTT |
| | | CGCAGGAGATGACGCACCCCGTGCCGTC |
| | | TTCCCCTCGATCGTCGGTCGCCCAAGGCA |
| | | TCAAGGAGTCATGGTCGGTATGGGACAA |
| | | AAGGACTCATACGTAGGAGATGAAGCCC |
| | | AAAGCAAAAGAGGTATCCTCACCCTGAA |
| | | ATACCCCATCGAACACGGTATCATCACCA |
| | | ACTGGGATGAGTTTAAACCCTCTAGCTGC |
| | | TTTACAAAGTACTGGTTCCCTTTCCAGCG |
| | | GGATGCTTTATCTAAACGCAATGAGAGA |
| | | GGTATTCCTCAGGCCACATCGCTTCCTAG |
| | | TTCCGCTGGGATCCATCGTTGGCGGCCGA |
| | | AGCCGCCATTCCATAGTGAGTTCTGGCGC |
| | | GCCTCATCCCAGTTGGTGATGATACCGTG |
| | | TTCGATGGGGTATTTCAGGGTGAGGATAC |
| | | CTCTTTTGCTTTGGGCTTCATCTCCTACGT |
| | | ATGAGTCCTTTTGTCCCATACCGACCATG |
| | | ACTCCTTGATGCCTTGGGCGACCGACGAT |
| | | CGAGGGGAAGACGGCACGGGGTGCGTCA |
| | | TCTCCTGCGAAACCGGCTTTGCACATACC |
| | | GGATCCATTGTCTACGACAAGAGCCGCTA |
| | | CATCGTCGTCACACATGTTGTCTTTTGAG |
| | | GTTGGACACTGCTCACTAGACAGAAAAA |
| | | CCTCGTGCCGGACCGAATACCCGGTCTGA |
| | | ACGAGGGCGGCCGCGGTACCCAAGAAGT |
| | | ACTTAGAGTTAATTAAGGAGTTCAAACAT |
| | | GAGGATCACCCATGTCGAAGCTCCCACA |
| | | CCCTAGCATAACCCCTTGGGGCCTCTAAA |
| | | CGGGTCTTGAGGGGTTTTTTGCTGAAAGG |
| | | AGGAACTATATCCGGATATCCACAGGAC |
| | | GGGTGTGGTCGCCATGATCGCGTAGTCGA |
| | | TAGTGGCTCCAAGTAGCGAAGCGAGCAG |
| | | GACTGGGCGGCGGGCATGCATCGTCCATT |
| | | CCGACAGCATCGCCAGTCACTATGGCGTG |
| | | CTGCTAGCGCTATATGCGTTGATGCAATT |
| | | TCTATGCGCACCCGTTCTCGGAGCACTGT |
| | | CCGACCGCTTTGGCCGCCGCCCAGTCCTG |
| | | CTCGCTTCGCTACTTGGAGCCACTATCGA |
| | | CTACGCGATCATGGCGACCACACCCGTCC |
| | | TGTGGATCCAGATCTCGATCCCGCGAAAT |
| | | TAATACGACTCACTATAGGGAGACCACA |
| | | ACGGTTTCCCTCTAGATCACAAGTTTGTA |
| | | CAAAAAAGCAGGCTAAGAAGGAGATATA |
| | | CATATGGCGTCTAACTTTACCCAATTCGT |
| | | TCTGGTTGATAACGGCGGTACGGGTGAC |
| | | GTTACCGTAGCTCCGTCCAACTTCGCCAA |
| | | CGGTGTTGCGGAATGGATTAGCTCTAACA |
| | | GCCGCTCTCAGGCCTACAAAGTCACGTGC |
| | | TCCGTTCGTCAGTCTAGCGCGCAGAATCG |
| | | CAAATACACCATCAAAGTTGAAGTACCG |
| | | AAAGTCGCAACGCAGACCGTAGGCGGCG |
| | | TAGAACTCCCAGTTGCGGCCTGGCGCTCT |
| | | TACCTCAACATGGAACTGACTATTCCGAT |
| | | TTTTGCGACGAACTCCGACTGCGAACTGA |
| | | TTGTTAAGGCAATGCAGGGCCTGCTGAA |
| | | AGACGGTAATCCGATCCCATCTGCAATCG |
| | | CTGCTAACTCTGGCATTTACTAATAAGCG |
| | | GACGCGCTGCCACCGCTGAGCAATAACT |
| | | AGCATAACCCCTTGGGGCCTCTAAACGG |
| | | GTCTTGAGGGGTTTTTTGCTGAAAGGAGG |
| | | AACTATATCCGGCATGCACCATTCCTTGC |
| | | GGCGGCGGTGCTCAACGGCCTCAACCTA |
| | | CTACTGGGCTGCTTCCTAATGCAGGAGTC |
| | | GCATAAGGGAGAGCgtcgacAGAAGGAGAT |
| | | ATACATATGaaaccatatcagcgccagtttattGaatttgcg |
| | | cttagcaagcaggtgttaaagtttggcgagtttacgctgaaatccgg |
| | | gcgcaaaagcccctatttcttcaacgccgggctgtttaataccgggc |
| | | gcgatctggcactgttaggccgtttttacgctgaagcgttggtggatt |
| | | ccggcattgagttcgatctgctgtttggccctgcttacaaagggatcc |
| | | cgattgccaccacaaccgctgtggcactggcggagcatcacgacc |
| | | tggacctgccgtactgctttaaccgcaaagaagcaaaagaccacg |
| | | gtgaaggcggcaatctggttggtagcgcgttacaaggacgcgtaat |
| | | gctggtagatgatgtgatcaccgccggaacggcgattcgcgagtc |
| | | gatggagattattcaggccaatggcgcgacgcttgctggcgtgttga |
| | | tttcgctcgatcgtcaggaacgcgggcgcggcgagatttcggcgat |
| | | tcaggaagttgagcgtgattacaactgcaaagtgatctctatcatcac |
| | | cctgaaagacctgattgcttacctggaagagaagccggaaatggc |
| | | ggaacatctggcggcggttaaggcctatcgcgaagagtttggcgttt |
| | | aacctaggCAGAACGCAGAAGCtGTCTGATAA |
| | | AACAGAATTTGCCTGGCGGCAGTAGCGC |
| | | GGTGGTCCCACCTGACCCCATGCCGAACT |
| | | CAGAAGTGAAACGCCGTAGCGCCGATGG |
| | | TAGTGTGGGGTCTCCCCATGCGAGAGTAG |
| | | GGAACTGCCAGGCATCAAATAAAACGAA |
| | | AGGCTCAGTCGAAAGACTGGGCCTTTCGT |
| | | TTTATCTGTTGTTTGTCGGTGAACGCTCTC |
| | | CTGAGTAGGACAAATCCGCCGGGAGCGG |
| | | ATTTGAACGTTGCGAAGCAACGGCCCGG |
| | | AGGGTGGCGGGCAGGACGCCCGCCATAA |
| | | ACTGCCAGGCATCAAATTAAGCAGAAGG |
| | | CCATCCTGACGGATGGCCTTTTTGCGTTT |
| | | CTACtcgcgaCGCGAGGCTGGATGGCCTTCC |
| | | CCATTATGATTCTTCTCGCTTCCGGCGGC |
| | | ATCGGGATGCCCGCGTTGCAGGCCATGCT |
| | | GTCCAGGCAGGTAGATGACGACCATCAG |
| | | GGACAGCTTCAAGGATCGCTCGCGGCTCT |
| | | TACCAGCCTAACTTCGATCATTGGACCGC |
| | | TGATCGTCACGGCGATTTATGCCGCCTCG |
| | | GCGAGCACATGGAACGGGTTGGCATGGA |
| | | TTGTAGGCGCCGCCCTATACCTTGTCTGC |
| | | CTCCCCGCGTTGCGTCGCGGTGCATGGAG |
| | | CCGGGCCACCTCGACCTGAATGGAAGCC |
| | | GGCGGCACCTCGCTAACGGATTCACCACT |
| | | CCAAGAATTGGAGCCAATCAATTCTTGCG |
| | | GAGAACTGTGAATGCGCAAACCAACCCT |
| | | TGGCAGAACATATCCATCGCGTCCGCCAT |
| | | CTCCAGCAGCCGCACGCGGCGCATCTCG |
| | | GGCAGCGTTGGGTCCTGGCCACGGGTGC |
| | | GCATGATCGTGCTCCTGTCGTTGAGGACC |
| | | CGGCTAGGCTGGCGGGGTTGCCTTACTGG |
| | | TTAGCAGAATGAATCACCGATACGCGAG |
| | | CGAACGTGAAGCGACTGCTGCTGCAAAA |
| | | CGTCTGCGACCTGAGCAACAACATGAAT |
| | | GGTCTTCGGTTTCCGTGTTTCGTAAAGTC |
| | | TGGAAACGCGGAAGTCAGCGCCCTGCAC |
| | | CATTATGTTCCGGATCTGCATCGCAGGAT |
| | | GCTGCTGGCTACCCTGTGGAACACCTACA |
| | | TCTGTATTAACGAAGCGCTGGCATTGACC |
| | | CTGAGTGATTTTTCTCTGGTCCCGCCGCA |
| | | TCCATACCGCCAGTTGTTTACCCTCACAA |
| | | CGTTCCAGTAACCGGGCATGTTCATCATC |
| | | AGTAACCCGTATCGTGAGCATCCTCTCTC |
| | | GTTTCATCGGTATCATTACCCCCATGAAC |
| | | AGAAATCCCCCTTACACGGAGGCATCAG |
| | | TGACCAAACAGGAAAAAACCGCCCTTAA |
| | | CATGGCCCGCTTTATCAGAAGCCAGACAT |
| | | TAACGCTTCTGGAGAAACTCAACGAGCT |
| | | GGACGCGGATGAACAGGCAGACATCTGT |
| | | GAATCGCTTCACGACCACGCTGATGAGCT |
| | | TTACCGCAGCTGCCTCGCGCGTTTCGGTG |
| | | ATGACGGTGAAAACCTCTGACACATGCA |
| | | GCTCCCGGAGACGGTCACAGCTTGTCTGT |
| | | AAGCGGATGCCGGGAGCAGACAAGCCCG |
| | | TCAGGGCGCGTCAGCGGGTGTTGGCGGG |
| | | TGTCGGGGCGCAGCCATGACCCAGTCAC |
| | | GTAGCGATAGCGGAGTGTATACTGGCTTA |
| | | ACTATGCGGCATCAGAGCAGATTGTACTG |
| | | AGAGTGCACCATATGCGGTGTGAAATAC |
| | | CGCACAGATGCGTAAGGAGAAAATACCG |
| | | CATCAGGCGCTCTTCCGCTTCCTCGCTCA |
| | | CTGACTCGCTGCGCTCGGTCGTTCGGCTG |
| | | CGGCGAGCGGTATCAGCTCACTCAAAGG |
| | | CGGTAATACGGTTATCCACAGAATCAGG |
| | | GGATAACGCAGGAAAGAACATGTGAGCA |
| | | AAAGGCCAGCAAAAGGCCAGGAACCGTA |
| | | AAAAGGCCGCGTTGCTGGCGTTTTTCCAT |
| | | AGGCTCCGCCCCCCTGACGAGCATCACA |
| | | AAAATCGACGCTCAAGTCAGAGGTGGCG |
| | | AAACCCGACAGGACTATAAAGATACCAG |
| | | GCGTTTCCCCCTGGAAGCTCCCTCGTGCG |
| | | CTCTCCTGTTCCGACCCTGCCGCTTACCG |
| | | GATACCTGTCCGCCTTTCTCCCTTCGGGA |
| | | AGCGTGGCGCTTTCTCATAGCTCACGCTG |
| | | TAGGTATCTCAGTTCGGTGTAGGTCGTTC |
| | | GCTCCAAGCTGGGCTGTGTGCACGAACCC |
| | | CCCGTTCAGCCCGACCGCTGCGCCTTATC |
| | | CGGTAACTATCGTCTTGAGTCCAACCCGG |
| | | TAAGACACGACTTATCGCCACTGGCAGC |
| | | AGCCACTGGTAACAGGATTAGCAGAGCG |
| | | AGGTATGTAGGCGGTGCTACAGAGTTCTT |
| | | GAAGTGGTGGCCTAACTACGGCTACACT |
| | | AGAAGGACAGTATTTGGTATCTGCGCTCT |
| | | GCTGAAGCCAGTTACCTTCGGAAAAAGA |
| | | GTTGGTAGCTCTTGATCCGGCAAACAAAC |
| | | CACCGCTGGTAGCGGTGGTTTTTTTGTTT |
| | | GCAAGCAGCAGATTACGCGCAGAAAAAA |
| | | AGGATCTCAAGAAGATCCTTTGATCTTTT |
| | | CTACGGGGTCTGACGCTCAGTGGAACGA |
| | | AAACTCACGTTAAGGGATTTTGGTCATGA |
| | | GATTATCAAAAAGGATCTTCACCTAGATC |
| | | CTTTTAAATTAAAAATGAAGTTTTAAATC |
| | | AATCTAAAGTATATATGAGTAAACTTGGT |
| | | CTGACAGTTACCAATGCTTAATCAGTGAG |
| | | GCACCTATCTCAGCGATCTGTCTATTTCG |
| | | TTCATCCATAGTTGCCTGACTCCCCGTCG |
| | | TGTAGATAACTACGATACGGGAGGGCTT |
| | | ACCATCTGGCCCCAGTGCTGCAATGATAC |
| | | CGCGAGACCCACGCTCACCGGCTCCAGA |
| | | TTTATCAGCAATAAACCAGCCAGCCGGA |
| | | AGGGCCGAGCGCAGAAGTGGTCCTGCAA |
| | | CTTTATCCGCCTCCATCCAGTCTATTAATT |
| | | GTTGCCGGGAAGCTAGAGTAAGTAGTTC |
| | | GCCAGTTAATAGTTTGCGCAACGTTGTTG |
| | | CCATTGCTGCAGGCATCGTGGTGTCACGC |
| | | TCGTCGTTTGGTATGGCTTCATTCAGCTC |
| | | CGGTTCCCAACGATCAAGGCGAGTTACAT |
| | | GATCCCCCATGTTGTGCAAAAAAGCGGTT |
| | | AGCTCCTTCGGTCCTCCGATCGTTGTCAG |
| | | AAGTAAGTTGGCCGCAGTGTTATCACTCA |
| | | TGGTTATGGCAGCACTGCATAATTCTCTT |
| | | ACTGTCATGCCATCCGTAAGATGCTTTTC |
| | | TGTGACTGGTGAGTACTCAACCAAGTCAT |
| | | TCTGAGAATAGTGTATGCGGCGACCGAG |
| | | TTGCTCTTGCCCGGCGTCAACACGGGATA |
| | | ATACCGCGCCACATAGCAGAACTTTAAA |
| | | AGTGCTCATCATTGGAAAACGTTCTTCGG |
| | | GGCGAAAACTCTCAAGGATCTTACCGCTG |
| | | TTGAGATCCAGTTCGATGTAACCCACTCG |
| | | TGCACCCAACTGATCTTCAGCATCTTTTA |
| | | CTTTCACCAGCGTTTCTGGGTGAGCAAAA |
| | | ACAGGAAGGCAAAATGCCGCAAAAAAGG |
| | | GAATAAGGGCGACACGGAAATGTTGAAT |
| | | ACTCATACTCTTCCTTTTTCAATATTATTG |
| | | AAGCATTTATCAGGGTTATTGTCTCATGA |
| | | GCGGATACATATTTGAATGTATTTAGAAA |
| | | AATAAACAAATAGGGGTTCCGCGCACAT |
| | | TTCCCCGAAAAGTGCCACCTGACGTCTAA |
| | | GAAACCATTATTATCATGACATTAACCTA |
| | | TAAAAATAGGCGTATCACGAGGCCCTTTC |
| | | GTCTTCAAGAA |
| SEQ ID NO: 38 | APSE10772 | GGATCCTctagtCTACAATCCATGCCAACCC |
| | | GTTCCATGTGCTCGCCGAGGCGGCATAAA |
| | | TCGCCGTGACGATCAGCGGTCCAATGATC |
| | | GAAGTTAGGCTGGTAAGAGCCGCGAGCG |
| | | ATCCTTGAAGCTGTCCCTGATGGTCGTCA |
| | | TCTACCTGCCTGGACAGCATGGCCTGCAA |
| | | CGCGGGCATCCCGATGCCGCCGGAAGCG |
| | | AGAAGAATCATAATGGGGAAGGCCATCC |
| | | AGCCTCGCGtcgcgaGTAGAAACGCAAAAA |
| | | GGCCATCCGTCAGGATGGCCTTCTGCTTA |
| | | ATTTGATGCCTGGCAGTTTATGGCGGGCG |
| | | TCCTGCCCGCCACCCTCCGGGCCGTTGCT |
| | | TCGCAACGTTCAAATCCGCTCCCGGCGGA |
| | | TTTGTCCTACTCAGGAGAGCGTTCACCGA |
| | | CAAACAACAGATAAAACGAAAGGCCCAG |
| | | TCTTTCGACTGAGCCTTTCGTTTTATTTGA |
| | | TGCCTGGCAGTTCCCTACTCTCGCATGGG |
| | | GAGACCCCACACTACCATCGGCGCTACG |
| | | GCGTTTCACTTCTGAGTTCGGCATGGGGT |
| | | CAGGTGGGACCACCGCGCTACTGCCGCC |
| | | AGGCAAATTCTGTTTTATCAGACaGCTTCT |
| | | GCGTTCTGcctaggttaaacgccaaactcttcgcgataggcc |
| | | ttaaccgccgccagatgttccgccatttccggcttctcttccaggtaa |
| | | gcaatcaggtctttcagggtgatgatagagatcactttgcagttgtaat |
| | | cacgctcaacttcctgaatcgccgaaatctcgccgcgcccgcgttc |
| | | ctgacgatcgagcgaaatcaacacgccagcaagcgtcgcgccatt |
| | | ggcctgaataatctccatcgactcgcgaatcgccgttccggcggtg |
| | | atcacatcatctaccagcattacgcgtccttgtaacgcgctaccaacc |
| | | agattgccgccttcaccgtggtcttttgcttctttgcggttaaagcagt |
| | | acggcaggtccaggtcgtgatgctccgccagtgccacagcggttgt |
| | | ggtggcaatcgggatccctttgtaagcagggccaaacagcagatc |
| | | gaactcaatgccggaatccaccaacgcttcagcgtaaaaacggcct |
| | | aacagtgccagatcgcgcccggtattaaacagcccggcgttgaag |
| | | aaataggggcttttgcgcccggatttcagcgtaaactcgccaaacttt |
| | | aacacctgcttgctaagcgcaaattCaataaactggcgctgatatgg |
| | | tttCATATGTATATCTCCTTCTgtcgacGCTCTC |
| | | CCTTATGCGACTCCTGCATTAGGAAGCAG |
| | | CCCAGTAGTAGGTTGAGGCCGTTGAGCA |
| | | CCGCCGCCGCAAGGAATGGTGCATGCCG |
| | | GATATAGTTCCTCCTTTCAGCAAAAAACC |
| | | CCTCAAGACCCGTTTAGAGGCCCCAAGG |
| | | GGTTATGCTAGTTATTGCTCAGCGGTGGC |
| | | AGCGCGTCCGCTTATTAGTAAATGCCAGA |
| | | GTTAGCAGCGATTGCAGATGGGATCGGA |
| | | TTACCGTCTTTCAGCAGGCCCTGCATTGC |
| | | CTTAACAATCAGTTCGCAGTCGGAGTTCG |
| | | TCGCAAAAATCGGAATAGTCAGTTCCATG |
| | | TTGAGGTAAGAGCGCCAGGCCGCAACTG |
| | | GGAGTTCTACGCCGCCTACGGTCTGCGTT |
| | | GCGACTTTCGGTACTTCAACTTTGATGGT |
| | | GTATTTGCGATTCTGCGCGCTAGACTGAC |
| | | GAACGGAGCACGTGACTTTGTAGGCCTG |
| | | AGAGCGGCTGTTAGAGCTAATCCATTCCG |
| | | CAACACCGTTGGCGAAGTTGGACGGAGC |
| | | TACGGTAACGTCACCCGTACCGCCGTTAT |
| | | CAACCAGAACGAATTGGGTAAAGTTAGA |
| | | CGCCATATGTATATCTCCTTCTTAGCCTG |
| | | CTTTTTTGTACAAACTTGTGATCTAGAGG |
| | | GAAACCGTTGTGGTCTCCCTATAGTGAGT |
| | | CGTATTAATTTCGCGGGATCGAGATCTGG |
| | | ATCCACAGGACGGGTGTGGTCGCCATGA |
| | | TCGCGTAGTCGATAGTGGCTCCAAGTAGC |
| | | GAAGCGAGCAGGACTGGGCGGCGGCCAA |
| | | AGCGGTCGGACAGTGCTCCGAGAACGGG |
| | | TGCGCATAGAAATTGCATCAACGCATATA |
| | | GCGCTAGCAGCACGCCATAGTGACTGGC |
| | | GATGCTGTCGGAATGGACGATGCATGCC |
| | | CGCCGCCCAGTCCTGCTCGCTTCGCTACT |
| | | TGGAGCCACTATCGACTACGCGATCATGG |
| | | CGACCACACCCGTCCTGTGGATATCCGGA |
| | | TATAGTTCCTCCTTTCAGCAAAAAACCCC |
| | | TCAAGACCCGTTTAGAGGCCCCAAGGGG |
| | | TTATGCTAGGGTGTGGGAGCTTCGACATG |
| | | GGTGATCCTCATGTTTGAACTCCTTAATT |
| | | AACTCTAAGTACTTCTTGGGTACCGCGGC |
| | | CGCCCTCGTTCAGACCGGGTATTCGGTCC |
| | | GCGATCTCTCTCCCTCGACTCTAACACCA |
| | | GCGAAAGTAACAGCCAATCAAGATGTGT |
| | | GACGATGATGTTGCGGCATTAGTCGTGGA |
| | | CAATGGGTCCGGTATGTGCAAGGCTGGA |
| | | TTCGCGGGGGATGATGCACCACGCGCTGT |
| | | GTTTCCCAGCATCGTCGGTCGTCCTCGTC |
| | | ATCAGGGTGTGATGGTCGGTATGGGTCA |
| | | AAAAGACAGTTATGTTGGCGACGAGGCG |
| | | CAAAGTAAGAGAGGTATATTGACACTAA |
| | | AGTATCCTATAGAACATGGCATTATTACT |
| | | AATTGGGATGACATGGGGCGCGCCAGAA |
| | | CTCACTATGGAATGGCGGCTTCGGCCGCC |
| | | AACGATGGATCCCAGCGGAACTAGGAAG |
| | | CGATGTGGCCTGAGGAATACCTCTCTCAT |
| | | TGCGTTTAGATAAAGCATCCCGCTGGAAA |
| | | GGGAACCAGTACTTTGTAAAGCAGCTAG |
| | | AGGGTTTAAACCCATGTCATCCCAATTAG |
| | | TAATAATGCCATGTTCTATAGGATACTTT |
| | | AGTGTCAATATACCTCTCTTACTTTGCGC |
| | | CTCGTCGCCAACATAACTGTCTTTTTGAC |
| | | CCATACCGACCATCACACCCTGATGACGA |
| | | GGACGACCGACGATGCTGGGAAACACAG |
| | | CGCGTGGTGCATCATCCCCCGCGAATCCA |
| | | GCCTTGCACATACCGGACCCATTGTCCAC |
| | | GACTAATGCCGCAACATCATCGTCACACA |
| | | TCTTGATTGGCTGTTACTTTCGCTGGTGTT |
| | | AGAGTCGAGGGAGAGAGATCGGCGATCG |
| | | CACATGGGTAATCCTCATGTCTCCCTATA |
| | | GTGAGTCGTATTAATTTCGGGAGAATTCT |
| | | TGAAGACGAAAGGGCCTCGTGATACGCC |
| | | TATTTTTATAGGTTAATGTCATGATAATA |
| | | ATGGTTTCTTAGACGTCAGGTGGCACTTT |
| | | TCGGGGAAATGTGCGCGGAACCCCTATTT |
| | | GTTTATTTTTCTAAATACATTCAAATATGT |
| | | ATCCGCTCATGAGACAATAACCCTGATAA |
| | | ATGCTTCAATAATATTGAAAAAGGAAGA |
| | | GTATGAGTATTCAACATTTCCGTGTCGCC |
| | | CTTATTCCCTTTTTTGCGGCATTTTGCCTT |
| | | CCTGTTTTTGCTCACCCAGAAACGCTGGT |
| | | GAAAGTAAAAGATGCTGAAGATCAGTTG |
| | | GGTGCACGAGTGGGTTACATCGAACTGG |
| | | ATCTCAACAGCGGTAAGATCCTTGAGAGT |
| | | TTTCGCCCCGAAGAACGTTTTCCAATGAT |
| | | GAGCACTTTTAAAGTTCTGCTATGTGGCG |
| | | CGGTATTATCCCGTGTTGACGCCGGGCAA |
| | | GAGCAACTCGGTCGCCGCATACACTATTC |
| | | TCAGAATGACTTGGTTGAGTACTCACCAG |
| | | TCACAGAAAAGCATCTTACGGATGGCAT |
| | | GACAGTAAGAGAATTATGCAGTGCTGCC |
| | | ATAACCATGAGTGATAACACTGCGGCCA |
| | | ACTTACTTCTGACAACGATCGGAGGACCG |
| | | AAGGAGCTAACCGCTTTTTTGCACAACAT |
| | | GGGGGATCATGTAACTCGCCTTGATCGTT |
| | | GGGAACCGGAGCTGAATGAAGCCATACC |
| | | AAACGACGAGCGTGACACCACGATGCCT |
| | | GCAGCAATGGCAACAACGTTGCGCAAAC |
| | | TATTAACTGGCGAACTACTTACTCTAGCT |
| | | TCCCGGCAACAATTAATAGACTGGATGG |
| | | AGGCGGATAAAGTTGCAGGACCACTTCT |
| | | GCGCTCGGCCCTTCCGGCTGGCTGGTTTA |
| | | TTGCTGATAAATCTGGAGCCGGTGAGCGT |
| | | GGGTCTCGCGGTATCATTGCAGCACTGGG |
| | | GCCAGATGGTAAGCCCTCCCGTATCGTAG |
| | | TTATCTACACGACGGGGAGTCAGGCAAC |
| | | TATGGATGAACGAAATAGACAGATCGCT |
| | | GAGATAGGTGCCTCACTGATTAAGCATTG |
| | | GTAACTGTCAGACCAAGTTTACTCATATA |
| | | TACTTTAGATTGATTTAAAACTTCATTTTT |
| | | AATTTAAAAGGATCTAGGTGAAGATCCTT |
| | | TTTGATAATCTCATGACCAAAATCCCTTA |
| | | ACGTGAGTTTTCGTTCCACTGAGCGTCAG |
| | | ACCCCTTAATAAGATGATCTTCTTGAGAT |
| | | CGTTTTGGTCTGCGCGTAATCTCTTGCTCT |
| | | GAAAACGAAAAAACCGCCTTGCAGGGCG |
| | | GTTTTTCGAAGGTTCTCTGAGCTACCAAC |
| | | TCTTTGAACCGAGGTAACTGGCTTGGAGG |
| | | AGCGCAGTCACCAAAACTTGTCCTTTCAG |
| | | TTTAGCCTTAACCGGCGCATGACTTCAAG |
| | | ACTAACTCCTCTAAATCAATTACCAGTGG |
| | | CTGCTGCCAGTGGTGCTTTTGCATGTCTTT |
| | | CCGGGTTGGACTCAAGACGATAGTTACC |
| | | GGATAAGGCGCAGCGGTCGGACTGAACG |
| | | GGGGGTTCGTGCATACAGTCCAGCTTGGA |
| | | GCGAACTGCCTACCCGGAACTGAGTGTC |
| | | AGGCGTGGAATGAGACAAACGCGGCCAT |
| | | AACAGCGGAATGACACCGGTAAACCGAA |
| | | AGGCAGGAACAGGAGAGCGCACGAGGG |
| | | AGCCGCCAGGGGGAAACGCCTGGTATCT |
| | | TTATAGTCCTGTCGGGTTTCGCCACCACT |
| | | GATTTGAGCGTCAGATTTCGTGATGCTTG |
| | | TCAGGGGGGCGGAGCCTATGGAAAAACG |
| | | GCTTTGCCGCGGCCCTCTCACTTCCCTGTT |
| | | AAGTATCTTCCTGGCATCTTCCAGGAAAT |
| | | CTCCGCCCCGTTCGTAAGCCATTTCCGCT |
| | | CGCCGCAGTCGAACGACCGAGCGTAGCG |
| | | AGTCAGTGAGCGAGGAAGCGGAATATAT |
| | | CCTGTATCACATATTCTGCTGACGCACCG |
| | | GTGCAGCCTTTTTTCTCCTGCCACATGAA |
| | | GCACTTCACTGACACCCTCATCAGTGCCA |
| | | ACATAGTAAGCCAGTATACACTCCGCTAG |
| | | CGCTGAGGTCTGCCTCGTGAAGAAGGTGT |
| | | TGCTGACTCATACCAGGCCTGAATCGCCC |
| | | CATCATCCAGCCAGAAAGTGAGGGAGCC |
| | | ACGGTTGATGAGAGCTTTGTTGTAGGTGG |
| | | ACCAGTTGGTGATTTTGAACTTTTGCTTT |
| | | GCCACGGAACGGTCTGCGTTGTCGGGAA |
| | | GATGCGTGATCTGATCCTTCAACTCAGCA |
| | | AAAGTTCGATTTATTCAACAAAGCCACGT |
| | | TGTGTCTCAAAATCTCTGATGTTACATTG |
| | | CACAAGATAAAAATATATCATCATGAAC |
| | | AATAAAACTGTCTGCTTACATAAACAGTA |
| | | ATACAAGGGGTGTTATGAGCCATATTCAA |
| | | CGGGAAACGTCTTGCTCGAGGCCGCGATT |
| | | AAATTCCAACATGGATGCTGATTTATATG |
| | | GGTATAAATGGGCTCGCGATAATGTCGG |
| | | GCAATCAGGTGCGACAATCTATCGATTGT |
| | | ATGGGAAGCCCGATGCGCCAGAGTTGTTT |
| | | CTGAAACATGGCAAAGGTAGCGTTGCCA |
| | | ATGATGTTACAGATGAGATGGTCAGACT |
| | | AAACTGGCTGACGGAATTTATGCCTCTTC |
| | | CGACCATCAAGCATTTTATCCGTACTCCT |
| | | GATGATGCATGGTTACTCACCACTGCGAT |
| | | CCCCGGGAAAACAGCATTCCAGGTATTA |
| | | GAAGAATATCCTGATTCAGGTGAAAATA |
| | | TTGTTGATGCGCTGGCAGTGTTCCTGCGC |
| | | CGGTTGCATTCGATTCCTGTTTGTAATTGT |
| | | CCTTTTAACAGCGATCGCGTATTTCGTCT |
| | | CGCTCAGGCGCAATCACGAATGAATAAC |
| | | GGTTTGGTTGATGCGAGTGATTTTGATGA |
| | | CGAGCGTAATGGCTGGCCTGTTGAACAA |
| | | GTCTGGAAAGAAATGCATAAGCTTTTGCC |
| | | ATTCTCACCGGATTCAGTCGTCACTCATG |
| | | GTGATTTCTCACTTGATAACCTTATTTTTG |
| | | ACGAGGGGAAATTAATAGGTTGTATTGA |
| | | TGTTGGACGAGTCGGAATCGCAGACCGA |
| | | TACCAGGATCTTGCCATCCTATGGAACTG |
| | | CCTCGGTGAGTTTTCTCCTTCATTACAGA |
| | | AACGGCTTTTTCAAAAATATGGTATTGAT |
| | | AATCCTGATATGAATAAATTGCAGTTTCA |
| | | TTTGATGCTCGATGAGTTTTTCTAATCAG |
| | | AATTGGTTAATTGGTTGTAACACTGGCAG |
| | | AGCATTACGCTGACTTGACGGGACGGCG |
| | | GCTTTGTTGAATAAATCGAACTTTTGCTG |
| | | AGTTGAAGGATCAGATCACGCATCTTCCC |
| | | GACAACGCAGACCGTTCCGTGGCAAAGC |
| | | AAAAGTTCAAAATCACCAACTGGTCCAC |
| | | CTACAACAAAGCTCTCATCAACCGTGGCT |
| | | CCCTCACTTTCTGGCTGGATGATGGGGCG |
| | | ATTCAGGCCTGGTATGAGTCAGCAACACC |
| | | TTCTTCACGAGGCAGACCTCAGCGCTCAA |
| | | AGATGCAGGGGTAAAAGCTAACCGCATC |
| | | TTTACCGACAAGGCATCCGGCAGTTCAAC |
| | | AGATCGGGAAGGGCTGGATTTGCTGAGG |
| | | ATGAAGGTGGAGGAAGGTGATGTCATTC |
| | | TGGTGAAGAAGCTCGACCGTCTTGGCCGC |
| | | GACACCGCCGACATGATCCAACTGATAA |
| | | AAGAGTTTGATGCTCAGGGTGTAGCGGTT |
| | | CGGTTTATTGACGACGGGATCAGTACCGA |
| | | CGGTGATATGGGGCAAATGGTGGTCACC |
| | | ATCCTGTCGGCTGTGGCACAGGCTGAACG |
| | | CCGGAGGATCAAGTCGGTCAAGCCAAGC |
| | | GCAACCAGCGGCACCGCCGCGAGCAACG |
| | | TCGCAAGGGCGATCAGGGGACGATTTTT |
| | | GCGAAGAATTTCCACGGTAAGAATCCAA |
| | | TCTCTCGAATTTAGGGTGAAAGAAGCTTG |
| | | GCATAGGGGTGTGCACGAACTCGGTGGA |
| | | GGAAATTTCCGCGGGGCAAGGCTTCGCG |
| | | AAGCGGAGTCGCGGCAGTGGCTTTGAAG |
| | | ATCTTTGGGAGCAGTCCTTGTGCGCTTAC |
| | | GAGGTGAGCCGGTGGGGAACCGTTATCT |
| | | GCCTATGGTGTGAGCCCCCCTAGAGAGCT |
| | | TCAAGAGCAATCAGCCCGACCTAGAAAG |
| | | GAGGCCAAGAGAGAGACCCCTACGGGGG |
| | | GAACCGTTTTCTGCCTACGAGATGGCACA |
| | | TTTACTGGGAAGCTTTACGGCGTCCTCGT |
| | | GGAAGTTCAATGCCCGCAGACTTAAGTG |
| | | CTCTATTCACGGTCTGACGTGACACGCTA |
| | | AATTCAGACATAGCTTCATTGATTGTCGG |
| | | CCACGAGCCAGTCTCTCCCTCAACAGTCA |
| | | TAAACCAACCTGCAATGGTCAAGCGATTT |
| | | CCTTTAGCTTTCCTAGCTTGTCGTTGACTG |
| | | GACTTAGCTAGTTTTTCTCGCTGTGCTCG |
| | | GGCGTACTCACTGTTTGGGTCTTTCCAGC |
| | | GTTCTGCGGCCTTTTTACCGCCACGTCTTC |
| | | CCATAGTGGCCAGAGCTTTTCGCCCTCGG |
| | | CTGCTCTGCGTCTCTGTCTGACGAGCAGG |
| | | GACGACTGGCTGGCCTTTAGCGACGTAGC |
| | | CGCGCACACGTCGCGCCATCGTCTGGCGG |
| | | TCACGCATCGGCGGCAGATCAGGCTCAC |
| | | GGCCGTCTGCTCCGACCGCCTGAGCGACG |
| | | GTGTAGGCACGCTCGTAGGCGTCGATGAT |
| | | CTTGGTGTCTTTTAGGCGCTCACCAGCCG |
| | | CTTTTAACTGGTATCCCACAGTCAAAGCG |
| | | TGGCGAAAAGCCGTCTCATCACGGGCGG |
| | | CACGCCCTGGAGCAGTCCAGAGGACACG |
| | | GACGCCGTCGATCAGCTCTCCAGACGCTT |
| | | CAGCGGCGCTCGGCAGGCTTGCTTCAAGC |
| | | GTGGCAAGTGCTTTTGCTTCCGCAGTGGC |
| | | TTTTCTTGCCGCTTCGATACGTGCCCGTCC |
| | | GCTAGAAAACTCCTGCTCATAGCGTTTTT |
| | | TAGGTTTTTCTGTGCCTGAGATCATGCGA |
| | | GCAACCTCCATAAGATCAGCTAGGCGAT |
| | | CCACGCGATTGTGCTGGGCATGCCAGCG |
| | | GTACGCGGTGGGATCGTCGGAGACGTGC |
| | | AGTGGCCACCGGCTCAGCCTATGTGAAA |
| | | AAGCCTGGTCAGCGCCGAAAACGCGGGT |
| | | CATTTCCTCGGTCGTTGCAGCCAGCAGGC |
| | | GCATATTCGGGCTGCTCATGCCTGCTGCG |
| | | GCATACACCGGATCAATGAGCCAGATGA |
| | | GCTGGCATTTCCCGCTCAGTGGATTCACG |
| | | CCGATCCAAGCTGGCGCTTTTTCCAGGCG |
| | | TGCCCAGCGCTCCAAAATCGCGTAGACCT |
| | | CGGGGTTTACGTGCTCGATTTTCCCGCCG |
| | | GCCTGGTGGCTCGGCACATCAATGTCCAG |
| | | GACAAGCACGGCTGCGTGCTGCGCGTGC |
| | | GTCAGAGCAACATACTGGCACCGGGCAA |
| | | GCGATTTTGAACCAACTCGGTATAACTTC |
| | | GGCTGTGTTTCTCCCGTGTCCGGGTCTTT |
| | | GATCCAAGCGCTGGCGAAGTCGCGGGTC |
| | | TTGCTGCCCTGGAAATTTTCTCTGCCCAG |
| | | GTGAGCGAGGAATTCGCGGCGGTCTTCG |
| | | CTCGTCCAGCCACGTGATCGCAGCGCGA |
| | | GCTCGGGATGGGTGTCGAACAGATCAGC |
| | | GGAAAATTTCCAGGCCGGTGTGTCAATGT |
| | | CTCGTGAATCCGCTAGAGTCATTTTTGAG |
| | | CGCTTTCTCCCAGGTTTGGACTGGGGGTT |
| | | AGCCGACGCCCTGTGAGTTACCGCTCACG |
| | | GGGCGTTCAACATTTTTCAGGTATTCGTG |
| | | CAGCTTATCGCTTCTTGCCGCCTGTGCGC |
| | | TTTTTCGACGCGCGACGCTGCTGCCGATT |
| | | CGGTGCAGGTGGTGGCGGCGCTGACACG |
| | | TCCTGGGCGGCCACGGCCACACGAAACG |
| | | CGGCATTTACGATGTTTGTCATGCCTGCG |
| | | GGCACCGCGCCACGATCGCGGATAATTCT |
| | | CGCTGCCGCTTCCAGCTCTGTGACGACCA |
| | | TGGCCAAAATTTCGCTCGGGGGACGCACT |
| | | TCCAGCGCCATTTGCGACCTAGCCGCCTC |
| | | CAGCTCCTCGGCGTGGCGTTTGTTGGCGC |
| | | GCTCGCGGCTGGCTGCGGCACGACACGC |
| | | ATCTGAGCAATATTTTGCGCGCCGTCCTC |
| | | GCGGGTCAGGCCGGGGAGGAATCAGGCC |
| | | ACCGCAGTAGGCGCAACTGATTCGATCCT |
| | | CCACTACTGTGCGTCCTCCTGGCGCTGCC |
| | | GAGCACGCAGCTCGTCAGCCAGCTCCTCA |
| | | AGATCCGCCACGAGAGTTTCTAGGTCGCT |
| | | CGCGGCACTGGCCCAGTCTCGTGATGCTG |
| | | GCGCGTCCGTCGTATCGAGAGCTCGGAA |
| | | AAATCCGATCACCGTTTTTAAATCGACGG |
| | | CAGCATCGAGCGCGTCGGACTCCAGCGC |
| | | GACATCAGAGAGATCCATAGCTGATGAT |
| | | TCGGGCCAATTTTGGTACTTCGTCGTGAA |
| | | GGTCATGACACCATTATAACGAACGTTCG |
| | | TTAAAGTTTTTGGCGGAAAATCACGCGGC |
| | | ACGAAAATTTTCACGAAGCGGGACTTTGC |
| | | GCAGCTCAGGGGTGCTAAAAATTTTGTAT |
| | | CGCACTTGATTTTTCCGAAAGACAGATTA |
| | | TCTGCAAACGGTGTGTCGTATTTCTGGCT |
| | | TGGTTTTTAAAAAATCTGGAATCGAAAAT |
| | | TTGCGGGGCGACCGAGAAGTTTTTTACAA |
| | | AAGGCAAAAACTTTTTCGGGATCGACAG |
| | | AAATAAAACGATCGACGGTACGCAACAA |
| | | AAAAGCGTCAGGATCGCCGTAGAGCGAT |
| | | TGAAGACCGTCAACCAAAGGGGAAGCCT |
| | | CCAATCGACGCGACGCGCGCTCTACGGC |
| | | GATCCTGACGCAGATTTTTAGCTATCTGT |
| | | CGCAGCGCCCTCAGGGACAAGCCACCCG |
| | | CACAACGTCGCGAGGGCGATCAGCGACG |
| | | CCGCAGGG |
| SEQ ID NO: 39 | APSE10797 | GGATCCTctagtCTACAATCCATGCCAACCC |
| | | GTTCCATGTGCTCGCCGAGGCGGCATAAA |
| | | TCGCCGTGACGATCAGCGGTCCAATGATC |
| | | GAAGTTAGGCTGGTAAGAGCCGCGAGCG |
| | | ATCCTTGAAGCTGTCCCTGATGGTCGTCA |
| | | TCTACCTGCCTGGACAGCATGGCCTGCAA |
| | | CGCGGGCATCCCGATGCCGCCGGAAGCG |
| | | AGAAGAATCATAATGGGGAAGGCCATCC |
| | | AGCCTCGCGtcgcgaGTAGAAACGCAAAAA |
| | | GGCCATCCGTCAGGATGGCCTTCTGCTTA |
| | | ATTTGATGCCTGGCAGTTTATGGCGGGCG |
| | | TCCTGCCCGCCACCCTCCGGGCCGTTGCT |
| | | TCGCAACGTTCAAATCCGCTCCCGGCGGA |
| | | TTTGTCCTACTCAGGAGAGCGTTCACCGA |
| | | CAAACAACAGATAAAACGAAAGGCCCAG |
| | | TCTTTCGACTGAGCCTTTCGTTTTATTTGA |
| | | TGCCTGGCAGTTCCCTACTCTCGCATGGG |
| | | GAGACCCCACACTACCATCGGCGCTACG |
| | | GCGTTTCACTTCTGAGTTCGGCATGGGGT |
| | | CAGGTGGGACCACCGCGCTACTGCCGCC |
| | | AGGCAAATTCTGTTTTATCAGACaGCTTCT |
| | | GCGTTCTGcctaggttaaacgccaaactcttcgcgataggcc |
| | | ttaaccgccgccagatgttccgccatttccggcttctcttccaggtaa |
| | | gcaatcaggtctttcagggtgatgatagagatcactttgcagttgtaat |
| | | cacgctcaacttcctgaatcgccgaaatctcgccgcgcccgcgttc |
| | | ctgacgatcgagcgaaatcaacacgccagcaagcgtcgcgccatt |
| | | ggcctgaataatctccatcgactcgcgaatcgccgttccggcggtg |
| | | atcacatcatctaccagcattacgcgtccttgtaacgcgctaccaacc |
| | | agattgccgccttcaccgtggtcttttgcttctttgcggttaaagcagt |
| | | acggcaggtccaggtcgtgatgctccgccagtgccacagcggttgt |
| | | ggtggcaatcgggatccctttgtaagcagggccaaacagcagatc |
| | | gaactcaatgccggaatccaccaacgcttcagcgtaaaaacggcct |
| | | aacagtgccagatcgcgcccggtattaaacagcccggcgttgaag |
| | | aaataggggcttttgcgcccggatttcagcgtaaactcgccaaacttt |
| | | aacacctgcttgctaagcgcaaattCaataaactggcgctgatatgg |
| | | tttCATATGTATATCTCCTTCTgtcgacGCTCTC |
| | | CCTTATGCGACTCCTGCATTAGGAAGCAG |
| | | CCCAGTAGTAGGTTGAGGCCGTTGAGCA |
| | | CCGCCGCCGCAAGGAATGGTGCATGCCG |
| | | GATATAGTTCCTCCTTTCAGCAAAAAACC |
| | | CCTCAAGACCCGTTTAGAGGCCCCAAGG |
| | | GGTTATGCTAGTTATTGCTCAGCGGTGGC |
| | | AGCGCGTCCGCTTATTAGTAAATGCCAGA |
| | | GTTAGCAGCGATTGCAGATGGGATCGGA |
| | | TTACCGTCTTTCAGCAGGCCCTGCATTGC |
| | | CTTAACAATCAGTTCGCAGTCGGAGTTCG |
| | | TCGCAAAAATCGGAATAGTCAGTTCCATG |
| | | TTGAGGTAAGAGCGCCAGGCCGCAACTG |
| | | GGAGTTCTACGCCGCCTACGGTCTGCGTT |
| | | GCGACTTTCGGTACTTCAACTTTGATGGT |
| | | GTATTTGCGATTCTGCGCGCTAGACTGAC |
| | | GAACGGAGCACGTGACTTTGTAGGCCTG |
| | | AGAGCGGCTGTTAGAGCTAATCCATTCCG |
| | | CAACACCGTTGGCGAAGTTGGACGGAGC |
| | | TACGGTAACGTCACCCGTACCGCCGTTAT |
| | | CAACCAGAACGAATTGGGTAAAGTTAGA |
| | | CGCCATATGTATATCTCCTTCTTAGCCTG |
| | | CTTTTTTGTACAAACTTGTGATCTAGAGG |
| | | GAAACCGTTGTGGTCTCCCTATAGTGAGT |
| | | CGTATTAATTTCGCGGGATCGAGATCTGG |
| | | ATCCACAGGACGGGTGTGGTCGCCATGA |
| | | TCGCGTAGTCGATAGTGGCTCCAAGTAGC |
| | | GAAGCGAGCAGGACTGGGCGGCGGCCAA |
| | | AGCGGTCGGACAGTGCTCCGAGAACGGG |
| | | TGCGCATAGAAATTGCATCAACGCATATA |
| | | GCGCTAGCAGCACGCCATAGTGACTGGC |
| | | GATGCTGTCGGAATGGACGATGCATGCC |
| | | CGCCGCCCAGTCCTGCTCGCTTCGCTACT |
| | | TGGAGCCACTATCGACTACGCGATCATGG |
| | | CGACCACACCCGTCCTGTGGATATCCGGA |
| | | TATAGTTCCTCCTTTCAGCAAAAAACCCC |
| | | TCAAGACCCGTTTAGAGGCCCCAAGGGG |
| | | TTATGCTAGGGTGTGGGAGCTTCGACATG |
| | | GGTGATCCTCATGTTTGAACTCCTTAATT |
| | | AACTCTAAGTACTTCTTGGGTACCGCGGC |
| | | CGCCCTCGTTCAGACCGGGTATTCGGTCC |
| | | GCGCACGAGGTTTTTCTGTCTAGTGAGCA |
| | | GTGTCCAACCTCAAAAGACAACATGTGT |
| | | GACGACGATGTAGCGGCTCTTGTCGTAGA |
| | | CAATGGATCCGGTATGTGCAAAGCCGGTT |
| | | TCGCAGGAGATGACGCACCCCGTGCCGT |
| | | CTTCCCCTCGATCGTCGGTCGCCCAAGGC |
| | | ATCAAGGAGTCATGGTCGGTATGGGACA |
| | | AAAGGACTCATACGTAGGAGATGAAGCC |
| | | CAAAGCAAAAGAGGTATCCTCACCCTGA |
| | | AATACCCCATCGAACACGGTATCATCACC |
| | | AACTGGGATGAGGCGCGCCAGAACTCAC |
| | | TATGGAATGGCGGCTTCGGCCGCCAACG |
| | | ATGGATCCCAGCGGAACTAGGAAGCGAT |
| | | GTGGCCTGAGGAATACCTCTCTCATTGCG |
| | | TTTAGATAAAGCATCCCGCTGGAAAGGG |
| | | AACCAGTACTTTGTAAAAGCTAGAGGGTT |
| | | TAAACTCATCCCAGTTGGTGATGATACCG |
| | | TGTTCGATGGGGTATTTCAGGGTGAGGAT |
| | | ACCTCTTTTGCTTTGGGCTTCATCTCCTAC |
| | | GTATGAGTCCTTTTGTCCCATACCGACCA |
| | | TGACTCCTTGATGCCTTGGGCGACCGACG |
| | | ATCGAGGGGAAGACGGCACGGGGTGCGT |
| | | CATCTCCTGCGAAACCGGCTTTGCACATA |
| | | CCGGATCCATTGTCTACGACAAGAGCCGC |
| | | TACATCGTCGTCACACATGTTGTCTTTTG |
| | | AGGTTGGACACTGCTCACTAGACAGAAA |
| | | AACCTCGTGCGGCGATCGCACATGGGTA |
| | | ATCCTCATGTCTCCCTATAGTGAGTCGTA |
| | | TTAATTTCGGGAGAATTCTTGAAGACGAA |
| | | AGGGCCTCGTGATACGCCTATTTTTATAG |
| | | GTTAATGTCATGATAATAATGGTTTCTTA |
| | | GACGTCAGGTGGCACTTTTCGGGGAAAT |
| | | GTGCGCGGAACCCCTATTTGTTTATTTTTC |
| | | TAAATACATTCAAATATGTATCCGCTCAT |
| | | GAGACAATAACCCTGATAAATGCTTCAAT |
| | | AATATTGAAAAAGGAAGAGTATGAGTAT |
| | | TCAACATTTCCGTGTCGCCCTTATTCCCTT |
| | | TTTTGCGGCATTTTGCCTTCCTGTTTTTGC |
| | | TCACCCAGAAACGCTGGTGAAAGTAAAA |
| | | GATGCTGAAGATCAGTTGGGTGCACGAG |
| | | TGGGTTACATCGAACTGGATCTCAACAGC |
| | | GGTAAGATCCTTGAGAGTTTTCGCCCCGA |
| | | AGAACGTTTTCCAATGATGAGCACTTTTA |
| | | AAGTTCTGCTATGTGGCGCGGTATTATCC |
| | | CGTGTTGACGCCGGGCAAGAGCAACTCG |
| | | GTCGCCGCATACACTATTCTCAGAATGAC |
| | | TTGGTTGAGTACTCACCAGTCACAGAAAA |
| | | GCATCTTACGGATGGCATGACAGTAAGA |
| | | GAATTATGCAGTGCTGCCATAACCATGAG |
| | | TGATAACACTGCGGCCAACTTACTTCTGA |
| | | CAACGATCGGAGGACCGAAGGAGCTAAC |
| | | CGCTTTTTTGCACAACATGGGGGATCATG |
| | | TAACTCGCCTTGATCGTTGGGAACCGGAG |
| | | CTGAATGAAGCCATACCAAACGACGAGC |
| | | GTGACACCACGATGCCTGCAGCAATGGC |
| | | AACAACGTTGCGCAAACTATTAACTGGC |
| | | GAACTACTTACTCTAGCTTCCCGGCAACA |
| | | ATTAATAGACTGGATGGAGGCGGATAAA |
| | | GTTGCAGGACCACTTCTGCGCTCGGCCCT |
| | | TCCGGCTGGCTGGTTTATTGCTGATAAAT |
| | | CTGGAGCCGGTGAGCGTGGGTCTCGCGG |
| | | TATCATTGCAGCACTGGGGCCAGATGGTA |
| | | AGCCCTCCCGTATCGTAGTTATCTACACG |
| | | ACGGGGAGTCAGGCAACTATGGATGAAC |
| | | GAAATAGACAGATCGCTGAGATAGGTGC |
| | | CTCACTGATTAAGCATTGGTAACTGTCAG |
| | | ACCAAGTTTACTCATATATACTTTAGATT |
| | | GATTTAAAACTTCATTTTTAATTTAAAAG |
| | | GATCTAGGTGAAGATCCTTTTTGATAATC |
| | | TCATGACCAAAATCCCTTAACGTGAGTTT |
| | | TCGTTCCACTGAGCGTCAGACCCCTTAAT |
| | | AAGATGATCTTCTTGAGATCGTTTTGGTC |
| | | TGCGCGTAATCTCTTGCTCTGAAAACGAA |
| | | AAAACCGCCTTGCAGGGCGGTTTTTCGAA |
| | | GGTTCTCTGAGCTACCAACTCTTTGAACC |
| | | GAGGTAACTGGCTTGGAGGAGCGCAGTC |
| | | ACCAAAACTTGTCCTTTCAGTTTAGCCTT |
| | | AACCGGCGCATGACTTCAAGACTAACTCC |
| | | TCTAAATCAATTACCAGTGGCTGCTGCCA |
| | | GTGGTGCTTTTGCATGTCTTTCCGGGTTG |
| | | GACTCAAGACGATAGTTACCGGATAAGG |
| | | CGCAGCGGTCGGACTGAACGGGGGGTTC |
| | | GTGCATACAGTCCAGCTTGGAGCGAACT |
| | | GCCTACCCGGAACTGAGTGTCAGGCGTG |
| | | GAATGAGACAAACGCGGCCATAACAGCG |
| | | GAATGACACCGGTAAACCGAAAGGCAGG |
| | | AACAGGAGAGCGCACGAGGGAGCCGCCA |
| | | GGGGGAAACGCCTGGTATCTTTATAGTCC |
| | | TGTCGGGTTTCGCCACCACTGATTTGAGC |
| | | GTCAGATTTCGTGATGCTTGTCAGGGGGG |
| | | CGGAGCCTATGGAAAAACGGCTTTGCCG |
| | | CGGCCCTCTCACTTCCCTGTTAAGTATCTT |
| | | CCTGGCATCTTCCAGGAAATCTCCGCCCC |
| | | GTTCGTAAGCCATTTCCGCTCGCCGCAGT |
| | | CGAACGACCGAGCGTAGCGAGTCAGTGA |
| | | GCGAGGAAGCGGAATATATCCTGTATCA |
| | | CATATTCTGCTGACGCACCGGTGCAGCCT |
| | | TTTTTCTCCTGCCACATGAAGCACTTCAC |
| | | TGACACCCTCATCAGTGCCAACATAGTAA |
| | | GCCAGTATACACTCCGCTAGCGCTGAGGT |
| | | CTGCCTCGTGAAGAAGGTGTTGCTGACTC |
| | | ATACCAGGCCTGAATCGCCCCATCATCCA |
| | | GCCAGAAAGTGAGGGAGCCACGGTTGAT |
| | | GAGAGCTTTGTTGTAGGTGGACCAGTTGG |
| | | TGATTTTGAACTTTTGCTTTGCCACGGAA |
| | | CGGTCTGCGTTGTCGGGAAGATGCGTGAT |
| | | CTGATCCTTCAACTCAGCAAAAGTTCGAT |
| | | TTATTCAACAAAGCCACGTTGTGTCTCAA |
| | | AATCTCTGATGTTACATTGCACAAGATAA |
| | | AAATATATCATCATGAACAATAAAACTGT |
| | | CTGCTTACATAAACAGTAATACAAGGGG |
| | | TGTTATGAGCCATATTCAACGGGAAACGT |
| | | CTTGCTCGAGGCCGCGATTAAATTCCAAC |
| | | ATGGATGCTGATTTATATGGGTATAAATG |
| | | GGCTCGCGATAATGTCGGGCAATCAGGT |
| | | GCGACAATCTATCGATTGTATGGGAAGCC |
| | | CGATGCGCCAGAGTTGTTTCTGAAACATG |
| | | GCAAAGGTAGCGTTGCCAATGATGTTAC |
| | | AGATGAGATGGTCAGACTAAACTGGCTG |
| | | ACGGAATTTATGCCTCTTCCGACCATCAA |
| | | GCATTTTATCCGTACTCCTGATGATGCAT |
| | | GGTTACTCACCACTGCGATCCCCGGGAAA |
| | | ACAGCATTCCAGGTATTAGAAGAATATCC |
| | | TGATTCAGGTGAAAATATTGTTGATGCGC |
| | | TGGCAGTGTTCCTGCGCCGGTTGCATTCG |
| | | ATTCCTGTTTGTAATTGTCCTTTTAACAGC |
| | | GATCGCGTATTTCGTCTCGCTCAGGCGCA |
| | | ATCACGAATGAATAACGGTTTGGTTGATG |
| | | CGAGTGATTTTGATGACGAGCGTAATGGC |
| | | TGGCCTGTTGAACAAGTCTGGAAAGAAA |
| | | TGCATAAGCTTTTGCCATTCTCACCGGAT |
| | | TCAGTCGTCACTCATGGTGATTTCTCACT |
| | | TGATAACCTTATTTTTGACGAGGGGAAAT |
| | | TAATAGGTTGTATTGATGTTGGACGAGTC |
| | | GGAATCGCAGACCGATACCAGGATCTTG |
| | | CCATCCTATGGAACTGCCTCGGTGAGTTT |
| | | TCTCCTTCATTACAGAAACGGCTTTTTCA |
| | | AAAATATGGTATTGATAATCCTGATATGA |
| | | ATAAATTGCAGTTTCATTTGATGCTCGAT |
| | | GAGTTTTTCTAATCAGAATTGGTTAATTG |
| | | GTTGTAACACTGGCAGAGCATTACGCTGA |
| | | CTTGACGGGACGGCGGCTTTGTTGAATAA |
| | | ATCGAACTTTTGCTGAGTTGAAGGATCAG |
| | | ATCACGCATCTTCCCGACAACGCAGACCG |
| | | TTCCGTGGCAAAGCAAAAGTTCAAAATC |
| | | ACCAACTGGTCCACCTACAACAAAGCTCT |
| | | CATCAACCGTGGCTCCCTCACTTTCTGGC |
| | | TGGATGATGGGGCGATTCAGGCCTGGTAT |
| | | GAGTCAGCAACACCTTCTTCACGAGGCA |
| | | GACCTCAGCGCTCAAAGATGCAGGGGTA |
| | | AAAGCTAACCGCATCTTTACCGACAAGG |
| | | CATCCGGCAGTTCAACAGATCGGGAAGG |
| | | GCTGGATTTGCTGAGGATGAAGGTGGAG |
| | | GAAGGTGATGTCATTCTGGTGAAGAAGC |
| | | TCGACCGTCTTGGCCGCGACACCGCCGAC |
| | | ATGATCCAACTGATAAAAGAGTTTGATGC |
| | | TCAGGGTGTAGCGGTTCGGTTTATTGACG |
| | | ACGGGATCAGTACCGACGGTGATATGGG |
| | | GCAAATGGTGGTCACCATCCTGTCGGCTG |
| | | TGGCACAGGCTGAACGCCGGAGGATCAA |
| | | GTCGGTCAAGCCAAGCGCAACCAGCGGC |
| | | ACCGCCGCGAGCAACGTCGCAAGGGCGA |
| | | TCAGGGGACGATTTTTGCGAAGAATTTCC |
| | | ACGGTAAGAATCCAATCTCTCGAATTTAG |
| | | GGTGAAAGAAGCTTGGCATAGGGGTGTG |
| | | CACGAACTCGGTGGAGGAAATTTCCGCG |
| | | GGGCAAGGCTTCGCGAAGCGGAGTCGCG |
| | | GCAGTGGCTTTGAAGATCTTTGGGAGCAG |
| | | TCCTTGTGCGCTTACGAGGTGAGCCGGTG |
| | | GGGAACCGTTATCTGCCTATGGTGTGAGC |
| | | CCCCCTAGAGAGCTTCAAGAGCAATCAG |
| | | CCCGACCTAGAAAGGAGGCCAAGAGAGA |
| | | GACCCCTACGGGGGGAACCGTTTTCTGCC |
| | | TACGAGATGGCACATTTACTGGGAAGCTT |
| | | TACGGCGTCCTCGTGGAAGTTCAATGCCC |
| | | GCAGACTTAAGTGCTCTATTCACGGTCTG |
| | | ACGTGACACGCTAAATTCAGACATAGCTT |
| | | CATTGATTGTCGGCCACGAGCCAGTCTCT |
| | | CCCTCAACAGTCATAAACCAACCTGCAAT |
| | | GGTCAAGCGATTTCCTTTAGCTTTCCTAG |
| | | CTTGTCGTTGACTGGACTTAGCTAGTTTTT |
| | | CTCGCTGTGCTCGGGCGTACTCACTGTTT |
| | | GGGTCTTTCCAGCGTTCTGCGGCCTTTTT |
| | | ACCGCCACGTCTTCCCATAGTGGCCAGAG |
| | | CTTTTCGCCCTCGGCTGCTCTGCGTCTCTG |
| | | TCTGACGAGCAGGGACGACTGGCTGGCC |
| | | TTTAGCGACGTAGCCGCGCACACGTCGCG |
| | | CCATCGTCTGGCGGTCACGCATCGGCGGC |
| | | AGATCAGGCTCACGGCCGTCTGCTCCGAC |
| | | CGCCTGAGCGACGGTGTAGGCACGCTCG |
| | | TAGGCGTCGATGATCTTGGTGTCTTTTAG |
| | | GCGCTCACCAGCCGCTTTTAACTGGTATC |
| | | CCACAGTCAAAGCGTGGCGAAAAGCCGT |
| | | CTCATCACGGGCGGCACGCCCTGGAGCA |
| | | GTCCAGAGGACACGGACGCCGTCGATCA |
| | | GCTCTCCAGACGCTTCAGCGGCGCTCGGC |
| | | AGGCTTGCTTCAAGCGTGGCAAGTGCTTT |
| | | TGCTTCCGCAGTGGCTTTTCTTGCCGCTTC |
| | | GATACGTGCCCGTCCGCTAGAAAACTCCT |
| | | GCTCATAGCGTTTTTTAGGTTTTTCTGTGC |
| | | CTGAGATCATGCGAGCAACCTCCATAAG |
| | | ATCAGCTAGGCGATCCACGCGATTGTGCT |
| | | GGGCATGCCAGCGGTACGCGGTGGGATC |
| | | GTCGGAGACGTGCAGTGGCCACCGGCTC |
| | | AGCCTATGTGAAAAAGCCTGGTCAGCGC |
| | | CGAAAACGCGGGTCATTTCCTCGGTCGTT |
| | | GCAGCCAGCAGGCGCATATTCGGGCTGC |
| | | TCATGCCTGCTGCGGCATACACCGGATCA |
| | | ATGAGCCAGATGAGCTGGCATTTCCCGCT |
| | | CAGTGGATTCACGCCGATCCAAGCTGGC |
| | | GCTTTTTCCAGGCGTGCCCAGCGCTCCAA |
| | | AATCGCGTAGACCTCGGGGTTTACGTGCT |
| | | CGATTTTCCCGCCGGCCTGGTGGCTCGGC |
| | | ACATCAATGTCCAGGACAAGCACGGCTG |
| | | CGTGCTGCGCGTGCGTCAGAGCAACATA |
| | | CTGGCACCGGGCAAGCGATTTTGAACCA |
| | | ACTCGGTATAACTTCGGCTGTGTTTCTCC |
| | | CGTGTCCGGGTCTTTGATCCAAGCGCTGG |
| | | CGAAGTCGCGGGTCTTGCTGCCCTGGAAA |
| | | TTTTCTCTGCCCAGGTGAGCGAGGAATTC |
| | | GCGGCGGTCTTCGCTCGTCCAGCCACGTG |
| | | ATCGCAGCGCGAGCTCGGGATGGGTGTC |
| | | GAACAGATCAGCGGAAAATTTCCAGGCC |
| | | GGTGTGTCAATGTCTCGTGAATCCGCTAG |
| | | AGTCATTTTTGAGCGCTTTCTCCCAGGTTT |
| | | GGACTGGGGGTTAGCCGACGCCCTGTGA |
| | | GTTACCGCTCACGGGGCGTTCAACATTTT |
| | | TCAGGTATTCGTGCAGCTTATCGCTTCTT |
| | | GCCGCCTGTGCGCTTTTTCGACGCGCGAC |
| | | GCTGCTGCCGATTCGGTGCAGGTGGTGGC |
| | | GGCGCTGACACGTCCTGGGCGGCCACGG |
| | | CCACACGAAACGCGGCATTTACGATGTTT |
| | | GTCATGCCTGCGGGCACCGCGCCACGATC |
| | | GCGGATAATTCTCGCTGCCGCTTCCAGCT |
| | | CTGTGACGACCATGGCCAAAATTTCGCTC |
| | | GGGGGACGCACTTCCAGCGCCATTTGCG |
| | | ACCTAGCCGCCTCCAGCTCCTCGGCGTGG |
| | | CGTTTGTTGGCGCGCTCGCGGCTGGCTGC |
| | | GGCACGACACGCATCTGAGCAATATTTTG |
| | | CGCGCCGTCCTCGCGGGTCAGGCCGGGG |
| | | AGGAATCAGGCCACCGCAGTAGGCGCAA |
| | | CTGATTCGATCCTCCACTACTGTGCGTCC |
| | | TCCTGGCGCTGCCGAGCACGCAGCTCGTC |
| | | AGCCAGCTCCTCAAGATCCGCCACGAGA |
| | | GTTTCTAGGTCGCTCGCGGCACTGGCCCA |
| | | GTCTCGTGATGCTGGCGCGTCCGTCGTAT |
| | | CGAGAGCTCGGAAAAATCCGATCACCGT |
| | | TTTTAAATCGACGGCAGCATCGAGCGCGT |
| | | CGGACTCCAGCGCGACATCAGAGAGATC |
| | | CATAGCTGATGATTCGGGCCAATTTTGGT |
| | | ACTTCGTCGTGAAGGTCATGACACCATTA |
| | | TAACGAACGTTCGTTAAAGTTTTTGGCGG |
| | | AAAATCACGCGGCACGAAAATTTTCACG |
| | | AAGCGGGACTTTGCGCAGCTCAGGGGTG |
| | | CTAAAAATTTTGTATCGCACTTGATTTTTC |
| | | CGAAAGACAGATTATCTGCAAACGGTGT |
| | | GTCGTATTTCTGGCTTGGTTTTTAAAAAA |
| | | TCTGGAATCGAAAATTTGCGGGGCGACC |
| | | GAGAAGTTTTTTACAAAAGGCAAAAACT |
| | | TTTTCGGGATCGACAGAAATAAAACGAT |
| | | CGACGGTACGCAACAAAAAAGCGTCAGG |
| | | ATCGCCGTAGAGCGATTGAAGACCGTCA |
| | | ACCAAAGGGGAAGCCTCCAATCGACGCG |
| | | ACGCGCGCTCTACGGCGATCCTGACGCA |
| | | GATTTTTAGCTATCTGTCGCAGCGCCCTC |
| | | AGGGACAAGCCACCCGCACAACGTCGCG |
| | | AGGGCGATCAGCGACGCCGCAGGG |
| SEQ ID NO: 40 | APSE10822 | ACCCTGGGCCAACTTTTGGCGAAAATGA |
| | | GACGTTGATCGGCACGTAAGAGGTTCCA |
| | | ACTTTCACCATAATGAAATAAGATCACTA |
| | | CCGGGCGTATTTTTTGAGTTATCGAGATT |
| | | TTCAGGAGCTAAGGAAGCTAAAATGGAG |
| | | AAAAAAATCACTGGATATACCACCGTTG |
| | | ATATATCCCAATGGCATCGTAAAGAACAT |
| | | TTTGAGGCATTTCAGTCAGTTGCTCAATG |
| | | TACCTATAACCAGACCGTTCAGCTGGATA |
| | | TTACGGCCTTTTTAAAGACCGTAAAGAAA |
| | | AATAAGCACAAGTTTTATCCGGCCTTTAT |
| | | TCACATTCTTGCCCGCCTGATGAATGCTC |
| | | ATCCGGAATTCCGTATGGCAATGAAAGA |
| | | CGGTGAGCTGGTGATATGGGATAGTGTTC |
| | | ACCCTTGTTACACCGTTTTCCATGAGCAA |
| | | ACTGAAACGTTTTCATCGCTCTGGAGTGA |
| | | ATACCACGACGATTTCCGGCAGTTTCTAC |
| | | ACATATATTCGCAAGATGTGGCGTGTTAC |
| | | GGTGAAAACCTGGCCTATTTCCCTAAAGG |
| | | GTTTATTGAGAATATGTTTTTCGTCTCAG |
| | | CCAATCCCTGGGTGAGTTTCACCAGTTTT |
| | | GATTTAAACGTGGCCAATATGGACAACTT |
| | | CTTCGCCCCCGTTTTCACCATGGGCAAAT |
| | | ATTATACGCAAGGCGACAAGGTGCTGAT |
| | | GCCGCTGGCGATTCAGGTTCATCATGCCG |
| | | TTTGTGATGGCTTCCATGTCGGCAGAATG |
| | | CTTAATGAATTACAACAGTACTGCGATGA |
| | | GTGGCAGGGCGGGGCGTAATTTTTTTAAG |
| | | GCAGTTATTGGTGCCCTTAAACGCCTGGT |
| | | TGCTACGCCTGAATAAGTGATAATAAGC |
| | | GGATGAATGGCAGAAATTCGAAAGCAAA |
| | | TTCGACCCGGTCGTCGGTTCAGGGCAGGG |
| | | TCGTTAAATAGCCGCTTGGATCCTGAAAG |
| | | GTCAGGGCCAGGGCTGGATCACCGCAGA |
| | | GTACGGCATGCTGCCACGTTCTACCCACA |
| | | CCCGTAACGCTCGTGAAGCGGCGAAAGG |
| | | TAAGCAGGGTGGACGCACAATGGAAATC |
| | | CAGCGTCTGATCGCCCGTGCTCTTCGCGC |
| | | GGCAGTAGATTTGAAAGCGCTGGGTGAG |
| | | TTCACCATTACGCTGGACTGCGACGTGCT |
| | | TCAGGCTGATGGTGGCACGCGTACCGCGT |
| | | CGATTACGGGTGCCTGCGTGGCGCTGGTA |
| | | GATGCGCTACAGAAGCTGGTGGAAAACG |
| | | GCAAGCTGAAAACCAATCCGATGAAAGG |
| | | GATGGTAGCCGCAGTTTCTGTCGGAATTG |
| | | TGAACGGCGAAGCGGTTTGCGATCTGGA |
| | | ATACGTTGAAGACTCTGCCGCAGAGACC |
| | | GACATGAACGTAGTGATGACCGAAGACG |
| | | GGCGCATCATTGAAGTGCAGGGGACGGC |
| | | AGAAGGCGAGCCGTTCACCCATGAAGAG |
| | | CTACTCATCTTGTTGGCTCTGGCCCGAGG |
| | | GGAATCGAATCCATTGTAGCGACGCAGA |
| | | AGGCGGCGCTGGCAAACTGATTTTTAAG |
| | | GCGACTGATGAGTCGCCTTTTTTTTGTCT |
| | | GTAGAAAAGTAAGATGAGGAGCGAAGGC |
| | | AAAGTGATCTCTATCATCACCCTGAAAGA |
| | | CCTGATTGCTTACCTGGAAGAGAAGCCG |
| | | GAAATGGCGGAACATCTGGCGGCGGTTA |
| | | AGGCCTATCGCGAAGAGTTTGGCGTTTAA |
| | | AGAAACTCGCCGGATGAAAAGTCATCCG |
| | | GCGTCATATTACTGCAACTGTGCCGCAAT |
| | | TAGCGGCCAGCGGGCGTCAAAATCATCC |
| | | GTCGGGCGGTATTTAAATTCGCTGCGGAC |
| | | AAAACGTGACAGCATACCTTCACAGAAG |
| | | GCCAGGATCTGGCTTGCCAGCAGGGTTTC |
| | | ATCGGTGGTGTAACCTTCACCCTCACGCA |
| | | TTCTCTTTTCACGCAATACCTGGCGCAGC |
| | | TGCGCTTCAATACGCTCGAACAGCTGGTT |
| | | GATGCGCCCTTGCAGGCGATCCTGTTCAA |
| | | ACATTAGCGCATGACCAGTGAGGATGCG |
| | | GGTCAGGCCAGGATTACGCTCACCAAAA |
| | | CCGAGAAGCAGCAACACAATCAGACGCA |
| | | GGCGCGCTGTGGTGTCTTTCTCATCTTTC |
| | | AGAATCAGGTTGATGCGAGTAATCAGGC |
| | | TATCTTCGATAAACTCAATCAGGCTATCG |
| | | AACATGCGGGTCTTACTGGGGAAGTGGC |
| | | GATACAGTGCCGCTTCGGATCCTTTTTAA |
| | | CCCATCACATATACCTGCCGTTCACTATT |
| | | ATTTAGTGAAATGAGATATTATGATATTT |
| | | TCTGAATTGTGATTAAAAAGGCAACTTTA |
| | | TGCCCATGCAACAGAAACTATAAAAAAT |
| | | ACAGAGAATGAAAAGAAACAGATAGATT |
| | | TTTTAGTTCTTTAGGCCCGTAGTCTGCAA |
| | | ATCCTTTTATGATTTTCTATCAAACAAAA |
| | | GAGGAAAATAGACCAGTTGCAATCCAAA |
| | | CGAGAGTCTAATAGAATGAGGTCGAAAA |
| | | GTAAATCGCGCGGGTTTGTTACTGATAAA |
| | | GCAGGCAAGACCTAAAATGTGTAAAGGG |
| | | CAAAGTGTATACTTTGGCGTCACCCCTTA |
| | | CATATTTTAGGTCTTTTTTTATTGTGCGTA |
| | | ACTAACTTGCCATCTTCAAACAGGAGGGC |
| | | TGGAAGAAGCAGACCGCTAACACAGTAC |
| | | ATAAAAAAGGAGACATGAACGATGAACA |
| | | TCAAAAAGTTTGCAAAACAAGCAACAGT |
| | | ATTAACCTTTACTACCGCACTGCTGGCAG |
| | | GAGGCGCAACTCAAGCGTTTGCGAAAGA |
| | | AACGAACCAAAAGCCATATAAGGAAACA |
| | | TACGGCATTTCCCATATTACACGCCATGA |
| | | TATGCTGCAAATCCCTGAACAGCAAAAA |
| | | AATGAAAAATATCAAGTTTCTGAATTTGA |
| | | TTCGTCCACAATTAAAAATATCTCTTCTG |
| | | CAAAAGGCCTGGACGTTTGGGACAGCTG |
| | | GCCATTACAAAACGCTGACGGCACTGTC |
| | | GCAAACTATCACGGCTACCACATCGTCTT |
| | | TGCATTAGCCGGAGATCCTAAAAATGCG |
| | | GATGACACATCGATTTACATGTTCTATCA |
| | | AAAAGTCGGCGAAACTTCTATTGACAGCT |
| | | GGAAAAACGCTGGCCGCGTCTTTAAAGA |
| | | CAGCGACAAATTCGATGCAAATGATTCTA |
| | | TCCTAAAAGACCAAACACAAGAATGGTC |
| | | AGGTTCAGCCACATTTACATCTGACGGAA |
| | | AAATCCGTTTATTCTACACTGATTTCTCC |
| | | GGTAAACATTACGGCAAACAAACACTGA |
| | | CAACTGCACAAGTTAACGTATCAGCATCA |
| | | GACAGCTCTTTGAACATCAACGGTGTAGA |
| | | GGATTATAAATCAATCTTTGACGGTGACG |
| | | GAAAAACGTATCAAAATGTACAGCAGTT |
| | | CATCGATGAAGGCAACTACAGCTCAGGC |
| | | GACAACCATACGCTGAGAGATCCTCACT |
| | | ACGTAGAAGATAAAGGCCACAAATACTT |
| | | AGTATTTGAAGCAAACACTGGAACTGAA |
| | | GATGGCTACCAAGGCGAAGAATCTTTATT |
| | | TAACAAAGCATACTATGGCAAAAGCACA |
| | | TCATTCTTCCGTCAAGAAAGTCAAAAACT |
| | | TCTGCAAAGCGATAAAAAACGCACGGCT |
| | | GAGTTAGCAAACGGCGCTCTCGGTATGAT |
| | | TGAGCTAAACGATGATTACACACTGAAA |
| | | AAAGTGATGAAACCGCTGATTGCATCTA |
| | | ACACAGTAACAGATGAAATTGAACGCGC |
| | | GAACGTCTTTAAAATGAACGGCAAATGG |
| | | TACCTGTTCACTGACTCCCGCGGATCAAA |
| | | AATGACGATTGACGGCATTACGTCTAACG |
| | | ATATTTACATGCTTGGTTATGTTTCTAATT |
| | | CTTTAACTGGCCCATACAAGCCGCTGAAC |
| | | AAAACTGGCCTTGTGTTAAAAATGGATCT |
| | | TGATCCTAACGATGTAACCTTTACTTACT |
| | | CACACTTCGCTGTACCTCAAGCGAAAGG |
| | | AAACAATGTCGTGATTACAAGCTATATGA |
| | | CAAACAGAGGATTCTACGCAGACAAACA |
| | | ATCAACGTTTGCGCCGAGCTTCCTGCTGA |
| | | ACATCAAAGGCAAGAAAACATCTGTTGT |
| | | CAAAGACAGCATCCTTGAACAAGGACAA |
| | | TTAACAGTTAACAAATAAAAACGCAAAA |
| | | GAAAATGCCGATGGAGCAATGGCGATGA |
| | | CGCATCCTCACGATAATATCCGGGTAGGC |
| | | GCAATCACTTTCGTCTACTCCGTTACAAA |
| | | GCGAGGCTGGGTATTTCCCGGCCTTTCTG |
| | | TTATCCGAAATCCACTGAAAGCACAGCG |
| | | GCTGGCTGAGGAGATAAATAATAAACGA |
| | | GGGGCTGTATGCACAAAGCATCTTCTGTT |
| | | GAGTTAAGAACGAGTATCGAGATGGCAC |
| | | ATAGCCTTGCTCAAATTGGAATCAGGTTT |
| | | GTGCCAATACCAGTAGAAACAGACGAAG |
| | | AATCCATGGGTATGGACAGTTTTCCCTTT |
| | | GATATGTAACGGTGAACAGTTGTTCTACT |
| | | TTTGTTTGTTAGTCTTGATGCTTCACTGAT |
| | | AGATACAAGAGCCATAAGAACCTCAGAT |
| | | CCTTCCGTATTTAGCCAGTATGTTCTCTA |
| | | GTGTGGTTCGTTGTTTTTGCGTGAGCCAT |
| | | GAGAACGAACCATTGAGATCATACTTACT |
| | | TTGCATGTCACTCAAAAATTTTGCCTCAA |
| | | AACTGGTGAGCTGAATTTTTGCAGTTAAA |
| | | GCATCGTGTAGTGTTTTTCTTAGTCCGTTA |
| | | TGTAGGTAGGAATCTGATGTAATGGTTGT |
| | | TGGTATTTTGTCACCATTCATTTTTATCTG |
| | | GTTGTTCTCAAGTTCGGTTACGAGATCCA |
| | | TTTGTCTATCTAGTTCAACTTGGAAAATC |
| | | AACGTATCAGTCGGGCGGCCTCGCTTATC |
| | | AACCACCAATTTCATATTGCTGTAAGTGT |
| | | TTAAATCTTTACTTATTGGTTTCAAAACC |
| | | CATTGGTTAAGCCTTTTAAACTCATGGTA |
| | | GTTATTTTCAAGCATTAACATGAACTTAA |
| | | ATTCATCAAGGCTAATCTCTATATTTGCC |
| | | TTGTGAGTTTTCTTTTGTGTTAGTTCTTTT |
| | | AATAACCACTCATAAATCCTCATAGAGTA |
| | | TTTGTTTTCAAAAGACTTAACATGTTCCA |
| | | GATTATATTTTATGAATTTTTTTAACTGGA |
| | | AAAGATAAGGCAATATCTCTTCACTAAA |
| | | AACTAATTCTAATTTTTCGCTTGAGAACT |
| | | TGGCATAGTTTGTCCACTGGAAAATCTCA |
| | | AAGCCTTTAACCAAAGGATTCCTGATTTC |
| | | CACAGTTCTCGTCATCAGCTCTCTGGTTG |
| | | CTTTAGCTAATACACCATAAGCATTTTCC |
| | | CTACTGATGTTCATCATCTGAACGTATTG |
| | | GTTATAAGTGAACGATACCGTCCGTTCTT |
| | | TCCTTGTAGGGTTTTCAATCGTGGGGTTG |
| | | AGTAGTGCCACACAGCATAAAATTAGCTT |
| | | GGTTTCATGCTCCGTTAAGTCATAGCGAC |
| | | TAATCGCTAGTTCATTTGCTTTGAAAACA |
| | | ACTAATTCAGACATACATCTCAATTGGTC |
| | | TAGGTGATTTTAATCACTATACCAATTGA |
| | | GATGGGCTAGTCAATGATAATTACTAGTC |
| | | CTTTTCCTTTGAGTTGTGGGTATCTGTAA |
| | | ATTCTGCTAGACCTTTGCTGGAAAACTTG |
| | | TAAATTCTGCTAGACCCTCTGTAAATTCC |
| | | GCTAGACCTTTGTGTGTTTTTTTTGTTTAT |
| | | ATTCAAGTGGTTATAATTTATAGAATAAA |
| | | GAAAGAATAAAAAAAGATAAAAAGAAT |
| | | AGATCCCAGCCCTGTGTATAACTCACTAC |
| | | TTTAGTCAGTTCCGCAGTATTACAAAAGG |
| | | ATGTCGCAAACGCTGTTTGCTCCTCTACA |
| | | AAACAGACCTTAAAACCCTAAAGGCTTA |
| | | AGTAGCACCCTCGCAAGCTCGGTTGCGGC |
| | | CGCAATCGGGCAAATCGCTGAATATTCCT |
| | | TTTGTCTCCGACCATCAGGCACCTGAGTC |
| | | GCTGTCTTTTTCGTGACATTCAGTTCGCTG |
| | | CGCTCACGGCTCTGGCAGTGAATGGGGG |
| | | TAAATGGCACTACAGGCGCCTTTTATGGA |
| | | TTCATGCAAGGAAACTACCCATAATACA |
| | | AGAAAAGCCCGTCACGGGCTTCTCAGGG |
| | | CGTTTTATGGCGGGTCTGCTATGTGGTGC |
| | | TATCTGACTTTTTGCTGTTCAGCAGTTCCT |
| | | GCCCTCTGATTTTCCAGTCTGACCACTTC |
| | | GGATTATCCCGTGACAGGTCATTCAGACT |
| | | GGCTAATGCACCCAGTAAGGCAGCGGTA |
| | | TCATCAACGGGGTCTG |
| SEQ ID NO: 41 | APSE10826 | ACCCTGGGCCAACTTTTGGCGAAAATGA |
| | | GACGTTGATCGGCACGTAAGAGGTTCCA |
| | | ACTTTCACCATAATGAAATAAGATCACTA |
| | | CCGGGCGTATTTTTTGAGTTATCGAGATT |
| | | TTCAGGAGCTAAGGAAGCTAAAATGGAG |
| | | AAAAAAATCACTGGATATACCACCGTTG |
| | | ATATATCCCAATGGCATCGTAAAGAACAT |
| | | TTTGAGGCATTTCAGTCAGTTGCTCAATG |
| | | TACCTATAACCAGACCGTTCAGCTGGATA |
| | | TTACGGCCTTTTTAAAGACCGTAAAGAAA |
| | | AATAAGCACAAGTTTTATCCGGCCTTTAT |
| | | TCACATTCTTGCCCGCCTGATGAATGCTC |
| | | ATCCGGAATTCCGTATGGCAATGAAAGA |
| | | CGGTGAGCTGGTGATATGGGATAGTGTTC |
| | | ACCCTTGTTACACCGTTTTCCATGAGCAA |
| | | ACTGAAACGTTTTCATCGCTCTGGAGTGA |
| | | ATACCACGACGATTTCCGGCAGTTTCTAC |
| | | ACATATATTCGCAAGATGTGGCGTGTTAC |
| | | GGTGAAAACCTGGCCTATTTCCCTAAAGG |
| | | GTTTATTGAGAATATGTTTTTCGTCTCAG |
| | | CCAATCCCTGGGTGAGTTTCACCAGTTTT |
| | | GATTTAAACGTGGCCAATATGGACAACTT |
| | | CTTCGCCCCCGTTTTCACCATGGGCAAAT |
| | | ATTATACGCAAGGCGACAAGGTGCTGAT |
| | | GCCGCTGGCGATTCAGGTTCATCATGCCG |
| | | TTTGTGATGGCTTCCATGTCGGCAGAATG |
| | | CTTAATGAATTACAACAGTACTGCGATGA |
| | | GTGGCAGGGCGGGGCGTAATTTTTTTAAG |
| | | GCAGTTATTGGTGCCCTTAAACGCCTGGT |
| | | TGCTACGCCTGAATAAGTGATAATAAGC |
| | | GGATGAATGGCAGAAATTCGAAAGCAAA |
| | | TTCGACCCGGTCGTCGGTTCAGGGCAGGG |
| | | TCGTTAAATAGCCGCTTGGATCCTGAAAG |
| | | GTCAGGGCCAGGGCTGGATCACCGCAGA |
| | | GTACGGCATGCTGCCACGTTCTACCCACA |
| | | CCCGTAACGCTCGTGAAGCGGCGAAAGG |
| | | TAAGCAGGGTGGACGCACAATGGAAATC |
| | | CAGCGTCTGATCGCCCGTGCTCTTCGCGC |
| | | GGCAGTAGATTTGAAAGCGCTGGGTGAG |
| | | TTCACCATTACGCTGGACTGCGACGTGCT |
| | | TCAGGCTGATGGTGGCACGCGTACCGCGT |
| | | CGATTACGGGTGCCTGCGTGGCGCTGGTA |
| | | GATGCGCTACAGAAGCTGGTGGAAAACG |
| | | GCAAGCTGAAAACCAATCCGATGAAAGG |
| | | GATGGTAGCCGCAGTTTCTGTCGGAATTG |
| | | TGAACGGCGAAGCGGTTTGCGATCTGGA |
| | | ATACGTTGAAGACTCTGCCGCAGAGACC |
| | | GACATGAACGTAGTGATGACCGAAGACG |
| | | GGCGCATCATTGAAGTGCAGGGGACGGC |
| | | AGAAGGCGAGCCGTTCACCCATGAAGAG |
| | | CTACTCATCTTGTTGGCTCTGGCCCGAGG |
| | | GGAATCGAATCCATTGTAGCGACGCAGA |
| | | AGGCGGCGCTGGCAAACTGATTTTTAAG |
| | | GCGACTGATGAGTCGCCTTTTTTTTGTCT |
| | | GTAGAAAAGTAAGATGAGGAGCGAAGGC |
| | | AAAGTGATCTCTATCATCACCCTGAAAGA |
| | | CCTGATTGCTTACCTGGAAGAGAAGCCG |
| | | GAAATGGCGGAACATCTGGCGGCGGTTA |
| | | AGGCCTATCGCGAAGAGTTTGGCGTTTAA |
| | | AGAAACTCGCCGGATGAAAAGTCATCCG |
| | | GCGTCATATTACTGCAACTGTGCCGCAAT |
| | | TAGCGGCCAGCGGGCGTCAAAATCATCC |
| | | GTCGGGCGGTATTTAAATTCGCTGCGGAC |
| | | AAAACGTGACAGCATACCTTCACAGAAG |
| | | GCCAGGATCTGGCTTGCCAGCAGGGTTTC |
| | | ATCGGTGGTGTAACCTTCACCCTCACGCA |
| | | TTCTCTTTTCACGCAATACCTGGCGCAGC |
| | | TGCGCTTCAATACGCTCGAACAGCTGGTT |
| | | GATGCGCCCTTGCAGGCGATCCTGTTCAA |
| | | ACATTAGCGCATGACCAGTGAGGATGCG |
| | | GGTCAGGCCAGGATTACGCTCACCAAAA |
| | | CCGAGAAGCAGCAACACAATCAGACGCA |
| | | GGCGCGCTGTGGTGTCTTTCTCATCTTTC |
| | | AGAATCAGGTTGATGCGAGTAATCAGGC |
| | | TATCTTCGATAAACTCAATCAGGCTATCG |
| | | AACATGCGGGTCTTACTGGGGAAGTGGC |
| | | GATACAGTGCCGCTTCGGATCCTTTTTAA |
| | | CCCATCACATATACCTGCCGTTCACTATT |
| | | ATTTAGTGAAATGAGATATTATGATATTT |
| | | TCTGAATTGTGATTAAAAAGGCAACTTTA |
| | | TGCCCATGCAACAGAAACTATAAAAAAT |
| | | ACAGAGAATGAAAAGAAACAGATAGATT |
| | | TTTTAGTTCTTTAGGCCCGTAGTCTGCAA |
| | | ATCCTTTTATGATTTTCTATCAAACAAAA |
| | | GAGGAAAATAGACCAGTTGCAATCCAAA |
| | | CGAGAGTCTAATAGAATGAGGTCGAAAA |
| | | GTAAATCGCGCGGGTTTGTTACTGATAAA |
| | | GCAGGCAAGACCTAAAATGTGTAAAGGG |
| | | CAAAGTGTATACTTTGGCGTCACCCCTTA |
| | | CATATTTTAGGTCTTTTTTTATTGTGCGTA |
| | | ACTAACTTGCCATCTTCAAACAGGAGGGC |
| | | TGGAAGAAGCAGACCGCTAACACAGTAC |
| | | ATAAAAAAGGAGACATGAACGATGAACA |
| | | TCAAAAAGTTTGCAAAACAAGCAACAGT |
| | | ATTAACCTTTACTACCGCACTGCTGGCAG |
| | | GAGGCGCAACTCAAGCGTTTGCGAAAGA |
| | | AACGAACCAAAAGCCATATAAGGAAACA |
| | | TACGGCATTTCCCATATTACACGCCATGA |
| | | TATGCTGCAAATCCCTGAACAGCAAAAA |
| | | AATGAAAAATATCAAGTTTCTGAATTTGA |
| | | TTCGTCCACAATTAAAAATATCTCTTCTG |
| | | CAAAAGGCCTGGACGTTTGGGACAGCTG |
| | | GCCATTACAAAACGCTGACGGCACTGTC |
| | | GCAAACTATCACGGCTACCACATCGTCTT |
| | | TGCATTAGCCGGAGATCCTAAAAATGCG |
| | | GATGACACATCGATTTACATGTTCTATCA |
| | | AAAAGTCGGCGAAACTTCTATTGACAGCT |
| | | GGAAAAACGCTGGCCGCGTCTTTAAAGA |
| | | CAGCGACAAATTCGATGCAAATGATTCTA |
| | | TCCTAAAAGACCAAACACAAGAATGGTC |
| | | AGGTTCAGCCACATTTACATCTGACGGAA |
| | | AAATCCGTTTATTCTACACTGATTTCTCC |
| | | GGTAAACATTACGGCAAACAAACACTGA |
| | | CAACTGCACAAGTTAACGTATCAGCATCA |
| | | GACAGCTCTTTGAACATCAACGGTGTAGA |
| | | GGATTATAAATCAATCTTTGACGGTGACG |
| | | GAAAAACGTATCAAAATGTACAGCAGTT |
| | | CATCGATGAAGGCAACTACAGCTCAGGC |
| | | GACAACCATACGCTGAGAGATCCTCACT |
| | | ACGTAGAAGATAAAGGCCACAAATACTT |
| | | AGTATTTGAAGCAAACACTGGAACTGAA |
| | | GATGGCTACCAAGGCGAAGAATCTTTATT |
| | | TAACAAAGCATACTATGGCAAAAGCACA |
| | | TCATTCTTCCGTCAAGAAAGTCAAAAACT |
| | | TCTGCAAAGCGATAAAAAACGCACGGCT |
| | | GAGTTAGCAAACGGCGCTCTCGGTATGAT |
| | | TGAGCTAAACGATGATTACACACTGAAA |
| | | AAAGTGATGAAACCGCTGATTGCATCTA |
| | | ACACAGTAACAGATGAAATTGAACGCGC |
| | | GAACGTCTTTAAAATGAACGGCAAATGG |
| | | TACCTGTTCACTGACTCCCGCGGATCAAA |
| | | AATGACGATTGACGGCATTACGTCTAACG |
| | | ATATTTACATGCTTGGTTATGTTTCTAATT |
| | | CTTTAACTGGCCCATACAAGCCGCTGAAC |
| | | AAAACTGGCCTTGTGTTAAAAATGGATCT |
| | | TGATCCTAACGATGTAACCTTTACTTACT |
| | | CACACTTCGCTGTACCTCAAGCGAAAGG |
| | | AAACAATGTCGTGATTACAAGCTATATGA |
| | | CAAACAGAGGATTCTACGCAGACAAACA |
| | | ATCAACGTTTGCGCCGAGCTTCCTGCTGA |
| | | ACATCAAAGGCAAGAAAACATCTGTTGT |
| | | CAAAGACAGCATCCTTGAACAAGGACAA |
| | | TTAACAGTTAACAAATAAAAACGCAAAA |
| | | GAAAATGACGTCCGGTATTACCCGGCAT |
| | | GACAGGAGTAAAAATGGCTATCGACGAA |
| | | AACAAACAGAAAGCGTTGGCGGCAGCAC |
| | | TGGGCCAGATTGAGAAACAATTTGGTAA |
| | | AGGCTCCATCATGCGCCTGGGTGAAGAC |
| | | CGTTCCATGGATGTGGAAACCATCTCTAC |
| | | CGGTTCGCTTTCACTGGATATCGCGCTTG |
| | | GGGCAGGTGGTCTGCCGATGGGCCGTAT |
| | | CGTCGAAATCTACGGACCGGAATCTTCCG |
| | | GTAAAACCACGCTGACGCTGCAGGTGAT |
| | | CGCCGCAGCGCAGCGTGAAGGTAAAACC |
| | | TGTGCGTTTATCGATGCTGAACACGCGCT |
| | | GGACCCAATCTACGCACGTAAACTGGGC |
| | | GTCGATATCGATAACCTGCTGTGCTCCCA |
| | | GCCGGACACCGGCGAGCAGGCACTGGAA |
| | | ATCTGTGACGCCCTGGCGCGTTCTGGCGC |
| | | AGTAGACGTTATCGTCGTTGACTCCGTGG |
| | | CGGCACTGACGCCGAAAGCGGAAATCGA |
| | | AGGCGAAATCGGCGACTCTCACATGGGC |
| | | CTTGCGGCACGTATGATGAGCCAGGCGA |
| | | TGCGTAAGCTGGCGGGTAACCTGAAGCA |
| | | GTCCAACACGCTGCTGATCTTCATCAACC |
| | | AGATCCGTATGAAAATTGGTGTGATGTTC |
| | | GGTAACCCGGAAACCACTACCGGTGGTA |
| | | ACGCGCTGAAATTCTACGCCTCTGTTCGT |
| | | CTCGACATCCGTCGTATCGGCGCGGTGAA |
| | | AGAGGGCGAAAACGTGGTGGGTAGCGAA |
| | | ACCCGCGTGAAAGTGGTGAAGAACAAAA |
| | | TCGCTGCGCCGTTTAAACAGGCTGAATTC |
| | | CAGATCCTCTACGGCGAAGGTATCAACTT |
| | | CTACGGCGAACTGGTTGACCTGGGCGTA |
| | | AAAGAGAAGCTGATCGAGAAAGCAGGCG |
| | | CGTGGTACAGCTACAAAGGTGAGAAGAT |
| | | CGGTCAGGGTAAAGCGAATGCGACTGCC |
| | | TGGCTGAAAGATAACCCGGAAACCGCGA |
| | | AAGAGATCGAGAAGAAAGTACGTGAGTT |
| | | GCTGCTGAGCAACCCGAACTCAACGCCG |
| | | GATTTCTCTGTAGATGATAGCGAAGGCGT |
| | | AGCAGAAACTAACGAAGATTTTTAATCGT |
| | | CTTGTTTGATACATAAGGGCCCGATGGAG |
| | | CAATGGCGATGACGCATCCTCACGATAAT |
| | | ATCCGGGTAGGCGCAATCACTTTCGTCTA |
| | | CTCCGTTACAAAGCGAGGCTGGGTATTTC |
| | | CCGGCCTTTCTGTTATCCGAAATCCACTG |
| | | AAAGCACAGCGGCTGGCTGAGGAGATAA |
| | | ATAATAAACGAGGGGCTGTATGCACAAA |
| | | GCATCTTCTGTTGAGTTAAGAACGAGTAT |
| | | CGAGATGGCACATAGCCTTGCTCAAATTG |
| | | GAATCAGGTTTGTGCCAATACCAGTAGA |
| | | AACAGACGAAGAATCCATGGGTATGGAC |
| | | AGTTTTCCCTTTGATATGTAACGGTGAAC |
| | | AGTTGTTCTACTTTTGTTTGTTAGTCTTGA |
| | | TGCTTCACTGATAGATACAAGAGCCATAA |
| | | GAACCTCAGATCCTTCCGTATTTAGCCAG |
| | | TATGTTCTCTAGTGTGGTTCGTTGTTTTTG |
| | | CGTGAGCCATGAGAACGAACCATTGAGA |
| | | TCATACTTACTTTGCATGTCACTCAAAAA |
| | | TTTTGCCTCAAAACTGGTGAGCTGAATTT |
| | | TTGCAGTTAAAGCATCGTGTAGTGTTTTT |
| | | CTTAGTCCGTTATGTAGGTAGGAATCTGA |
| | | TGTAATGGTTGTTGGTATTTTGTCACCATT |
| | | CATTTTTATCTGGTTGTTCTCAAGTTCGGT |
| | | TACGAGATCCATTTGTCTATCTAGTTCAA |
| | | CTTGGAAAATCAACGTATCAGTCGGGCG |
| | | GCCTCGCTTATCAACCACCAATTTCATAT |
| | | TGCTGTAAGTGTTTAAATCTTTACTTATTG |
| | | GTTTCAAAACCCATTGGTTAAGCCTTTTA |
| | | AACTCATGGTAGTTATTTTCAAGCATTAA |
| | | CATGAACTTAAATTCATCAAGGCTAATCT |
| | | CTATATTTGCCTTGTGAGTTTTCTTTTGTG |
| | | TTAGTTCTTTTAATAACCACTCATAAATC |
| | | CTCATAGAGTATTTGTTTTCAAAAGACTT |
| | | AACATGTTCCAGATTATATTTTATGAATT |
| | | TTTTTAACTGGAAAAGATAAGGCAATATC |
| | | TCTTCACTAAAAACTAATTCTAATTTTTC |
| | | GCTTGAGAACTTGGCATAGTTTGTCCACT |
| | | GGAAAATCTCAAAGCCTTTAACCAAAGG |
| | | ATTCCTGATTTCCACAGTTCTCGTCATCA |
| | | GCTCTCTGGTTGCTTTAGCTAATACACCA |
| | | TAAGCATTTTCCCTACTGATGTTCATCAT |
| | | CTGAACGTATTGGTTATAAGTGAACGATA |
| | | CCGTCCGTTCTTTCCTTGTAGGGTTTTCAA |
| | | TCGTGGGGTTGAGTAGTGCCACACAGCAT |
| | | AAAATTAGCTTGGTTTCATGCTCCGTTAA |
| | | GTCATAGCGACTAATCGCTAGTTCATTTG |
| | | CTTTGAAAACAACTAATTCAGACATACAT |
| | | CTCAATTGGTCTAGGTGATTTTAATCACT |
| | | ATACCAATTGAGATGGGCTAGTCAATGAT |
| | | AATTACTAGTCCTTTTCCTTTGAGTTGTGG |
| | | GTATCTGTAAATTCTGCTAGACCTTTGCT |
| | | GGAAAACTTGTAAATTCTGCTAGACCCTC |
| | | TGTAAATTCCGCTAGACCTTTGTGTGTTTT |
| | | TTTTGTTTATATTCAAGTGGTTATAATTTA |
| | | TAGAATAAAGAAAGAATAAAAAAAGATA |
| | | AAAAGAATAGATCCCAGCCCTGTGTATA |
| | | ACTCACTACTTTAGTCAGTTCCGCAGTAT |
| | | TACAAAAGGATGTCGCAAACGCTGTTTGC |
| | | TCCTCTACAAAACAGACCTTAAAACCCTA |
| | | AAGGCTTAAGTAGCACCCTCGCAAGCTC |
| | | GGTTGCGGCCGCAATCGGGCAAATCGCT |
| | | GAATATTCCTTTTGTCTCCGACCATCAGG |
| | | CACCTGAGTCGCTGTCTTTTTCGTGACAT |
| | | TCAGTTCGCTGCGCTCACGGCTCTGGCAG |
| | | TGAATGGGGGTAAATGGCACTACAGGCG |
| | | CCTTTTATGGATTCATGCAAGGAAACTAC |
| | | CCATAATACAAGAAAAGCCCGTCACGGG |
| | | CTTCTCAGGGCGTTTTATGGCGGGTCTGC |
| | | TATGTGGTGCTATCTGACTTTTTGCTGTTC |
| | | AGCAGTTCCTGCCCTCTGATTTTCCAGTC |
| | | TGACCACTTCGGATTATCCCGTGACAGGT |
| | | CATTCAGACTGGCTAATGCACCCAGTAAG |
| | | GCAGCGGTATCATCAACGGGGTCTG |
| SEQ ID NO: 42 | APSE10835 | AATTCCGAAATTAATACGACTCACTATAG |
| | | GGAGACATGAGGATTACCCATGTGCGAT |
| | | CGCGCACGAGGTTTTTCTGTCTAGTGAGC |
| | | AGTGTCCAACCTCAAAAGACAACATGTG |
| | | TGACGACGATGTAGCGGCTCTTGTCGTAG |
| | | ACAATGGATCCGGTATGTGCAAAGCCGG |
| | | TTTCGCAGGAGATGACGCACCCCGTGCCG |
| | | TCTTCCCCTCGATCGTCGGTCGCCCAAGG |
| | | CATCAAGGAGTCATGGTCGGTATGGGAC |
| | | AAAAGGACTCATACGTAGGAGATGAAGC |
| | | CCAAAGCAAAAGAGGTATCCTCACCCTG |
| | | AAATACCCCATCGAACACGGTATCATCAC |
| | | CAACTGGGATGAGTTTAAACCCTCTAGCT |
| | | TTTACAAAGTACTGGTTCCCTTTCCAGCG |
| | | GGATGCTTTATCTAAACGCAATGAGAGA |
| | | GGTATTCCTCAGGCCACATCGCTTCCTAG |
| | | TTCCGCTGGGATCCATCGTTGGCGGCCGA |
| | | AGCCGCCATTCCATAGTGAGTTCTGGCGC |
| | | GCCTCATCCCAGTTGGTGATGATACCGTG |
| | | TTCGATGGGGTATTTCAGGGTGAGGATAC |
| | | CTCTTTTGCTTTGGGCTTCATCTCCTACGT |
| | | ATGAGTCCTTTTGTCCCATACCGACCATG |
| | | ACTCCTTGATGCCTTGGGCGACCGACGAT |
| | | CGAGGGGAAGACGGCACGGGGTGCGTCA |
| | | TCTCCTGCGAAACCGGCTTTGCACATACC |
| | | GGATCCATTGTCTACGACAAGAGCCGCTA |
| | | CATCGTCGTCACACATGTTGTCTTTTGAG |
| | | GTTGGACACTGCTCACTAGACAGAAAAA |
| | | CCTCGTGCCGGACCGAATACCCGGTCTGA |
| | | ACGAGGGCGGCCGCGGTACCCAAGAAGT |
| | | ACTTAGAGTTAATTAAGGAGTTCAAACAT |
| | | GAGGATCACCCATGTCGAAGCTCCCACA |
| | | CCCTAGCATAACCCCTTGGGGCCTCTAAA |
| | | CGGGTCTTGAGGGGTTTTTTGCTGAAAGG |
| | | AGGAACTATATCCAGATATCTCGATCCCG |
| | | CGAAATTAATACGACTCACTATAGGGAG |
| | | ACCACAACGGTTTCCCTCTAGATCACAAG |
| | | TTTGTACAAAAAAGCAGGCTAAGAAGGA |
| | | GATATACATATGGCGTCTAACTTTACCCA |
| | | ATTCGTTCTGGTTGATAACGGCGGTACGG |
| | | GTGACGTTACCGTAGCTCCGTCCAACTTC |
| | | GCCAACGGTGTTGCGGAATGGATTAGCTC |
| | | TAACAGCCGCTCTCAGGCCTACAAAGTCA |
| | | CGTGCTCCGTTCGTCAGTCTAGCGCGCAG |
| | | AATCGCAAATACACCATCAAAGTTGAAG |
| | | TACCGAAAGTCGCAACGCAGACCGTAGG |
| | | CGGCGTAGAACTCCCAGTTGCGGCCTGGC |
| | | GCTCTTACCTCAACATGGAACTGACTATT |
| | | CCGATTTTTGCGACGAACTCCGACTGCGA |
| | | ACTGATTGTTAAGGCAATGCAGGGCCTGC |
| | | TGAAAGACGGTAATCCGATCCCATCTGCA |
| | | ATCGCTGCTAACTCTGGCATTTACTAATA |
| | | AGCGGACGCGCTGCCACCGCTGAGCAAT |
| | | AACTAGCATAACCCCTTGGGGCCTCTAAA |
| | | CGGGTCTTGAGGGGTTTTTTGCTGAAAGG |
| | | AGGAACTATATCCGGTCGACTTTATAGCT |
| | | AGCTCAGCCCTTGGTACAATGCTAGCTAC |
| | | TAGAGTAAGATGAGGAGCGAAGGCATGA |
| | | AACCATATCAGCGCCAGTTTATTGAATTT |
| | | GCGCTTAGCAAGCAGGTGTTAAAGTTTGG |
| | | CGAGTTTACGCTGAAATCCGGGCGCAAA |
| | | AGCCCCTATTTCTTCAACGCCGGGCTGTT |
| | | TAATACCGGGCGCGATCTGGCACTGTTAG |
| | | GCCGTTTTTACGCTGAAGCGTTGGTGGAT |
| | | TCCGGCATTGAGTTCGATCTGCTGTTTGG |
| | | CCCTGCTTACAAAGGGATCCCGATTGCCA |
| | | CCACAACCGCTGTGGCACTGGCGGAGCA |
| | | TCACGACCTGGACCTGCCGTACTGCTTTA |
| | | ACCGCAAAGAAGCAAAAGACCACGGTGA |
| | | AGGCGGCAATCTGGTTGGTAGCGCGTTAC |
| | | AAGGACGCGTAATGCTGGTAGATGATGT |
| | | GATCACCGCCGGAACGGCGATTCGCGAG |
| | | TCGATGGAGATTATTCAGGCCAATGGCGC |
| | | GACGCTTGCTGGCGTGTTGATTTCGCTCG |
| | | ATCGTCAGGAACGCGGGCGCGGCGAGAT |
| | | TTCGGCGATTCAGGAAGTTGAGCGTGATT |
| | | ACAACTGCAAAGTGATCTCTATCATCACC |
| | | CTGAAAGACCTGATTGCTTACCTGGAAGA |
| | | GAAGCCGGAAATGGCGGAACATCTGGCG |
| | | GCGGTTAAGGCCTATCGCGAAGAGTTTG |
| | | GCGTTTAATCCGGACCTAGGCAGAACGC |
| | | AGAAGCTGTCTGATAAAACAGAATTTGC |
| | | CTGGCGGCAGTAGCGCGGTGGTCCCACCT |
| | | GACCCCATGCCGAACTCAGAAGTGAAAC |
| | | GCCGTAGCGCCGATGGTAGTGTGGGGTCT |
| | | CCCCATGCGAGAGTAGGGAACTGCCAGG |
| | | CATCAAATAAAACGAAAGGCTCAGTCGA |
| | | AAGACTGGGCCTTTCGTTTTATCTGTTGTT |
| | | TGTCGGTGAACGCTCTCCTGAGTAGGACA |
| | | AATCCGCCGGGAGCGGATTTGAACGTTG |
| | | CGAAGCAACGGCCCGGAGGGTGGCGGGC |
| | | AGGACGCCCGCCATAAACTGCCAGGCAT |
| | | CAAATTAAGCAGAAGGCCATCCTGACGG |
| | | ATGGCCTTTTTGCGTTTCTACTCGCGAATT |
| | | ACGACGCGAGGCTGGATGGCCTTCCCCAT |
| | | TATGATTCTTCTCGCTTCCGGCGGCATCG |
| | | GGATGCCCGCGTTGCAGGCCATGCTGTCC |
| | | AGGCAGGTAGATGACGACCATCAGGGAC |
| | | AGCTTCAAGGATCGCTCGCGGCTCTTACC |
| | | AGCCTAACTTCGATCATTGGACCGCTGAT |
| | | CGTCACGGCGATTTATGCCGCCTCGGCGA |
| | | GCACATGGAACGGGTTGGCATGGATTGT |
| | | AGGCGCCGCCCTATACCTTGTCTGCCTCC |
| | | CCGCGTTGCGTCGCGGTGCATGGAGCCG |
| | | GGCCACCTCGACCTGAATGGAAGCCGGC |
| | | GGCACCTCGCTAACGGATTCACCACTCCA |
| | | AGAATTGGAGCCAATCAATTCTTGCGGA |
| | | GAACTGTGAATGCGCAAACCAACCCTTG |
| | | GCAGAACATATCCATCGCGTCCGCCATCT |
| | | CCAGCAGCCGCACGCGGCGCATCTCGGG |
| | | CAGCGTTGGGTCCTGGCCACGGGTGCGC |
| | | ATGATCGTGCTCCTGTCGTTGAGGACCCG |
| | | GCTAGGCTGGCGGGGTTGCCTTACTGGTT |
| | | AGCAGAATGAATCACCGATACGCGAGCG |
| | | AACGTGAAGCGACTGCTGCTGCAAAACG |
| | | TCTGCGACCTGAGCAACAACATGAATGG |
| | | TCTTCGGTTTCCGTGTTTCGTAAAGTCTG |
| | | GAAACGCGGAAGTCAGCGCCCTGCACCA |
| | | TTATGTTCCGGATCTGCATCGCAGGATGC |
| | | TGCTGGCTACCCTGTGGAACACCTACATC |
| | | TGTATTAACGAAGCGCTGGCATTGACCCT |
| | | GAGTGATTTTTCTCTGGTCCCGCCGCATC |
| | | CATACCGCCAGTTGTTTACCCTCACAACG |
| | | TTCCAGTAACCGGGCATGTTCATCATCAG |
| | | TAACCCGTATCGTGAGCATCCTCTCTCGT |
| | | TTCATCGGTATCATTACCCCCATGAACAG |
| | | AAATCCCCCTTACACGGAGGCATCAGTG |
| | | ACCAAACAGGAAAAAACCGCCCTTAACA |
| | | TGGCCCGCTTTATCAGAAGCCAGACATTA |
| | | ACGCTTCTGGAGAAACTCAACGAGCTGG |
| | | ACGCGGATGAACAGGCAGACATCTGTGA |
| | | ATCGCTTCACGACCACGCTGATGAGCTTT |
| | | ACCGCAGCTGCCTCGCGCGTTTCGGTGAT |
| | | GACGGTGAAAACCTCTGACACATGCAGC |
| | | TCCCGGAGACGGTCACAGCTTGTCTGTAA |
| | | GCGGATGCCGGGAGCAGACAAGCCCGTC |
| | | AGGGCGCGTCAGCGGGTGTTGGCGGGTG |
| | | TCGGGGCGCAGCCATGACCCAGTCACGT |
| | | AGCGATAGCGGAGTGTATACTGGCTTAA |
| | | CTATGCGGCATCAGAGCAGATTGTACTGA |
| | | GAGTGCACCATATGCGGTGTGAAATACC |
| | | GCACAGATGCGTAAGGAGAAAATACCGC |
| | | ATCAGGCGCTCTTCCGCTTCCTCGCTCAC |
| | | TGACTCGCTGCGCTCGGTCGTTCGGCTGC |
| | | GGCGAGCGGTATCAGCTCACTCAAAGGC |
| | | GGTAATACGGTTATCCACAGAATCAGGG |
| | | GATAACGCAGGAAAGAACATGTGAGCAA |
| | | AAGGCCAGCAAAAGGCCAGGAACCGTAA |
| | | AAAGGCCGCGTTGCTGGCGTTTTTCCATA |
| | | GGCTCCGCCCCCCTGACGAGCATCACAA |
| | | AAATCGACGCTCAAGTCAGAGGTGGCGA |
| | | AACCCGACAGGACTATAAAGATACCAGG |
| | | CGTTTCCCCCTGGAAGCTCCCTCGTGCGC |
| | | TCTCCTGTTCCGACCCTGCCGCTTACCGG |
| | | ATACCTGTCCGCCTTTCTCCCTTCGGGAA |
| | | GCGTGGCGCTTTCTCATAGCTCACGCTGT |
| | | AGGTATCTCAGTTCGGTGTAGGTCGTTCG |
| | | CTCCAAGCTGGGCTGTGTGCACGAACCCC |
| | | CCGTTCAGCCCGACCGCTGCGCCTTATCC |
| | | GGTAACTATCGTCTTGAGTCCAACCCGGT |
| | | AAGACACGACTTATCGCCACTGGCAGCA |
| | | GCCACTGGTAACAGGATTAGCAGAGCGA |
| | | GGTATGTAGGCGGTGCTACAGAGTTCTTG |
| | | AAGTGGTGGCCTAACTACGGCTACACTA |
| | | GAAGGACAGTATTTGGTATCTGCGCTCTG |
| | | CTGAAGCCAGTTACCTTCGGAAAAAGAG |
| | | TTGGTAGCTCTTGATCCGGCAAACAAACC |
| | | ACCGCTGGTAGCGGTGGTTTTTTTGTTTG |
| | | CAAGCAGCAGATTACGCGCAGAAAAAAA |
| | | GGATCTCAAGAAGATCCTTTGATCTTTTC |
| | | TACGGGGTCTGACGCTCAGTGGAACGAA |
| | | AACTCACGTTAAGGGATTTTGGTCATGAG |
| | | ATTATCAAAAAGGATCTTCACCTAGATCC |
| | | TTTTAAATTAAAAATGAAGTTTTAAATCA |
| | | ATCTAAAGTATATATGAGTAAACTTGGTC |
| | | TGACAGTTACCAATGCTTAATCAGTGAGG |
| | | CACCTATCTCAGCGATCTGTCTATTTCGTT |
| | | CATCCATAGTTGCCTGACTCCCCGTCGTG |
| | | TAGATAACTACGATACGGGAGGGCTTAC |
| | | CATCTGGCCCCAGTGCTGCAATGATACCG |
| | | CGAGACCCACGCTCACCGGCTCCAGATTT |
| | | ATCAGCAATAAACCAGCCAGCCGGAAGG |
| | | GCCGAGCGCAGAAGTGGTCCTGCAACTTT |
| | | ATCCGCCTCCATCCAGTCTATTAATTGTT |
| | | GCCGGGAAGCTAGAGTAAGTAGTTCGCC |
| | | AGTTAATAGTTTGCGCAACGTTGTTGCCA |
| | | TTGCTGCAGGCATCGTGGTGTCACGCTCG |
| | | TCGTTTGGTATGGCTTCATTCAGCTCCGG |
| | | TTCCCAACGATCAAGGCGAGTTACATGAT |
| | | CCCCCATGTTGTGCAAAAAAGCGGTTAGC |
| | | TCCTTCGGTCCTCCGATCGTTGTCAGAAG |
| | | TAAGTTGGCCGCAGTGTTATCACTCATGG |
| | | TTATGGCAGCACTGCATAATTCTCTTACT |
| | | GTCATGCCATCCGTAAGATGCTTTTCTGT |
| | | GACTGGTGAGTACTCAACCAAGTCATTCT |
| | | GAGAATAGTGTATGCGGCGACCGAGTTG |
| | | CTCTTGCCCGGCGTCAACACGGGATAATA |
| | | CCGCGCCACATAGCAGAACTTTAAAAGT |
| | | GCTCATCATTGGAAAACGTTCTTCGGGGC |
| | | GAAAACTCTCAAGGATCTTACCGCTGTTG |
| | | AGATCCAGTTCGATGTAACCCACTCGTGC |
| | | ACCCAACTGATCTTCAGCATCTTTTACTTT |
| | | CACCAGCGTTTCTGGGTGAGCAAAAACA |
| | | GGAAGGCAAAATGCCGCAAAAAAGGGA |
| | | ATAAGGGCGACACGGAAATGTTGAATAC |
| | | TCATACTCTTCCTTTTTCAATATTATTGAA |
| | | GCATTTATCAGGGTTATTGTCTCATGAGC |
| | | GGATACATATTTGAATGTATTTAGAAAAA |
| | | TAAACAAATAGGGGTTCCGCGCACATTTC |
| | | CCCGAAAAGTGCCACCTGACGTCTAAGA |
| | | AACCATTATTATCATGACATTAACCTATA |
| | | AAAATAGGCGTATCACGAGGCCCTTTCGT |
| | | CTTCAAG |
| SEQ ID NO: 43 | APSE10836 | AATtCCGAAATTAATACGACTCACTATAG |
| | | GGAGACATGAGGATTACCCATGTGCGAT |
| | | CGCGCACGAGGTTTTTCTGTCTAGTGAGC |
| | | AGTGTCCAACCTCAAAAGACAACATGTG |
| | | TGACGACGATGTAGCGGCTCTTGTCGTAG |
| | | ACAATGGATCCGGTATGTGCAAAGCCGG |
| | | TTTCGCAGGAGATGACGCACCCCGTGCCG |
| | | TCTTCCCCTCGATCGTCGGTCGCCCAAGG |
| | | CATCAAGGAGTCATGGTCGGTATGGGAC |
| | | AAAAGGACTCATACGTAGGAGATGAAGC |
| | | CCAAAGCAAAAGAGGTATCCTCACCCTG |
| | | AAATACCCCATCGAACACGGTATCATCAC |
| | | CAACTGGGATGAGTTTAAACCCTCTAGCT |
| | | TTTACAAAGTACTGGTTCCCTTTCCAGCG |
| | | GGATGCTTTATCTAAACGCAATGAGAGA |
| | | GGTATTCCTCAGGCCACATCGCTTCCTAG |
| | | TTCCGCTGGGATCCATCGTTGGCGGCCGA |
| | | AGCCGCCATTCCATAGTGAGTTCTGGCGC |
| | | GCCTCATCCCAGTTGGTGATGATACCGTG |
| | | TTCGATGGGGTATTTCAGGGTGAGGATAC |
| | | CTCTTTTGCTTTGGGCTTCATCTCCTACGT |
| | | ATGAGTCCTTTTGTCCCATACCGACCATG |
| | | ACTCCTTGATGCCTTGGGCGACCGACGAT |
| | | CGAGGGGAAGACGGCACGGGGTGCGTCA |
| | | TCTCCTGCGAAACCGGCTTTGCACATACC |
| | | GGATCCATTGTCTACGACAAGAGCCGCTA |
| | | CATCGTCGTCACACATGTTGTCTTTTGAG |
| | | GTTGGACACTGCTCACTAGACAGAAAAA |
| | | CCTCGTGCCGGACCGAATACCCGGTCTGA |
| | | ACGAGGGCGGCCGCGGTACCCAAGAAGT |
| | | ACTTAGAGTTAATTAAGGAGTTCAAACAT |
| | | GAGGATCACCCATGTCGAAGCTCCCACA |
| | | CCCTAGCATAACCCCTTGGGGCCTCTAAA |
| | | CGGGTCTTGAGGGGTTTTTTGCTGAAAGG |
| | | AGGAACTATATCCaGATATCTCGATCCCG |
| | | CGAAATTAATACGACTCACTATAGGGAG |
| | | ACCACAACGGTTTCCCTCTAGATCACAAG |
| | | TTTGTACAAAAAAGCAGGCTAAGAAGGA |
| | | GATATACATATGGCGTCTAACTTTACCCA |
| | | ATTCGTTCTGGTTGATAACGGCGGTACGG |
| | | GTGACGTTACCGTAGCTCCGTCCAACTTC |
| | | GCCAACGGTGTTGCGGAATGGATTAGCTC |
| | | TAACAGCCGCTCTCAGGCCTACAAAGTCA |
| | | CGTGCTCCGTTCGTCAGTCTAGCGCGCAG |
| | | AATCGCAAATACACCATCAAAGTTGAAG |
| | | TACCGAAAGTCGCAACGCAGACCGTAGG |
| | | CGGCGTAGAACTCCCAGTTGCGGCCTGGC |
| | | GCTCTTACCTCAACATGGAACTGACTATT |
| | | CCGATTTTTGCGACGAACTCCGACTGCGA |
| | | ACTGATTGTTAAGGCAATGCAGGGCCTGC |
| | | TGAAAGACGGTAATCCGATCCCATCTGCA |
| | | ATCGCTGCTAACTCTGGCATTTACTAATA |
| | | AGCGGACGCGCTGCCACCGCTGAGCAAT |
| | | AACTAGCATAACCCCTTGGGGCCTCTAAA |
| | | CGGGTCTTGAGGGGTTTTTTGCTGAAAGG |
| | | AGGAACTATATCCGGtcgaCTTTATAGCTA |
| | | GCTCAGCCCTTGGTACAATGCTAGCtactaga |
| | | GTAAGATGAGGAGCGAAGGCATGAAACC |
| | | ATATCAGCGCCAGTTTATTGaatttgcgcttagca |
| | | agcaggtgttaaagtttggcgagtttacgctgaaatccgggcgcaa |
| | | aagcccctatttcttcaacgccgggctgtttaataccgggcgcgatct |
| | | ggcactgttaggccgtttttacgctgaagcgttggtggattccggcat |
| | | tgagttcgatctgctgtttggccctgcttacaaagggatcccgattgc |
| | | caccacaaccgctgtggcactggcggagcatcacgacctggacct |
| | | gccgtactgctttaaccgcaaagaagcaaaagaccacggtgaagg |
| | | cggcaatctggttggtagcgcgttacaaggacgcgtaatgctggta |
| | | gatgatgtgatcaccgccggaacggcgattcgcgagtcgatggag |
| | | attattcaggccaatggcgcgacgcttgctggcgtgttgatttcgctc |
| | | gatcgtcaggaacgcgggcgcggcgagatttcggcgattcaggaa |
| | | gttgagcgtgattacaactgcaaagtgatctctatcatcaccctgaaa |
| | | gacctgattgcttacctggaagagaagccggaaatggcggaacat |
| | | ctggcggcggttaaggcctatcGCGAAGAGTTTGGCG |
| | | TTTAATCCGGAcctaggCAGAACGCAGAAG |
| | | CtGTCTGATAAAACAGAATTTGCCTGGCG |
| | | GCAGTAGCGCGGTGGTCCCACCTGACCCC |
| | | ATGCCGAACTCAGAAGTGAAACGCCGTA |
| | | GCGCCGATGGTAGTGTGGGGTCTCCCCAT |
| | | GCGAGAGTAGGGAACTGCCAGGCATCAA |
| | | ATAAAACGAAAGGCTCAGTCGAAAGACT |
| | | GGGCCTTTCGTTTTATCTGTTGTTTGTCGG |
| | | TGAACGCTCTCCTGAGTAGGACAAATCCG |
| | | CCGGGAGCGGATTTGAACGTTGCGAAGC |
| | | AACGGCCCGGAGGGTGGCGGGCAGGACG |
| | | CCCGCCATAAACTGCCAGGCATCAAATTA |
| | | AGCAGAAGGCCATCCTGACGGATGGCCT |
| | | TTTTGCGTTTCTACtcgCGAATTAcgaCGCG |
| | | AGGCTGGATGGCCTTCCCCATTATGATTC |
| | | TTCTCGCTTCCGGCGGCATCGGGATGCCC |
| | | GCGTTGCAGGCCATGCTGTCCAGGCAGGT |
| | | AGATGACGACCATCAGGGACAGCTTCAA |
| | | GGATCGCTCGCGGCTCTTACCAGCCTAAC |
| | | TTCGATCATTGGACCGCTGATCGTCACGG |
| | | CGATTTATGCCGCCTCGGCGAGCACATGG |
| | | AACGGGTTGGCATGGATTGTAGGCGCCG |
| | | CCCTATACCTTGTCTGCCTCCCCGCGTTG |
| | | CGTCGCGGTGCATGGAGCCGGGCCACCT |
| | | CGACCTGAATGGAAGCCGGCGGCACCTC |
| | | GCTAACGGATTCACCACTCCAAGAATTGG |
| | | AGCCAATCAATTCTTGCGGAGAACTGTGA |
| | | ATGCGCAAACCAACCCTTGGCAGAACAT |
| | | ATCCATCGCGTCCGCCATCTCCAGCAGCC |
| | | GCACGCGGCGCATCTCGGGCAGCGTTGG |
| | | GTCCTGGCCACGGGTGCGCATGATCGTGC |
| | | TCCTGTCGTTGAGGACCCGGCTAGGCTGG |
| | | CGGGGTTGCCTTACTGGTTAGCAGAATGA |
| | | ATCACCGATACGCGAGCGAACGTGAAGC |
| | | GACTGCTGCTGCAAAACGTCTGCGACCTG |
| | | AGCAACAACATGAATGGTCTTCGGTTTCC |
| | | GTGTTTCGTAAAGTCTGGAAACGCGGAA |
| | | GTCAGCGCCCTGCACCATTATGTTCCGGA |
| | | TCTGCATCGCAGGATGCTGCTGGCTACCC |
| | | TGTGGAACACCTACATCTGTATTAACGAA |
| | | GCGCTGGCATTGACCCTGAGTGATTTTTC |
| | | TCTGGTCCCGCCGCATCCATACCGCCAGT |
| | | TGTTTACCCTCACAACGTTCCAGTAACCG |
| | | GGCATGTTCATCATCAGTAACCCGTATCG |
| | | TGAGCATCCTCTCTCGTTTCATCGGTATC |
| | | ATTACCCCCATGAACAGAAATCCCCCTTA |
| | | CACGGAGGCATCAGTGACCAAACAGGAA |
| | | AAAACCGCCCTTAACATGGCCCGCTTTAT |
| | | CAGAAGCCAGACATTAACGCTTCTGGAG |
| | | AAACTCAACGAGCTGGACGCGGATGAAC |
| | | AGGCAGACATCTGTGAATCGCTTCACGAC |
| | | CACGCTGATGAGCTTTACCGCAGCTGCCT |
| | | CGCGCGTTTCGGTGATGACGGTGAAAAC |
| | | CTCTGACACATGCAGCTCCCGGAGACGGT |
| | | CACAGCTTGTCTGTAAGCGGATGCCGGG |
| | | AGCAGACAAGCCCGTCAGGGCGCGTCAG |
| | | CGGGTGTTGGCGGGTGTCGGGGCGCAGC |
| | | CATGACCCAGTCACGTAGCGATAGCGGA |
| | | GTGTATACTGGCTTAACTATGCGGCATCA |
| | | GAGCAGATTGTACTGAGAGTGCACCATA |
| | | TGCGGTGTGAAATACCGCACAGATGCGT |
| | | AAGGAGAAAATACCGCATCAGGCGCTCT |
| | | TCCGCTTCCTCGCTCACTGACTCGCTGCG |
| | | CTCGGTCGTTCGGCTGCGGCGAGCGGTAT |
| | | CAGCTCACTCAAAGGCGGTAATACGGTT |
| | | ATCCACAGAATCAGGGGATAACGCAGGA |
| | | AAGAACATGTGAGCAAAAGGCCAGCAAA |
| | | AGGCCAGGAACCGTAAAAAGGCCGCGTT |
| | | GCTGGCGTTTTTCCATAGGCTCCGCCCCC |
| | | CTGACGAGCATCACAAAAATCGACGCTC |
| | | AAGTCAGAGGTGGCGAAACCCGACAGGA |
| | | CTATAAAGATACCAGGCGTTTCCCCCTGG |
| | | AAGCTCCCTCGTGCGCTCTCCTGTTCCGA |
| | | CCCTGCCGCTTACCGGATACCTGTCCGCC |
| | | TTTCTCCCTTCGGGAAGCGTGGCGCTTTC |
| | | TCATAGCTCACGCTGTAGGTATCTCAGTT |
| | | CGGTGTAGGTCGTTCGCTCCAAGCTGGGC |
| | | TGTGTGCACGAACCCCCCGTTCAGCCCGA |
| | | CCGCTGCGCCTTATCCGGTAACTATCGTC |
| | | TTGAGTCCAACCCGGTAAGACACGACTTA |
| | | TCGCCACTGGCAGCAGCCACTGGTAACA |
| | | GGATTAGCAGAGCGAGGTATGTAGGCGG |
| | | TGCTACAGAGTTCTTGAAGTGGTGGCCTA |
| | | ACTACGGCTACACTAGAAGGACAGTATTT |
| | | GGTATCTGCGCTCTGCTGAAGCCAGTTAC |
| | | CTTCGGAAAAAGAGTTGGTAGCTCTTGAT |
| | | CCGGCAAACAAACCACCGCTGGTAGCGG |
| | | TGGTTTTTTTGTTTGCAAGCAGCAGATTA |
| | | CGCGCAGAAAAAAAGGATCTCAAGAAGA |
| | | TCCTTTGATCTTTTCTACGGGGTCTGACG |
| | | CTCAGTGGAACGAAAACTCACGTTAAGG |
| | | GATTTTGGTCATGACATTAACCTATAAAA |
| | | ATAGGCGTATCACGAGGCCCTTTCGTCTT |
| | | CAAG |
| SEQ ID NO: 44 | Genomic sequence of | CCCTTAGGAGTTGGCATGGCGAATATGTT |
| | HT115(DE3)-ΔpyrE | TGCCCTGATTCTGGTGATTGCCACACTGG |
| | surrounding mini- | TGACGGGCATTTTATGGTGCGTGGATAAA |
| | Tn10 insertion in the | TTCTTTTTCGCACCTAAACGGCGGGAACG |
| | rnc-era-recO operon | TCAGGCAGCGGCGCAGGCGGCTGCCGGG |
| | | GACTCACTGGATAAAGCAACGTTGAAAA |
| | | AGGTTGCGCCGAAGCCTGGCTGGCTGGA |
| | | AACCGGTGCTTCTGTTTTTCCGGTACTGG |
| | | CTATCGTATTGATTGTGCGTTCGTTTATTT |
| | | ATGAACCGTTCCAGATCCCGTCAGGTTCG |
| | | ATGATGCCGACTCTGTTAATTGGTGATTT |
| | | TATTCTGGTAGAGAAGTTTGCTTATGGCA |
| | | TTAAAGATCCTATCTACCAGAAAACGCTG |
| | | ATCGAAACCGGTCATCCGAAACGCGGCG |
| | | ATATCGTGGTCTTTAAATATCCGGAAGAT |
| | | CCAAAGCTTGATTACATCAAGCGCGCGGT |
| | | GGGTTTACCGGGCGATAAAGTCACTTACG |
| | | ATCCGGTCTCAAAAGAGCTGACGATTCA |
| | | ACCGGGATGCAGTTCCGGCCAGGCGTGT |
| | | GAAAACGCGCTGCCGGTCACCTACTCAA |
| | | ACGTGGAACCGAGCGATTTCGTTCAGACC |
| | | TTCTCACGCCGTAATGGTGGGGAAGCGA |
| | | CCAGCGGATTCTTTGAAGTGCCGAAAAA |
| | | CGAAACCAAAGAAAATGGAATTCGTCTT |
| | | TCCGAGCGTAAAGAGACACTGGGTGATG |
| | | TGACGCACCGCATTCTGACAGTGCCGATT |
| | | GCGCAGGATCAGGTGGGGATGTATTACC |
| | | AGCAGCCAGGGCAACAACTGGCAACCTG |
| | | GATTGTTCCTCCGGGACAATACTTCATGA |
| | | TGGGCGACAACCGCGACAACAGCGCGGA |
| | | CAGCCGTTACTGGGGCTTTGTGCCGGAAG |
| | | CGAATCTGGTCGGTCGGGCAACGGCTATC |
| | | TGGATGAGCTTCGATAAGCAAGAAGGCG |
| | | AATGGCCGACTGGTCTGCGCTTAAGTCGC |
| | | ATTGGCGGCATCCATTAATAGCCATCTTC |
| | | GTTCACGTTGTCGCCGTTATGGCGACAAC |
| | | GTGAATTATTTATGAGATAAATCTCCCGT |
| | | GGCTAACGACATCCCCCGTCGTTGTGTAT |
| | | AGAATATTCCCCCGAAGTTTAAGGTTGGC |
| | | ACCTCCAGGTTGCCACGGCACACGAAAC |
| | | AGCGTTGGTCCCCATATACCGGTAAACTG |
| | | AAACTGCAGCGAAGCAGTTAGCAGAACC |
| | | ATGTATATCAGGTCTGTTTCGTGTGCTGA |
| | | ATTGTTGACGCATTTATTTATTGGTATCG |
| | | CATGAACCCCATCGTAATTAATCGGCTTC |
| | | AACGGAAGCTGGGCTCTGATGAATCCCCT |
| | | AATGATTTTGGTAAAAATCATTAAGTTAA |
| | | GGTGGATACACATCTTGTCATATGATCTC |
| | | GGGAAAAGCGTTGGTGACCAAAGGTGCC |
| | | TTTTATCATCACTTTAAAAATAAAAAACA |
| | | ATTACTCAGTGCCTGTTATAAGCAGCAAT |
| | | TAATTATGATTGATGCCTACATCACAACA |
| | | AAAACTGATTTAACAAATGGTTGGTCTGC |
| | | CTTAGAAAGTATATTTGAACATTATCTTG |
| | | ATTATATTATTGATAATAATAAAAACCTT |
| | | ATCCCTATCCAAGAAGTGATGCCTATCAT |
| | | TGGTTGGAATGAACTTGAAAAAATTAGC |
| | | CTTGAATACATTACTGGTAAGGTAAACGC |
| | | CATTGTCAGCAAATTGATCCAAGAGAAC |
| | | CAACTTAAAGCTTATGATGATGATGTGCT |
| | | TAAAAACTTACTCAATGGCTGGTTTATGC |
| | | ATATCGCAATACATGCGAAAAACCTAAA |
| | | AGAGCTTGCCGATAAAAAAGGCCAATTT |
| | | ATTGCTATTTACCGCGGCTTTTTATTGAG |
| | | CTTGAAAGATAAATAAAATAGATAGGTT |
| | | TTATTTGAAGCTAAATCTTCTTTATCGTA |
| | | AAAAATGCCCTCTTGGGTTATCAAGAGG |
| | | GTCATTATATTTCGCGGAATAACATCATT |
| | | TGGTGACGAAATAACTAAGCACTTGTCTC |
| | | CTGTTTACTCCCCTGAGCTTGAGGGGTTA |
| | | ACATGAAGGTCATCGATAGCAGGATAAT |
| | | AATACAGTAAAACGCTAAACCAATAATC |
| | | CAAATCCAGCCATCCCAAATTGGTAGTGA |
| | | ATGATTATAAATAACAGCAAACAGTAAT |
| | | GGGCCAATAACACCGGTTGCATTGGTAA |
| | | GGCTCACCAATAATCCCTGTAAAGCACCT |
| | | TGCTGATGACTCTTTGTTTGGATAGACAT |
| | | CACTCCCTGTAATGCAGGTAAAGCGATCC |
| | | CACCACCAGCCAATAAAATTAAAACAGG |
| | | GAAAACTAACCAACCTTCAGATATAAAC |
| | | GCTAAAAAGGCAAATGCACTACTATCTG |
| | | CAATAAATCCGAGCAGTACTGCCGTTTTT |
| | | TCGCCCCATTTAGTGGCTATTCTTCCTGCC |
| | | ACAAAGGCTTGGAATACTGAGTGTAAAA |
| | | GACCAAGACCCGCTAATGAAAAGCCAAC |
| | | CATCATGCTATTCCATCCAAAACGATTTT |
| | | CGGTAAATAGCACCCACACCGTTGCGGG |
| | | AATTTGGCCTATCAATTGCGCTGAAAAAT |
| | | AAATAATCAACAAAATGGGCATCGTTTTA |
| | | AATAAAGTGATGTATACCGAATTCGATTG |
| | | CGTCTCAACCCCTACTTCGGTATCTGTAT |
| | | TATCACGTGTATTTTTGGTTTCACGGAAC |
| | | CAAAACATAACCACAAGGAAAGTGACAA |
| | | TATTTAGCAACGCAGCGATAAAAAAGGG |
| | | ACTATGCGGTGAAATCTCTCCTGCAAAAC |
| | | CACCAATAATAGGCCCCGCTATTAAACCA |
| | | AGCCCAAAACTTGCCCCTAACCAACCGA |
| | | ACCACTTCACGCGTTGAGAAGCTGAGGT |
| | | GGTATCGGCAATGACCGATGCCGCGACA |
| | | GCCCCAGTAGCTCCTGTGATCCCTGAAAG |
| | | CAAACGGCCTAAATACAGCATCCAAAGC |
| | | GCACTTGAAAAAGCCAGCAATAAGTAAT |
| | | CCAGCGATGCGCCTATTAATGACAACAA |
| | | CAGCACTGGGCGCCGACCAAATCGGTCA |
| | | GACATTTTTCCAAGCCAAGGAGCAAAGA |
| | | TAACCTGCATTAACGCATAAAGTGCAAG |
| | | CAATACGCCAAAGTGGTTAGCGATATCTT |
| | | CCGAAGCAATAAATTCACGTAATAACGTT |
| | | GGCAAGACTGGCATGATAAGGCCAATCC |
| | | CCATGGCATCGAGTAACGTAATTACCAAT |
| | | GCGATCTTTGTCGAACTATTCATTTCACTT |
| | | TTCTCTATCACTGATAGGGAGTGGTAAAA |
| | | TAACTCTATCAATGATAGAGTGTCAACAA |
| | | AAATTAGGAATTAATGATGTCTAGATTAG |
| | | ATAAAAGTAAAGTGATTAACAGCGCATT |
| | | AGAGCTGCTTAATGAGGTCGGAATCGAA |
| | | GGTTTAACAACCCGTAAACTCGCCCAGA |
| | | AGCTAGGTGTAGAGCAGCCTACATTGTAT |
| | | TGGCATGTAAAAAATAAGCGGGCTTTGCT |
| | | CGACGCCTTAGCCATTGAGATGTTAGATA |
| | | GGCACCATACTCACTTTTGCCCTTTAGAA |
| | | GGGGAAAGCTGGCAAGATTTTTTACGTA |
| | | ATAACGCTAAAAGTTTTAGATGTGCTTTA |
| | | CTAAGTCATCGCGATGGAGCAAAAGTAC |
| | | ATTTAGGTACACGGCCTACAGAAAAACA |
| | | GTATGAAACTCTCGAAAATCAATTAGCCT |
| | | TTTTATGCCAACAAGGTTTTTCACTAGAG |
| | | AATGCATTATATGCACTCAGCGCTGTGGG |
| | | GCATTTTACTTTAGGTTGCGTATTGGAAG |
| | | ATCAAGAGCATCAAGTCGCTAAAGAAGA |
| | | AAGGGAAACACCTACTACTGATAGTATG |
| | | CCGCCATTATTACGACAAGCTATCGAATT |
| | | ATTTGATCACCAAGGTGCAGAGCCAGCCT |
| | | TCTTATTCGGCCTTGAATTGATCATATGC |
| | | GGATTAGAAAAACAACTTAAATGTGAAA |
| | | GTGGGTCTTAAAAGCAGCATAACCTTTTT |
| | | CCGTGATGGTAACTTCACGGTAACCAAG |
| | | ATGTCGAGTTAACCACCCTTTAGATTCAT |
| | | AAAGCGAAAATAATGCGGCTCCAACGTA |
| | | CCCACCTAAATGGAAACGGCGTTCACTCC |
| | | AATCTAAACACGCACAACAGATTTTACGT |
| | | GAATGTTTGGAAGGAACGTCAATTCCCAT |
| | | TTCATGAAAATATTGAATACCACTTAATG |
| | | TGATCATTGAACCATTTTCAGTGATCCAT |
| | | TGCTGTTGACAAAGGGAATCATAGATCAT |
| | | ATGACAAGATGTGTATCTACCTTAACTTA |
| | | ATGATTTTGATAAAAATCATTAGGGGATT |
| | | CATCAGAGCTGGGCTACACTTTTAATCAT |
| | | CAGGAACTGTTGCAGCAGGCATTAACTC |
| | | ATCGTAGTGCCAGCAGTAAACATAACGA |
| | | GCGTTTAGAATTTTTAGGCGACTCTATTC |
| | | TGAGCTACGTTATCGCCAATGCGCTTTAT |
| | | CACCGTTTCCCTCGTGTGGATGAAGGCGA |
| | | TATGAGCCGGATGCGCGCCACGCTGGTCC |
| | | GTGGCAATACGCTGGCGGAACTGGCGCG |
| | | CGAATTTGAGTTAGGCGAGTGCTTACGTT |
| | | TAGGGCCAGGTGAACTTAAAAGCGGTGG |
| | | ATTTCGTCGTGAGTCAATTCTCGCCGACA |
| | | CCGTCGAAGCATTAATTGGTGGCGTATTC |
| | | CTCGACAGTGATATTCAAACCGTCGAGA |
| | | AATTAATCCTCAACTGGTATCAAACTCGT |
| | | TTGGACGAAATTAGCCCAGGCGATAAAC |
| | | AAAAAGATCCGAAAACGCGCTTGCAAGA |
| | | ATATTTGCAGGGTCGCCATCTGCCGCTGC |
| | | CGACTTATCTGGTAGTCCAGGTACGTGGC |
| | | GAAGCGCACGATCAGGAATTTACTATCC |
| | | ACTGCCAGGTCAGCGGCCTGAGTGAACC |
| | | GGTGGTTGGCACAGGTTCAAGCCGTCGTA |
| | | AGGCTGAGCAGGCTGCCGCCGAACAGGC |
| | | GTTGAAAAAACTGGAGCTGGAATGAGCA |
| | | TCGATAAAAGTTACTGCGGATTTATTGCC |
| | | ATCGTCGGACGTCCGAACGTTGGCAAATC |
| | | CACATTGTTGAACAAACTGCTGGGGCAG |
| | | AAAATCTCCATCACTTCCCGCAAGGCGCA |
| | | GACAACTCGTCACCGCATTGTGGGGATCC |
| | | ATACTGAAGGCGCGTATCAGGCGATCTA |
| | | CGTCGATACACCGGGCCTGCATATGGAA |
| | | GAAAAACGCGCCATTAACCGCCTGATGA |
| | | ACAAAGCGGCGAGCAGCTCTATTGGCGA |
| | | TGTTGAGCTGGTGATTTTTGTCGTTGAAG |
| | | GCACCCGCTGGACGCCGGACGACGAAAT |
| | | GGTGCTCAACAAACTGCGCGAAGGCAAA |
| | | GCGCCGGTAATCCTCGCGGTGAACAAAG |
| | | TGGACAACGTGCAGGAGAAAGCCGATCT |
| | | GCTGCCGCACCTGCAGTTCCTGGCAAGCC |
| | | AGATGAACTTCCTCGATATCGTGCCAATC |
| | | TCTGCCGAAACCGGGCTGAATGTTGACAC |
| | | TATTGCGGCAATCGTGCGTAAGCATCTAC |
| | | CTGAAGCGACTCATCACTTCCCGGAAGAT |
| | | TACATCACCGATCGCTCACAGCGTTTTAT |
| | | GGCGTCTGAAATCATCCGCGAAAAACTG |
| | | ATGCGTTTCCTCGGCGCTGAACTGCCGTA |
| | | CTCCGTGACCGTGGAGATCGAACGTTTCG |
| | | TCTCTAACGAACGCGGTGGTTATGACATC |
| | | AACGGTTTGATTCTCGTTGAGCGTGAAGG |
| | | GCAGAAGAAGATGGTCATTGGCAACAAA |
| | | GGGGCCAAGATCAAAACCATCGGGATTG |
| | | AAGCGCGTAAAGACATGCAGGAAATGTT |
| | | CGAAGCGCCTGTTCACCTTGAACTGTGGG |
| | | TAAAAGTGAAATCCGGTTGGGCCGACGA |
| | | CGAACGCGCACTGCGCAGTCTCGGTTACG |
| | | TTGACGATCTTTAAGAGTAACTCCGATGG |
| | | AAGGCTGGCAGCGCGCATTTGTCCTGCAT |
| | | AGTCGCCCGTGGAGCGAAACCAGCCTGA |
| | | TGCTGGACGTCTTCACGGAGGAATCGGG |
| | | GCGCGTGCGTCTGGTTGCCAAAGGCGCA |
| | | CGCTCTAAACGCTCTACCCTGAAAGGTGC |
| | | ATTACAGCCTTTCACCCCTCTCTTGCTAC |
| | | GTTTTGGCGGGCGTGGCGAAGTCAAAAC |
| | | GCTGCGCAGTGCTGAAGCCGTCTCGCTGG |
| | | CGCTGCCATTAAGCGGTATCACGCTTTAC |
| | | AGCGGTCTGTACATCAACGAACTTCTCTC |
| | | CCGCGTACTGGAATACGAGACGCGCTTCT |
| | | CTGAACTTTTTTTCGATTACTTGCACTGCA |
| | | TTCAGTCTCTTGCAGGGGTCACTGGTACG |
| | | CCAGAACCCGCGCTGCGCCGCTTTGAACT |
| | | GGCACTGCTCGGGCATCTGGGTTATGGCG |
| | | TCAATTTTACCCATTGTGCGGGTAGCGGC |
| | | GAGCCGGTAGATGACACCATGACGTATC |
| | | GTTATCGCGAAGAAAAAGGGTTTATCGC |
| | | AAGCGTCGTTATCGACAATAAAACGTTCA |
| | | CCGGAAGGCAGTTAAAAGCGTTAAACGC |
| | | ACGGGAATTTCCTGACGCAGACACACTG |
| | | CGCGCCGCGAAACGCTTTACCCGCATGGC |
| | | GCTTAAGCCGTATCTTGGCGGTAAACCTT |
| | | TAAAGAGCAGGGAACTGTTCCGGCAGTT |
| | | TATGCCTAAGCGAACGGTGAAAACACAT |
| | | TATGAATGATGAGGATTGTC |

*Bold: RBS; italics: pyrE CDS; underline: T1 terminator; bold and italics: T2 terminator

TABLE 6
AminoAcid Sequence of MS2 CP

| | | |
|---|---|---|
| SEQ ID | MS2 CP | MASNFTQFVLVDNGGTGDVTVAPSNF |
| NO. 9 | | ANGVAEWISSNSRSQAYKVTCSVRQSS |
| | | AQNRKYTIKVEVPKVATQTVGGVELPV |
| | | AAWRSYLNMELTIPIFATNSDCELIVKA |
| | | MQGLLKDGNPIPSAIAANSGIY |

1. A plasmid vector comprising a nucleic acid sequence comprising: an inducible bacterial promoter operably linked to a stem-loop sequence having a 3′ end and a 5′ end and comprising a target recombinant RNA sequence; a first pac site sequence at the 3′ end, and a second pac site sequence at the 5′ end of the stem-loop sequence; an MS2 capsid protein (CP) expression cassette; and, pyrE coding sequence with a ribosome binding site (RBS) at its 5′ end and T1-T2 terminators at its 3′ end downstream of the MS2 CP expression cassette.

2. The vector of claim 1, wherein the inducible promoter comprises a coliphage T7 promoter with a sequence of SEQ ID NO. 1.

3. The vector of claim 1, wherein expression of the pyrE coding sequence comprising RBS-pyrE cds-T1-T2 with a sequence of SEQ ID NO 2 is driven by: (a) an coliphage T7 promoter comprising the sequence of SEQ ID NO: 1; wherein the coliphage T7 promoter is an upstream promoter or a dedicated promoter; or (b) a dedicated J23115 promoter comprising the sequence of SEO ID NO: 33.

4. (canceled)

5. (canceled)

6. (canceled)

7. The vector of claim 1, wherein the target recombinant RNA is selected from a dsRNA, a siRNA, a shRNA, a hpRNA, and a miRNA.

8. The vector of claim 1, wherein the first and second pac site sequences each comprise a sequence of SEQ ID NO. 3 and SEQ ID NO: 4, respectively.

9. The vector of claim 1, comprising the sequences of SEQ ID NO: 1, 2, 3, 4, 5, 6, 7, 8, 33, or any combinations thereof.

10. The vector of claim 1, wherein the vector does not comprise an antibiotic resistance Amp-r gene or a tetracycline resistance gene.

11. The vector of claim 1, comprising the sequence of SEQ ID NO: 43.

12. A gram-positive or a gram-negative bacterial cell comprising the vector of claim 1.

13. The bacterial cell of claim 12, wherein the bacterial cell is an E. coli cell or a C. glutamicum cell.

14. (canceled)

15. The E. coli cell of claim 13, wherein the E. coli cell is an RNAselII-deficient E. coli strain.

16. The E. coli cell of claim 15, wherein the RNAselII-deficient E. coli strain is HT115(DE3).

17. The E. coli cell of claim 15, wherein the E. coli strain is a uracil auxotroph.

18. A bacterial culture comprising a population of bacterial cells of claim 12 in a bioreactor.

19. The bacterial culture of claim 18, comprising the target dsRNA in amount of at least about 4 g/L, 5 g/L, 6 g/L, 7 g/L, 8 g/L, 9 g/L, 10 g/L, 11 g/L or 12 g/L.

20. The bacterial culture of claim 18, wherein the bioreactor is selected from a fed-batch system, a semi-continuous system and a continuous culture system.

21. (canceled)

22. A method for producing a target recombinant RNA comprising maintaining the bacterial culture of claim 18 in a bioreactor for a time and under conditions sufficient to yield the target recombinant RNA in an amount of at least about 4 g/L, 5 g/L, 6 g/L, 7 g/L, 8 g/L, 9 g/L, 10 g/L, 11 g/L or 12 g/L.

23. (canceled)

24. (canceled)

25. The method of claim 22, further comprising harvesting the target recombinant RNA.

26. (canceled)

27. The method of claim 22, wherein the target recombinant RNA is selected from a dsRNA, a siRNA, a shRNA, a hpRNA, and a miRNA.

28. The method of claim 22, wherein the method produces an antibiotic marker free recombinant RNA.

29. (canceled)


SEQUENCE LISTING (44 sequences)

SEQ ID NO: 1 - DNA, 19 bp, synthetic construct
  source 1..19: mol_type = other DNA
taatacgactcactatagg

SEQ ID NO: 2 - DNA, 1023 bp, synthetic construct
  source 1..1023: mol_type = other DNA
agaaggagatatacatatgaaaccatatcagcgccagtttattgaatttgcgcttagcaagcaggtgttaaagtttggcgagtttacgctgaaatccgggcgcaaaagcccctatttcttcaacgccgggctgtttaataccgggcgcgatctggcactgttaggccgtttttacgctgaagcgttggtggattccggcattgagttcgatctgctgtttggccctgcttacaaagggatcccgattgccaccacaaccgctgtggcactggcggagcatcacgacctggacctgccgtactgctttaaccgcaaagaagcaaaagaccacggtgaaggcggcaatctggttggtagcgcgttacaaggacgcgtaatgctggtagatgatgtgatcaccgccggaacggcgattcgcgagtcgatggagattattcaggccaatggcgcgacgcttgctggcgtgttgatttcgctcgatcgtcaggaacgcgggcgcggcgagatttcggcgattcaggaagttgagcgtgattacaactgcaaagtgatctctatcatcaccctgaaagacctgattgcttacctggaagagaagccggaaatggcggaacatctggcggcggttaaggcctatcgcgaagagtttggcgtttaacctaggcagaacgcagaagctgtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttt

SEQ ID NO: 3 - DNA, 19 bp, synthetic construct
  source 1..19: mol_type = other DNA
acatgaggatcacccatgt

SEQ ID NO: 4 - DNA, 19 bp, synthetic construct
  source 1..19: mol_type = other DNA
acatgaggatcacccatgt

SEQ ID NO: 5 - DNA, 393 bp, synthetic construct
  source 1..393: mol_type = other DNA
atggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaa

SEQ ID NO: 6 - DNA, 48 bp, synthetic construct
  source 1..48: mol_type = other DNA
ctagcataaccccttggggcctctaaacgggtcttgaggggttttttg

SEQ ID NO: 7 - DNA, 1208 bp, synthetic construct
  source 1..1208: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcac

SEQ ID NO: 8 - DNA, 966 bp, synthetic construct
  source 1..966: mol_type = other DNA
cgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 9 - protein, 130 aa, synthetic construct
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 10 - DNA, 25 bp, synthetic construct
  source 1..25: mol_type = other DNA
atttaggatccagatctcgatcccg

SEQ ID NO: 11 - DNA, 27 bp, synthetic construct
  source 1..27: mol_type = other DNA
tttatgcatgccggatatagttcctcc

SEQ ID NO: 12 - DNA, 28 bp, synthetic construct
  source 1..28: mol_type = other DNA
cgtcttcaagaattccgaaattaatacg

SEQ ID NO: 13 - DNA, 26 bp, synthetic construct
  source 1..26: mol_type = other DNA
ataatgcgcatgcccgccgcccagtc

SEQ ID NO: 14 - DNA, 46 bp, synthetic construct
  source 1..46: mol_type = other DNA
gaggattacccatgtgcgatcgcgcacgaggtttttctgtctagtg

SEQ ID NO: 15 - DNA, 120 bp, synthetic construct
  source 1..120: mol_type = other DNA
cgatgtggcctgaggaatacctctctcattgcgtttagataaagcatcccgctggaaagggaaccagtactttgtaaagcagctagagggtttaaactcatcccagttggtgatgatacc

SEQ ID NO: 16 - DNA, 117 bp, synthetic construct
  source 1..117: mol_type = other DNA
gagaggtattcctcaggccacatcgcttcctagttccgctgggatccatcgttggcggccgaagccgccattccatagtgagttctggcgcgcctcatcccagttggtgatgatacc

SEQ ID NO: 17 - DNA, 46 bp, synthetic construct
  source 1..46: mol_type = other DNA
gttcagaccgggtattcggtccggcacgaggtttttctgtctagtg

SEQ ID NO: 18 - DNA, 23 bp, synthetic construct
  source 1..23: mol_type = other DNA
cggaccgaatacccggtctgaac

SEQ ID NO: 19 - DNA, 23 bp, synthetic construct
  source 1..23: mol_type = other DNA
gcgatcgcacatgggtaatcctc

SEQ ID NO: 20 - DNA, 47 bp, synthetic construct
  source 1..47: mol_type = other DNA
gaggattacccatgtgcgatcgccgatctctctccctcgactctaac

SEQ ID NO: 21 - DNA, 120 bp, synthetic construct
  source 1..120: mol_type = other DNA
cgatgtggcctgaggaatacctctctcattgcgtttagataaagcatcccgctggaaagggaaccagtactttgtaaagcagctagagggtttaaacccatgtcatcccaattagtaata

SEQ ID NO: 22 - DNA, 117 bp, synthetic construct
  source 1..117: mol_type = other DNA
gagaggtattcctcaggccacatcgcttcctagttccgctgggatccatcgttggcggccgaagccgccattccatagtgagttctggcgcgccccatgtcatcccaattagtaata

SEQ ID NO: 23 - DNA, 47 bp, synthetic construct
  source 1..47: mol_type = other DNA
gttcagaccgggtattcggtccgcgatctctctccctcgactctaac

SEQ ID NO: 24 - DNA, 39 bp, synthetic construct
  source 1..39: mol_type = other DNA
attagtcgacaatcctaggcagaacgcagaagctgtctg

SEQ ID NO: 25 - DNA, 32 bp, synthetic construct
  source 1..32: mol_type = other DNA
ataatcgcgagtagaaacgcaaaaaggccatc

SEQ ID NO: 26 - DNA, 54 bp, synthetic construct
  source 1..54: mol_type = other DNA
attgtcgacagaaggagatatacatatgaaaccatatcagcgccagtttattga

SEQ ID NO: 27 - DNA, 33 bp, synthetic construct
  source 1..33: mol_type = other DNA
taaatccctaggttaaacgccaaactcttcgcg

SEQ ID NO: 28 - DNA, 29 bp, synthetic construct
  source 1..29: mol_type = other DNA
actccttaattaactctaagtacttcttg

SEQ ID NO: 29 - DNA, 26 bp, synthetic construct
  source 1..26: mol_type = other DNA
gagttaattaaggagttcaaacatgg

SEQ ID NO: 30 - DNA, 23 bp, synthetic construct
  source 1..23: mol_type = other DNA
tgtggatatccggatatagttcc

SEQ ID NO: 31 - DNA, 20 bp, synthetic construct
  source 1..20: mol_type = other DNA
atcatggcgaccacacccgt

SEQ ID NO: 32 - DNA, 23 bp, synthetic construct
  source 1..23: mol_type = other DNA
actgggttgaaggctctcaaggg

SEQ ID NO: 33 - DNA, 35 bp, synthetic construct
  source 1..35: mol_type = other DNA
tttatagctagctcagcccttggtacaatgctagc

SEQ ID NO: 34 - DNA, 6407 bp, synthetic construct
  source 1..6407: mol_type = other DNA
ttccgaaattaatacgactcactatagggagacatgaggattacccatgtgcgatcgccgatctctctccctcgactctaacaccagcgaaagtaacagccaatcaagatgtgtgacgatgatgttgcggcattagtcgtggacaatgggtccggtatgtgcaaggctggattcgcgggggatgatgcaccacgcgctgtgtttcccagcatcgtcggtcgtcctcgtcatcagggtgtgatggtcggtatgggtcaaaaagacagttatgttggcgacgaggcgcaaagtaagagaggtatattgacactaaagtatcctatagaacatggcattattactaattgggatgacatgggtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgccccatgtcatcccaattagtaataatgccatgttctataggatactttagtgtcaatatacctctcttactttgcgcctcgtcgccaacataactgtctttttgacccataccgaccatcacaccctgatgacgaggacgaccgacgatgctgggaaacacagcgcgtggtgcatcatcccccgcgaatccagccttgcacataccggacccattgtccacgactaatgccgcaacatcatcgtcacacatcttgattggctgttactttcgctggtgttagagtcgagggagagagatcgcggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgacagaaggagatatacatatgaaaccatatcagcgccagtttattgaatttgcgcttagcaagcaggtgttaaagtttggcgagtttacgctgaaatccgggcgcaaaagcccctatttcttcaacgccgggctgtttaataccgggcgcgatctggcactgttaggccgtttttacgctgaagcgttggtggattccggcattgagttcgatctgctgtttggccctgcttacaaagggatcccgattgccaccacaaccgctgtggcactggcggagcatcacgacctggacctgccgtactgctttaaccgcaaagaagcaaaagaccacggtgaaggcggcaatctggttggtagcgcgttacaaggacgcgtaatgctggtagatgatgtgatcaccgccggaacggcgattcgcgagtcgatggagattattcaggccaatggcgcgacgcttgctggcgtgttgatttcgctcgatcgtcaggaacgcgggcgcggcgagatttcggcgattcaggaagttgagcgtgattacaactgcaaagtgatctctatcatcaccctgaaagacctgattgcttacctggaagagaagccggaaatggcggaacatctggcggcggttaaggcctatcgcgaagagtttggcgtttaacctaggcagaacgcagaagctgtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctactcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 35 - DNA, 6434 bp, synthetic construct
  source 1..6434: mol_type = other DNA
ttccgaaattaatacgactcactatagggagacatgaggattacccatgtgcgatcgccgatctctctccctcgactctaacaccagcgaaagtaacagccaatcaagatgtgtgacgatgatgttgcggcattagtcgtggacaatgggtccggtatgtgcaaggctggattcgcgggggatgatgcaccacgcgctgtgtttcccagcatcgtcggtcgtcctcgtcatcagggtgtgatggtcggtatgggtcaaaaagacagttatgttggcgacgaggcgcaaagtaagagaggtatattgacactaaagtatcctatagaacatggcattattactaattgggatgacatgggtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgccccatgtcatcccaattagtaataatgccatgttctataggatactttagtgtcaatatacctctcttactttgcgcctcgtcgccaacataactgtctttttgacccataccgaccatcacaccctgatgacgaggacgaccgacgatgctgggaaacacagcgcgtggtgcatcatcccccgcgaatccagccttgcacataccggacccattgtccacgactaatgccgcaacatcatcgtcacacatcttgattggctgttactttcgctggtgttagagtcgagggagagagatcgcggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgactaatacgactcactatagggagaccacagaaggagatatacatatgaaaccatatcagcgccagtttattgaatttgcgcttagcaagcaggtgttaaagtttggcgagtttacgctgaaatccgggcgcaaaagcccctatttcttcaacgccgggctgtttaataccgggcgcgatctggcactgttaggccgtttttacgctgaagcgttggtggattccggcattgagttcgatctgctgtttggccctgcttacaaagggatcccgattgccaccacaaccgctgtggcactggcggagcatcacgacctggacctgccgtactgctttaaccgcaaagaagcaaaagaccacggtgaaggcggcaatctggttggtagcgcgttacaaggacgcgtaatgctggtagatgatgtgatcaccgccggaacggcgattcgcgagtcgatggagattattcaggccaatggcgcgacgcttgctggcgtgttgatttcgctcgatcgtcaggaacgcgggcgcggcgagatttcggcgattcaggaagttgagcgtgattacaactgcaaagtgatctctatcatcaccctgaaagacctgattgcttacctggaagagaagccggaaatggcggaacatctggcggcggttaaggcctatcgcgaagagtttggcgtttaacctaggcagaacgcagaagctgtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctactcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 36 - DNA, 8837 bp, synthetic construct
  source 1..8837: mol_type = other DNA
ggatcctctagagtcgacgctctcccttatgcgactcctgcattaggaagcagcccagtagtaggttgaggccgttgagcaccgccgccgcaaggaatggtgcatgccggatatagttcctcctttcagcaaaaaacccctcaagacccgtttagaggccccaaggggttatgctagttattgctcagcggtggcagcgcgtccgcttattagtaaatgccagagttagcagcgattgcagatgggatcggattaccgtctttcagcaggccctgcattgccttaacaatcagttcgcagtcggagttcgtcgcaaaaatcggaatagtcagttccatgttgaggtaagagcgccaggccgcaactgggagttctacgccgcctacggtctgcgttgcgactttcggtacttcaactttgatggtgtatttgcgattctgcgcgctagactgacgaacggagcacgtgactttgtaggcctgagagcggctgttagagctaatccattccgcaacaccgttggcgaagttggacggagctacggtaacgtcacccgtaccgccgttatcaaccagaacgaattgggtaaagttagacgccatatgtatatctccttcttagcctgcttttttgtacaaacttgtgatctagagggaaaccgttgtggtctccctatagtgagtcgtattaatttcgcgggatcgagatctggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatgcatgcccgccgcccagtcctgctcgcttcgctacttggagccactatcgactacgcgatcatggcgaccacacccgtcctgtggatatccggatatagttcctcctttcagcaaaaaacccctcaagacccgtttagaggccccaaggggttatgctagggtgtgggagcttcgacatgggtgatcctcatgtttgaactccttaattaactctaagtacttcttgggtaccgcggccgccctcgttcagaccgggtattcggtccgcgatctctctccctcgactctaacaccagcgaaagtaacagccaatcaagatgtgtgacgatgatgttgcggcattagtcgtggacaatgggtccggtatgtgcaaggctggattcgcgggggatgatgcaccacgcgctgtgtttcccagcatcgtcggtcgtcctcgtcatcagggtgtgatggtcggtatgggtcaaaaagacagttatgttggcgacgaggcgcaaagtaagagaggtatattgacactaaagtatcctatagaacatggcattattactaattgggatgacatggggcgcgccagaactcactatggaatggcggcttcggccgccaacgatggatcccagcggaactaggaagcgatgtggcctgaggaatacctctctcattgcgtttagataaagcatcccgctggaaagggaaccagtactttgtaaagcagctagagggtttaaacccatgtcatcccaattagtaataatgccatgttctataggatactttagtgtcaatatacctctcttactttgcgcctcgtcgccaacataactgtctttttgacccataccgaccatcacaccctgatgacgaggacgaccgacgatgctgggaaacacagcgcgtggtgcatcatcccccgcgaatccagccttgcacataccggacccattgtccacgactaatgccgcaacatcatcgtcacacatcttgattggctgttactttcgctggtgttagagtcgagggagagagatcggcgatcgcacatgggtaatcctcatgtctccctatagtgagtcgtattaatttcgggagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacaccgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggaggatcaagtcggtcaagccaagcgcaaccagcggcaccgccgcgagcaacgtcgcaagggcgatcaggggacgatttttgcgaagaatttccacggtaagaatccaatctctcgaatttagggtgaaagaagcttggcataggggtgtgcacgaactcggtggaggaaatttccgcggggcaaggcttcgcgaagcggagtcgcggcagtggctttgaagatctttgggagcagtccttgtgcgcttacgaggtgagccggtggggaaccgttatctgcctatggtgtgagcccccctagagagcttcaagagcaatcagcccgacctagaaaggaggccaagagagagacccctacggggggaaccgttttctgcctacgagatggcacatttactgggaagctttacggcgtcctcgtggaagttcaatgcccgcagacttaagtgctctattcacggtctgacgtgacacgctaaattcagacatagcttcattgattgtcggccacgagccagtctctccctcaacagtcataaaccaacctgcaatggtcaagcgatttcctttagctttcctagcttgtcgttgactggacttagctagtttttctcgctgtgctcgggcgtactcactgtttgggtctttccagcgttctgcggcctttttaccgccacgtcttcccatagtggccagagcttttcgccctcggctgctctgcgtctctgtctgacgagcagggacgactggctggcctttagcgacgtagccgcgcacacgtcgcgccatcgtctggcggtcacgcatcggcggcagatcaggctcacggccgtctgctccgaccgcctgagcgacggtgtaggcacgctcgtaggcgtcgatgatcttggtgtcttttaggcgctcaccagccgcttttaactggtatcccacagtcaaagcgtggcgaaaagccgtctcatcacgggcggcacgccctggagcagtccagaggacacggacgccgtcgatcagctctccagacgcttcagcggcgctcggcaggcttgcttcaagcgtggcaagtgcttttgcttccgcagtggcttttcttgccgcttcgatacgtgcccgtccgctagaaaactcctgctcatagcgttttttaggtttttctgtgcctgagatcatgcgagcaacctccataagatcagctaggcgatccacgcgattgtgctgggcatgccagcggtacgcggtgggatcgtcggagacgtgcagtggccaccggctcagcctatgtgaaaaagcctggtcagcgccgaaaacgcgggtcatttcctcggtcgttgcagccagcaggcgcatattcgggctgctcatgcctgctgcggcatacaccggatcaatgagccagatgagctggcatttcccgctcagtggattcacgccgatccaagctggcgctttttccaggcgtgcccagcgctccaaaatcgcgtagacctcggggtttacgtgctcgattttcccgccggcctggtggctcggcacatcaatgtccaggacaagcacggctgcgtgctgcgcgtgcgtcagagcaacatactggcaccgggcaagcgattttgaaccaactcggtataacttcggctgtgtttctcccgtgtccgggtctttgatccaagcgctggcgaagtcgcgggtcttgctgccctggaaattttctctgcccaggtgagcgaggaattcgcggcggtcttcgctcgtccagccacgtgatcgcagcgcgagctcgggatgggtgtcgaacagatcagcggaaaatttccaggccggtgtgtcaatgtctcgtgaatccgctagagtcatttttgagcgctttctcccaggtttggactgggggttagccgacgccctgtgagttaccgctcacggggcgttcaacatttttcaggtattcgtgcagcttatcgcttcttgccgcctgtgcgctttttcgacgcgcgacgctgctgccgattcggtgcaggtggtggcggcgctgacacgtcctgggcggccacggccacacgaaacgcggcatttacgatgtttgtcatgcctgcgggcaccgcgccacgatcgcggataattctcgctgccgcttccagctctgtgacgaccatggccaaaatttcgctcgggggacgcacttccagcgccatttgcgacctagccgcctccagctcctcggcgtggcgtttgttggcgcgctcgcggctggctgcggcacgacacgcatctgagcaatattttgcgcgccgtcctcgcgggtcaggccggggaggaatcaggccaccgcagtaggcgcaactgattcgatcctccactactgtgcgtcctcctggcgctgccgagcacgcagctcgtcagccagctcctcaagatccgccacgagagtttctaggtcgctcgcggcactggcccagtctcgtgatgctggcgcgtccgtcgtatcgagagctcggaaaaatccgatcaccgtttttaaatcgacggcagcatcgagcgcgtcggactccagcgcgacatcagagagatccatagctgatgattcgggccaattttggtacttcgtcgtgaaggtcatgacaccattataacgaacgttcgttaaagtttttggcggaaaatcacgcggcacgaaaattttcacgaagcgggactttgcgcagctcaggggtgctaaaaattttgtatcgcacttgatttttccgaaagacagattatctgcaaacggtgtgtcgtatttctggcttggtttttaaaaaatctggaatcgaaaatttgcggggcgaccgagaagttttttacaaaaggcaaaaactttttcgggatcgacagaaataaaacgatcgacggtacgcaacaaaaaagcgtcaggatcgccgtagagcgattgaagaccgtcaaccaaaggggaagcctccaatcgacgcgacgcgcgctctacggcgatcctgacgcagatttttagctatctgtcgcagcgccctcagggacaagccacccgcacaacgtcgcgagggcgatcagcgacgccgcaggg

SEQ ID NO: 37 - DNA, 6395 bp, synthetic construct
  source 1..6395: mol_type = other DNA
ttccgaaattaatacgactcactatagggagacatgaggattacccatgtgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcgggcatgcatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccagatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggcatgcaccattccttgcggcggcggtgctcaacggcctcaacctactactgggctgcttcctaatgcaggagtcgcataagggagagcgtcgacagaaggagatatacatatgaaaccatatcagcgccagtttattgaatttgcgcttagcaagcaggtgttaaagtttggcgagtttacgctgaaatccgggcgcaaaagcccctatttcttcaacgccgggctgtttaataccgggcgcgatctggcactgttaggccgtttttacgctgaagcgttggtggattccggcattgagttcgatctgctgtttggccctgcttacaaagggatcccgattgccaccacaaccgctgtggcactggcggagcatcacgacctggacctgccgtactgctttaaccgcaaagaagcaaaagaccacggtgaaggcggcaatctggttggtagcgcgttacaaggacgcgtaatgctggtagatgatgtgatcaccgccggaacggcgattcgcgagtcgatggagattattcaggccaatggcgcgacgcttgctggcgtgttgatttcgctcgatcgtcaggaacgcgggcgcggcgagatttcggcgattcaggaagttgagcgtgattacaactgcaaagtgatctctatcatcaccctgaaagacctgattgcttacctggaagagaagccggaaatggcggaacatctggcggcggttaaggcctatcgcgaagagtttggcgtttaacctaggcagaacgcagaagctgtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctactcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 38 - DNA, 10105 bp, synthetic construct
  source 1..10105: mol_type = other DNA
ggatcctctagtctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgagtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagacagcttctgcgttctgcctaggttaaacgccaaactcttcgcgataggccttaaccgccgccagatgttccgccatttccggcttctcttccaggtaagcaatcaggtctttcagggtgatgatagagatcactttgcagttgtaatcacgctcaacttcctgaatcgccgaaatctcgccgcgcccgcgttcctgacgatcgagcgaaatcaacacgccagcaagcgtcgcgccattggcctgaataatctccatcgactcgcgaatcgccgttccggcggtgatcacatcatctaccagcattacgcgtccttgtaacgcgctaccaaccagattgccgccttcaccgtggtcttttgcttctttgcggttaaagcagtacggcaggtccaggtcgtgatgctccgccagtgccacagcggttgtggtggcaatcgggatccctttgtaagcagggccaaacagcagatcgaactcaatgccggaatccaccaacgcttcagcgtaaaaacggcctaacagtgccagatcgcgcccggtattaaacagcccggcgttgaagaaataggggcttttgcgcccggatttcagcgtaaactcgccaaactttaacacctgcttgctaagcgcaaattcaataaactggcgctgatatggtttcatatgtatatctccttctgtcgacgctctcccttatgcgactcctgcattaggaagcagcccagtagtaggttgaggccgttgagcaccgccgccgcaaggaatggtgcatgccggatatagttcctcctttcagcaaaaaacccctcaagacccgtttagaggccccaaggggttatgctagttattgctcagcggtggcagcgcgtccgcttattagtaaatgccagagttagcagcgattgcagatgggatcggattaccgtctttcagcaggccctgcattgccttaacaatcagttcgcagtcggagttcgtcgcaaaaatcggaatagtcagttccatgttgaggtaagagcgccaggccgcaactgggagttctacgccgcctacggtctgcgttgcgactttcggtacttcaactttgatggtgtatttgcgattctgcgcgctagactgacgaacggagcacgtgactttgtaggcctgagagcggctgttagagctaatccattccgcaacaccgttggcgaagttggacggagctacggtaacgtcacccgtaccgccgttatcaaccagaacgaattgggtaaagttagacgccatatgtatatctccttcttagcctgcttttttgtacaaacttgtgatctagagggaaaccgttgtggtctccctatagtgagtcgtattaatttcgcgggatcgagatctggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatgcatgcccgccgcccagtcctgctcgcttcgctacttggagccactatcgactacgcgatcatggcgaccacacccgtcctgtggatatccggatatagttcctcctttcagcaaaaaacccctcaagacccgtttagaggccccaaggggttatgctagggtgtgggagcttcgacatgggtgatcctcatgtttgaactccttaattaactctaagtacttcttgggtaccgcggccgccctcgttcagaccgggtattcggtccgcgatctctctccctcgactctaacaccagcgaaagtaacagccaatcaagatgtgtgacgatgatgttgcggcattagtcgtggacaatgggtccggtatgtgcaaggctggattcgcgggggatgatgcaccacgcgctgtgtttcccagcatcgtcggtcgtcctcgtcatcagggtgtgatggtcggtatgggtcaaaaagacagttatgttggcgacgaggcgcaaagtaagagaggtatattgacactaaagtatcctatagaacatggcattattactaattgggatgacatggggcgcgccagaactcactatggaatggcggcttcggccgccaacgatggatcccagcggaactaggaagcgatgtggcctgaggaatacctctctcattgcgtttagataaagcatcccgctggaaagggaaccagtactttgtaaagcagctagagggtttaaacccatgtcatcccaattagtaataatgccatgttctataggatactttagtgtcaatatacctctcttactttgcgcctcgtcgccaacataactgtctttttgacccataccgaccatcacaccctgatgacgaggacgaccgacgatgctgggaaacacagcgcgtggtgcatcatcccccgcgaatccagccttgcacataccggacccattgtccacgactaatgccgcaacatcatcgtcacacatcttgattggctgttactttcgctggtgttagagtcgagggagagagatcggcgatcgcacatgggtaatcctcatgtctccctatagtgagtcgtattaatttcgggagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacaccgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggaggatcaagtcggtcaagccaagcgcaaccagcggcaccgccgcgagcaacgtcgcaagggcgatcaggggacgatttttgcgaagaatttccacggtaagaatccaatctctcgaatttagggtgaaagaagcttggcataggggtgtgcacgaactcggtggaggaaatttccgcggggcaaggcttcgcgaagcggagtcgcggcagtggctttgaagatctttgggagcagtccttgtgcgcttacgaggtgagccggtggggaaccgttatctgcctatggtgtgagcccccctagagagcttcaagagcaatcagcccgacctagaaaggaggccaagagagagacccctacggggggaaccgttttctgcctacgagatggcacatttactgggaagctttacggcgtcctcgtggaagttcaatgcccgcagacttaagtgctctattcacggtctgacgtgacacgctaaattcagacatagcttcattgattgtcggccacgagccagtctctccctcaacagtcataaaccaacctgcaatggtcaagcgatttcctttagctttcctagcttgtcgttgactggacttagctagtttttctcgctgtgctcgggcgtactcactgtttgggtctttccagcgttctgcggcctttttaccgccacgtcttcccatagtggccagagcttttcgccctcggctgctctgcgtctctgtctgacgagcagggacgactggctggcctttagcgacgtagccgcgcacacgtcgcgccatcgtctggcggtcacgcatcggcggcagatcaggctcacggccgtctgctccgaccgcctgagcgacggtgtaggcacgctcgtaggcgtcgatgatcttggtgtcttttaggcgctcaccagccgcttttaactggtatcccacagtcaaagcgtggcgaaaagccgtctcatcacgggcggcacgccctggagcagtccagaggacacggacgccgtcgatcagctctccagacgcttcagcggcgctcggcaggcttgcttcaagcgtggcaagtgcttttgcttccgcagtggcttttcttgccgcttcgatacgtgcccgtccgctagaaaactcctgctcatagcgttttttaggtttttctgtgcctgagatcatgcgagcaacctccataagatcagctaggcgatccacgcgattgtgctgggcatgccagcggtacgcggtgggatcgtcggagacgtgcagtggccaccggctcagcctatgtgaaaaagcctggtcagcgccgaaaacgcgggtcatttcctcggtcgttgcagccagcaggcgcatattcgggctgctcatgcctgctgcggcatacaccggatcaatgagccagatgagctggcatttcccgctcagtggattcacgccgatccaagctggcgctttttccaggcgtgcccagcgctccaaaatcgcgtagacctcggggtttacgtgctcgattttcccgccggcctggtggctcggcacatcaatgtccaggacaagcacggctgcgtgctgcgcgtgcgtcagagcaacatactggcaccgggcaagcgattttgaaccaactcggtataacttcggctgtgtttctcccgtgtccgggtctttgatccaagcgctggcgaagtcgcgggtcttgctgccctggaaattttctctgcccaggtgagcgaggaattcgcggcggtcttcgctcgtccagccacgtgatcgcagcgcgagctcgggatgggtgtcgaacagatcagcggaaaatttccaggccggtgtgtcaatgtctcgtgaatccgctagagtcatttttgagcgctttctcccaggtttggactgggggttagccgacgccctgtgagttaccgctcacggggcgttcaacatttttcaggtattcgtgcagcttatcgcttcttgccgcctgtgcgctttttcgacgcgcgacgctgctgccgattcggtgcaggtggtggcggcgctgacacgtcctgggcggccacggccacacgaaacgcggcatttacgatgtttgtcatgcctgcgggcaccgcgccacgatcgcggataattctcgctgccgcttccagctctgtgacgaccatggccaaaatttcgctcgggggacgcacttccagcgccatttgcgacctagccgcctccagctcctcggcgtggcgtttgttggcgcgctcgcggctggctgcggcacgacacgcatctgagcaatattttgcgcgccgtcctcgcgggtcaggccggggaggaatcaggccaccgcagtaggcgcaactgattcgatcctccactactgtgcgtcctcctggcgctgccgagcacgcagctcgtcagccagctcctcaagatccgccacgagagtttctaggtcgctcgcggcactggcccagtctcgtgatgctggcgcgtccgtcgtatcgagagctcggaaaaatccgatcaccgtttttaaatcgacggcagcatcgagcgcgtcggactccagcgcgacatcagagagatccatagctgatgattcgggccaattttggtacttcgtcgtgaaggtcatgacaccattataacgaacgttcgttaaagtttttggcggaaaatcacgcggcacgaaaattttcacgaagcgggactttgcgcagctcaggggtgctaaaaattttgtatcgcacttgatttttccgaaagacagattatctgcaaacggtgtgtcgtatttctggcttggtttttaaaaaatctggaatcgaaaatttgcggggcgaccgagaagttttttacaaaaggcaaaaactttttcgggatcgacagaaataaaacgatcgacggtacgcaacaaaaaagcgtcaggatcgccgtagagcgattgaagaccgtcaaccaaaggggaagcctccaatcgacgcgacgcgcgctctacggcgatcctgacgcagatttttagctatctgtcgcagcgccctcagggacaagccacccgcacaacgtcgcgagggcgatcagcgacgccgcaggg

SEQ ID NO: 39 - DNA, 10093 bp, synthetic construct
  source 1..10093: mol_type = other DNA
ggatcctctagtctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgagtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagacagcttctgcgttctgcctaggttaaacgccaaactcttcgcgataggccttaaccgccgccagatgttccgccatttccggcttctcttccaggtaagcaatcaggtctttcagggtgatgatagagatcactttgcagttgtaatcacgctcaacttcctgaatcgccgaaatctcgccgcgcccgcgttcctgacgatcgagcgaaatcaacacgccagcaagcgtcgcgccattggcctgaataatctccatcgactcgcgaatcgccgttccggcggtgatcacatcatctaccagcattacgcgtccttgtaacgcgctaccaaccagattgccgccttcaccgtggtcttttgcttctttgcggttaaagcagtacggcaggtccaggtcgtgatgctccgccagtgccacagcggttgtggtggcaatcgggatccctttgtaagcagggccaaacagcagatcgaactcaatgccggaatccaccaacgcttcagcgtaaaaacggcctaacagtgccagatcgcgcccggtattaaacagcccggcgttgaagaaataggggcttttgcgcccggatttcagcgtaaactcgccaaactttaacacctgcttgctaagcgcaaattcaataaactggcgctgatatggtttcatatgtatatctccttctgtcgacgctctcccttatgcgactcctgcattaggaagcagcccagtagtaggttgaggccgttgagcaccgccgccgcaaggaatggtgcatgccggatatagttcctcctttcagcaaaaaacccctcaagacccgtttagaggccccaaggggttatgctagttattgctcagcggtggcagcgcgtccgcttattagtaaatgccagagttagcagcgattgcagatgggatcggattaccgtctttcagcaggccctgcattgccttaacaatcagttcgcagtcggagttcgtcgcaaaaatcggaatagtcagttccatgttgaggtaagagcgccaggccgcaactgggagttctacgccgcctacggtctgcgttgcgactttcggtacttcaactttgatggtgtatttgcgattctgcgcgctagactgacgaacggagcacgtgactttgtaggcctgagagcggctgttagagctaatccattccgcaacaccgttggcgaagttggacggagctacggtaacgtcacccgtaccgccgttatcaaccagaacgaattgggtaaagttagacgccatatgtatatctccttcttagcctgcttttttgtacaaacttgtgatctagagggaaaccgttgtggtctccctatagtgagtcgtattaatttcgcgggatcgagatctggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatgcatgcccgccgcccagtcctgctcgcttcgctacttggagccactatcgactacgcgatcatggcgaccacacccgtcctgtggatatccggatatagttcctcctttcagcaaaaaacccctcaagacccgtttagaggccccaaggggttatgctagggtgtgggagcttcgacatgggtgatcctcatgtttgaactccttaattaactctaagtacttcttgggtaccgcggccgccctcgttcagaccgggtattcggtccgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgaggcgcgccagaactcactatggaatggcggcttcggccgccaacgatggatcccagcggaactaggaagcgatgtggcctgaggaatacctctctcattgcgtttagataaagcatcccgctggaaagggaaccagtactttgtaaaagctagagggtttaaactcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgcggcgatcgcacatgggtaatcctcatgtctccctatagtgagtcgtattaatttcgggagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacaccgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggaggatcaagtcggtcaagccaagcgcaaccagcggcaccgccgcgagcaacgtcgcaagggcgatcaggggacgatttttgcgaagaatttccacggtaagaatccaatctctcgaatttagggtgaaagaagcttggcataggggtgtgcacgaactcggtggaggaaatttccgcggggcaaggcttcgcgaagcggagtcgcggcagtggctttgaagatctttgggagcagtccttgtgcgcttacgaggtgagccggtggggaaccgttatctgcctatggtgtgagcccccctagagagcttcaagagcaatcagcccgacctagaaaggaggccaagagagagacccctacggggggaaccgttttctgcctacgagatggcacatttactgggaagctttacggcgtcctcgtggaagttcaatgcccgcagacttaagtgctctattcacggtctgacgtgacacgctaaattcagacatagcttcattgattgtcggccacgagccagtctctccctcaacagtcataaaccaacctgcaatggtcaagcgatttcctttagctttcctagcttgtcgttgactggacttagctagtttttctcgctgtgctcgggcgtactcactgtttgggtctttccagcgttctgcggcctttttaccgccacgtcttcccatagtggccagagcttttcgccctcggctgctctgcgtctctgtctgacgagcagggacgactggctggcctttagcgacgtagccgcgcacacgtcgcgccatcgtctggcggtcacgcatcggcggcagatcaggctcacggccgtctgctccgaccgcctgagcgacggtgtaggcacgctcgtaggcgtcgatgatcttggtgtcttttaggcgctcaccagccgcttttaactggtatcccacagtcaaagcgtggcgaaaagccgtctcatcacgggcggcacgccctggagcagtccagaggacacggacgccgtcgatcagctctccagacgcttcagcggcgctcggcaggcttgcttcaagcgtggcaagtgcttttgcttccgcagtggcttttcttgccgcttcgatacgtgcccgtccgctagaaaactcctgctcatagcgttttttaggtttttctgtgcctgagatcatgcgagcaacctccataagatcagctaggcgatccacgcgattgtgctgggcatgccagcggtacgcggtgggatcgtcggagacgtgcagtggccaccggctcagcctatgtgaaaaagcctggtcagcgccgaaaacgcgggtcatttcctcggtcgttgcagccagcaggcgcatattcgggctgctcatgcctgctgcggcatacaccggatcaatgagccagatgagctggcatttcccgctcagtggattcacgccgatccaagctggcgctttttccaggcgtgcccagcgctccaaaatcgcgtagacctcggggtttacgtgctcgattttcccgccggcctggtggctcggcacatcaatgtccaggacaagcacggctgcgtgctgcgcgtgcgtcagagcaacatactggcaccgggcaagcgattttgaaccaactcggtataacttcggctgtgtttctcccgtgtccgggtctttgatccaagcgctggcgaagtcgcgggtcttgctgccctggaaattttctctgcccaggtgagcgaggaattcgcggcggtcttcgctcgtccagccacgtgatcgcagcgcgagctcgggatgggtgtcgaacagatcagcggaaaatttccaggccggtgtgtcaatgtctcgtgaatccgctagagtcatttttgagcgctttctcccaggtttggactgggggttagccgacgccctgtgagttaccgctcacggggcgttcaacatttttcaggtattcgtgcagcttatcgcttcttgccgcctgtgcgctttttcgacgcgcgacgctgctgccgattcggtgcaggtggtggcggcgctgacacgtcctgggcggccacggccacacgaaacgcggcatttacgatgtttgtcatgcctgcgggcaccgcgccacgatcgcggataattctcgctgccgcttccagctctgtgacgaccatggccaaaatttcgctcgggggacgcacttccagcgccatttgcgacctagccgcctccagctcctcggcgtggcgtttgttggcgcgctcgcggctggctgcggcacgacacgcatctgagcaatattttgcgcgccgtcctcgcgggtcaggccggggaggaatcaggccaccgcagtaggcgcaactgattcgatcctccactactgtgcgtcctcctggcgctgccgagcacgcagctcgtcagccagctcctcaagatccgccacgagagtttctaggtcgctcgcggcactggcccagtctcgtgatgctggcgcgtccgtcgtatcgagagctcggaaaaatccgatcaccgtttttaaatcgacggcagcatcgagcgcgtcggactccagcgcgacatcagagagatccatagctgatgattcgggccaattttggtacttcgtcgtgaaggtcatgacaccattataacgaacgttcgttaaagtttttggcggaaaatcacgcggcacgaaaattttcacgaagcgggactttgcgcagctcaggggtgctaaaaattttgtatcgcacttgatttttccgaaagacagattatctgcaaacggtgtgtcgtatttctggcttggtttttaaaaaatctggaatcgaaaatttgcggggcgaccgagaagttttttacaaaaggcaaaaactttttcgggatcgacagaaataaaacgatcgacggtacgcaacaaaaaagcgtcaggatcgccgtagagcgattgaagaccgtcaaccaaaggggaagcctccaatcgacgcgacgcgcgctctacggcgatcctgacgcagatttttagctatctgtcgcagcgccctcagggacaagccacccgcacaacgtcgcgagggcgatcagcgacgccgcaggg

SEQ ID NO: 40 - DNA, 6182 bp, synthetic construct
  source 1..6182: mol_type = other DNA
accctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttggatcctgaaaggtcagggccagggctggatcaccgcagagtacggcatgctgccacgttctacccacacccgtaacgctcgtgaagcggcgaaaggtaagcagggtggacgcacaatggaaatccagcgtctgatcgcccgtgctcttcgcgcggcagtagatttgaaagcgctgggtgagttcaccattacgctggactgcgacgtgcttcaggctgatggtggcacgcgtaccgcgtcgattacgggtgcctgcgtggcgctggtagatgcgctacagaagctggtggaaaacggcaagctgaaaaccaatccgatgaaagggatggtagccgcagtttctgtcggaattgtgaacggcgaagcggtttgcgatctggaatacgttgaagactctgccgcagagaccgacatgaacgtagtgatgaccgaagacgggcgcatcattgaagtgcaggggacggcagaaggcgagccgttcacccatgaagagctactcatcttgttggctctggcccgaggggaatcgaatccattgtagcgacgcagaaggcggcgctggcaaactgatttttaaggcgactgatgagtcgccttttttttgtctgtagaaaagtaagatgaggagcgaaggcaaagtgatctctatcatcaccctgaaagacctgattgcttacctggaagagaagccggaaatggcggaacatctggcggcggttaaggcctatcgcgaagagtttggcgtttaaagaaactcgccggatgaaaagtcatccggcgtcatattactgcaactgtgccgcaattagcggccagcgggcgtcaaaatcatccgtcgggcggtatttaaattcgctgcggacaaaacgtgacagcataccttcacagaaggccaggatctggcttgccagcagggtttcatcggtggtgtaaccttcaccctcacgcattctcttttcacgcaatacctggcgcagctgcgcttcaatacgctcgaacagctggttgatgcgcccttgcaggcgatcctgttcaaacattagcgcatgaccagtgaggatgcgggtcaggccaggattacgctcaccaaaaccgagaagcagcaacacaatcagacgcaggcgcgctgtggtgtctttctcatctttcagaatcaggttgatgcgagtaatcaggctatcttcgataaactcaatcaggctatcgaacatgcgggtcttactggggaagtggcgatacagtgccgcttcggatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgcaaaagaaaatgccgatggagcaatggcgatgacgcatcctcacgataatatccgggtaggcgcaatcactttcgtctactccgttacaaagcgaggctgggtatttcccggcctttctgttatccgaaatccactgaaagcacagcggctggctgaggagataaataataaacgaggggctgtatgcacaaagcatcttctgttgagttaagaacgagtatcgagatggcacatagccttgctcaaattggaatcaggtttgtgccaataccagtagaaacagacgaagaatccatgggtatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgaacgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcggttgcggccgcaatcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacggggtctg

SEQ ID NO: 41 - DNA, 7302 bp, synthetic construct
  source 1..7302: mol_type = other DNA
accctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttggatcctgaaaggtcagggccagggctggatcaccgcagagtacggcatgctgccacgttctacccacacccgtaacgctcgtgaagcggcgaaaggtaagcagggtggacgcacaatggaaatccagcgtctgatcgcccgtgctcttcgcgcggcagtagatttgaaagcgctgggtgagttcaccattacgctggactgcgacgtgcttcaggctgatggtggcacgcgtaccgcgtcgattacgggtgcctgcgtggcgctggtagatgcgctacagaagctggtggaaaacggcaagctgaaaaccaatccgatgaaagggatggtagccgcagtttctgtcggaattgtgaacggcgaagcggtttgcgatctggaatacgttgaagactctgccgcagagaccgacatgaacgtagtgatgaccgaagacgggcgcatcattgaagtgcaggggacggcagaaggcgagccgttcacccatgaagagctactcatcttgttggctctggcccgaggggaatcgaatccattgtagcgacgcagaaggcggcgctggcaaactgatttttaaggcgactgatgagtcgccttttttttgtctgtagaaaagtaagatgaggagcgaaggcaaagtgatctctatcatcaccctgaaagacctgattgcttacctggaagagaagccggaaatggcggaacatctggcggcggttaaggcctatcgcgaagagtttggcgtttaaagaaactcgccggatgaaaagtcatccggcgtcatattactgcaactgtgccgcaattagcggccagcgggcgtcaaaatcatccgtcgggcggtatttaaattcgctgcggacaaaacgtgacagcataccttcacagaaggccaggatctggcttgccagcagggtttcatcggtggtgtaaccttcaccctcacgcattctcttttcacgcaatacctggcgcagctgcgcttcaatacgctcgaacagctggttgatgcgcccttgcaggcgatcctgttcaaacattagcgcatgaccagtgaggatgcgggtcaggccaggattacgctcaccaaaaccgagaagcagcaacacaatcagacgcaggcgcgctgtggtgtctttctcatctttcagaatcaggttgatgcgagtaatcaggctatcttcgataaactcaatcaggctatcgaacatgcgggtcttactggggaagtggcgatacagtgccgcttcggatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgcaaaagaaaatgacgtccggtattacccggcatgacaggagtaaaaatggctatcgacgaaaacaaacagaaagcgttggcggcagcactgggccagattgagaaacaatttggtaaaggctccatcatgcgcctgggtgaagaccgttccatggatgtggaaaccatctctaccggttcgctttcactggatatcgcgcttggggcaggtggtctgccgatgggccgtatcgtcgaaatctacggaccggaatcttccggtaaaaccacgctgacgctgcaggtgatcgccgcagcgcagcgtgaaggtaaaacctgtgcgtttatcgatgctgaacacgcgctggacccaatctacgcacgtaaactgggcgtcgatatcgataacctgctgtgctcccagccggacaccggcgagcaggcactggaaatctgtgacgccctggcgcgttctggcgcagtagacgttatcgtcgttgactccgtggcggcactgacgccgaaagcggaaatcgaaggcgaaatcggcgactctcacatgggccttgcggcacgtatgatgagccaggcgatgcgtaagctggcgggtaacctgaagcagtccaacacgctgctgatcttcatcaaccagatccgtatgaaaattggtgtgatgttcggtaacccggaaaccactaccggtggtaacgcgctgaaattctacgcctctgttcgtctcgacatccgtcgtatcggcgcggtgaaagagggcgaaaacgtggtgggtagcgaaacccgcgtgaaagtggtgaagaacaaaatcgctgcgccgtttaaacaggctgaattccagatcctctacggcgaaggtatcaacttctacggcgaactggttgacctgggcgtaaaagagaagctgatcgagaaagcaggcgcgtggtacagctacaaaggtgagaagatcggtcagggtaaagcgaatgcgactgcctggctgaaagataacccggaaaccgcgaaagagatcgagaagaaagtacgtgagttgctgctgagcaacccgaactcaacgccggatttctctgtagatgatagcgaaggcgtagcagaaactaacgaagatttttaatcgtcttgtttgatacataagggcccgatggagcaatggcgatgacgcatcctcacgataatatccgggtaggcgcaatcactttcgtctactccgttacaaagcgaggctgggtatttcccggcctttctgttatccgaaatccactgaaagcacagcggctggctgaggagataaataataaacgaggggctgtatgcacaaagcatcttctgttgagttaagaacgagtatcgagatggcacatagccttgctcaaattggaatcaggtttgtgccaataccagtagaaacagacgaagaatccatgggtatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgaacgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcggttgcggccgcaatcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacggggtctg

SEQ ID NO: 42 - DNA, 6081 bp, synthetic construct
  source 1..6081: mol_type = other DNA
aattccgaaattaatacgactcactatagggagacatgaggattacccatgtgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagcttttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccagatatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggtcgactttatagctagctcagcccttggtacaatgctagctactagagtaagatgaggagcgaaggcatgaaaccatatcagcgccagtttattgaatttgcgcttagcaagcaggtgttaaagtttggcgagtttacgctgaaatccgggcgcaaaagcccctatttcttcaacgccgggctgtttaataccgggcgcgatctggcactgttaggccgtttttacgctgaagcgttggtggattccggcattgagttcgatctgctgtttggccctgcttacaaagggatcccgattgccaccacaaccgctgtggcactggcggagcatcacgacctggacctgccgtactgctttaaccgcaaagaagcaaaagaccacggtgaaggcggcaatctggttggtagcgcgttacaaggacgcgtaatgctggtagatgatgtgatcaccgccggaacggcgattcgcgagtcgatggagattattcaggccaatggcgcgacgcttgctggcgtgttgatttcgctcgatcgtcaggaacgcgggcgcggcgagatttcggcgattcaggaagttgagcgtgattacaactgcaaagtgatctctatcatcaccctgaaagacctgattgcttacctggaagagaagccggaaatggcggaacatctggcggcggttaaggcctatcgcgaagagtttggcgtttaatccggacctaggcagaacgcagaagctgtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctactcgcgaattacgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaag

SEQ ID NO: 43 - DNA, 4968 bp, synthetic construct
  source 1..4968: mol_type = other DNA
aattccgaaattaatacgactcactatagggagacatgaggattacccatgtgcgatcgcgcacgaggtttttctgtctagtgagcagtgtccaacctcaaaagacaacatgtgtgacgacgatgtagcggctcttgtcgtagacaatggatccggtatgtgcaaagccggtttcgcaggagatgacgcaccccgtgccgtcttcccctcgatcgtcggtcgcccaaggcatcaaggagtcatggtcggtatgggacaaaaggactcatacgtaggagatgaagcccaaagcaaaagaggtatcctcaccctgaaataccccatcgaacacggtatcatcaccaactgggatgagtttaaaccctctagcttttacaaagtactggttccctttccagcgggatgctttatctaaacgcaatgagagaggtattcctcaggccacatcgcttcctagttccgctgggatccatcgttggcggccgaagccgccattccatagtgagttctggcgcgcctcatcccagttggtgatgataccgtgttcgatggggtatttcagggtgaggatacctcttttgctttgggcttcatctcctacgtatgagtccttttgtcccataccgaccatgactccttgatgccttgggcgaccgacgatcgaggggaagacggcacggggtgcgtcatctcctgcgaaaccggctttgcacataccggatccattgtctacgacaagagccgctacatcgtcgtcacacatgttgtcttttgaggttggacactgctcactagacagaaaaacctcgtgccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccagatatctcgatcccgcgaaattaatacgactcactatagggagaccacaacggtttccctctagatcacaagtttgtacaaaaaagcaggctaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccggtcgactttatagctagctcagcccttggtacaatgctagctactagagtaagatgaggagcgaaggcatgaaaccatatcagcgccagtttattgaatttgcgcttagcaagcaggtgttaaagtttggcgagtttacgctgaaatccgggcgcaaaagcccctatttcttcaacgccgggctgtttaataccgggcgcgatctggcactgttaggccgtttttacgctgaagcgttggtggattccggcattgagttcgatctgctgtttggccctgcttacaaagggatcccgattgccaccacaaccgctgtggcactggcggagcatcacgacctggacctgccgtactgctttaaccgcaaagaagcaaaagaccacggtgaaggcggcaatctggttggtagcgcgttacaaggacgcgtaatgctggtagatgatgtgatcaccgccggaacggcgattcgcgagtcgatggagattattcaggccaatggcgcgacgcttgctggcgtgttgatttcgctcgatcgtcaggaacgcgggcgcggcgagatttcggcgattcaggaagttgagcgtgattacaactgcaaagtgatctctatcatcaccctgaaagacctgattgcttacctggaagagaagccggaaatggcggaacatctggcggcggttaaggcctatcgcgaagagtttggcgtttaatccggacctaggcagaacgcagaagctgtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctactcgcgaattacgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtcttcaag

SEQ ID NO: 44 - DNA, 6515 bp, synthetic construct
  source 1..6515: mol_type = other DNA
cccttaggagttggcatggcgaatatgtttgccctgattctggtgattgccacactggtgacgggcattttatggtgcgtggataaattctttttcgcacctaaacggcgggaacgtcaggcagcggcgcaggcggctgccggggactcactggataaagcaacgttgaaaaaggttgcgccgaagcctggctggctggaaaccggtgcttctgtttttccggtactggctatcgtattgattgtgcgttcgtttatttatgaaccgttccagatcccgtcaggttcgatgatgccgactctgttaattggtgattttattctggtagagaagtttgcttatggcattaaagatcctatctaccagaaaacgctgatcgaaaccggtcatccgaaacgcggcgatatcgtggtctttaaatatccggaagatccaaagcttgattacatcaagcgcgcggtgggtttaccgggcgataaagtcacttacgatccggtctcaaaagagctgacgattcaaccgggatgcagttccggccaggcgtgtgaaaacgcgctgccggtcacctactcaaacgtggaaccgagcgatttcgttcagaccttctcacgccgtaatggtggggaagcgaccagcggattctttgaagtgccgaaaaacgaaaccaaagaaaatggaattcgtctttccgagcgtaaagagacactgggtgatgtgacgcaccgcattctgacagtgccgattgcgcaggatcaggtggggatgtattaccagcagccagggcaacaactggcaacctggattgttcctccgggacaatacttcatgatgggcgacaaccgcgacaacagcgcggacagccgttactggggctttgtgccggaagcgaatctggtcggtcgggcaacggctatctggatgagcttcgataagcaagaaggcgaatggccgactggtctgcgcttaagtcgcattggcggcatccattaatagccatcttcgttcacgttgtcgccgttatggcgacaacgtgaattatttatgagataaatctcccgtggctaacgacatcccccgtcgttgtgtatagaatattcccccgaagtttaaggttggcacctccaggttgccacggcacacgaaacagcgttggtccccatataccggtaaactgaaactgcagcgaagcagttagcagaaccatgtatatcaggtctgtttcgtgtgctgaattgttgacgcatttatttattggtatcgcatgaaccccatcgtaattaatcggcttcaacggaagctgggctctgatgaatcccctaatgattttggtaaaaatcattaagttaaggtggatacacatcttgtcatatgatctcgggaaaagcgttggtgaccaaaggtgccttttatcatcactttaaaaataaaaaacaattactcagtgcctgttataagcagcaattaattatgattgatgcctacatcacaacaaaaactgatttaacaaatggttggtctgccttagaaagtatatttgaacattatcttgattatattattgataataataaaaaccttatccctatccaagaagtgatgcctatcattggttggaatgaacttgaaaaaattagccttgaatacattactggtaaggtaaacgccattgtcagcaaattgatccaagagaaccaacttaaagcttatgatgatgatgtgcttaaaaacttactcaatggctggtttatgcatatcgcaatacatgcgaaaaacctaaaagagcttgccgataaaaaaggccaatttattgctatttaccgcggctttttattgagcttgaaagataaataaaatagataggttttatttgaagctaaatcttctttatcgtaaaaaatgccctcttgggttatcaagagggtcattatatttcgcggaataacatcatttggtgacgaaataactaagcacttgtctcctgtttactcccctgagcttgaggggttaacatgaaggtcatcgatagcaggataataatacagtaaaacgctaaaccaataatccaaatccagccatcccaaattggtagtgaatgattataaataacagcaaacagtaatgggccaataacaccggttgcattggtaaggctcaccaataatccctgtaaagcaccttgctgatgactctttgtttggatagacatcactccctgtaatgcaggtaaagcgatcccaccaccagccaataaaattaaaacagggaaaactaaccaaccttcagatataaacgctaaaaaggcaaatgcactactatctgcaataaatccgagcagtactgccgttttttcgccccatttagtggctattcttcctgccacaaaggcttggaatactgagtgtaaaagaccaagacccgctaatgaaaagccaaccatcatgctattccatccaaaacgattttcggtaaatagcacccacaccgttgcgggaatttggcctatcaattgcgctgaaaaataaataatcaacaaaatgggcatcgttttaaataaagtgatgtataccgaattcgattgcgtctcaacccctacttcggtatctgtattatcacgtgtatttttggtttcacggaaccaaaacataaccacaaggaaagtgacaatatttagcaacgcagcgataaaaaagggactatgcggtgaaatctctcctgcaaaaccaccaataataggccccgctattaaaccaagcccaaaacttgcccctaaccaaccgaaccacttcacgcgttgagaagctgaggtggtatcggcaatgaccgatgccgcgacagccccagtagctcctgtgatccctgaaagcaaacggcctaaatacagcatccaaagcgcacttgaaaaagccagcaataagtaatccagcgatgcgcctattaatgacaacaacagcactgggcgccgaccaaatcggtcagacatttttccaagccaaggagcaaagataacctgcattaacgcataaagtgcaagcaatacgccaaagtggttagcgatatcttccgaagcaataaattcacgtaataacgttggcaagactggcatgataaggccaatccccatggcatcgagtaacgtaattaccaatgcgatctttgtcgaactattcatttcacttttctctatcactgatagggagtggtaaaataactctatcaatgatagagtgtcaacaaaaattaggaattaatgatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcacggtaaccaagatgtcgagttaaccaccctttagattcataaagcgaaaataatgcggctccaacgtacccacctaaatggaaacggcgttcactccaatctaaacacgcacaacagattttacgtgaatgtttggaaggaacgtcaattcccatttcatgaaaatattgaataccacttaatgtgatcattgaaccattttcagtgatccattgctgttgacaaagggaatcatagatcatatgacaagatgtgtatctaccttaacttaatgattttgataaaaatcattaggggattcatcagagctgggctacacttttaatcatcaggaactgttgcagcaggcattaactcatcgtagtgccagcagtaaacataacgagcgtttagaatttttaggcgactctattctgagctacgttatcgccaatgcgctttatcaccgtttccctcgtgtggatgaaggcgatatgagccggatgcgcgccacgctggtccgtggcaatacgctggcggaactggcgcgcgaatttgagttaggcgagtgcttacgtttagggccaggtgaacttaaaagcggtggatttcgtcgtgagtcaattctcgccgacaccgtcgaagcattaattggtggcgtattcctcgacagtgatattcaaaccgtcgagaaattaatcctcaactggtatcaaactcgtttggacgaaattagcccaggcgataaacaaaaagatccgaaaacgcgcttgcaagaatatttgcagggtcgccatctgccgctgccgacttatctggtagtccaggtacgtggcgaagcgcacgatcaggaatttactatccactgccaggtcagcggcctgagtgaaccggtggttggcacaggttcaagccgtcgtaaggctgagcaggctgccgccgaacaggcgttgaaaaaactggagctggaatgagcatcgataaaagttactgcggatttattgccatcgtcggacgtccgaacgttggcaaatccacattgttgaacaaactgctggggcagaaaatctccatcacttcccgcaaggcgcagacaactcgtcaccgcattgtggggatccatactgaaggcgcgtatcaggcgatctacgtcgatacaccgggcctgcatatggaagaaaaacgcgccattaaccgcctgatgaacaaagcggcgagcagctctattggcgatgttgagctggtgatttttgtcgttgaaggcacccgctggacgccggacgacgaaatggtgctcaacaaactgcgcgaaggcaaagcgccggtaatcctcgcggtgaacaaagtggacaacgtgcaggagaaagccgatctgctgccgcacctgcagttcctggcaagccagatgaacttcctcgatatcgtgccaatctctgccgaaaccgggctgaatgttgacactattgcggcaatcgtgcgtaagcatctacctgaagcgactcatcacttcccggaagattacatcaccgatcgctcacagcgttttatggcgtctgaaatcatccgcgaaaaactgatgcgtttcctcggcgctgaactgccgtactccgtgaccgtggagatcgaacgtttcgtctctaacgaacgcggtggttatgacatcaacggtttgattctcgttgagcgtgaagggcagaagaagatggtcattggcaacaaaggggccaagatcaaaaccatcgggattgaagcgcgtaaagacatgcaggaaatgttcgaagcgcctgttcaccttgaactgtgggtaaaagtgaaatccggttgggccgacgacgaacgcgcactgcgcagtctcggttacgttgacgatctttaagagtaactccgatggaaggctggcagcgcgcatttgtcctgcatagtcgcccgtggagcgaaaccagcctgatgctggacgtcttcacggaggaatcggggcgcgtgcgtctggttgccaaaggcgcacgctctaaacgctctaccctgaaaggtgcattacagcctttcacccctctcttgctacgttttggcgggcgtggcgaagtcaaaacgctgcgcagtgctgaagccgtctcgctggcgctgccattaagcggtatcacgctttacagcggtctgtacatcaacgaacttctctcccgcgtactggaatacgagacgcgcttctctgaactttttttcgattacttgcactgcattcagtctcttgcaggggtcactggtacgccagaacccgcgctgcgccgctttgaactggcactgctcgggcatctgggttatggcgtcaattttacccattgtgcgggtagcggcgagccggtagatgacaccatgacgtatcgttatcgcgaagaaaaagggtttatcgcaagcgtcgttatcgacaataaaacgttcaccggaaggcagttaaaagcgttaaacgcacgggaatttcctgacgcagacacactgcgcgccgcgaaacgctttacccgcatggcgcttaagccgtatcttggcggtaaacctttaaagagcagggaactgttccggcagtttatgcctaagcgaacggtgaaaacacattatgaatgatgaggattgtc